SEQUENCE LISTING (44 sequences)

SEQ ID NO: 1 - protein, 598 aa, Artificial Sequence
  IE1/2/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 2 - DNA, 2081 bp, Artificial Sequence
  DNA encoding IE1/2/FKBP Fusion
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagagtcctctgccaagagaaagatggaccctgataatcctgacgagggcccttcctccaaggtgccacggtacgtgtcggggtttgtgccccccctttttttttaataaaattgtattaatgttatatacatatctcctgtatgtgacccatgtgcttatgactctatttctcatgtgtttaggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggatacttatatgtgttgttatcctcctctacagtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 3 - protein, 594 aa, Artificial Sequence
  UL37x1/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPESPVYVNLLGSVGLLAFWYFSYRWIQRKRLEDPLPPWLRKKKACALTRRSRHRLRRQHGVIDGENSETERSVDLVAALLAEAGEESVTEDTEREDTEEEREDEEEENEARTPEVNPIDAEGLSGLAREACEALKKALRRHRFLWQRRQRARMLQHNGPQQSHHAAVFCRVHGLRGFQVSVWLLLTLLWSTGHGVSVRCTYHGTDVNRTSNTTSMNCHLNCTRNHTQIYNGPCLGTEARLPLNVTFNQSRRKWHSVMLKFGFQYHLEGWFPLRVLNESREINVTEVHGEVACFRNDTNVTVGQLTLNFTGHSYVLRAIAHTSPFESYVRWEETNVTDNATSSENTTTVMSTLTKYAESDYIFLQDMCPRFLKRTVKLTRNKTKHNVTVTGNNMTTLPVWTPECKGWTYWTTLSVMWRNRRSALLRAKSRALGHWALLSICTVAAGSIALLSLFCILLIGLRRDLLEDFRYICRDEGSSSTKNDVHRIV

SEQ ID NO: 4 - DNA, 3118 bp, Artificial Sequence
  DNA encoding UL37x1/FKBP Fusion
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaatctccagtctacgtgaatctgttaggtagcgtgggcctgctggccttttggtactttagctaccgctggatccagcgaaaacgccttgaagatcctctcccgccttggttaagaaagaaaaaagcatgtgcgctcacccggcgttctcggcaccggttacgccgccaacatggcgtaattgacggtgagaactcggagaccgagcggtccgtggacttggtggccgcgctcttggccgaggccggggaggagagcgtaacggaggacacagagcgggaggacacggaggaggaaagggaggatgaagaagaagaaaacgaggctcggacgcccgaggtgaaccccatcgacgccgagggcctctcaggacttgcgcgagaagcctgcgaagccctcaaaaaggccctgcggagacaccggttcctgtggcaacggcggcaacgagctcggatgctgcagcacaacggcccccagcagtctcaccagtaagcaggggtaaacaaaaaaaccacagactccgggatagtataagaagggactttaccgctattgctgctattcatagagaaggatagaaaggatgactacgaccacgcatagcactgccgctatcatgagcctgctggacgaggccgagtggcgacagacgcagatggacgtggggggactgatccaggcgtcggcgttgggcaaggtggcgcttcgctacgcggtgaggaaactgatgaagcgcggagccaggctgcggcacgactcgggtctctacgtgtgcatctgcgacccttcgtacgagtttctgcagatgaacctgagcaagatcagttggctggagcgacactgccccccgttggaccaggagctcatcatgttcggggtcatcgaggcctgggaggaagcttcggtgcggcccacgcgtcagctggtgttgttcatgacgcccaagtgggacgtgttcgcctacgactccggtatcctgtttttcctggcgcccagcatggcgcagttctggcacggtgctatcgtgctggagtattggaacgctttgttcccggtggaggtgcgctcccacgtccgtcagcacgcgcacactatggacgacctggtgatggtcttccaccagctggactacgaaaagcaggtgctggaagcgcgtcgcgacaagaatacggaggggccgcgcacttttgccaagtcggtgaacagctacgtgcgcgccatcctggaatctgagcggcggatccgcgaagggaaaattcctatgaccttcgtggatcgcgactcgctgcgagccaattcgttggcccacatccaggccaccggggctcagccttctcacgccccggcgcaacgggtgctgtccgcgccgccgagcctgccgttgcccgtgtcggaagaagatcccgctgccgccgccaccccttcgtcctccgccgccaccacccccccgtcctctgtcgtcccggcctccgtcgagagcgagttgtcctcgtcgccgccgctgccgccggtcgtggtgaaggatgtcgtgtacacggccggggagggcgacgtggtacagatggtggtcgtggtctagaaggagaactttgctgctagatgaccatgttcagcttttttttttgtagtattttttcatagttgctatacctcagttatcccccctattagccccacatgctgcttaataaacatacttgtttcattttctttctagtgccgcggttttttgtcgcgtccacgggctgcgcgggtttcaggtaaaaacaacgtgtgtgataaacgtgggtttttacctacgggaaaggggagggtagaaacgtgagtctccgtcaataaaaagtcccgtgtcgtctccacgtaggtttctgtgtggctgttgttgacgctgctttggagcacgggacacggtgtttcggtccgatgtacgtaccacggtacggacgttaatcgcacttccaacaccacgagcatgaactgccacctaaactgcacacgcaatcacacgcaaatctacaacggcccctgtctcgggaccgaagccagactgcccttgaatgtaacgtttaatcaatcacgacggaaatggcacagcgtcatgcttaaatttggttttcagtatcacctggaaggctggtttcccctgcgtgtcttgaacgagagtcgtgagataaacgtcacggaagttcacggagaagtagcatgttttaggaatgacacgaacgtgaccgtgggacaactgacgctcaacttcaccggccacagctacgtgttgcgggcaatagcgcacacgagtccgttcgaatcatacgtacgttgggaagaaacaaacgtcactgacaacgctacttcttcagaaaacactaccaccgtgatgtcaacactgacgaaatacgcggaaagtgattatatctttctacaagacatgtgcccgcgatttctcaagaggaccgttaagctcacgagaaacaaaacaaaacataacgtgacagttacgggaaataacatgacaacgcttcccgtctggacaccggaatgcaaaggatggacgtactggaccacgttgtccgtcatgtggagaaaccgccgttcggctctcctccgagccaaaagtcgcgccttgggacactgggcgttgttgagcatatgtactgtggcggcgggcagcatcgcgttgctcagcctcttttgcatcctcctcatcggacttcgtcgcgatttgcttgaagatttccgctacatttgtcgggacgagggaagttccagcacaaaaaacgacgttcatcggatcgtctga

SEQ ID NO: 5 - protein, 540 aa, Artificial Sequence
  UL44/FKBP Fusion protein
MDRKTRLSEPPTLALRLKPYKTAIQQLRSVIRALKENTTVTFLPTPSLILQTVRSHCVSKITFNSSCLYITDKSFQPKTINNSTPLLGNFMYLTSSKDLTKFYVQDISDLSAKISMCAPDFNMEFSSACVHGQDIVRESENSAVHVDLDFGVVADLLKWIGPHTRVKRNVKKAPCPTGTVQILVHAGPPAIKFILTNGSELEFTANNRVSFHGVKNMRINVQLKNFYQTLLNCAVTKLPCTLRIVTEHDTLLYVASRNGLFAVENFLTEEPFQRGDPFDKNYVGNSGKSRGGGGGGGSLSSLANAGGLHDDGPGLDNDLMNEPMGLGGLGGGGGGGGKKHDRGGGGGSGTRKMSSGGGGGDHDHGLSSKEKYEQHKITSYLTSKGGSGGGGGGGGGGLDRNSGNYFNDAKEESDSEDSVTFEFVPNTKKQKCGGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 6 - DNA, 1623 bp, Artificial Sequence
  DNA encoding UL44/FKBP Fusion
atggatcgcaagacgcgcctctcggagccgccgacgctggcgctgcggctgaagccgtacaagacggctatccagcagctgcgatctgtgatccgtgcgctcaaggagaacaccacggttaccttcttgcccacgccgtcgcttatcttgcaaacggtacgcagtcactgcgtgtcaaaaatcacttttaacagctcatgcctctacatcactgacaagtcgtttcagcccaagaccattaacaattccacgccgctgctgggtaatttcatgtacctgacttccagcaaggacctgaccaagttctacgtgcaggacatctcggacctgtcggccaagatctccatgtgcgcgcccgatttcaatatggagttcagctcggcctgcgtgcacggccaagacattgtgcgcgaaagcgagaattcggccgtgcacgtggatctagatttcggcgtggtggccgacctgcttaagtggatcgggccgcatacccgcgtcaagcgtaacgttaaaaaagcgccctgccctacgggcaccgtgcagattctggtgcacgccggtccaccggccatcaagtttatcctgaccaacggcagcgagctggaattcacagccaataaccgcgtcagtttccacggcgtgaaaaacatgcgtatcaacgtgcagctgaagaacttctaccagacgctgctcaattgcgccgtcaccaaactgccgtgcacgttgcgtatagttacggagcacgacacgctgttgtacgtggccagccgcaacggtctgttcgccgtggagaattttctcaccgaggaacctttccagcgtggcgatcccttcgacaaaaattacgtcgggaacagcggcaagtcgcgtggcggcggcggtggtggcggcagcctctcttcgctggccaatgccggcggtctgcatgacgacggcccgggtctggataacgatctcatgaacgagcccatgggtctcggcggtctgggaggaggtggcggcggtggcggcaagaagcacgaccgcggtggcggcggtggttccggtacgcggaaaatgagcagcggtggcggcggcggtgatcacgaccacggtctttcctccaaggaaaaatacgagcagcacaagatcaccagctacctgacgtccaaaggtggatcgggcggcggcggaggaggaggaggcggcggtttggatcgcaactccggcaattacttcaacgacgcgaaagaggagagcgacagcgaggattctgtaacgttcgagttcgtccctaacaccaagaagcaaaagtgcggctagggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaa

SEQ ID NO: 7 - protein, 264 aa, Artificial Sequence
  UL51/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPESWAKQRVPFLDDDDGEEENDVQDDVDSPVPTRPLVIDEDAEPAAGTSGGLEGGGGDDEDGEDGHALPDLDDDLLLQFEPMLPRVYDLLLPSLDARLNFVNAGQKYAAFLKYVHGDCATCSHGEILREKTQLLTAIVSKLMDINGILEGKDESAPGK

SEQ ID NO: 8 - DNA, 794 bp, Artificial Sequence
  DNA encoding UL51/FKBP Fusion protein
tgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaatcctgggctaagcagcgggtgccgtttctggacgatgacgacggagaggaggaaaacgacgtgcaggatgacgtggattctccggtgccgacgcgaccgctggtgatcgacgaggacgcggagcccgcggctggtacgagcggtgggctcgaggggggaggtggtgacgacgaggacggtgaagacggacacgcgctacccgatcttgacgacgatctgctattacagttcgagccgatgctgccgcgcgtctacgatctgttgctgccctctctggacgcgcgcttaaatttcgtgaacgcgggtcagaagtacgccgccttcctcaagtacgtgcacggcgactgcgcgacctgcagtcacggagagatcctccgtgagaagacgcaactattaacggcgatcgtcagcaagctcatggacattaacggaatcctggagggaaaagacgagtcggcgccgggtaaataa

SEQ ID NO: 9 - protein, 775 aa, Artificial Sequence
  UL52/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPENPSTHVSSNGPTTPPHGPHTTFLPPTSPAPSTSSVAAATLCSPQRQAVSRYSGWSTEYTQWHSDLTTELLWHAHPRQVPMDEALAAAAAASYQVNPQHPANRYRHYEFQTLSLGTSEVDELLNCCAEETTCGGTQSTVLTNATNTTSCGGAVAGSSNVGPAGASAACDLDAELAGLETSAADFEQLRRLCAPLAIDTRCNLCAIISICLKQDCDQSWLLEYSLLCFKCSYAPRAALSTLIIMSEFTHLLQQHFSDLRIDDLFRHHVLTVFDFHLHFFINRCFEKQVGDAVDNENVTLNHLAVVRAMVMGEDTVPYNKPRRHPQQKQKNNPYHVEVPQELIDNFLEHSSPSRDRFVQLLFYMWAGTGVMSTTPLTELTHTKFARLDALSTASEREDARMMIEEEEDEEGGEKGGDDPGRHNGGGTSGGFSESTLKKNVGPIYLCPVPAFFTKNQTSTVCLLCELMACSYYDNVVLRELYRRVVSYCQNNVKMVDRIQLVLADLLRECTSPLGAAHEDVARCGLEAPTSPGGDSDYHGLSGVDGALARPDPVFCHVLRQAGVTGIYKHFFCDPQCAGNIRVTNEAVLFGRLHPHHVQEVKLAICHDNYYISRLPRRVWLCITLFKAFQITKRTYKGKVHLADFMRDFTQLLESCDIKLVDPTYVIDKYV

SEQ ID NO: 10 - DNA, 2328 bp, Artificial Sequence
  DNA encoding UL52/FKBP Fusion protein
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaaatccgagtacccacgtgagcagtaacggcccaacgactccccctcacgggccccacaccacgtttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtaaatcctcaacaccccgccaaccgttaccgtcattacgaattccagacgctcagcctcggcacctcggaggtagacgaactgctcaactgttgtgcggaagaaaccacgtgcggcggcacgcaatccaccgtactcaccaatgcgaccaacaccactagctgcggcggagccgtcgccggcagtagcaacgtaggacccgccggcgcttcggccgcctgcgacctagatgcagaactggccggcctcgaaacctcggcggccgactttgaacaactgcggcgactgtgcgcgccgctggccatcgacacgcgctgtaacctatgcgccatcatcagcatctgcctcaaacaggactgcgaccagagctggctcctcgagtacagcttgctgtgcttcaaatgcagttacgcgccccgtgcggcgctcagcacgctcatcatcatgtccgagtttacgcatctgctgcagcagcacttttccgatctgcgcatcgacgacctgttccgacaccacgttctcacggtcttcgatttccacctgcactttttcatcaatcgttgctttgaaaaacaagtgggcgacgcggttgataacgagaatgtcaccctgaaccatctggccgtggtgcgggccatggtcatgggtgaagacacggtgccttacaacaagcctcggcgccacccgcaacagaagcaaaaaaacaacccttatcacgtcgaagtgccgcaagaactgatcgacaactttctagaacacagctcacctagccgcgaccgcttcgtgcagctgcttttctatatgtgggccggcaccggcgtcatgagcaccacgccactcacggaactcacgcacactaagttcgcgcgactagacgcgttatccacggcctcggaaagagaagacgcaaggatgatgatagaagaagaggaggatgaagaaggaggagaaaaaggaggagacgatccgggccgtcacaacggcggtggcaccagcggggggttcagcgagagcacgctaaaaaaaaacgtgggtcccatttacctatgtcccgtacccgctttttttaccaagaaccaaaccagtaccgtgtgtctgctgtgcgaactcatggcctgctcctattacgataacgtcgtcctgcgcgagctgtaccgccgcgtcgtctcgtattgtcagaacaatgtgaagatggtggaccgcattcagctggtattggccgatctgttgcgcgaatgcacgtcgccgctcggcgcggcacacgaggacgtggcgcgctgtggactcgaagcacccacctcgcccggaggcgactcggactaccacggcctgagcggcgtcgacggcgcactggcgcgacccgacccggtattttgccacgtcctgcgtcaggcaggcgtcacgggcatctacaagcactttttctgcgacccgcagtgcgccggcaacatccgcgtcaccaacgaggccgtgctcttcggacgcctgcacccccaccacgtccaggaggtgaaactggccatctgtcacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctag

SEQ ID NO: 11 - protein, 483 aa, Artificial Sequence
  UL53/FKBP Fusion protein
MSSVSGVRTPRERRSALRSLLRKRRQRELASKVASTVNGATSANNHGEPPSPADARPRLTLHDLHDIFREHPELELKYLNMMKMAITGKESICLPFNFHSHRQHTCLDISPYGNEQVSRIACTSCEDNRILPTASDAMVAFINQTSNIMKNRNFYYGFCKSSELLKLSTNQPPIFQIYYLLHAANHDIVPFMHAEDGRLHMHVIFENPDVHIPCDCITQMLTAAREDYSVTLNIVRDHVVISVLCHAVSASSVKIDVTILQRKIDEMDIPNDVSESFERYKELIQELCQSSGNNLYEEATSSYAIRSPLTASPLHVVSTNGCGPSSSSQSTPPHLHPPSQATQPHHYSHHQSQSQQHHHRPQSPPPPLFLNSIRAPGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 12 - DNA, 1452 bp, Artificial Sequence
  DNA encoding UL53/FKBP Fusion protein
atgtctagcgtgagcggcgtgcgcacgccgcgcgaacgacgctcggccttgcgctccctgctccgcaagcgccgccaacgcgagctggccagcaaagtggcgtcgacggtgaacggcgctacgtcggccaacaaccacggcgaaccgccgtcgccggccgacgcgcgcccgcgcctcacgctgcacgacctgcacgacatcttccgcgagcaccccgaactggagctcaagtaccttaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttattacgggttctgtaagagcagcgagctactcaagctctccaccaaccagccgcccatcttccaaatttattacctgctgcacgccgccaaccacgacatcgtgccctttatgcacgccgaggacggccggttgcacatgcacgtcatcttcgaaaaccccgacgtgcacatcccctgcgactgcatcacgcagatgctcacggcggcgcgcgaagactacagcgtcacgctcaacatcgtgcgcgaccacgtcgttatcagcgtgctgtgtcacgccgtctcggccagcagcgtcaagatcgacgtgactattttgcaacgcaagattgacgagatggacattcccaacgacgtgagcgagtcctttgagcgctacaaagagctcattcaggagctgtgtcagtccagcggcaacaacctatacgaggaggccacgtcgtcctacgcgatacggtctcccttaaccgcgtcgccgttgcacgtagtttccaccaacggctgcggcccctcctcctcgtcccagtccacgccgcctcatctccacccgccgtcgcaggcgacgcagccccaccactactctcaccaccagtctcagtctcagcagcatcatcaccgtccccagtcaccaccgccgccgctgtttctcaacagcattcgtgcgccttgaggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaa

SEQ ID NO: 13 - protein, 957 aa, Artificial Sequence
  UL56/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEEMNLLQKLCVVCSKCNEYAMELECLKYCDPNVLLAESTPFKRNAAAIVYLYRKIYPEVVAQNRTQSSLLTLYLEMLLKALHEDTALLDRALMAYSRQPDRAAFYRTVLRLDRCDRHHTVELQFTDNVRFSVSLATLNDIERFLCKMNYVYGILAPEAGLEVCAQLLELLRRLCGISPVARQEVYVEGTTCAQCYEELTIIPNQGRSLNKRLQGLLCNHIAVHRPSSQSDVNIQTVEQDLLDLTTRIPHLAGVLSALKSLFSSSSAYHSYIQEAEEALREYNLFTDIPERIYSLSDFTYWSRTSEVIVKRVGITIQQLNVYHQLCRALMNGISRHLYGEDVEDIFVLGEKALDGEERMFVGSVFAAPNRIIDLITSLSIQAFEDNPVFNKLHESNEMYTKIKHILEEIRRPLPDGTGGDGPEGEAIHLRGREAMSGTGTTLMTASNSSNSSTHSQRNNGGGGRARGGGKKVVGGGVNGQDGDGSENGLRVRNCDEHEALDLVDARSRIHNVTREVNVRKRAYLQKVSEVGYGKVIRCIKTQERLTSKLIDVNLVGPLCLDFISKLMNGFLYRSQYHQDQDVVDVGDQFTYDEHLYVVNNLIHKSLPVESLPLLGQQIYELCNGPLFTHCTDRYPLSHNVDMAYACDNAGVLPHVKDDLVKCAEGTVYPSEWMVVKYMGFFNFSDCQDLNVLQKEMWMHVRELVLSVALYNETFGKQLSIACLRDELHPDRDVILTYNKEWPLLLRHEGSLYKSKDLYLLLYRHLSRPDESGDVPTAPVAKPSTLTAAAAVSGVFREPDRPWLPSPYPSSSTAGVSRRVRATRKRPRRASSLLDLARDEHGIQDLVPGSLR

SEQ ID NO: 14 - DNA, 2874 bp, Artificial Sequence
  DNA encoding UL56/FKBP Fusion protein
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagagatgaatttgttacagaaactatgcgtagtgtgttcgaaatgcaacgaatacgccatggagctggagtgtctaaagtactgcgatccgaacgtgttactggcggagtccacgccgttcaagagaaacgcggcggctatagtgtatctgtaccggaagatctacccggaggtggtggcgcagaatcgtacgcagagttcgttgctgacgctctacctggagatgctgctgaaggcgctgcacgaggatacggctttgctggatcgggcgctgatggcctactcgcgccagccggaccgggcggccttctaccgtaccgtcctccgtttggatcgctgcgatcgccatcacaccgtggagctccagtttacggacaacgtccgtttcagcgtcagtctggccacactcaacgacatcgagcgcttcctgtgcaaaatgaactacgtgtacgggatcctggcgccggaggccggcctggaggtctgcgcgcagttgctggagctcctccgtcgcctatgcggcatctcgccggtggcgcgtcaggaagtgtacgtcgaagggacgacatgcgcccaatgctacgaggagctgaccatcatcccgaatcagggccgctcgctgaacaagcggctgcagggcttgctgtgcaaccatatagcggtccaccgtccgtcaagccagtccgatgtgaatatccagacggtggagcaggacctgctggacctgacaacgcgcatcccccacttggctggagtcctttcggccctcaaaagcctattctcttcttcatcggcctaccacagctacatccaggaggcggaggaggcgctgagggagtacaacctgtttacggatataccggaacgaatatattccttgtcggattttacctactggtcccgtacctcggaggttatcgtcaagcgggtgggcatcaccatccagcagctaaatgtgtatcaccagctgtgccgggcgctcatgaacggcatcagtcgccatctgtacggggaggacgtggaggacatcttcgtgctcggggaaaaggcgttggacggggaggagcgcatgttcgtggggtcggtctttgccgcccccaacaggatcatcgacctcatcacatccctcagcattcaagctttcgaggacaacccggtgttcaacaagctccacgaaagcaacgagatgtacaccaaaatcaagcatattctcgaggagattcgacgtccgctgccagatggcacggggggcgacggccccgagggcgaggctattcacctgcgtggacgggaggcgatgtcggggacgggtacgactttgatgacggccagcaacagcagcaacagcagtactcacagtcagaggaataacggtggcggcggccgggctcgtggaggaggcaagaaagtggtagggggaggggtgaatgggcaggacggcgacggcagcgagaacggcttacgggtgcgcaactgcgacgaacacgaggctctcgacttggtggacgcgcgctcccgcatccacaacgtcactcgcgaggtgaacgtgcgcaaacgcgcctacctgcagaaggtctcggaggtgggctatggcaaggtgatccgttgcatcaaaacgcaggagcgtctgactagcaagctcatcgatgtcaatctagtggggcccttgtgtctggactttatctctaagctcatgaatgggtttctgtaccgcagccaataccaccaggaccaagacgtggtggacgtgggtgatcagttcacctacgatgagcacctgtatgtggtcaataacctgatccacaagagtctgcctgtggaatccctcccgctactgggtcagcagatctacgagttgtgtaacgggcccctcttcacccactgcaccgatcgttatcccctctctcacaatgtggacatggcctatgcctgcgacaacgcgggcgtactaccccacgtcaaggacgatttggtcaaatgcgcggaaggtaccgtgtatcccagtgagtggatggtggtgaagtatatgggttttttcaatttttcggactgtcaggacctaaacgtgctgcagaaggagatgtggatgcacgtgcgggagctcgtgctctccgtcgcactatataatgaaactttcgggaaacaactctcgatcgcgtgcctgcgcgacgaactgcacccggacagagatgtgattctcacgtataacaaagagtggccgctgctgcttcgtcacgaaggaagtctttataagtccaaagatctatatctcctcctctacaggcatctgtccagaccggatgagagtggcgacgtgccaacagctcccgtggccaagccctccaccctgacggccgccgcggccgtctcgggtgtcttcagggagccggaccgaccttggctgccaagtccgtatccctcctcctcgactgcgggtgtttcccggagggtccgcgcgacacgcaagagaccacgacgcgcctcatcgctgctggatttggcccgcgacgaacatggaatccaggatctggtgcctggtagtctgcgttaa

SEQ ID NO: 15 - protein, 749 aa, Artificial Sequence
  UL77/FKBP Fusion protein
MSLLHTFWRLPVAVFFEPHEENVLRCPERVLRRLLEDAAVTMRGGGWREDVLMDRVRKRYLRQELRDLGHRVQTYCEDLEGRVSEAEALLNQQCELDEGPSPRTLLQPPCRPRSSSPGTGVAGASAVPHGLYSRHDAITGPAAAPSDVVAPSDAVAASAAAGASSTWLAQCAERPLPGNVPSYFGITQNDPFIRFHTDFRGEVVNTMFENASTWTFSFGIWYYRLKRGLYTQPRWKRVYHLAQMDNFSISQELLLGVVNALENVTVYPTYDCVLSDLEAAACLLAAYGHALWEGRDPPDSVATVLGELPQLLPRLADDVSREIAAWEGPVAAGNNYYAYRDSPDLRYYMPLSGGRHYHPGTFDRHVLVRLFHKRGVIQHLPGYGTITEELVQERLSGQVRDDVLSLWSRRLLVGKLGRDVPVFVHEQQYLRSGLTCLAGLLLLWKVTNADSVFAPRTGKFTLADLLGSDAVAGGGLPGGRAGGEEEGYGGRHGRVRNFEFLVRYYIGPWYARDPAVTLSQLFPGLALLAVTESVRSGWDPSRREDSAGGGDGGGAVLMQLSKSNPVADYMFAQSSKQYGDLRRLEVHDALLFHYEHGLGRLLSVTLPRHRVSTLGSSLFNVNDIYELLYFLVLGFLPSVAVLGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 16 - DNA, 2250 bp, Artificial Sequence
  DNA encoding UL77/FKBP Fusion protein
atgagtctgttgcacaccttttggcggctacccgtcgccgtcttcttcgaaccgcacgaggaaaacgtgctgcgctgccccgagcgcgtgcttcggcggttgctggaggacgcggcggtgacaatgcgcggcgggggctggcgcgaggacgtgctcatggaccgggtgcgcaaacggtatctgcgtcaggagctcagggatctgggtcacagggtgcagacttactgcgaggatctcgaagggcgcgtgtccgaggcggaggcgctgttgaaccagcagtgcgagctcgacgaaggaccgtcgccgcggacgctgctacaaccaccgtgtcgtccgcgttcttcgtccccagggaccggcgtggcaggagcttctgccgtcccacacggtctttatagtcggcacgatgccatcacgggacccgccgccgccccgtctgacgtggtcgccccgtctgacgcggtcgccgcgtcagcggccgccggtgcttcttctacctggctggcgcagtgcgccgagcggccgttgcccgggaacgtacctagctactttggaatcacgcagaacgatccctttatccgctttcacaccgattttcgcggcgaggtggtcaacaccatgttcgagaatgcctctacttggactttctcctttggtatctggtactatcggctcaagcgggggttgtacacgcaaccacggtggaaacgagtgtaccatctggcgcagatggacaacttttccatttcgcaggagctgctgctcggcgtggtcaacgctttggaaaacgtgacggtgtatccgacgtacgactgtgtactctccgatttggaagccgccgcctgtctgctggccgcctacggacatgcgctttgggagggccgcgatccgccggactccgtggcgacggtgttgggtgagctccctcagctgttgccgcgtctggccgacgacgtgagtcgtgagattgccgcttgggaaggccccgtcgccgcgggtaacaactattacgcgtatcgcgactcgcccgatctacgctactacatgcccctaagcggtggtcgtcactatcacccgggcacttttgatcgtcacgtgctggtgcggcttttccacaaacgcggcgttattcagcatttgccgggctacgggacgataacggaggagctggtgcaagagcgtctgtcgggccaggtgcgcgacgacgtgctttctctctggagtcgacgtctgctggtcggcaagctgggtcgcgacgtgcccgtctttgtgcacgaacagcaatatctgcgttcgggcctgacctgcctggctggcctgctgttgttgtggaaggtgaccaacgcggatagcgtcttcgctccgcgcacgggcaaatttacgttggccgacctgctgggttcggatgccgtagccggcggcgggttgcccggggggcgcgcgggcggcgaagaggagggctacgggggacggcacgggcgggtacgtaactttgagtttctggtacggtactacatcgggccgtggtacgcgcgcgaccccgcggtcacgctgtcgcagctctttcccggcctggctctgttggccgtgaccgagagcgtgcgcagcggctgggatccctcacgtcgcgaggacagcgccggaggtggcgacggcggcggcgccgtgctcatgcagctcagcaagagcaaccccgtggccgactacatgttcgcgcagagctccaaacagtacggcgatttacgtcgcttagaggtacacgatgccctgctctttcactacgaacacgggctagggcggctgttgtcagtgaccctgccgcgtcaccgtgtgtccactctgggctcgtccctctttaacgtcaacgatatttacgaactgttgtactttttagtgttggggtttcttccgagcgtggcggtgttgtaaggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaa

SEQ ID NO: 17 - protein, 402 aa, Artificial Sequence
  UL79/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEMARDEENPAVPRVRTGKFSFTCANHLILQISEKMSRGQPLSSLRLEELKIVRLICVLLFHRGLETLLLRETMNNLGVSDHAVLSRKTPQPYWPHLYRELRQAFPGLDFEAAVFDETRAARLSQRLCHPRLSGGLLTRFVQRHTGLPVVFPEDLARNGNILFSLGTLYGHRLFRLAAFFTRHWGAEAYEPLIRIICQKMWYFYLIGTGKMRITPDAFEIQRSRHETGIFTFIMEDYRTFAGTLSRHPHRPHPQQQQHHHPGPPHPPLSHPASSCLSPEAVLAARALHMPTLANDV

SEQ ID NO: 18 - DNA, 1209 bp, Artificial Sequence
  DNA encoding UL79/FKBP Fusion protein
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaatggcccgcgacgaagagaaccccgccgtcccgcgggtccgcacgggcaaattctcctttacttgcgccaatcatctaatattacagattagcgagaagatgtcgcgcggacagccgctgagctcgctgcgtttggaagaactcaagatcgtacgcctcatctgcgtcctcctctttcaccgcggtctcgaaacgctgctactgcgcgaaaccatgaacaacctgggtgtctcggaccacgccgtgcttagtcgcaagacgccgcaaccctactggcctcatctgtaccgcgaactgcgccaggccttcccggggctggactttgaggcggccgtgttcgatgaaacgcgcgccgcccgtctcagccagcgcctgtgtcacccgcgcttgagcggcggactgctgacgcgctttgtgcagcgccacaccggcctgccggtcgttttccccgaagacctggcgcgcaacggcaacatcctcttctccctaggcacgctctacggacaccgcttgtttcgtctggcggccttcttcacgcgccactggggtgccgaagcgtacgaacccttgattcgcatcatctgtcaaaaaatgtggtacttttatctcatcggcaccggcaagatgcgcattacccccgacgccttcgagatccagcggagtcgacacgagacgggcatttttacttttattatggaagattacagaacgttcgccggcacgctgtcccggcacccgcaccgtccgcacccacaacagcagcagcaccaccaccccggtcccccccatcctcctctttctcaccctgcctcgtcctgtctcagcccagaggccgtactggccgcccgcgcccttcatatgccgacgctggctaacgacgtgtga

SEQ ID NO: 19 - protein, 693 aa, Artificial Sequence
  UL84/FKBP Fusion protein
MPRVDPNLRNRARRPRARRGGGGGVGSNSSRHSGKCRRQRRALSAPPLTFLATTTTTTMMGVASTDDDSLLLKTPDELDKYSGSPQTILTLTDKHDIRQPRVHRGTYHLIQLHLDLRPEELRDPFQILLSTPLQLGEANDESQTAPATLQEEETAASHEPEKKKEKQEKKEEDEDDRNDDRERGILCVVSNEDSDVRPAFSLFPARPGCHILRSVIDQQLTRMAIVRLSLNLFALRIITPLLKRLPLRRKAAHHTALHDCLALHLPELTFEPTLDINNVTENAASVADTAESTDADLTPTLTVRVRHALCWHRVEGGISGPRGLTSRISARLSETTAKTLGPSVFGRLELDPNESPPDLTLSSLTLYQDGILRFNVTCDRTEAPADPVAFRLRLRRETVRRPFFSDAPLPYFVPPRSGAADEGLEVRVPYELTLKNSHTLRIYRRFYGPYLGVFVPHNRQGLKMPVTVWLPRSWLELTVLVSDENGATFPRDALLGRLYFISSKHTLNRGCLSAMTHQVKSTLHSRSTSHSPSQQQLSVLGASIALEDLLPMRLASPETEPQDCKLTENTTEKTSPVTLAMVCGDLGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 20 - DNA, 2082 bp, Artificial Sequence
  DNA encoding UL84/FKBP Fusion protein
atgccacgcgtcgaccccaaccttcggaatcgggcccgccggccacgagccagacgaggcggcggcggtggcgttggcagcaatagcagccgacacagcggaaaatgccgccgccaacgccgagctctgtcggcgccgccgctcactttcctcgccaccactaccaccacgaccatgatgggcgtcgccagtaccgacgacgacagtctcctcctgaaaacgccggacgagctggacaagtacagcggctcgccgcagaccatcctcacactgacggataaacacgacatccgtcagcctcgggtgcaccgcggcacctaccatctgatccagttgcacctcgacctccgacccgaagaattgcgggatcccttccagattctgctctctacgccgctgcaattgggggaagcgaacgacgagtctcaaaccgcccccgcgacgttgcaagaagaagaaacggcggcttcccacgagcccgagaaaaaaaaggaaaaacaagagaagaaagaagaggacgaggatgaccgcaacgacgatcgtgaacgcggcattctatgcgtggtctctaacgaggattctgacgtgcgcccggccttctctctctttcccgcacgcccaggctgccatatcctgcgctcggtaattgaccaacaactgacgcgcatggccatcgtgcgcctatcactcaatctcttcgcgctccgtatcatcacgccgctgttgaaacggctaccgctacgacgtaaagccgcgcatcacacggcgttacacgactgtctggcgctgcatctgccagaactcacgttcgagccgacgctggatataaacaacgtaacggagaacgcggcttccgtcgctgataccgcggaatcaacggacgcggatctgacgcccacgctgacggtgcgcgtacgacacgcgctgtgctggcatcgagtggaaggcggcatctcggggccgcgtggactcaccagccgtatctcggcgcgcctctcggaaaccacggccaagacattgggaccctccgtctttggacgattggagctagacccgaacgaatcaccgccggacctgacgctgtcgtcactcacgctataccaagacggcatattacgtttcaacgtgacctgcgaccgcaccgaggcgccagccgacccagtggcgtttcgcctgcggctgcgacgcgaaacggtgcgacgacccttcttttcggacgcgccactgccttactttgtaccgccacgctccggcgcggcggacgagggactggaggtgcgcgtcccttacgaattgacgctgaagaactcgcacacgttacgtatctaccgccgcttttacgggccttatctgggtgtttttgtaccacacaaccgtcagggactcaaaatgcccgttacggtctggctaccgcgctcctggttggaattaaccgtactggtgagcgacgagaacggcgccacgttcccacgggacgcgctcctggggcgcctctattttatctcgtcaaagcatacgctgaatcggggttgcctgtcagcaatgacgcaccaagtcaaatccacgctacactcgcggtccacatcccattcgccgtcgcaacagcagctctcggtgctgggcgcttccatcgcgctggaggacctgctgcccatgcgactggcgtccccggagacggaaccgcaagactgtaagcttacggaaaatacgacagagaagacgagtcctgtcactttagccatggtctgcggcgatctcggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaataa

SEQ ID NO: 21 - protein, 1048 aa, Artificial Sequence
  UL87/FKBP Fusion protein
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEAGAAPRRLGCDALIVVGGSAMPRRVLHVPVHVRACNLTQELSTGEDARFCRPRPVNVERVRAVFAALYRACPIHVRTEPERVKLVLGRLLLGPVAVPCFCDGEVEGHGEHLVPTTQFCRGPLLYVHRRCCCGSVTAGRALSYHVLENHVATHVLRGLLSLTEWNRELPSLFCDCPGGGGASGTEERYAMACLPRDLSLHLDDYPYLMVEIGRVLSVSEVDDYVTAVSGYLGEAAAPRIQVHYKLLFGLNVRPQAPCALDATRDFFLLELQKLWLGVEYHHEVTSEFFGRVLAQLHRDRARVMMALRLPEQTVCHLSTFVLSRFKRQVLYFKLQVSYGKCRTGHADRSGGGGNGGNQGHHNLLCYRRLSVTFADTDTVWRNLFYVYYELARDLGSHGTEDRPVSRGYGVSCASRTSRLSPSESTVVSANGHALSSTALPTTSAGHKLSLPRDPAADRVRRYVCIISRLMYARYGERWRKHCQRRSETGEEEEEETLESGETDATPPFDFTGQQLRRAYQEHRRRKHLAVQRYAPCRRKLIGGMEFAEVTGVSLDRIAVNAFNTNRVINMKAALSSIAASGLGVRAPRLPKNMTHSFVMYKHTFKEPACTVSTFVSNDAVYINSLNVNIRGSYPEFLYSLGVYRLHVNIDHFFLPAVVCNSNSSLDVHGLEDQAVIRSERSKVYWTTNFPCMISHTNNVNVGWFKAATAIVPRVSGADLEAILLKELSCIKNMRDVCIDYGLHRVFTQLELRNSYQIPFLAKQLVLFLRACLLKLHGREKRLQLDRLVFEAAQRGLFDYSKNLTAHTKIKHTCALIGSRLANNVPKILARNKKVKLDHLGRNANVLTVCRHVEAHKIPRTRLKVLVEVLGALQSISGTPHTREVIHQTLFRLCSAAAATSGLCSSPPPLCVSSSSSVPSVPTSVSVDGSSEPTSPRARFASR

SEQ ID NO: 22 - DNA, 3147 bp, Artificial Sequence
  DNA encoding UL87/FKBP Fusion protein
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagccggcgctgcgccgcgccgcctcggctgtgacgctctaatagtcgttggcggctccgctatgccgcgccgggttttacacgtccccgtgcacgttcgcgcctgcaacctcacccaagagctatcgacgggcgaggacgcccgcttttgtcgtccgcgacccgttaacgtcgaacgggtgcgcgctgtttttgcggctctctaccgtgcctgtccgatacacgtgaggaccgagcccgagcgtgtcaagctggtactgggtcgtctgttactgggacccgtggccgtaccctgtttttgcgacggtgaagtggagggccacggtgaacatctggtacctacgacgcagttttgtcgcgggccgctgctctacgtgcaccgacgttgttgttgcggatccgtgaccgccgggcgcgcgctgtcctaccacgttctcgaaaaccacgtggccacgcatgtgctacgcggattgctctcgctgacggaatggaatcgagaattgccgagcctcttttgcgactgtcctggcggcggtggcgcctcgggaaccgaggaacgctacgctatggcctgcctgccgcgcgacctcagcctgcacctggacgactatccttacctgatggtggaaatcggacgcgtactcagtgtcagcgaggtagacgactacgtaaccgccgtctccggctacctgggcgaggccgcggcgccgcgcatccaggttcactacaagctgctctttggactcaacgtgcgtccgcaagcgccgtgcgcgttggacgctacacgcgacttttttctgctggagctgcaaaagctttggctgggcgttgaatatcaccacgaagtcacgtcggagtttttcggtcgcgtactggctcagctgcatcgcgaccgcgcccgcgtcatgatggcgcttcgcttgcccgagcagacggtgtgccacctgagcaccttcgttctcagtcgcttcaagcgacaggtactgtacttcaagctacaggtgagctacggcaagtgccggactggtcacgctgacagaagtgggggaggggggaacggtggaaatcagggacaccacaacctactgtgttatcgacgccttagcgtcacatttgccgacacagacacggtgtggagaaaccttttctacgtttattacgaactagctcgggatctggggtcccatgggacggaggaccgacccgtaagccgcggttacggtgtttcttgcgcttcgaggacgtcgcgactgtcaccgtcagaatcgacggtggtttcggcgaacggacacgcgctgtcttccaccgcgctcccgacgacgagcgcgggtcacaagctgtcactgccgcgcgacccggccgcagatcgcgttcgacgttacgtatgcattatctcgcgtctcatgtacgctcggtacggggagagatggcgtaaacactgtcaacggcggtcggagacgggagaagaggaggaggaagagacgctggaatcgggggagactgacgccacgccgccatttgactttacggggcagcagctgcgccgggcctatcaggaacaccgacgtcgtaaacatctagccgtgcagcgttacgcgccgtgccgtcgtaagctcatcggcgggatggagtttgccgaggtgacgggcgtgagtctagaccgcatcgccgtcaacgctttcaacaccaaccgcgttatcaatatgaaggctgcgctctcgtccatcgccgcgtcgggtctcggcgtacgcgcgccgcggcttcccaagaacatgacccacagttttgtgatgtacaagcacacctttaaggagcccgcttgcaccgtcagcacttttgtttccaacgacgccgtctacatcaactcgctcaacgtcaatattcgcggttcctaccccgagtttctgtactcgctgggcgtgtaccggctgcacgttaatatcgatcacttttttctgccggccgtggtgtgcaacagcaactcctcgctggacgtgcatgggctggaggaccaggcggtgattcgctcggagcgcagcaaggtgtactggaccaccaactttccgtgcatgatctcgcatactaacaacgtcaacgtgggctggttcaaagcggctacggccattgtgccgcgcgtctcgggcgccgacctggaagccattctgctcaaagaactctcgtgcatcaagaacatgcgcgacgtgtgcatcgattacggtctgcaccgtgttttcacgcaactagagctgcgcaattcgtaccagatccccttcctggccaagcagttagtgctgtttctgcgtgcttgcctgctcaagctgcacggtcgagagaagcggctgcagttggaccgcctagtatttgaggcggcacagcggggtctctttgactacagcaagaacctcacggcgcacaccaagatcaagcacacttgtgcgctcatcggcagtcgtctagccaacaacgtgcccaagatcctggcccggaacaaaaaagtcaaattggatcacctgggccggaacgccaacgtgctgacggtgtgtcggcacgtggaagcccacaagatccctcgcacgcgcctcaaagtgttagtcgaggtgctgggcgcgttgcagagtatcagcggtacgccgcacacgcgcgaagtgatccaccagacgttgtttcgattgtgctcggcggccgcagccacatcgggcctgtgttcatcccctcccccattgtgtgtgtcctcatcttcctccgtcccttctgtcccaacctccgtcagcgttgacggcagttctgaacccacgtcgccgcgagcgcggtttgcatcacgatga

SEQ ID NO: 23 - protein, 1063 aa, Artificial Sequence
  UL105/FKBP Fusion protein
MSMTASSSTPRPTPKYDDALILNLSSAAKIERIVDKVKSLSRERFAPEDFSFQWFRSISRVERTTDNNPSAATTAAATTTVHSSASSSAAAAASSEAGGTRVPCVDRWPFFPFRALLVTGTAGAGKTSSIQVLAANLDCVITGTTVIAAQNLSAILNRTRSAQVKTIYRVFGFVSKHVPLADSAVSHETLERYRVCEPHEETTIQRLQINDLLAYWPVIADIVDKCLNMWERKAASASAAAAAAACEDLSELCESNIIVIDECGLMLRYMLQVVVFFYYFYNALGDTRLYRERRVPCIICVGSPTQTEALESRYDHYTQNKSVRKGVDVLSALIQNEVLINYCDIADNWVMFIHNKRCTDLDFGDLLKYMEFGIPLKEEHVAYVDRFVRPPSSIRNPSYAAEMTRLFLSHVEVQAYFKRLHEQIRLSERHRLFDLPVYCVVNNRAYQELCELADPLGDSPQPVELWFRQNLARIINYSQFVDHNLSSEITKEALRPAADVVATNNSSVQAHGGGGSVIGSTGGNDETAFFQDDDTTTAPDSRETLLTLRITYIKGSSVGVNSKVRACVIGYQGTVERFVDILQKDTFIERTPCEQAAYAYSLVSGLLFSAMYYFYVSPYTTEEMLRELARVELPDVSSLCAAAAATAAAPAWSGGENPINNHVDADSSQGGQSVPVSQRMEHGQEETHDIPCLSNHHDDSDAITDAELMDHTSLYADPFFLKYVKPPSLALLSFEETVHMYTTFRDIFLKRYQLMQRLTGGRFATLPLVTYNRRNVVFKANCQISSQTGSFVGMLSHVSPAQTYTLEGYTSDNVLSLPSDRHRIHPEVVQRGLSRLVLRDALGFLFVLDVNVSRFVESAQGKSLHVCTTVDYGLTSRTAMTIAKSQGLSLEKVAVDFGDHPKNLKMSHIYVAMSRVTDPEHLMMNVNPLRLPYEKNTAITPYICRALKDKRTTLIFGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 24 - DNA, 3192 bp, Artificial Sequence
  DNA encoding UL105/FKBP Fusion protein
atgtcgatgacggcctcgtcatccacgccgcggcccacgcccaagtacgacgacgccttgatcctcaacctctcgtcggccgccaagatcgaacggatcgtcgacaaggtcaagtccctctcgcgcgagcgctttgcgcccgaggatttttcgttccagtggtttcgctccatcagtcgcgttgaacgaacgacagataacaacccctctgccgcaactaccgccgcggcaacgacgaccgttcactcctccgcctcctcttctgccgccgctgccgcttcgtccgaggccggcggcacgcgcgtgccctgcgtcgaccgttggcccttctttcccttccgcgcgctgctcgtcaccggcacggcgggcgccggcaagacttccagcatccaggtgctggcggccaatctagattgcgtgatcaccggtaccacggtgatcgccgcgcagaacctcagcgcgatcctcaaccgcactcgctcggcgcaggtcaagaccatctaccgcgtcttcggcttcgtcagcaagcacgtgccgctggctgacagcgccgttagccacgagacgctggaacgctaccgcgtgtgcgagccgcacgaggagaccaccatccagcgcctgcagatcaacgatctgctcgcctactggccggtcatcgccgacatcgtggacaaatgcttaaatatgtgggagcgcaaggccgcttcggcctccgccgcggccgcagccgccgcctgcgaggacctctcggagctgtgcgagagcaatatcatcgtcatcgacgagtgcggccttatgctgcgctacatgctgcaggtggtggtgtttttttactacttttacaacgccctgggcgacacgcgactttaccgcgaacgccgcgtgccctgcatcatctgcgtcggttcgcccacgcagaccgaggcgctggagagccgctacgaccactacacgcaaaacaagagcgtgcgcaagggcgttgacgtgctctcggcgctgattcagaacgaggtgctcatcaactactgcgacatcgccgacaactgggtcatgtttattcacaacaagcgttgcaccgacctggactttggcgacctgctcaagtacatggagttcggtatcccgctcaaggaggagcacgtggcctacgtggatcgcttcgtgcggccgcccagctccatccgcaacccctcgtacgccgccgagatgacgcggctttttctctcacacgtcgaggtgcaggcttacttcaagcggctgcacgagcagatccgcctgagcgagcgccaccgtctctttgatctgcccgtctactgcgtggtcaacaaccgcgcgtaccaggagctctgcgagctggccgacccgctgggcgactcgccgcagcccgtcgagctctggttccgccagaacttggcgcgcatcattaactactcgcagtttgtcgaccacaacctctccagcgagatcaccaaggaggcgctgcgccccgcggccgacgtcgttgccaccaacaactcctccgtccaggctcacggagggggaggatctgtaatcgggagcaccggcggcaacgacgagacggcgtttttccaggacgatgataccaccactgcgcccgatagccgtgagacgctgctcaccttgcgcattacctacatcaagggcagttcggtgggagtcaactctaaggtgcgggcctgtgttatcggataccagggcacggtcgaacgtttcgtggacatcttgcaaaaggacacgtttatcgaacgcacgccctgcgagcaggcggcctacgcctactcgttagtttcgggcctgctcttctcggccatgtactacttctacgtgtcgccctacacgaccgaggagatgttgcgtgagctggcgcgcgttgagctgcccgacgtgagttcgctctgcgccgctgccgccgccacggccgccgctcccgcttggagcgggggagagaatccgataaataatcacgtcgacgcggattcttctcagggcggccagagcgtgccggtatctcaacggatggaacatggccaagaggagacccacgacatcccctgcctgtccaaccaccatgacgactcggacgccatcacggacgccgaactcatggatcacaccagtctgtacgcggatcccttttttctcaaatacgtcaagccacctagcctggcgctgctttctttcgaggagacggtgcacatgtacactaccttccgcgacatttttctcaagcgctaccagctcatgcagcgtctcacgggcggtcgcttcgccacgttgccgctcgttacctacaatcgccgtaacgtggtgttcaaggccaactgtcagatcagctcgcagaccggctccttcgtgggcatgctttcgcatgtgtcgccggcgcagacgtacacgctcgagggctacaccagcgacaacgtgctcagtctgcccagtgaccgccaccgcatccaccccgaggtggtgcagcgcggcctttcgcggctggtgctacgcgatgcgcttgggttcctctttgtgctcgacgttaacgtttcgcgcttcgtcgagtcggcgcagggcaagagtctgcacgtgtgcaccaccgtggactacggcctcacttcgcgcacggccatgaccatcgccaagagtcagggcctgtcgctcgagaaggtggccgtggactttggggaccatcccaagaacctcaagatgagccacatctacgtggccatgtcgcgagtcacggaccccgaacacctcatgatgaacgttaacccgttgcgactgccctatgagaagaacaccgctatcaccccctatatctgtcgcgcgctcaaagacaaacgcaccacgcttattttttgaggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaa

SEQ ID NO: 25 - DNA, 321 bp, Artificial Sequence
  DNA encoding FKBP protein
ggagtgcaggtggaaaccatctccccaggagacgggcgcaccttccccaagcgcggccagacctgtgtggtgcactacaccgggatgcttgaagatggaaagaaattcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacttgaa

SEQ ID NO: 26 - protein, 107 aa, Artificial Sequence
  FKBP (F36V, L106P)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 27 - DNA, 321 bp, Artificial Sequence
  DNA encoding FKBP (F36V, L106P)
ggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaa

SEQ ID NO: 28 - DNA, 239127 bp, Artificial Sequence
  rdCMV -ddUL51
gggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgagggagtgttgagggccgtaagcgtgttgtgtccgacgctgcctgcgcactgccggtgcgtgtcgtcccacggtatttgttgtcggcaccgggcttcgggacggtgtttcggcgcgctgccggtgcgttccacggtccttgcctgtgtcgtttcggcgctgcgcttgtcgggggttttcgagcgttctggccgccggcgatgccggggtgttgcggagacggggggtgtgcgggacggtgttggggccggggacgggggttgcgctggggccggggctgttcgcgccgcgtaggggaggttacgttggggacggggacagtttgcggcgcggaccagggaacccacctcacctatttaacctccacccactacaacacacacatgccgcacaatcatgccagccacagacacaaacagcacccacaccacgccgcttcacccagacgcccaacacacgttacccttacaccacagcaacacacaaccgcatgtccaaacctcggacaaacacgccgacgaagaacaccgcacacagatggagctcgacgccgcagactacgctgcttgcgcacaggcccgccaacacctctacgatcaaacacaacccctactactcgcataccccaacaccaacccacaggacagcgctcattttcccacagagaatcaacatcaactcacgcatccacttcacaacattggcgagggcgcagcactcggctaccccgtcccccgcgcggaaatccgccgcggcggtggcgactgggccgacagcgcaagcgactttgacgccgactgctggtgcatgtggggacgcttcggaaccatgggccgccaacctgtcgtcaccttactgttggcgcgccaacgcgacggcctcgctgactggaacgtcgtacgctgccgcggcacaggctttcgcgcacacgattccgaggacggcgtctctgtctggcgtcagcacctggtttttttactcggaggccacggccgccgtgtacagttagaacgtccatccgcgggagaagcccaagctcgaggcctcttgccacgcatccggatcacccccatctccacatctccacgtcggaaaccgccgcaccccgccacatccaccgcatcgcaccacccacatgcttcgcctcggtcagatcacacgctttttcctgtcccatctacaccctcagccacggttcacaatccccgaaactacgccgtccaacttcacgccgaaacgacccgcacatggcgctgggcacaacgcggtgaacgtggcgcgtggatgccggccgagacatttacgtgtccaaaggataaacgtccctggtagacggggtagggggatctaccagcccagggatcgcgtctttcgccgccacgctgcttcaccgatatccaataaacccatcccctcgccacgacgtctccgcgtatctttgtagcctcaagaatccgtccccacgtccacccatcccgagcactccacacgccataacaaaccacggacacgacaaatgcatgcaaacttctcatttattgtgtctactactctgtgttgctacagggagtgaagagggtgaaggcaaagaaaaaaaaaaggaacaaaataatagattagcagaaggaataatccgtgcgaccgagcttgtgcttcttttcttataaggaggcaaatatactagggaaaacataagaataggaagaaaccgaggtttgggagaaaagctgagataaaatagcgcattttccatacagaggttgttgtttttgtggatcctaagaggtttcaagtgcgaatctcaaagttctcacgagaatattgtcttcaagtatcgacaactgtggtccaagatttttttttggtctttttaggttctgcgagggacatcacgatggatcgttgcgatgaagtcacgcgtacgcctctggtgtggcgcggtgtcgtgacaggagagtgtgttttcagtgcagagctgtcttgattcctatatccgagtatctgttttctcgtaaggacggtaatcttctttggtgtaagtacatctaaaagctgcaaactatattttaagggctgtctctaggtgtactttgatgctggagtttttcgctgtgttgatgtgaataaatctactactactattatatgcagaaagagtgattatgccgagacaagattgcattggctgaactgtttcaaaaacgcctacactctacttatccgtaaacctaaggtaatactatgtgtaagttgtttttttttctttttgtagtaaaatggtgatacgtgcaattaaaactgtattccatgtttccatcctttcatttcaactttaaaggcggctttgagagcgaagaagtgcgaggataaaaatggatgactccttcgtgtccagggagtcgactactgcaacgctgattgattaaaagatggtctccgatgatgatgttgttattgatcgaatcatggtgcagaacggcgacggagaggagcgtgtccgccgccgggaaggtggtctctttctcttttcttttttcaagaaatcttccatgtgtttatcgtagtgatcgaaatcgactgatctcgggttctttttgttggtttcttttcggttaatcatgtattgttttctttttttacagaaagatactttttttcatgagcaattcctcgcccggcgccggcatgccgaggtggggccactgcgatcagcggcatgccgacgccgacccggggatcttggattcaccgttttctctcttctctctctacatacagaccgggtggcaggagcggtaaggaatcatcgtcgtctttcattcttcgatgattatggtaatactaaatcttatctaggagcatatacatctaagattggagtactagtagtcgtttgtggtttctatttttttttatatttatctatgacagtttttctgtttttcgttttgataataatataataaaaactcatggacgtgaaatctggcttggttgtggtgatttcattctcattattgttgttttctttccgtcttgcggatgaagatgttgcgatgcggttgttgttggtgttgctatacaccgagagagatgatctttttgttcttctggttcatttcctatgattgtttggctgctgaccgacgcgtcaggatgtgcagggcatgcggggaatcaggaccggacacgggataatttcatctacctatacggagatcgcggtcctcgccatgaggatcgcgacaggcgcgtcgagggggcaggaacacccttgcggattgacattcttggtggtgtttcgttgttgtcggtagttgttgttgacgatgaggataaataaaaatgaccttgtttttgttctgttttctcttgttgggaatcgtcgactttgaattcttcgagttatcggaaagctgaggtacccaaatgtctgtagcttttttctttttaccctcttgtttatcatctgcgattcgtggtaggtaggagagggaaatgataatccgagattaaggaaaggagaagataaaaaataaaaaaaaataataaaacagaagccgaccggccgccgacccgttccccaggaccagcctacgaggaacggataacgcggtggcgacggcagcggtggtggcgctgggggtggcggcagtggtactgctgatggtagtcgggacggaggagaggcgatgcatacatacacgcgtgcatgctgcatgggtggatggtacggccgggagacgcggaagagaaactcacataaaaaggtgacaaaaagagcggttgaaaaaagaaaacaagattcgaccagacagaagagaaggaccggggcttggcgacccttccacgactgctgttgtcatctcggctcctccgtcttctcccggccacgggcggctaagtcaccgccgttctccccatccgtccgagcgccgaccgaccagccggccgattcgcccgccggggcttctggagaacgccggggcagcagcgatctggggaagctgctaaacccctgcgtttttatatggtagctctgccgagcgcgggctgacgcgttgggtaagcggaaagacgtgtgtgacgaaaaggggtcccatggtatttcacgtgacgatgaggagatacggtttggagcacatacggtttagaaaaagggagttgtcgtgacaagggctgagggacctctgtctccatgtgtgtataaaaagcaaggcacgttcataatgtaaaaaagaacacgttgtaaacaagctattgctgtatcattcggctgactatgcttcattcggactgattttcttttcctaacggcgtaacttaaagtgattaacgtatgatatttgttccccagagttatactatagtcatcatcctaaaattcagatataaatgaacacatgtcgtatgggattattaagaaaccgaaactctccacagttcaccatcttcttcgtcattcaaccgatgacccactccgtacaacgaatcagtctgctgcgtcatattgcaaagcacaagcgacgtatgcgaacaacttgaaacacaggctgttgtattgacgaccgttgtaccattattagtcaccaccgttatcccatgtttcccacccgatggaaaaccgtcttctatcatcaactgtggtaagatttcgaccctgcgaggtattcagtttcctcatatccataacctggattttatcattaaaccccaatattaaacacttttttagtaccccccacccaccaaaaaatgtgactggaccggttcctagcagctctgggagccatgttcaggttgaaccacagctacagcgaaaccgagtccagtgaccggtaaccacgtccagcccctgcgtatgtaccagtccaagcacgtccggtcattgttctacacaggaaatctaactaggtcaacgcaattttattccaccgttacgcagaatactaacaaaaaaacacacaaatttaacgaattacacgtagtttattacatgaaaactgtaagaacaccaattcactaagcgatacaacatttagctgacttccaagtgccacacatcaccactgtattcatccatgttttcaccgaaccaacgagacagatcgaagaagccagaatctcccgactttaaattacataaatccaacgtattatgaccacagctcgacacacaaatagttgcgttactattcacagtagcattacctatacccgtaacgttgcacaaccactgatcaccattgttaccaaaaacggttttccacttagttgtcaacggatctttcctatgcgtaatggtaaaattactaccagtcgtcgcttttagctcattacgagtattatccgcatccacatatatcaacgtcatagctaggcacgctataagtaccccccccccacaatggaatgttgccaaaccggttctttcccgttatagccatagcgttcccaggcaaaagcaaacgccaaacctaatgcagtgaaaagcgcttgcagccagaaccagcttatgtaccagccacaatcacatccggttattgtttccacaggaaatcctaccaggcaaagccccgcttgttttgttcctatcttgtttagcaattcgtaaactgtcagcctagcgacgtccgtttagatcaaaagtcacgtatatagcgacgctgtttccatccgtttccccgtcccgccgtttccgaacaacccacccgggttcagacaaccgaccaccaacagaaatatacacacagaccactgggagttcagttaaagatttcatcaggtttattttggctgctgctagtcttttgcttcttagaaaaaaaatacccatatagagaaataatgatagtttgacaacacatatggcagggatttcttcttcatcaataagatatgcaattcccccagggagagactttcaacaattgaatttacaaaaacaaaattacatcaggagaaagagaggatacattaataaatatattatatctggtgtatatactgaatgctgctggttcataaggtaacgatgctactttttttaattccaagatggtttttctttgttagtcttttgttgacttgctggttcctaaaagttcgcaaaaacgattgtgtgaagattttatgacgttggttgactagttcatgagattctgctgtacgtgtgatggttattcgctggttcgttctaagatgagtatcgtactgtgtctgcgatggtcgtctcttactggcattctctcggctgcctcttgctttcatgattgaaaaggaaaaaaggactccgagggcgcggtcatcttttacttttcggttttctcgttggcgggtcagaggtagtcagatcatgagactgtcgtggtcgatgaaactgtgtctgctcaagtgacgtccatttcttgtacggagaaaaaagtcatcgggataaataaggctatacaaggcgttgtcaagcgtgcggctctaaacaaattaagcgatacaaaattacagtaatacgaataataaattacccccctccccctgtggtcccccgagacgagagccacccatcgtgtactctcgcaccacccacgaccacagagggagacgggacgaagagacgacgcacagcgccatctcctcctggaggccggcgacgttaactgctacagctgcggcggcgaagacagctgcgatttgtcggccgacatgccgatggtatgggcggcggcggcaatggccgcggcagcggggaggagaggagagagaagaggagcggggcgtccgaaggcgaggatggcatggtctcgccggagcgcccggcttttatggaacactcgcgtccggttgggtatcacccacaggaagatgagtcacaacttccaaaccatcttgagacccgagtaacggtttacaggtcgcacgccagtcagctaaaaacagcggacagtcccacgctgtttctgttgtggctctctccagtttcctcatcaccgtcccggtctccgtcgtcatcggaagaataccacccgctctcatgcggcagtcgatcggcctcgacgaacgagacgcggcgacgcctctccacggccgactggttgtggtggtgaaagaagagcaccagcaatcccaggaggagcaacaagccctcacatgtccaggaggtcggggagagggcctgtcggagatggccgtgaggcatcacgtacggcagctgaggagaaacggagaagaaaggaaaattaccgtcaggggccggggttcttattagagaaacagcacgtaggtcaggatccagatgctaatggcaatcatgatgacgatgatcatgcaggccaagacgcggcgcaccaatgccgaatccaatagccgccgtgcctccggttggtggccggcggcatctagagacatgatttgggggggaccggcggcgcaaaaagacagggagatggacagtgtcacggtgttttgttataattaggacatggggaccggaagccgagacagagtactacagggtgttgaagggtaacgtgagggagatcatgtcatgggcgggctgaagaccgtgcggggaggattgacgtgtgcggtgcttgtggaacacggtgttttaatatgtatccgcgtgtaatgcacgcggtgtgctttctggcactcagcttggtaagctatgtggccgtctgcgccgaaaccaaagtcgccaccaactgtctcgtgaaatcagaagatacccatttgacgtgcaagtgcagtccgaataacacatcatctaataccggcaatggcagcaagtgccacgcgatgtgcaaatgccggatcacagaacccattaccatgctaggcgcatactcggcctggggcgcgggctcgttcgtggctacgctgatagtcctgctggtggtcttctttgtaatttacgcgcgcgaggaggagaaaaacaacacgggcaccgaggtagatcaatgtctggcctatcggagcctgacacgcaaaaagctggaacaacacgcggctaaaaagcagaacatctacgaacggattccataccgaccctccagacagaaagataactccccgttgatcgaaccgacgggcacagacgacgaagaggacgaggacgacaacgtctgataaggaaggcgagaacgtgttttgcaccatgcagacctacagcacccccctcacgcttgtcatagtcacgtcgctgtttttgttcacaactcagggaagttcatcgaacgccgtcgaaccaaccaaaaaacccctaaagctcgccaactaccgtgccacctgcgaggaccgtacacgcacgctggttaccaggcttaacactagccatcacagcgtagtctggcagcgttatgatatctacagcagatacatgcgtcgtatgccgccactttgtatcattacagacgcctataaagaaaccacgcgtcagggcggtgcggcgttcgcgtgcacgcgccaaaatctgacgctgtacaatctcacggttaaagatacgggagtctacctcctgcaggatcagtataccggcgatgtcgaggctttctacctcatcatccacccacgtagcttctgccgagccttggaaacgcgtcgatgcttttatccgggaccagggagagttgtggttacggattcccaagaggcagaccgggcaattatctcggatttaaaacgccagtggtccggcctctcactccattgcgcctgggtttcgggaatgatgatctttgttggcgcgctggtcatctgcttcctgcgatcgcaacgaatcggggaacaggacgctgaacatctgcggacggacctagatacggaacctttgttgttgacggtggacggggatttacagtaaaagatgcgtgtcgcctgccgaagacctcaccatctcacgtacaggcatacggcgtatacaatcataatattctatattctgcatagagttacatgcaacagtactactaccaatactgcatccatcacatcacccaacactgcttctaccacctttgtgaccagcgtattttctactccgaataacaacacatcaacgacgccacacacatctgtcacctcacaagcgtcaaccattggcaacatcaccaacgttacctccgacttgagtactttcacaaccgtatattctacattcaatacatcatatgctaatatatccaatacggctgccactacagaattgatttcaacaaataccaacactatattatcttttaccaacgtaacagcaaacgctacatcatcttataacacaacaatcaccgtaactatcacgtcagatgaaacttcgcacaacgtatccactaatactgcacttataagcacgccatggcttacaaattgcagcgccacaacgtacaccacgtacaaccgtactaactcttccaacgcttgtcacacagagacaacaatcatacgtttcaaagaaactaatacaacaggaatagaagggagtaatgtcaccataaaaggtaattctacgtgggattgtctttcagtcgcctggatacgacattacaatcgatccacacacggacatcatctaggtcatcgtaagaacgcacatacccaatcttggtattggttacgcatccttacctctcatactgtatgtcattctcaacatgaaagaccttcactgtaccatgacttatgtcgttcgtgcaacaacacagaactacatctgtacgatctaaatatcaccaattccggcaggtacagcagacgttgttttaaagaaaattacttcacaggacatcacgaagatgaaaatttctacctattagtaacaccaaaaaatcatactgaagctattaatgctactttcgtttgccctagatacaacaccgatatcgaaaatgaagatagagagaaaggaagtcaacatactaacaatacacatcaccacaaacgtaatctctatcatagctcgcaaagaagccgcaccgtatggaccatcgtgttggtttgtatggcctgcatagttctgttttttgcacgacgagcctttaacaaaaagtaccatatgttgcaagacaccgtcagtgaatcagaattcattgttcgatatcacacagaacatgaagattgagctacgtttccgggcagacatcttatgaagctgaacaataaactaaaacattctgtaaggctcagcgttcaaaggaatattaatgcccattgagcgagaactaatattgcaatggactggcgatttacggttatgtggacgatactaatatccgcgttatcagaaagctgcaatcaaacctgttcctgtcaatgtccctgtagtactaccgttaactattccactagtactgagacagccacatcaacatacagtacaacagttatcagcaataaaagcacttcagaatctataaattgctctactgcaactgcaccagcaaccaccgtttctacaaaaccgtcgaaaacaaccacacagatatccacaacgacaaatacaaacgttgagactaccacatgtaccaacaccaccacgaccgttacttgtgatggtttcaattatacagtccataaaagatgcgaccgcagttacgaggtaatcaacgtaacaggatacgttggtggcaacataactctaaaaaatgcaatcagactgagaaatggcacaatgtagactggattcattatgagtaccccacgcataaaatgtgcgaattaggcaactatcaccaaacaacaccacggcacgacatatgttttgactgcaacgacacctccctaactatctacaacttaaccacaagaaacgctggaaaatataccaggcatcaccgtgataacggtcaagaagaaaattactacgtaacggtgttaattggagacacaacgttatccactcttggcacatgccctgtaagatataaagaatctaggaacactgaaaacaccattggaagtaacatcataaaaaccattgagaaagctaacattcccctgggaattcatgctgtatgggcaggcgtagtggtatcagtggcgcttatagcgttgtacatgggtagccatcgcattcccaaaaaaccgcattacaccaaacttcccaaatatgatccagatgaattttggactaaggcttaacatgcacatcaataaactttttttaaccaataacatgtctctgtttttttttgttaacaacctatgatataaagcggtatattcaatcattactaaacaaaaaaacatgggcatgcaatgcaacactaaattgttattgccagtcgcactaataccggttgtaatcatcctaattggtactctagtgcccatacttttacatgaacaaaaaaaggcgttttactggcgactttttctgcaaagtcaacatgtagaagcacccattacagtaacgcagggagacacagtctacctagatgctagcaataatccctgtaattattccagcttttggtaccacggtaattgcgaactttgtggatggaacggatatctacgcaatgttacacattactacacaaacacatcgtgttccccgcaattcatgtgcataaacgaaactaaaggtctgcagttatataatgtaacattaaacgattcaggtgcttatactgaacacgtttacgaatgtgatctttcatgtaacattactacttataacgaatatgaaatactcaattacttcgataactgtaactacaccataaatagcaccaagcatattatcaccgtggtgtcttcacgtcattctaaacaaacaaattcccacgtatccactcacgctggttgggcagccgccgtggtgacggtaattatgatctacgttttgatccactttaacgttccggcaactctgagacacaaactacgaactagaaacaacgtaaatcgcatagcgtgattacaaagtatcgacactaatttatccaagataaaatttgattactccgtgcggttctcaaaaactgtaaggtcccgcttttctactccatcatgaaggatcgcaatagaatactgctatgtatcatctttatttgcatcatgtgcctcatttgtatttactttaaacgtcgttgtgttcttactccgtctccagacaaagcggatctgcgagtggaatttccctcgttacccccgtgtatcggcatacaatgtgctgcatgagaacacgcgtgacacatagcgtacccctggacggtacagtttatgataacgtcattcaggggaagtatacattactatcgacgtgttatcacagaacacacagattttctgcgtgttttataaaagagcgtctcgaagcagcttgagccacactacggtccagatgacgagcgtaatcaaaaatatgccgcgcagtagtcgaaagccgtactgagcgtgcgaggcgggtagggtgccgaacgacggatatgcgtcgttgtcatcttcgactataaggatcgcgaccgagtcttcggccatggtaaacgtcaccctgtgtggctggtatgtagcgtatccggtttggaattgttctgctccagctcgggggatagtgaggaattctcaagggatacgggacccaatgactggataagagaagggtttttccccgtaagatgatcctcgtatcacatgaggtctggatatgtataaatgaagagtgaaataggcacagggaatcagatgccagcctcgtgatgcagccgctggttctctcggcgaagaaattgtcgtctctgttggcttgcaaatacatcccaccttaagcgatgagtccataaagcaccgttgtccgggtacggtgaaagtgactcggattgtagcacgtcccttttttttgtttttgtatcgcttatcgccactgacagtgcaatattttgatcgtgaggctgagtatggttatgatgcttagaacgtggagattattaccaatggtactacttgccgcgtactgttattgtgtttttgggacttgttcaatcggcacgacgactgctcccgtggaatggaagtctcccgaccgtcagattcctaagaatattacttgcgctaactactcagggaccatcaacggcaacgttacatttcgaggtcttcagaacaaaacggaagactttttgcactggttgttagggtggggtcataagtccatctgttcgttcttcccgaaactccagggcaactataacgaacaacattacagatatgaagtagcgaacctgacgtataactgcacctataaccgcttgacgttgctaaatctgacgacggaaaacagcggaaagtactattttaaaagggaagatgcgaatttcaccttttattactcttgttacaacctgaccgtgtcctaaagaacgcacgtgaagttccacagagccgcgtggctgtagctattgtgtttacgttgcttttgaaatgttaagcgtccctacggcgctaacatgtttctaggctactctgactgtgtagatcccggccttgctgtgtatcgtgtatctagatcacgcttaaagctcgtgttgtcttttgtgtggttggtcggtttgcgtctccatgattgtgccgcgttcgagtcctgctgttacgacatcaccgaggcggagagtaacaaggctatatcaagggacaaagcagcattcacctccagcgtgagcacccgtacaccgtccctggcgatcgcgcctcctcctgatcgatcgatgctgttgtcgcgggaggaagaactcgttccgtggagtcgtctcatcatcactaagcagttctacggaggcctgattttccacaccacctgggtcaccggcttcgtcttactaggacttttgacgcttttcgccagcctgtttcgcgtaccgcaatccatctgtcgtttctgcatagaccgtctccgggacatcgcccgtcctctgaaataccgctatcaacgtctcgtcgctaccgtgtagctagttagccagctgtgtatagtttgttgtgttttgcttttgcatatttgttttcagtcagagagtctgaaacggggtgggagggacttttacgggtaatgcatgctaagatgaacgggtgggctggggtgcgcttggtaactcactgtttgaatacgcgctcacgcacatatgtagcactcaacatgttagcttttgcccgcacgccccggggcgtgccgagctgcctttttaataaagtctgggtttccagatacgcgctggttctgattttgatggtttgtgcctctgaaagctctacgagctgggccgtgacatccaatcgactgcctaactgtagcacgataactacaacagcgggtcaagacgctgaattgcacggtccggcaccgttaagctgtaatgtgacccagtggggacgttacgagaatggaagcacacccgtattatggtgcactttatggggatcacgcacgcgagtctcattaggacaccgtgtagcgtttggctgttcttggaaaacattttttatttataacgtttctgaaagtagtggtggcacttattatcaaaaaggttacaactgcaccgacaaacatataacactatcttgtttcaacctaacggtggttcctcgagcggttcaaagcacaaccaccgtaatgacacccacggtggttacaaactccacattcagtgtgtcacttgttgcgtcgagactgacgacaaattccagcgcgtttagacacgctagttatcaacggcaacagcgtgtcggaaacgggacgttatccaagaacataactaacttggcattcacctacggcagctggggcgtcgcgatgctgctgttcgccgccgtgatggtgctcgttgatttgggtttgcctcaatcggcttggcgacgctggcgaagccacgtggacgatgaagaacgtggtttgttaatgtaggaaataaaaggcactgtttgagcatgactgtttccaaaccgtaacgtggtaaataaatcatggcttccgacgtgagctcccatcttctaacggttacacaatcccgttggacaatacatcatatgtacaataaactgttgattttggcgttgtttacccccgtgattctggaatccatcatctacgtgtctgggccacagggagggaacgttaccctggtatccaacttcacttcaaacatcagcgcacggtggtttcgctgggacggcaacgatagtcatctcatttgcttttacaaacgtggagagggtctttctacgccctatgtgggtttaagcctaagttgtgcggctaaccagatcactatcttcaacctcacgttaaacgactccggtcgttacggagcagaaggttttacgagaagcggcgaaaatgaaacgttcctgtggtataatttgaccgtgaaaccgaaacctttggaaactactacagctagtaacgtaacaaccatcgtcacgacgacaccaacggtgatcggcacgaaaagtaacgttacggggaacgccagtttagcaccacaactacgtgccgtcgctggattcttaaatcagacgcctcgggaaaacaacacgcacctggccttggtaggtgttatcgtatttatagctctaatagttgtttgtattatgggatggtggaagttgttatgtagtaaaccaaagttatagtgatgtgctttttatcagggagaaggttttgtgccaacaatgactaaccctgggctatatgcatcggaaaattataacggaaattatgaacttacggaagccgccaatacagcacgtacaaatagcagtgactgggtaacgttaggaaccagtgcgtcgctgttgagaagcacggagactgcggttaaccctagcaacgcgactacggttactccacaacctgtggaatacccagctggggaagtacaatatcaaagaacgaaaacacattattcttggatgctaattattgccataattctcatcatttttattatcatctgtctgcgagcacctcaaaaagtctacgatcgctggaaagacaataaacagtacggacaagtatttatgacggacacggagctgtgatgaactacaatgtatagatacacgtggctgctttggtggataacaatattgcttcgtatacaacagttctatcaatggtggaaaccagatacaacgtcatgcattcagaaaacgggatatgaaggtcaaaacctcagtctgcctcctagtaatgcattatcatctaaagactatactttttcatggtataaagattcacttaaagcccttaacatgttatgttattatactgaaaaacttgaagaaatagatagcaagccagatactatacgacgatgttttttgaatcatacattgtttcttattaatttaacaagtcactatagcgggatttactacttcgattctctatacacatatggttgggtattacggacacctctatgttacaatgtcactgtatattccatatatcaaacacacatccacacaactatattgctctatccgcctacgtccacatataattcattaactatatcatcatttacctcaaccaacttaacacataccgcggtccactatgccgccggtaacgttgaagcacaacacgatactgccaccccacatacaatgtggatcatacccttagttatcgttacaacaattatcgttttaatttgtttcaaatttccccagaaagcttggaataaattcacacaataccgatacaacagtatgctcaccgccgcttaaagaatcaccgtcgagaaaactaaaacgtaaaaagaatggccatgtacgtttatttttcagctcactgtttgaataccgtaaacataatgacgtacatatacgtgattatacaacaggtgtttgtgttatgcggcgactgattaaccatatcgtgaaccatgatcttttccgatggtccgtcatgaccgcaatgatattttacaggtattccgaaacctgtatggaggtcactgtcagagtaggtgatccagttaccctcggtagtggacatggttatcatccaggacaaaaagtacactggtataaccagtcatgcgtcggcattagcaacggcgaaaatacgcatcctatctgcacctacgaccctcctaaacctggtagacgaaagacaatgaaaaccactccgttaccatcaccactgttgtacgagtgtcacaattccacattaagcattcttcatgtaaacgtctcagatcccaaaaactattgcaggcgaaaatgtccaccaaacggtaactgtgaatttcccacatgttttacgttatcactgatttccagaacgacaaccaccagaaaacccggacaaaaaactacgttgtcgcgattaaaaaccacgccaaataaacatacgcagcacaaaagatccacgcgaagaacgtcacctagagattacaatgtaacgggtctgccgaaaggctttgcggactcgtttaccggtaacgtagaggcacatagagccaaagatgccgcacacagcgcatggatcctcattgtcatcatcattatcatagtcgtcattttatttttcttcaagattcctcaaagactcagagagaaatgggacaccagaggatacctttacaaaggaaccgacggcctgcccactacggactacttatcgtgagcggacggatatctccggtttcaaacccactgtttgaatatagggacagtccctacggaacctgagaacatgtggaaatcacctgtggtagaatgctgctcaggtacattacctttcatcgtgaaaaggtactttacctagcgatcgcatgcttctttggtatctacatcagtttccacgacgcatgcattctggtacctgctaaagtaggtactaacgtcacattgaacgcggtacatgtgcatgacggtgactatgtgtactggtcttttggtggaggtggagctaatagattaatgtgtcgctatacaccaaggctagacgaaattcacaaaaacaccaatcgaagtttttcatgtcttacaaatcacagtctccttctcatcaatgtaacggaagaatatactgattactatcgcaccatgaccacattcgtacatcagtcccataattggcacaaccacggcaacaaatggactttagacacatgttattatgtatacgttacccaaaacggaacacttcccactaccaccaccaaaaaacccactacgaccacgagaacgacaactaccaccacaacaaagaaaacaaccaccacgagcacgacaacgaccaccactaccaccaagaagacgacgacaagcactacccatcatcgacactccaatcccaaagaatccaccacccctaaaacccacgtagaacttcacgtcggtttaggagccacagcagcggaaacaccgttacaaccaagcccacagtaccaacacgtggctacacacgccctctgggttttagcggtcgtaatcgttattatcatcattatcattttctactttcgaataccgcaaaagctgtggctgctctggcagcatgacaagcacggcatcgtgctcatccctcaaaccgatctgtgagcaagtcgcgtaggaaatgattgcatgaaatcactgtgaaacgccaactccgtgccagctggcgcggcggacaggcctttgacgtatttgaagccaggcgcgctctcgataccgaaaggatccgagggggctttccaaagccgacgtccctgattcccttcataaagctgttgaccggccctagaaagaccaagagcatgctgtgggcccactgcggtcgcttcttgcgttatcatctgctcccgctgctgctgtgtagactgccattcttactccttttccagcggccgcagtgggcccacggcttggacattgtcgaggaggacgagtggctacgggagatacaaggagcgacgtaccagctgtccatagtgcgccaagccatgcagcacgccggattccaagtcagagcagcgtcggtcatgacgcggcgaaacgccgttgacctggaccgaccgccgctttggtcgggatcgctcccgcatttgcccgtctacgatgtgcgttccccgcggccgttgagaccgccgtcatcacagcatcacgccgtatcacccgaactgccgtcgcgagacgggatacgttggcagtaccaagagctgcagtatctggtggaagaacaacggcggcgaaatcagtcgcgcaatgcgattccgagaccctcgttcccccctccggatccaccatcgcagccggcagaggatgcacgagacgcggacgcagaacgtaccgaatcaccacatagtgcagaaagcaccgtcaggcacgacgcgagtgagaacgcagtgcggcgacggcacgaaagacggcgctataacgctctgacggtccgcagccgggactcgctgctcctgacgcgaatacgcttctccaaccaacggtgtttcggacgcgggcgtctgagacatcccgcgggaagcggtcccaacaccggcggaccgcgacccggcggtgcgggactccgtcaactacgccaacaactgacggtccgctggcagctgttccgcctacggtgccacggttggacacagcaggtctctagccagatcagaacccgctgggaggaaagcaacgtcgtgagccagacggccacgcgagtacgtacgtggttcgtggaaagaaccacgttttggcgtcgcacgtgggttccgagacagaacccggcggccgaagcgcaagaactggccgtcataccgccggcacccacggtgctccggcagaacgaggaaccacgtcaacagcttacgggagaggagacaagaaattcaacgcacactcaacgtgaagaagtggaggacgtttcgagagagggcgcgagagaagggaatgatgggagccgagcaagtggaaacgacgagagaaggaataatgcgggaagatatgatgatgatcatgaggttcaagagccgcaggtcacttatccagcgggacaaggagaactgaataggaggtcacaggaggagaacgaggaaggtggaccgtgtgaatcgccgccaatgacgacaaatacgctgaccgtggcctgtccgccccgagaacccccgcatcgtgccctgtttcgtctatgcttaggactgtgggtctcgagctacctggttcgacggcccatgacgatttagaatacaccgagccattcctttatttccccccatccccggtcgcttatgcgtgttaaacactaccaataaagataatctgccaatcgcaccttatatataatatgtggtcgcgtgtggtctttttaaggagctctgaaacacagacaggtatgggcggtggtcggctgccgccgctgtggctgccgctactgatcgcctggagcgagtggggcaactgctgcctcgatgcgcctccggtggtgcgttcgccctgtctgcagccggtgcgcgaccgcaaccgcgagcggaacccgggctcaccgcagttgctgccttacggcgaccgtctggaggtggcctgcatcttccccgcgcacgactggccagaggtctctatccgagtccacctctgctactggcccgagatcgtgcgttcgctggtggtggacgcacgcagcggtcaggtgttacacaacgacgccagctgttacatcgccggcgggcgctggcgcttcgaggacggcggcgcggcgcagcggctgagcctctcgtttcggctcatcaccgagaccgcgggcacctacacctgcgtgctgggcaacgagacccacagcctggcgaccgagaccacggcgctggtggccgacgtgcacgacctgcgccactcggaccgctcctgcgacctggctttcggatcgcgctcacagacgcggtacctgtggacgcccgatccctccaggttgcgcagtataaactgcggttgggagggtgaacggcaccgcgtagtccactacatccccggcacctcgggtttgctgccctcgtgcgaggaggacgagcgcgaactgtgcgtgcccttcatcagccagagcattgcggacaacaactgcagccgccggcatcgagttgacggcgctaggcggcgctatcatctacggagggattactggctgacggatccgaagatcgggttgctggccgcgggatcggtggccctgacctccctctgccacctgctgtgctactggtgttccgaatcgtaccggcgtctgaacaccgaggaggaaagcgaggcggcggaggaaactgccgcgggagaagcctctgcggtagcggcggcggccgtctctgaggaagagcagcggcgggagtaaacgaggagagccatgaagcggatgattcgcagtcacggcaggaaaacggaatgtcagatgacgagcgccggcgagcgacgcggctccgccgtcggtgcgcccatctgcggcagcggtacccgacgcggcagcggcgccaacgaacgccgcgactccgacgtcggtcccatcgcccacagtagcggtaccagacgcggttcggcgaatgaaacgtccgcctgtacgcggaccgatcaccagaaggcggacattgggctgtggttcatgtttctggtttttggactgtgttcgtggttggcgatgcggtatcgcgcacaataaattttgaatcgatgtcaaggaacgcgtgttttgtattttattgggaatattggcggggataaacctgtttcggatgtttacccttaatcttaccggggacctcgttgtcctctcctccttcttcctcggacaccgggctccatgctgacgtaggtaccgactggggtcaaaagcctgggtacttatgaggagcgcgcacaaaggaccgttaggcgccggcatggagcgtcgccgaggtacggtaccgctgggatgggtgttttttgttctttgcttatctgcctcttcctcgtgtgctgttgacctgggtagcaagtcctccaactcgacctgccgcttgaatgtgacggagttggcctcgatccatcctggggaaacgtggacgttacacgggatgtgtatttctatctgctactacgagaatgtgaccgaggacgagatcatcggcgtggcttttacttggcagcataacgagtctgtggttgacctgtggttgtaccagaacgacacggtgatccgcaatttcagcgacatcaccactaacatcttgcaagacggactgaaaatgcgaaccgtccctgtgactaaactgtacaccagccgcatggtcactaatcttaccgtgggccgctatgactgtttacgctgcgagaacggtacgacgaaaataatcgagcgcctctacgtccgattgggctcgctatatccgagaccgcccggatccgggctcgccaaacacccctccgtaagcgccgacgaggaactgtccgcgaccttggcgagagacatcgtgttggtctcagccatcactctgttcttcttcttgttggccctacggatcccccagcgactgtgtcagcggctgcgcattcgcctgccgcatcgataccagcggttacgcaccgaggactgaacggataaccgcaaaggccacgtgcaacgttcacgctgctataagaaggccatgtcccccgtggacgggtctctttgacacgagcgcggcacgccgttgccacgagcatggatcacgcgctcttcacacacttcgtcggccgaccccgtcactgtcggttggaaatgttgattctggacgaacaggtgtctaagagatcctgggacaccacggtttaccacaggcgccgcaaacatctacctcgacgtcgcgctccgtgcggcccccagaggcccgccgagattcccaaaagaagaaaaaaggcggccgtccttctattttggcacgatttgtgctggctgtttcgacgacttttctttcctcgggaggactcagagccactgatgtcggatccggcacggtctcccgaagaggaggagtaaacaacacacggctaagaggatacatcatcaaagaagataggaggggtcaaaacgcggactgaaagtatataacgccgatcatgtccgaggaactgttaataaaacgccatgatgacaatgtggtgtctgacgttgtttgtgctgtggatgttgagagtggtgggaatgcacgtgttgcgttacgggtacacggggattttcgatgatacatcgcatatgacgttgaccgttgtggggatttttgacgggcaacacttttttacctatcacgttaattccagcgataaagcgtcaagtcgggccaacggtaccatttcttggatggctaacgtctcggcggcctaccccacctacctggacggggaaagagccaaaggtgaccttatttttaaccaaaccgagcaaaacctgttagagctggaaattgcgttgggttaccggtcacagagcgtgctgacgtggacgcacgagtgtaataccacggaaaacggtagttttgtagccggttacgagggatttgggtgggacggggaaactttaatggagctcaaggataacctgacactatggacgggccccaattacgaaattagttggttgaagcaaaacaaaacgtacatcgacggtaaaattaaaaacatcagcgagggggatactacaatacaaaggaactatctcaagggtaattgcactcaatggtccgtcatttatagcgggtttcaaccccccgtcacccacccagtggtaaagggcggtgtccgaaaccagaatgacaacagagctgaagcattctgtacatcttacgggttctttccaggggaaattaatattacttttattcattacggtgataaggtgcccgaggatagcgagcctcaatgcaatccgctacttcccaccttggatgggactttccatcagggatgttacgtagccatcttttgcaatcaaaactacacctgccgcgttacacacggtaattggacggtggaaatccccatcagcgttacctcacctgacgacagttcctcgggggaggtccctgatcacccgacagctaacaaacgctataacaccatgaccatcagcagtgtcctcctagccctgcttttatgcgctttgctattcgcgttcctgcactactttaccaccttgaaacaatacctacgtaacctggcctttgcgtggcgctatcgcaaggtccggtcgtcatgaccagcaacgccctgtatgagctgtttcgacgtaggttaccgcgtgcccccgtcaacacggtcatgtttctcacgcgacgcactcgtgatgggttctgcggtcggttgacgtccatcgccacgaattcccactacactatgttcgtgttagatcacgggtccgtgcgcatcgagcgaccgagtcagtcagaagtggattgcgccagtttaatggaaacgctgaagcggattcggttacgaaattcgtgggtagcgtcagaagacgagctagatgtgagtcgcggggacgcgtgacacaaaacgcgttcaggattaacgtaggttttcgaaataacctacgtccgtgagtgacgcggtttcgtgttgaaacccgcgccggttctcacggtggtttatgatgaaaccggcgttggggatctacgcgggttcctcattcaacctgcgaaaagaggaagttgcggtaaaaccacgtcaataaagacgtcaatgacacctcaatgttgcgttggaacggtctttatatatacaaacgccgttatgttcagtgtccggcaagatgctcgggatacgggctatgctggtgatgctggattactactggatacagttgataacaaacaatgacactcgaagcaacaataccgataccatctttgtatctctccttaccggggccaacggagttactcgcacagccatcgggggtctgcattcaaactacaccaacttaaccgaggcattcagattcactccagcaaacacaacaactaactcttccacggagggtaattggagcgtgactaacctaacggagagttgcatcaaccgcggtgagtcctatctaactaccatctggcttctgaactgcgctgacaacaatacttattggtactctggaaatgcctataaccatacaattgacacttgtaaaaatacagtttcgggatatctcttcttcggcatgtgccagctatggaaagattgggttactaatgcttctcacgacactgtcagaattcagtcgttgggaaatgaaatacgctgcatgctgctccctagacagtataccctcaacgccacggtggaatggtacaacaaatctgaaggtgacgtaccagaagaatttatggactatgttatcctgacccccttggcagtgcttacatgcggactgcaggaagcttatatactcgacaagggtcgtagatacatgtatttgttttccgtgtcctgcgcgggaatcacaggtaccgtatctattatactcgtctccctatcgctgctcatcctcatctgttactatcgctgtggccggcttctgatatgcccacgcggctttgaactcttgccagaattcactgaggaagaggaggaaaaagaaaaattgttaacgtacaaggacattgaagtccaggtgcctatccgcacgcggcggctgctcgtcccttggatccgggagagcaaaatgtgggtactaccacccccgctgcctccacgacctccccacttaatagaattcccgccgtctcctccgccgtcgcctgggcccatgcacatggtggtctgcatgccagcatgacggactttgaactttgagccccaagcggtacggactacatattttccataaatctacactgaacttgagcacaaaaatactgacaatggactgaatatacagacttttatatgatccttgtacagatgtaaataaaatgtttttatttaaaactggtcccaatgttcttcgggaatcatggggtggggacgggggacgcggtaaggagcaaaaccgggtacatgggggggaacatcgtccagcagtagcaccagcggattgggtaggggttgctgcggaggtcggtcgatgacgatgtcgatctccatcggcagatccggcaacatctcttcgtctccctcaccgaccagcactcggcgctgttctggatgtatatgattttggaaaagcctccgacgagctcgcggcgcgtagaaagccaagcggcgcaagggccggcgagcccgaaagtccatgcgcacagatggcatgagtccttgagtgacggtggtgagctggggaacagggctacctcccatcgcgacggtgacagtggatccatgagagaggcgccgcacgctgcatggctaaataccgtgaatcccctgacgtcgtctttcgtcccgaacgcgtcatgttgggggcgaggcgtaaaccgtcgaggttgaaaaaccgcgtatctgcgacccgtccggactacgttgtttttcagaagcggccacatgacctcgagatgtcgtcacccaaggtatttaacggcacacagccagacgcgttcgtcagcagcgacgccgacaagacctcagcatggctcggaggctatggatcttgagcttactagccgtgaccttgacggtggctttggcggcaccttctcagaaatcgaagcgcaggtaaacggaatctggggaattcaacacaggtaagaaatacaaaaaataacgtgattgtgaacgcggttatcgtgtttttgcagcgtgacggtggaacaacccagtaccagcgctgatggtagtaataccacccccagcaagaacgtaactctcagtcagggggggtccaccaccgacggagacgaagattactccggggagtatgacgttttgattacagacggagatggcagcgaacatcagcaaccacaaaagactgatgaacacaaagaaaatcaagccaaagaaaatgaaaagaagattcagtaacagcagaccccaagggttaacgattatgttgactaccttgttttttattaaaaagctgtaaggttttgctctaaaaacaccccgcctccggtcttttttcttttgtattcggcacgcgaaacacggtttcttcccatagcctgtctaactagccttcccgtgagagtttatgaacatgtatctcaccagaatgctagtttgtagaggctatgcgggatgctgcggcggcgcgaccttccctctccacccagccccgtcaaaacacacgcgactcgagcggttcgtatgaaaaataaaaaacagctttttatttacaggaacggggaaaaaaaaggcacacggtccgtgggagacgcgggttcacgcgtcgtcaaaaagttggtggtccactccgtaaggacaggtaggcttatttagcttccgcatgctcctggttccgtaataaatgccgttttcgtggcagcgtgtcatgccgcgagtcacaaactccatcaaactgtcggccacgatgcaaacgtgctgattgttggcagcaaagacgcgcatacagtcgtccacgaagaggttgatcacgtcgtaggggctcaccaaccagcctaaaggttccacgtggttactgccgaccatgaccctccagtcgttaatctcgctccagtcgtacagccgaatcgtggagacgcgaatgacgctgtaatcacccatgaccatgagtcggccgcgatacgtagcacgccactgcgcgaacgcgtggatgtgcatgcagccggccagcgctctaagcgaggcggtgtgcggcagctcctctgggacggtgatgaagttgcagcgtcgcaaaccgatgttgagaaattcagtgatgctctcggccacaaaggtcaacgagtcagagtagatgtggtcggtccacaggtacatggcgcccgaggcgcccaggtacagttcagacggcacgttgtgatcgcccttgtgtttaagaaagttgtaggtgcagatgctgccgacgaaacgcagcggctcggggcagcagaggtagctggccagacgctgtgcatcccgtccttcgtcgcgcaccaagcgccagcgacgccggataacgaggcagcggtctttgggccagaccagggccacgcgttgcccgggtttccacggtcgcgacgtcttaggaggcctccagcggtcgagcagattgagaaaacagtccttgattaccgacatcgcggtcgcgcgtcggtggacaaaaagaaatcgggccgatccggaaaaaaaaaacgacggcaaaacaccgccgtgctcgagcgaagggtggcggagggccagaagaggcggccttgacggcgttggcagcgaaaaaattggcacgcgagtcaaacgggaagtagcgtcggtgttttatgccccaagcagcgtcgtcgtcactcgtggcgtcacagtcaacggtgctgacgtcctttggggcagtcgggcacgcgatcgtagatgccgttgtggccgctgaaacgtcggttttcaaacagcaggttaagtcccagacacatgaacgtgttgagattatctcccacccggatgtagcggtcgtcgcgcacgtcgcaggcgtagacggccccggtataggcgacgacgatggggataaggtcgacgggccagcgcaagtgaggaaagggcgcgttctcgcccttgaggctgacggttcccaggccgagaacgcgcattccgaaagcggttttgatgttgcgcagcaagtgaccgccttccacgctgttttcgaaacacctgaggttgcatagacgcagttccgttcccggcgggtacgtcaacggcatgaactgcccgtggtggcggatgatgaatcgcgccatggtatccaaaccgaggctccaggcgcgcaacagcgggcgaaagtagcgcttaaccaacgacgaggtcaggtagcgcatgcagtgcagggtctcgacggcgcgcagcccgacgcgcgcaaactccatgaggttgcgggccaggtagtagacggcggtgtcctcgcgtacatagcaaaaaacatagccctcgtccgagatgaggcacacagcggtcttcttctgctgatccggcgacaacacgccctcgttcacgaagcgacccacgaaggccaggcgcgtctggcaacacaggtagtgactccaagccttcacgtcctccggtttgaagtcctcgtccgtctcgatctcctgcagcactaggttccagcccggcggccagaccacgggcaacacctggcctgcgttgatgcgcacgtaagcttccagacagcccaggccgaactcggccgtgagcgccaggctagccagatcgctcatgtgacgcgccgagtcagtgggcgagcccgggggcccgtcgcacaccacgctccgtcttcttgtcctcaccgcggccagcgtggcgaggacactttccgcgcccgaggctgtatcttcggtttgcccgccggagccggccctcactatataacgtcccgcccgggtctcctccatgtatgcaggtaagcaactgagccgaacgcacctcagcagacgagaggatgtcgtcgcggcgtcgcagctcgtcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgtcgtcgcgtcggcaaccgcagatcgcagcgggcgcgcctcggggctcgccggcgacacccaaggccggcaagtcgcctaaagtctcgcgaccgcctagtgtgccctcgctgcccgagaacggcgccggcggcggtggcgacgataacagcagcagcggcggtagcagcagtcgcaccaccagtaacagtagcagaagtaccagtcccgtggcgccaggtgagccgtccgctgccgagggcgatgagttttccttctgcgacagcgacatcgaagactttgagcgcgaatgttaccgggtcagcgtggccgacaatctgggcttcgagcccagcgtggtcgcgccgcagcacgtcgagtatctcaaattcgtgctgcaagactttgacgtgcagcacctccgccgcctcaacgaatgcatacccatgccggccttcgcgctcaccagcctcgtcgaccccgtcttaaacaacgtagcgcctggcgagcgcgatctcacgcgtcggataatcacgcacgcggtgatcatcaactattactacgtggcgcaaaagaaagcgcgccacatggtggaggccatacggaccaccgtgcggggcgacacggtacgccgggtagccgcgcaggtcaacaaccagagccgttcggggcgtgcggccgcgctagcgcttcattttctcacgtcacgaaaaggagtgacggacggccagtacgccacgtctctgcggcggctggacgaagagctgcggcatcgcggcacgcccgaatcgccgcggctcaccgaggtttaccagacgctacgcgattacaacgtgctcttctataccgcccactacacctcgcgcggcgcgctctacctctatcggcaaaacctgcagcggctcaacgagaaccaccggggcatgctccggctgctttcggtcgaagagatatgcgaagagcacacgctcaacgatctggcgttcctagtaggcgtcgagcttatgatcacgcactttcaacgcaccattcgcgtgctgcgctgctatctccagcaccagctgcagagcatctcggagctgtgttacctcatctatgtacaactgccgtcgttgcgcgaagactacgcgcagcttagtgacgtgatctactgggccgtcagtcaaaactacgactacgcgctctacgcgagcacgccggcgttgtttgactttttacgcgtcgtgcgtcagcaggacgccttcatttgcaccgactacgtgtactgcgccctgcgtctgctggcctgtcccgacagacctattatcggtgacaccggcggcagcagtagctcccaacgcctcgtaggcgagtttatggtgcgcgatccgctgttgcgcgacccgcgcgccacccacctgcgccagaaactcatcacccgcgacatatgcgtggcgcggttgcaagcgcagccctcgagtcgacacattccggtcgaacacacgggtgtctcctccgtcaccctgctcaaaatctttagccaagtcccccccgacgaacgcgaagaagacacgttacgcgagatggctcttaaagcgtttatggaagcgaacggtaatcaccccgaacaaatctgccgatccccaccacccccgctgccaccgcgcgactatcctcaacgcgacgagcgggaccgtcaccgtcgcgaccgccgcgacagcggggaatactgttgctgatggtgggacgaaacagcagggcggaacagtttatgatagaaagtcacaggaaagtatgtgttgttttttttttaatgtaccaagaataaaaagtgcgtctacgaccaaagcggtgtgtggacgctcgtcctctctgtcttctccgggtttttttttcacgtgttttttccttcctattttgttacggcaacagcgctgatggcacgttgccggcttcgaacatcgcgtcggtgatttcttgcttgcccggcgtcacacggtgacgtagcagcacgcggctcacgtagcaggccgactcgcggatgacctggccgtcggcgtcgcgtcgcaggcccgagcggttgccgtgacgcagtctgccctgcgcagcgcgctccacgtcttcaaagtagctgtgtagcaggccgcgctccagcagctgcggcagcgagtcggcggcgcgcactacaaagttctcacggctgatctcgtagcacagcacgctgccgtcggccgccacgccggccacgctgcggtcccaactgaaaaggttggcgagtccgatggtgccgatgacgcgcaactgaccctgggtcaccaccagcagcttccagtattctacgtcgcgcggggtgaggatggtctcctccacgtcgcagacaaacaacgtgtagccgcgcggatagggcagatccaggtggcgaccgcgctggcggcgcataaaatcgtctaaattcaaaccgccgtcgggtgcgcgcctactcgtcatcgccgcgccttgtcggtcgatgaccccacggtgcttataacgcgccgccgcggcttcatgtggcgtgacctccgacctcgtgaggccgaaaacggcgtacatgaagacgctcaaacttttgaatgtgggcccggtagcgcaccgagggccccggggcggcgacgacggcgggcccgagttccagcggggccttgcggcggcagcggttggcgtggttgctcagctcggcgtccgagagcgccgagctgaactgcggcagccgcgtgcgatcctgcggcgcgtccccgtgtcgcagcgagtgccagagcaggcgctggacgcgcgccgtctcgggcgtcggcggcgcgcgacagccccggcgcagcttgaaaacgtgcaggcacagcagctcgcgcttgatgcgcagcgacacgctgcggtagtcgggaatccgctgcaccagctcgagaaagtcgcagaaggtctccacgaacgtgtcctcggtgaagcgaatgcgcttcagatcgtggacgtgtttgcgaaaccgcgacagttctcgacgttgcacggggttctgagcgagtcccttgcgcagcagcgcagcctcgcctttaaacagcctgatgagccgctgcacgtccccgctcaacatacgtatacacgccgtgtactcgtgacgtatactggcgcgcagcagccgaatgatacgcagggccagcacggcgttagaggccaggtacatggcgtagccgcgacgcgggttggcacaggcccagcccgcggggagcagaaagtagtcgtcgatcagcgtctgcgaccagtcggcgaagcccaggtcacgtgatacgctgtcctggacgcgggccacgtcgccggccgtgaggtggcggatcgccggcaggtgaaacgcgcccaggtgtcggttgcgctccagcctcagctcggcgtgctccaaacgggaatggtgggacgccaccgcggagggcgacaaagaggagtggtcattgccgtcgtggttaccgtcgtggttaccgccgttgtcgcgcccgtcgccgcactcgcaaaaggccgcgtagaggtccttcaatgccgcttcggctcgcgccataaacgtggcgtggaaaaaaacggcggcgcggtgcgtccggtacttgacgggcaacccgcggcacagggccgccggcaggcagcggccgatgagttcgcgctcctcgggctccagaaacaggcacagggtgccgtccaggcgcaggtacagctcctcggtcatcgagcatagctgccgcaagtaatgggtgcgcgtcccaaaggtcttgtaatcgagcaacgtgcacaccacgtattgccccgtggccacggccagagcgatgcgtttggcggcacgactgatctctggcaagtactgcgcctcgtgcaccagacggcggaaagcgccggcgttgagccagcgaaaatgctgcggatcgggcggcaagggcacgcctcgaagcgcggcccagacagcgaggtccgactcgagcgtcagaccgcggatgtcgtacttgccgtgcgccgtagcgcaggctgaatggaccagacagctgcggcgaatgtacaccatggcgtgcttgggatgtttgggcgccggcgttttctttttctgaccgccggcggccgccagatcctcgggcgtgcgacacaacaggccggcgcgcacagcctcctgtcgattacgaatcggcgtcaggtaggcgcgcaggaactggtgacaaaactcctcatcatcacgacagtcgtcgagatactcgtacgtggtgagcggatcgcgaaataggcgctcgtcaccgtcgtcatggtcttctttagcctgctcctccggctgctgggttggcagtggaggcggcggctgatccacggggttcatgactgagaggaagaagaaggtggcggcgaagcgacgcggagcgacggcggtaaagccagacaccggctatatagctagtcatcacagtctcctccttcacgacgcccccgtgccgctcacgctatccagcacgctacggcccgaaaacacgtactcgctgacgtcgtacgcgggcgatgtatggctgctcaccggtttcgcggcgacggttgcgctcgagtccaacggcgagaagcaaaaacgccgtgggcaacgaaaccagaaggagccctgacggataaaaccgcgcagcgtctcggccaacttaaccagcatcgtaccgtacagcagtacgtgaatgccgccatgcgcgtccataaatacggctttgttcacgggttccatccatccgatgactacaaaatgggcctgttctagcacgccgatcacgaaattgttggcctcgtcggcctcggccacgttccacgagccgaaagtgaaagtacaagcgggcgagccgcccaggcggatcttgctaccggcgtggagctgacatacgcgcagcagattggcgcggtcgtgcagtatctgggagagttcgtacatgcccgcaaaggtgtgcttaaaccacgcgccctctacgatctcatccacgtagtcgcgctcaaagaagctgtacacggcaaagaggccgttctcaaaaaactcgccgaacgagagccccagcacgtacaccttgtcctcgccgggcaggtacgcaaaggcgtgcccgtgcccggagacccagatctcgggcgccgtgtttgcgtccggcacgcattcgtacacactgacgaggccgataaagtacaagcggccagcctggcgcaggcacgagaagcgccggtaggtcttgtgatcgcgcaccaccccaaagtactgagtgtcgcccagcatgatgccgtgcagcggcggccagcacagcgggagccaacgacccgccgtggcgcgcacgtagcgctgcaggtgaaccccgctcgcacgctcgcgcggcttcgggcgcttgtgggtccaggcatcacgcagaccgcgccagatgctgctgaacttgggctgcccgcgcagatagagcgacgagagcgagtcaaagtagcccacgacgagcctgtcgggagacacaagagcgcgaaaatcaaacctagagcgacgacggtgaaaaaaccgaccagaagcgcgtgtctcaaacacgctactttcggttataaaaacaccgtcgccctatttctgggcgcgtgtacactgatgactcacctacgctttttgaacggcagtctcagctcgggattggcctcgtacagcgagctgcggtccacggggccgatgctctcgtagcgaaagtcgtcgatgagcagcgccagccccacgcgcacgaagcccctgaggtcgcgcgccagccgcaccaacttatcctgccccaccagcgccgcgtacacggtgcccgtgtcgccgcagagaatccgcacgcggtgaaagaaggtcttgtcctcggcgccctcaatttcgcccagcggcatgacgggctcgcgcgtgtacaacgaacgttgaaagcggcgcagcatcgaggccgagagccccagatcgcgcgccgtgcgcagcactagggaatgcttctcgggccagatgagggtcagttgcgcctcgcggtgcgcctctacgtaggcgcaacgagcggcggtgtcctcgcaggccagcaactcgcggaaagccagcagcgaacgtaggtagcggccgcgagcggaggcgcgcgagcggcggcacagctcggcccgatggtcgggatgcaccaagggcacgttaggttgcagacgcgcgcagatggattcgtgcaccgggtcgcagcggatcatgcccttggcaaaaaatccggccagatccgaggccaactcgtacaggcagtcctcttgcgcgtcgtaggcgaacacggcgccgtacgcgtccacgaacacctggtaccggcaggtggcgtgcgagaccgtgccaatgagatgcagagctcggaattcgccgaaaaagtcgttctggcagtgctccagatcgatctcggtcagcgagtgcggcgaatgctcgcccccgaccacgtagatgcactgcgagggccagcccagcgacacgcacgagccctcgaagcgccgcaagtaacgccgcaggccctcatagtcgcgtcgcacgcacaggtcggccaagtcgcgcgtgcaaaagacctcgggtaccaagcagcgtttgcgacgcggccgacgcgcgtgcccaggcagaggaggaaggcgcgacggcggcgacgacgaggaggaagacgccgtggccgccgagcagcccttgcgacggccggacatgccggcagtccgcgacgatccacaggagacaaaaaagcagaagcagcagtagcctcggcgacccgctccaccccgtcctccacacgctcagccgcgactgaacgccggggcgcgccgctacttgggtttttatagccatctgccccccgtctcgggcacccgggagcgatctacggagacctgacagcagttgggcaacacaagatagggaaatacaaagacacttttaataaaaaacgagactactttgtgtgtgtgctccgtaaactgtttattctccccctccgcttcgctctggatgggctccgggtccgtcaacacgcgactcgcgcggcaaaaggcacgctgttgacggcgcgagagcccgtcgtgatagtccatcatgccccggagatcgtgcacaaagcagctgtcgccgcgcagaaaccgacgcagcgtctccacgtgctgcagctgccggcgcgtatcaggagccgtcatcgctgatgtcgtcatcgccctgacaggcgcgtagatggcttcgcgagatcatgcgcgttttcaaccgccgtgacacatcaggtccatcttgagctggcgccgggcctcgcgcaggtgttgcacgcgttgtgagcgggaggcgagttcggcttcttgctcgaactcctgctgctcactgtccgagagggtgcgataaaaggcggcaaagtcctccaagtcggctacatgcgccctgggtctgacgctccaaagcgtacgcagtctgatgaagcggacccatcgagcgtcacggcacgccgtcttgaacgcggggcccgggaagaggttcttctccccggcgcgctcgggccggcgaggccgacgcggtttatatacaccgtctcggacggcgggacgccgagcccgcgccgcggccgctcatccggagacggcggaaaccgcgacgccggaggaaacggggaccggcaacgacggcggtggcggcgaccagattatgggggacaaacccacgcttgtgaccctgttgaccgtcgccgtgtcgtcgccgccaccgtcgtcgccgctgccgctcgtcagcttcacggagctgttgttaccgccgccgtccgtcgccgccgctgcggtggcggcgacagcgacgagcgaggtgggcgagaaaaccgcggagcaagaggtagcggctgcggatccggagaccgggaatgagagaagagaaaacagggagaacgaaggaggggagacgaggacgacaggcaccaccgcggtcaaaaggtcgcacgacggtatccctcgccaactggcagagcgcctgcggctgtgccgccacatggaccccgagcaggactatcgtctgccggcgcaggacgtggtgacctcgtggatcgaagcgctacgcgacgcggaccgcgacaactacggtcgctgcgtgcgccacgccaagattcaccgttcggcctcgcacctgacggcctacgagtcgtacttggtgtccatcaccgagcagtacaacacggcctcgaacgtgacggagaaagcttcgtacgtgcagggctgcatctttctctcgtttcccgtcatttacaacaacacgcagggctgcggctacaagtacgactggtccaacgtggtgacgcccaaggcggcgtacgccgagctcttctttctgctctgctccaccagcgagagctccgtggtgctgcaaccgctcatcaccaagggcgggctctgctcgtccatggcggtttacgacgaggaaaccatgcggcagtcgcaggcggtgcagatcggttttctgcacacacaactggtcatggtgcccttcgtgccgcacgcctgcccgcattacgccgtgcctttcacgacgccgggaaagccgggctgcggcggtgctccgagcggcgttgcggggttggaggaggcggcgccctttggacgggtcagcgtcacgcggcatggcgcgacgctgctatgtcgcgtggaccatctgacctggatcagtaagcgcgtaaccacgtacggacacaaaaaaattacgcgctacctcgcgcagttccgcggcacgatggacgacgacgaggcagcgctacccggcgaggacgaagcgtggatcgcgtccaaaaacgtgcagtacgaattcatgggtctcattttcaccgtcaacgtggattcactatgcgtggacgcggaacagcgccaactgctgggcaccgtggccacctccttctgtcaccgcgtctcggacaagatcacggcgcgcaacatgccgcgcgctttttccttctacctgctgacgagcgcgcagcgcgggtacgacctgcgattcagccgcaacccgtcactcttttttagcggcgacgcgctcaactgtccgcttctcaatgagcccaacgtgttttcgctcacggtgcacgcgccttacgatatccacttcggggtgcaaccgcggcagacggtggagttggacttgcgctacgtgcagatcacagaccggtgtttcttggtggccaacttgccacacgaggacgccttttacacggggctcagcgtgtggcgcggcggcgagccgctcaaagtcacgctgtggacgcgcacgcgttccatcgtgatcccgcagggcacccccatcgccacgttgtatcaaattaccgagggcgacggtaacgtgtactcgtacaaccaccacacggtgtttcggcagatgcacgccgccggaacaaccacgttctttctgggcgacatgcaattgcccgcggacaactttctcacgtctccccatccctgaccctccgtccgtcctcctttcccgacacgtcactatccgatggtttcattaaaaagtacgtctgcgtgtgtgtttcttaactattcctccgtgttcttaatcttctcgatcttttggaggatgttctgcacggcgtccgacggcgttttggcgccccccatgccggcagaacccggttgcggccccgtaccgctcttctggggcgacgataggtcgaaagccaccgttttcatgcccgtcgtgctcttgacgggggaacctacggcggcggtccccgtcgagcggcgtgattgcaaagccgcgctcgcccccggtttcaggatggagggggaggccacaggcggcgcattcgatacgctgcttttggccgtagacgacggtgggtaaacggtggttaccgcgggatacgtcggcgtggtcgaggcggcccggctgctgccggacaggcgacccggcgcgctaccgctcacggggaccgagggcggtcgacctaccaccgccttgccgcccaaagtaggtttcaaggaaggaacaccgacgcggctgccccggcctttcaccggagacgggggggcactcttggccggggacggagaggctgacgaaagcatggacagcggcgatgtggcgggggacacgacatcatcctccgtgggcgacaaaacggacgccgaggctgacggctgtcgagccgaagaagcggaagaggttcccgcgccagaagtcacgttccttgatgacgtcgttttagacgaagccggttgaggttgcaacagcgtggcgggtaccgtcgacggcgtgcccgacacctgtttctctagccttccctgaaccggcgtcgacgtcaccgtctgcgctcgggcggacgcgtgcggcgtcgcgactcgcttgcccagcaccggtttctggctcgtggatgtcgtcgtcattggagacgataacttagctttacgtattctggacggcgtcgactgctcgggcgtctgactgggaggcgaaatgacgtcgttgttgtaatcggacgacggtgttgtgtgtcccaggctgacgacggagccggtgtccgaggagtcgtcgtcttcctcctcgctgtcttcgaccggtgactctgcagtttggtcccttaaagcccaaacctcatcagcggcgtcccgagacgctgtttgtgtcaccgcggcgcgtggagtcgacggcctccgaggggtggtggacacggtgttttgagaagccgtggaagtcgtaggcatcctgaagggattgtaagccaggtgaggattcttgagggcccacgcgcgttcgcgcggccagttggcggggttcatatccccgggcaacggcgccgtcggagcccagggcgagttaccgttgaccggggtttgggtacccgcgaaggtaggtgtcggggccggagcgggggccgtggaaggattgacaggcgtcggcgtgaggatggcagcgccggcgccagcaggaacgttaactccggcgccgaacgtcaacgtcggttgctcgaacttgtacgcggtggtgacgggcggtttggcgctcgtctcggtatccgtgatgtccaccagcgtgtcggtgaaacgcggatcttgacggttggggggatagccatccgagctgtcggaatcctcgtcgcccgagaaaagatcccctctggtctccgtgagcggcctcacgtcccacgcgctgtcccgacggacccttcccgggctggccttggtcacctgcggggagacgagactgaaagccgcgtgacgctgttgttgttgcgggatgttcaagggaccgctggtcggtttctgactgcccgaggataacaggccgctgaaaatgctggaaacaccgccaccactagcggcgcccttgccgctagttcccggtttcttgatgggcgtaaagatgtttttctcgtcatcatcatcgtcgtcgtcctcatcggcactggagccaaagagcctccgggaggcgctcggtttacgtgccgggggcggtggttgctgctgacgttgctgcaggttctgctgcctctcctcccaagccttcagctgctgtttctcacgctgcaccacctcgtcgtccacccgtttctgccgctcgcgacgcttttcctcttcgtcgtaatagccgacggccgccgaacgggcagcgtgggcttcggcggccggtgccagagaaccatgggcctcgaagcggaacggtttgtgtcccttccagggactggcgatccagctccagccgtccagcggctgcgtggggacatgtttcttgggtaccgacgagaaggccgaaccgccgccgagcgagaggagattggcgtcatcgtcaaactccaacgacggcgagcgcgcgcccaaaaacgtgtgcgccgactgtgggaagctgtccacgtagatatcaaagtcctcgatgagcagctccaacagcgtgtcggccgagtcgccgttttccacggcgtgcttgaggatattgcgacagtagttggaatcaaaggaaaggcacatgcgcagctccttgaccagcagcttgcagcgctcctgaatgcgcgccagacatttgcgctccagctcctcccaagacctgcgcacgttcatgatgagacggcccgtgtacacgagcttgttgacggcgttgaccagcgccgtgttggcgtgccggtccaggttaaggtcgagcggtttcacgcagaacatgttacggcgcacaccctccaggttttcttcaatgcgctgcacctccgtatccttgaggtgcacaaaggcgatgggttccgtctggccgatggctgtgaccagcgtctcgcgcaccgacatcttggccagaatgaccgcgcttacgagcgcgcgttccacgatctcggcatcgtggcgcacgtccgtatcgaattcggtacggtctagcacagccaggtggtcacgcgccttaccacgatcaccgaacgggtaagtgtagccgcgacgcgccacggccacgcaacgcacctcgaactcctcgagcactgaggagaggtcggggttgtgaaaacgcagctcgcggtagtatcccaaccaaagcatgagctcgttgaacagcaccgtacgccggtgcaggcgtttttcgccacattttttcaggatcttggggtgtgcctcgagatccacgtcgggcttttgcgtgagatggcgcagaaagttgaccagggctaccacatcgcgccgctgtagaccgataaactgcaaactcatgctggcttttctccagaacccggaagcgtcgtcgccccggactgcgcccgcggtctgctattcgcccacgatggacaccatcatccacaactcggtgagcgccccacctagagggagggggggtagtttaatagcggaggcggatacgcggttttcttttaagcgccgctgacttgtttcttctgttttttcgccccgtgtgctgttccgcccagacccgcaacaacactcctccgcacatcaatgacacttgcaacatgacagggccgctattcgccattcgaaccaccgaagccgtactcaacacattcatcatcttcgtgggcggtccacttaacgccatagtgttgatcacgcagctgctcacgaatcgcgtgcttggctattcgacgcccaccatttacatgaccaacctctactctactaattttctcacgcttactgtgctaccctttatcgtactcagcaaccagtggctgttgccggccggcgtggcctcgtgtaaatttctatcggtgatctactactcaagctgcacagtgggctttgccaccgtagctctgatcgccgccgatcgttatcgcgtccttcataaacgaacatacgcacgccaatcataccgttcaacctatatgattttgctattgacatggctcgctggactaattttttccgtgcccgcagctgtttacaccacggtggtgatgcatcacgatgccaacgataccaataatactaatgggcacgccacctgtgtactgtacttcgtagctgaagaagtgcacacagtgctgctttcgtggaaagtgctgctgacgatggtatggggtgccgcacccgtgataatgatgacgtggttctacgcattcttctactcaaccgtacagcgcacgtcacagaaacaaaggagtcgtaccttaacctttgttagcgtgctactcatctccttcgtggcgctacaaactccctacgtctctctcatgatcttcaacagttatgccacaaccgcctggcccatgcagtgtgaacacctcacactgcgacgcaccattggcacgctggcgcgtgtggtgccccacctacactgcctcattaatcccatcctgtacgcgctgctgggtcatgattttctgcaacgcatgcggcagtgtttccgcggtcagttgctggaccgccgcgctttcctgagatcgcagcagaatcagcgagctacagcggagacaaatctagcggctggcaacaattcacaatcagtggctacgtcattagacaccaatagcaaaaactacaatcagcacgccaaacgcagcgtgtctttcaattttcccagcggtacgtggaaaggcggccagaaaaccgcgtccaacgacacatccacaaaaatcccccatcgactctcacaatcgcatcataacctcagcggggtatgagctttcctgttactttattcagaaagcaccagaacccgtcgccatttcccctcatatacggtacacgtccccctgatctgtcatcacggtacacagatttcgcccgactgcggacgccgacggccaatcgcgtggcgtaggagtggcgccccggcttcattataacgccacgtcggagcccctgcgcgccacaacgccgtccggcgcaacttctgtctcggcacggtacgataaaaacaacgtcccccgtcgacgttgttttctccgagcggtgatcgttcccgtccctctcctccctccgcggcccccacggcggcggcctgctcgcacggacctatactattaccgccccaccgccgtcgtcgtcatgaacttcatcatcaccacccgagacttctccaacgacgattcagtcctgcgagccgccgagatgcgtgacaacgtggcaggctcgatttccaaagcgtacaagggcacggtacgcgccgaaggcaagaagaagctgctgctgaagcacttgcccgtgccgcccggcggctgctcgcgccgcaacagcaacctcttcgttttctgcaccgagcgcgactaccgcaagttccaccagggcatcgcacagctcaagcgcgcgccggccgaactggacccccacgagatccagcaagtcacggccagtatccgctgccgcctgcagcccagtctccgcgagccgcccacgccggccgacgagctgcagacggctgtgtcgcgcgtgtgcgcgctcttcaaccagctggttttcacggcccagctgcgccactactgcgagcaccaggacaaggtggtgagctacgcgcgcgacgagctgactaaacgctgcggcgaaaaatcggcgctgggcgtggaagtgcatcaactggtagccctgctgccacacgagcgccaccgcgaactgtgccacgtcctcatcggcttgttgcaccagacgccgcacatgtgggcgcgctccatccgtctcatcggacacctgcgccactacctgcagaacagcttcctacacctgttgatgaactcaggtttggatatcgcacaagttttcgacggctgttaccacagcgaggcctaccgcatgctcttccagatcggtcatacggactcggtgtcggcggccctggaactctcacacggcgcggcggccgggccgcccgaggccgatgaaaacaacgacgagggagaggaggacgacgacgagctccgtcacagcgacccggcgccgcttcacgagtccaagaagccccgcaacgcccgtcgtccccgcacacgcgtgccgcctcacgagcaaaagcccgaagaaaacgaggaggaagaagaggagctgtttccctcctgcaaggcaaccgcagcattcctgcgggcagaaccctccgtctccaacgacgacggcaacggcggcgaacgctgcgacacgctagcgaccgccctgcggcatcgcgccgacgaagaagacggacctctagccagccagaccgctgtgcgggtcgccgcgaccccctcaccttcagtcaccccagcccttacccccgtcacgtcccccataaccccgttgtgtatttaacgtcactggagaacaataaagcgttgatttctcaagttccgctctggttttggtttcgttttcaaagggagccccatcatggcccaaggatcgcgagccccatcgggcccgccactgcccgttctccccgtggacgactggctcaactttcgggttgatctatttggggacgagcaccggcgcctgctgctcgaaatgttgacccagggctgctccaactttgtggggctgctcaactttggcgtgcccagccccgtatacgcgctggaggccctggtggacttccaggtgcgcaacgcttttatgaaggtaaagcccgtggcccaggagattatccgtatctgcatactcgctaaccactaccgcaacagccgcgacgtgttgcgggacctgcgcacgcagctcgacgtgctgtactcggatccgcttaagacgcggctgcttagagggctcatccggctctgccgcgctgcgcaaaccggcgtcaagcccgaggacatcagcgtgcacctaggcgccgacgatgtgacattcggcgtgctaaaacgagcgctggtccggctgcaccgggtacgcgacgcgctggggctgcgcgcgtctcccgaggccgaggcgcgctatccgcgcctcaccacctacaacctgctgttccacccaccgcccttcaccacggtcgaggcggtggatctgtgcgccgagaacctgtccgacgtaacacaacgtcgtaaccgaccgttgcgctgcctcacctccatcaaacgcccgggctcacgcaccctggaggacgcgctaaacgacatgtatctgttgttgacgctgcgacacttgcagctgcgacacgcgctggagctacaaatgatgcaggactgggtggtggaacgctgcaaccggctttgcgacgcgctttacttttgttacacgcaagcccccgagacgcggcagactttcgtcacgctggtgcgtgggctggaacttgcgcggcaacacagcagtccggccttccagccgatgctgtacaatctgttgcagctactgacgcaactgcacgaggccaacgtgtacctctgcccgggatatttacatttcagcgcgtacaagctgctgaaaaagatccaatcggtctcggacgcccgcgagcgcggcgagttcggggacgaggacgaagagcaggagaacgacggcgagccgcgcgaggcccagctcgatctcgaagccgatcccacggcgcgcgagggcgagctctttttcttctccaagaacctgtacggcaacggtgaggttttccgcgtgccagaacagcccagccgctacctgcgccgacgtatgttcgtggaacggcccgaaaccctgcagatcttttataacttccacgaaggcaagatcaccaccgagacgtatcacctccagcgcatctatagcatgatgatcgagggcgcctctcggcagacgggcctgacacccaagcgcttcatggaactcctcgacagagcgcctctgggccaggagtcggaacccgagatcacagaacatcgcgatttatttgccgatgtttttcgccgtcctgtgaccgacgcggcttcttcgtcgtccgcgtcttcgtcgtcgtcctcagcatctccgaattctgtttcgctgccgtctgccaggtcgtcatccacacgaaccaccacgcccgcgtccacgtacacctcggccgggacttcttctaccacaggtctcttgctctcttcttccttgtcggggtcgcacggcattagctccgcggacctggagcagccgccccggcaacgacgccgcatggtcagcgtgaccctcttttcgccctactcggtagcctacagccaccaccgacgtcaccgaaggcgacgcagcccgccacccgcaccccgagggccggcccacacacgcttccagggacccgacagcatgccgagcactagctacggcagcgacgtcgaagacccgcgggacgatctggccgaaaacctacggcatctctgaaagcggtttttcctctttttctacgtgtctgtctcaagatgagacgtcgatatcaataaaaataccgtcgacgtggtttttttaacagtgtggttttctttattgactagcgaagtacacagtttacgagtagaaaagacagggaaaggttatataaaatgctgtattatatacaaaaacatgcacataaacaaacgggaccatcgtgctcatcatcccctccttgatcagttgttcatgtaaacgtgtggcggggtgaggggcggcatgccgttggcggcgccgggaataatgtgccgtcgaccgacgtcgcacaccttgaaacgccgtcggcgcacgcagcggtcgcaggacgggatatcccagaggaagcccatgtaggtctcggggtcctcgtcgtgaaagcggtaggagagttcaaagtggtgcaacgagcccgtccgagctcgcagcttctggcgaacaccctccacgtcatcggtgcacagcgacagtgctgggctgtcacacagggcctgaagctcctgcggccacaggtgcgtggccaggggcgagtccgtcgtcaccagtttgacgcagtgcatcaggttctcggtgatggcgtcgtacaggcgactctcagcctcctcgtgcgtcatcacgtttcgaggcagcgacagctcgtcgtcgtcatcctcgtcaaacatgatcatggggtcaggggtttttttgggatgttgacaggtgggtgtcttttccagacgcacgatggcctcacgccggccgctgaaacggtggtttcggtgtcccttctttcccatgacgcaggtgaacataaccacgtcctcggccaaacggtagacggcgtccatggcggggtcgtagccgtagacgacgccgaaagtgtccaccaagacgtactggcgtacgaggaactctttgcgttctggcacctcgtggcccagcgcgcccaacaactggtggtaacaggtgatgcgcggcacggtacggatcatgagctccatggtctggatgctgccgcccgcgcggacgacgctgaaggatgtttccttgaacttcataacctctgtgttgtgggtccagaaggcgaaatgggtgtcgggacactcatcgaaagggtcgtcgatggtgtaggaagcgtagcctcgcttggtcacctcggccgacaggctctccacgtcaccgcggtagagcatgacggcgttccagtagtcgtcgtactgcaccatgggccgctggtagtcgcgcatagtgtggaagtggtcgcggtgacgaaagccgttccgcagaaagtccttcatggtgggtgccagctcgtagacgcagtcgcgcaggtcatcgtagcagtagatgccgccgcgctgcccgatgagcacgatgagttggtagcgcataaagcccggaccctcgacgaagccaaaggggtgcaggtactcctgacagcagacgtaagcacctggtagagaatagaaaaaatccacgcacgttgaaaacacctggaaagaacgtgcccgagcgaacgtcctctttccaggtgtcttcaacgacgtggggcttaccttgcgaacagacggtgcccatcttgcccacgaagggccccagggcgctgcgcgaacggagctggatgaagcagcgttcgggccaggccacgtgcagccgggtgccgcattcctgctccagaaagtcgttgagaccgttaaagtccccggctcggatggcgatgcagccgtaggccatcagcgtgtcccgtaggtcgtccatgacggactcctctaccttcgctcgccgacgctgcgcttctccagccaccgctgcggtcgacagactcctccgtccgccttcggagaactacggcgcggcggcacggcctttatagacactatcagcgttgacgtcagacgatccgatgaacgtcgttttttgtgctggaacttccctcgtcccgacaaatgtagcggaaatcttcaagcaaatcgcgacgaagtccgatgaggaggatgcaaaagaggctgagcaacgcgatgctgcccgccgccacagtacatatgctcaacaacgcccagtgtcccaaggcgcgacttttggctcggaggagagccgaacggcggtttctccacatgacggacaacgtggtccagtacgtccatcctttgcattccggtgtccagacgggaagcgttgtcatgttatttcccgtaactgtcacgttatgttttgttttgtttctcgtgagcttaacggtcctcttgagaaatcgcgggcacatgtcttgtagaaagatataatcactttccgcgtatttcgtcagtgttgacatcacggtggtagtgttttctgaagaagtagcgttgtcagtgacgtttgtttcttcccaacgtacgtatgattcgaacggactcgtgtgcgctattgcccgcaacacgtagctgtggccggtgaagttgagcgtcagttgtcccacggtcacgttcgtgtcattcctaaaacatgctacttctccgtgaacttccgtgacgtttatctcacgactctcgttcaagacacgcaggggaaaccagccttccaggtgatactgaaaaccaaatttaagcatgacgctgtgccatttccgtcgtgattgattaaacgttacattcaagggcagtctggcttcggtcccgagacaggggccgttgtagatttgcgtgtgattgcgtgtgcagtttaggtggcagttcatgctcgtggtgttggaagtgcgattaacgtccgtaccgtggtacgtacatcggaccgaaacaccgtgtcccgtgctccaaagcagcgtcaacaacagccacacagaaacctacgtggagacgacacgggactttttattgacggagactcacgtttctaccctcccctttcccgtaggtaaaaacccacgtttatcacacacgttgtttttacctgaaacccgcgcagcccgtggacgcgacaaaaaaccgcggcactagaaagaaaatgaaacaagtatgtttattaagcagcatgtggggctaataggggggataactgaggtatagcaactatgaaaaaatactacaaaaaaaaaagctgaacatggtcatctagcagcaaagttctccttctagaccacgaccaccatctgtaccacgtcgccctccccggccgtgtacacgacatccttcaccacgaccggcggcagcggcggcgacgaggacaactcgctctcgacggaggccgggacgacagaggacgggggggtggtggcggcggaggacgaaggggtggcggcggcagcgggatcttcttccgacacgggcaacggcaggctcggcggcgcggacagcacccgttgcgccggggcgtgagaaggctgagccccggtggcctggatgtgggccaacgaattggctcgcagcgagtcgcgatccacgaaggtcataggaattttcccttcgcggatccgccgctcagattccaggatggcgcgcacgtagctgttcaccgacttggcaaaagtgcgcggcccctccgtattcttgtcgcgacgcgcttccagcacctgcttttcgtagtccagctggtggaagaccatcaccaggtcgtccatagtgtgcgcgtgctgacggacgtgggagcgcacctccaccgggaacaaagcgttccaatactccagcacgatagcaccgtgccagaactgcgccatgctgggcgccaggaaaaacaggataccggagtcgtaggcgaacacgtcccacttgggcgtcatgaacaacaccagctgacgcgtgggccgcaccgaagcttcctcccaggcctcgatgaccccgaacatgatgagctcctggtccaacggggggcagtgtcgctccagccaactgatcttgctcaggttcatctgcagaaactcgtacgaagggtcgcagatgcacacgtagagacccgagtcgtgccgcagcctggctccgcgcttcatcagtttcctcaccgcgtagcgaagcgccaccttgcccaacgccgacgcctggatcagtccccccacgtccatctgcgtctgtcgccactcggcctcgtccagcaggctcatgatagcggcagtgctatgcgtggtcgtagtcatcctttctatccttctctatgaatagcagcaatagcggtaaagtcccttcttatactatcccggagtctgtggtttttttgtttacccctgcttactggtgagactgctgggggccgttgtgctgcagcatccgagctcgttgccgccgttgccacaggaaccggtgtctccgcagggcctttttgagggcttcgcaggcttctcgcgcaagtcctgagaggccctcggcgtcgatggggttcacctcgggcgtccgagcctcgttttcttcttcttcatcctccctttcctcctccgtgtcctcccgctctgtgtcctccgttacgctctcctccccggcctcggccaagagcgcggccaccaagtccacggaccgctcggtctccgagttctcaccgtcaattacgccatgttggcggcgtaaccggtgccgagaacgccgggtgagcgcacatgcttttttctttcttaaccaaggcgggagaggatcttcaaggcgttttcgctggatccagcggtagctaaagtaccaaaaggccagcaggcccacgctacctaacagattcacgtagactggagacataattaaagaaagaagtgaaacccgcgtgtgggtctcacgtcgtcttgaaacaccgtcttatatacatgaagatgccggacatgacgcgcccaagacacgtggggttttccccttaggcgacccggtttcttaagatgtttttcatcttcgcacgcgatgtactacatcaaagggtcggctgaccgaccgcattgacgcacagtttccgagtacgcgcgtctcggagcacctgacggtgagccacccaactcacgcggataggggacaacactgacgtgaggggcgattcacgtcactgacgggaataagacgggtgagggatttccacctttttcttaagtgtgactctctttacggtaaatcgcacctgtgacctcttaacccctcctccctggtacccgataaccgtgaaaaacacacaccacacgtcacgacaccgatcgattttctttattcttagtgtgatgataggtaagggcactcgtgaggatgtgcaattatcattatcaagcctttttcaaggcgtagtgatgatcgttgggcagaacccccaggctcctagcgatctgggaatagaaggaggagaacgagcccagggccagaatgcccacagtgtacatggcccaggtctccagaccgaacgtggcgggtcgcagcttcagatggtaggccacccgctccgagagttgtgaatgctcgttcaggcaacaggactgcaggtgggtgagcccaaaagcgctttcgtttacgccgcgcacgtgcaccgtctgggccgggcaatcctggtgttgcgcgcgaaagtggtcctgacaggaaattccgtctacgtggcggcgcgtgttgttacccacttcgatcaacaacgtgttatcggcaggatgatgcgagaacgcgacgacggtgttgttggaggtctggcggcaacagtacacgtcgagcgtcatgagggccatgtcgccttggtggtacacggcgtacgcccaaccctggaacacgagcggacataacggaccgtgagcggacgtcacggcggcggttgttaccgtcgtctcggcaggagaacacaataaactcctgatcctcatacacaggagtccaaccgtcagaattaaagtccgcggagccataaccgcgcaagtgaagccgatacgagtgttgctgaatttgttcattctgccgactgttgctcacgagcgttcggaggcggtgccacaggctgttggccattaaaaagtcctggcccgaatgacgacaagacagagcccgaggcgaagaaaaaggcgcccgtcatgaagacgtaggcaggggaattcccatatttttatggcttcttttaaaagtctgtatccgactccatccggcgcttttcccaaaccgtggtctcctcgtcgtccgactcggtacccaggaggtggtaagtcttttgccgcacgtagaaagctttcaacgtggagcaaaagatgagaataaagaccccgaaaacgaaacaaaccacgccgatcatgccgatgcagacgttcatgtcgacgtagccggcggtgctgttggcggtgcggcaaaagagtgtcatgtcgtgcgtgcacaaaaaacaacacacaccacaggccaggtcgtagcgtagttattattccgtagcagcaatgatggtacagtcaagcacatgctctatccccgttaccccgatgatgcttgcgtccccgttgttatattggcactgtcccggttaatcaccacggtgaacaccacggccaagaaaatgatccctaatatagcgaccactaagagagcaaaagtccatttccagccgttgtcaaagtacgcccccgtggtgggatgcatggtggcgggcatttccatcatgtccatgtcgaacgtgtgtcgcggcgacggcgaactaaccaggcagtacgggggtcgatagggcggtgggctgcagtcgggtggtggcggcggtggcgtggaaaccgtcgtcgggcacagacccatggcctgctcgtaggtggggggcgcgtcgtcgtgatcccggtcgcggagcatcggcgtgggctccatgtcggtggcagtgacggcgacggtggtaactgtggtggagacggtaccgacggcgtccgcggttcaccttcgagcaaagagccccttctttttgcgcaaacgacggcaaaacagttctctgggacaaccggtggcgcggtaagcgggtgccacgctttcagggtgggtaaaacagtcgcgggcgaagcagtagttgttgcagaaccgcaagaacccgacgcgaaagaagcccaggagtccgcgcgccagaaagtgcgcctgccgcgtctcgggatgcacgccgaagacggcgccgctctcgttcaccagtatggagatgtccaggcgctgctgcgactccaccggcacggcccgcaccacaaatacctgcagcacgttcagcgagcacgtctcttttaaccagttgccgtgggccggatcctcgtaagtctggctcccgttcaagacgaccgtcgtcagcgcctcattaccgtctcgccagctgaagatggaaccctcgcgcttcatgcacaggcgccacaaggccagcaggtcgcgcgccaacatgaactcgcgacccacgtcgccgccggtctcgaagcggacatagcccagttcttcgcgcagcggcgcgtagttgcgcaggccctcctgcacgaagccgcggaaaccggaccgcgacaccaggtacagcgattccaccacgggcgagtagacgtagacgcgaccgccttcgccgatgagtacgggtagcggtgggcggccgatggcttcgcaacgactcacagtgcccaccggcagcaggaacttgtcgcagcacaggaaggtcttctccaaacctttaatattgagatgtccaaagtaaccaacgcgtaacaggtcgcagtaggtgaagaaccaaccgtttggccagctgagacgcagcaccgtgccgctgacgcgacgaaccagcttctgcaggtccttgcgagcgtcggaggtgacagagcagcggaaggtctcgttaaccagctcgacagccagcgcgtcctccagcgtgcgttccttcatctcgtcgttgatgctctgacggcgccgccggatttcgtcgaaacgggccgcggaggcggcgaccgacgcggaggtcgtccgaacgccctctgtgacgctgccgtccggccagtcaagaaagctaaggctggcgctgcgccgcctaaagtgtccgatccgcgcgggacgtcgctgagggacggtggctggtctgctggggcgggtacggccgcgggtgtccgcggacacgttagttatacacggaattgagtcacgtggcacgttgccagctgaaaccgccgtcgtctccgccggcgttttctccatcacgggaccgcgccgtgcgcgcgttcccaggcacgcggcccgcgctctagccgcacttttgcttcttggtgttagggacgaactcgaacgttacagaatcctcgctgtcgctctcctctttcgcgtcgttgaagtaattgccggagttgcgatccaaaccgccgcctcctcctcctccgccgccgcccgatccacctttggacgtcaggtagctggtgatcttgtgctgctcgtatttttccttggaggaaagaccgtggtcgtgatcaccgccgccgccaccgctgctcattttccgcgtaccggaaccaccgccgccaccgcggtcgtgcttcttgccgccaccgccgccacctcctcccagaccgccgagacccatgggctcgttcatgagatcgttatccagacccgggccgtcgtcatgcagaccgccggcattggccagcgaagagaggctgccgccaccaccgccgccgccacgcgacttgccgctgttcccgacgtaatttttgtcgaagggatcgccacgctggaaaggttcctcggtgagaaaattctccacggcgaacagaccgttgcggctggccacgtacaacagcgtgtcgtgctccgtaactatacgcaacgtgcacggcagtttggtgacggcgcaattgagcagcgtctggtagaagttcttcagctgcacgttgatacgcatgtttttcacgccgtggaaactgacgcggttattggctgtgaattccagctcgctgccgttggtcaggataaacttgatggccggtggaccggcgtgcaccagaatctgcacggtgcccgtagggcagggcgcttttttaacgttacgcttgacgcgggtatgcggcccgatccacttaagcaggtcggccaccacgccgaaatctagatccacgtgcacggccgaattctcgctttcgcgcacaatgtcttggccgtgcacgcaggccgagctgaactccatattgaaatcgggcgcgcacatggagatcttggccgacaggtccgagatgtcctgcacgtagaacttggtcaggtccttgctggaagtcaggtacatgaaattacccagcagcggcgtggaattgttaatggtcttgggctgaaacgacttgtcagtgatgtagaggcatgagctgttaaaagtgatttttgacacgcagtgactgcgtaccgtttgcaagataagcgacggcgtgggcaagaaggtaaccgtggtgttctccttgagcgcacggatcacagatcgcagctgctggatagccgtcttgtacggcttcagccgcagcgccagcgtcggcggctccgagaggcgcgtcttgcgatccatcccggacagcgtgcaagtctcgactaaggagcgggcgcgagcgagcgaaagttttatagagagcacacacgacgaccgggaacgctgcgaagacgcccggcgtctaataatacagccgcgccgagccagcgggcccccgactaagaggcacagtacttatatactccgaccttaaagcgccagtggtaccacttgagcatcctggccagaagcacgtcgggcgtcatccccgagtcatagtagaaaaccagggccacgcactggtccacaaacacgctcaggttcacggccgccatttccacgtcgttttggatcgccggtgccgcctggaacagacactgcgtcgccttgccctcctcctggtgctgctccaaccacgcgtaattcaccacgggcacgcgcagcggcctccgcaccacggtggggaagtaacactcacggttgggcgggcacaatgaccacaccgtctcctcctcgaacacggtgccgcgcgaagcccacactgacggcgtcacgccccacagatgcgccacctcgtcgtcgggacccaccgccagaaactgacagttgcgcaatccgaactcgagcatgtcggcgcgcagcgcttcccagcgcgcgctggcgatggagagccgcggcaaccgatacaattcgaaaatgaatttgccctcttgatagatggtgcgttcgaaccactcgcagcgcggcaaacccgacttgcacaaatcgacgctagcgcgcaccgcggcaaagtacatgtgctcaaagatgcgctcgatcaagtcccaagaggcaaagtacgtgaaccctaaccgcatgagcgccgtgtgcaagccagccacgccgatgtgcagcggacgcagtttttccagcgcgctctctacccaccattcggacgccgacattagcgcgtccaagcgcgcgttgccccaaaccaccgcctcggtcaccaactcgcgcagcacgctcaaatcaaagtaacgtcgcgtgttccccaaaaccacgtcgggtagatgcagcttctgctcgtcgctacgcgcaaacacgcagcgagccacgttcaccgtcagccgctgcaccggcatgtcacactcgccaaagtggcacgacgccatatcgggactcaagcacggcggcaggcacacgctgtcggccataatcgagtacttgactacgtgatggacaaagaccaccgaggcacggcccttgagcgcgcacagcaacatctttttcagaaaatcgtccgtgttcacgaccaccttggggcacgattgctcgcagcgcgaatactctttctcgaaagccgactcctgacccaggtccgagagccgccgggagacaggccgcccaaacagcgagtagcgctgctcacgcgcacggtagcgcttcattaacacgctaggcacgttgaaagcgtagcaaacccccgtcaactccgacgtgctttctttgagaataaagttaatcacgcggatagcggccacgtcccacatgtccacaaacacacgtaccacgggtcgatgcacctccttctcgcgtatcaaatcgcagtatccccccaggcaacgaatcacgctgttcacatcggcgttaagtcgcgttacgttcaccgacacagaaacgccgcaactcaaggtactcatccacttgcacatggccgcccaactggcgtcacgcgagaaagggtcggccgagatcagaaagtcgtactgcggcacgcgatcgaaacccacggtagacatggtgaaggtggacagcgacagctgcccatcgcgacagcgcttcaacaccgattccaacacctcgccctcgaaacgcgcatccagatggaaacgatagatgcgcgagtgcctactgttctcgatagcggccgtcaacgccacggcgatgcgcaaaaacacgccgcccgggctctcgtcctgtccgtgcagttggcgacacaccttatccaaacacaaaatggccgcgtacaagccccagcaaccggccaattccacaaaacgcgccgtctcctcggccagcttgggtagatcctccatgtgacgcagcacaaaacggcgcaccgactcatcgcacagctccgaagcgtaacacagtggcgtgcggctttcacgcgcccagttggctttgaaataaaagcgacccaacagcaggtcgcaacgcggcgagtgacgaatcagacagggaccgtggcgcataatgagctgaaatagcctgaaactgcccaaaccggcactgtgccgcgacacggtgtccatctcgcgccacagcgcgttcctgtcggacggcagctcccgcgccggctcctgtacgccgcaaaagcgaaacttgccccagtagccgtgacaatgacactttttgcccatcaacatgcgcgtagcttgtatcggcggcgatactttgcagagcgaagccccgaaatcgtcctcctcctcgacactgtccagctccatcctggtcgcgccggccggattaaaggtgctcagaccgctactcacgcgtccaccgcgactgggcacggcgggaccgctgtcacgcgtcaacgacagcacagacggcgtgccgtcgggagacggcgactcgggacgccaactgacgacgccgccaccactcgtaaaacccgctacacatgctacaccgctcgatacgttggtatttccagcggacgcttccttgtcacccccgggcagcggcccctcctcgagctcgctgtcatctcccccgatagtatcagcggcgacctctgccgacgattcctccgtctcggtttccgcgctgcggcttggaatcctacctggccggcaccgatgtgcgggcaccgaggacacccgctgttcctcgtccgcgtcagccggattcataagtttacgaggaaaataacaaagaaatcaggtagatttcaataaagtgagtctagatggcgccgacaactacggtttataaagtctgtgtgcgatgtgtttttttcttctgtgtctcctccccgtatgctgtcagcgccgctcagacgaattctcgaaagtctcccaattcgacgctaaagttgtccaaacggacgacggacagtttgagttctttgtgtaccaggaacgaggtgtgaatgtcgtcagccaggcaccagcccagcttttgtatgaccccggtacacagagggatctggcgcgggcgcgtgatgcgacggttgacaaagctacagcgctcgcgggcgaactttccgcgtgcaacgtcgaccaaggtctgccagtgtgcgatgctggaggtgagcacgtagatgccgggacgtgtttcgggcccgtcatagtcatagacgatgattaaatacacgtattgcagccgtccccgggtctcttcccacgtcaggtacatgtctttcggtatcatcaacgcgaacacctccgttttgagcgtgttgtaaaggtagccgcgcatgacgcaggtgagcaacgaggtgatgcccagcgagacggtcttgacgcagcccagcgtctcgaggcggcggtgcagcagatgcgggcccaggtccagccactgcagcgcggcgcgcgcggccgaggccgtgtacacgctttcgagcaggcagcgcgtgctggccgagacgttggaggcgcgaatgcctaacaggtaaaggctaatgtagaggtgtcgcggcgagtcgcaacccgtctccatgcggatgagcagcgcgcccggctgcgcctcgaactctaccaggccctcgggcacgaagaaacgcgccgtgagcgcctggtgatcggcgtggtagagatagcgaaccgatatagtatttacctcgcgtttggctttgagcgccgtcactagttcattgtcctcgtcggccgggtcgcgcggccgtttggccaccgcgcgcgcgtccatgatggcgaggcgcacggtagatttcaaaaagttgatagagcagctgcgggcacgggccacggacaaagcggaggcgttaaataccgtgagccaattggagatcggcgcggtggatgcccaggacgtgaccgcgagcgccgtgcgcgccttcgtgggtgcgttgccgagctcgggctaccactttggcttcgtgcgtcagaacgtggtcttttacctcctaagccacgccacggtacagacggcgcgcgacccgctgtacgccgccgagcagttgcacgaacagctggaccgcttcctgcgacaccagcacgacggcggcggagacgaggaccggttgccgttctaccacaacggggccacgctgacggctttccagaagctgttgcagaccctgcgcgagatccagaccgtaatagccgaacagagcggcggcaccgcggcggcggcggacttgatcgccagtaacaacgcgtcgaccgagcgccgcggcaagaagggcggttcgagttccgggggccagcagccgctggtccgccgggtgatcacgcagctggaaacggctgccacggaggcgcggccctacgtcaattgtcgcgccgtggccgaactcctggacctgacctaccagcggctcatctactgggcctgcacgctcatgccctacgtgttgtttcggcgcgacaccgacaccgaactggacacggtgcttctgatgcattttttttacacacactaccgttcggttaacggcgatttggccgtggagtttcaaaactacgtcaagaacagcgtgcggcacatgagctctttcgtcagttccgatatcgacggcgaccagaagcccggtgccgaacacatgcgtgacgtcagctacaagctgttcgtgggtaatctgcaagcgcgtgacgccagcggcctcatgtttcccatcattagcacgcgcatctccaccgtgaacctttacctgtcgcccgaacgtatgtttttccacccgggtctgatctcgcgtctgttgagtgaggaagtttcgccgcgcgccaacctagacgcttacgcgcgcgtgtgcgatcgcgtgctggaagaccacttgcatacgccgcgacgcgtgcaacggctactagatctgacgcagatggtaatgcgactggtggaactgggtttcaatcacgatacctgcgcggcctacgcacaaatggcgctgatccagccggccagtcagaagagctcgctctttgtcagcgagattcgcgagaaactcatacagatcatctacaatttttacacgtttttcatgtgcctctatgtgtacagccccacgttcctgttcgaccaccggcggcggttgattttggagcagcatcgatccacgttgatcggctccaaggaggaactacagcacgtctggagcaacgtgacactgaacgtcaatacgcactttgcggttcagtacacggaagaagactttgaggcacatacgaagggtgccacggaggcggagcgcgagtacctgtatcgggacctgcacagcaagtggggcgtgcacctgtttaccttgcgtccgtctcgcggcgcggccggcgcggcctcgcctttgcctccgcttgacggcgtcacacgctccgacatcttacgcgaatgcgcgctcgttaatctgaacgaaggccgcgtcaactacgcctccctgctagccttcagccatcatcccgagttccccagcatcttcgcgcagttggtggtggtaactgagttctcggagatctttggtatcccgcagggcctgtttcaagccgtgggttcgccgcgtcttttcgcactcattcagctgtgccgtgtattgttgcccgagcaggtgacgctgtaccagaacctggtctccatctacaacctgaccaccttcgtcaagcacatcgacgccgcggtttttaagacggtacgcgattgcgtcttcgacatcgccacgactctcgagcacctcagcggtgtacccgtcacgcccaatgtggacctgctggccgagctcatggcgcgctccgtagcgcataacctgtacaccaccgtcaacccgctgatcgaggacgtgatgcgcagcagcgccggcagtctgagaaactatctgcgacatacgcgactctgtttcggtctggcgcgtggccgggcgcgcctctcggaggacggcgtgacggtgtacgtggaggtacaaggtcaatacggactacgcgtacccaccacgcgtttcgtagaacagttgcgcgagctggttcgccgcgatcggctgttggccgagaatctgcgcggcttgaacgagcgcctgctgagtgttcgcgtgcgcgtacgtcagatcagcagcgacacagaggaagtaagccgacacgccaagggtcaccgcacggtggcccagatgagcaaggcgctcaaaaagacggcctccaaaatcaaagtgttggaaacacgcgtgacattggcgctcgagcaggcgcaacgttccaatggcgccgtcgttaccgcggtgcaacgcgcgctagccgtctttgacgtactaagtcgcgagaacttggaacgccgcggcgcacagctctgtctgacggaagcgacgagcctactgcaccgacatcgcgcgctagcgccgatgacctggcccgcgggcacgggcgttgcggcggcggccgaagcggatcgcgccttacgcgagttcttggaggcgccctgggaatcggcgccccaaccgccgcgactccgcatgacgcccgacaccgatcacgaagaatcaacggcaggcgcgacgtccgtaccggaggtcctgggtgcgcgctacgaacccgcacacctggccgcgagcgacctattaaactggtacatcgtccccgtaagccaggcgcagcaggacatcttgtcttcgatcgacccgcccgccggctcgacatcggtgtccctgccgccggcctcgccatgaaagtcacacaggccagctgccaccagggcgacatcgctcgctttggagcgcgagcgggcaatcaatgcgtctgcaacggcatcatgttcctacacgccttgcacctgggtggaacgagcgccgtcctgcagaccgaggcgctggacgccatcatggaagagggcgcgcgtctggacgcgcggctagagcgcgagttgcaaaagaagctgcccgccggcgggcggctgccggtctacagactgggcgacgaagtgccgcgccgcctggagtcgcggttcggccggaccgtgcacgcgctctcgcggcccttcaacggcaccaccgagacgtgcgacctggacggctacatgtgtccgggcatcttcgactttctgcggtacgcgcacgccaaaccgcgtcccacctacgtactcgtcaccgtcaactcgttggcgcgcgccgtggtcttcaccgaggaccacatgttggtctttgatccgcacagctccgcggaatgtcacaacgccgccgtgtatcactgcgagggtctccatcaggtgctgatggtgctcacgggcttcggcgtgcagctgtcgcccgctttctactatgaggccctttttctctacatgctggatgtggcgaccgtaccagaggctgagatcgccgcgcgtttggtctccacctatcgcgaccgcgatatcgacctcaccggcgtcgtccgagaaagcgcggacacggcagcgacaacgaccaccgccgcaccttccttacctccgctgcccgaccccatcgtcgacccgggttgccctcctggcgtggcgcccagcattcccgtctacgatccctcgtcctcacccaaaaaaacacccgagaaacgccgcaaggacctcagcggtagcaaacacggaggcaaaaagaaacccccgtccacgacgtccaaaacactggccaccgcctcctcctccccctcagcgatagcggcggcctcttcttcgtccgcggtaccaccgtcctacagctgcggcgaaggggccctgccggccctgggccgctaccaacagctggtcgacgaggtagagcaggagttgaaggctctgacgctgccgccgttgcctgccaacaccagcgcctggacgttgcacgcggcgggtaccgaaagcggcgctaacgcggcaacggccacggcgccgtccttcgacgaagctttcctcaccgatcgtctccagcagctcatcatccatgccgtcaatcagcgctcgtgtctgcgtcgcccctgcggtccgcaatcggcggcgcagcaggcggtacgcgcctatctgggcctatccaagaaactggatgcctttctgctcaactggctgcaccacggcctggatctgcagcgcatgcacgactacctgagccacaagaccaccaaaggcacgtactcgacgctggatcgcgcactgctggagaaaatgcaagtcgtcttcgatccctacggacgtcagcacggcccggcgctcatcgcctgggtggaggagatgctgcgctacgtggaaagcaagcccactaacgaactgtctcaacgactgcaacgtttcgtaaccaagcgaccgatgcccgttagcgacagcttcgtctgcctgcgacccgtagactttcagcgtctgacgcaggtcatcgaacagcgacgtcgggtgttgcaacgtcaacgcgaggaataccacggcgtttacgagcacttggccggcctcatcaccagcatcgacattcacgacctagacgccagcgatctgaaccgacgcgaaattctgaaagcgctgcagccgttggacgacaacgccaagcaggaactctttcgcctgggcaacgccaaaatgctagagttgcagatggacctggaccgtctgagcacgcagctgctgacgcgcgtgcacaatcacatccttaacggctttttgccggtagaggacctgaagcagatggaacgcgtcgtcgagcaggtactgagactcttttacgacctgcgcgacctgaaactgtgtgacggcagctacgaagagggattcgtcgtcatacgcgaacaactgagctacctcatgacgggcactgtgcgcgacaacgtaccgctactgcaagagatcctgcagctgcgacacgcgtaccagcaagccacgcagcaaaacgagggtcgcctcacgcagatccacgacctgcttcatgtcatcgagacgctggtgcgcgacccgggcagccgcggctcggcgctgacactggccttggtacaggagcagctagctcagctggaagcgctaggcggcctgcagctacccgaagtgcagcagcgcctacagaacgcgcaactcgcgctaagccgcctctacgaagaggaagaggaaacgcagcgtttcctcgacggactctcgtacgacgatccgcccaacgaacagaccatcaagcgacacccacaattacgcgagatgttacgtcgcgacgaacagacgcgtctgcgactcatcaacgccgtactgagcatgttccacacattagtgatgcgactggcgcgcgacgagtcgccgcgaccgacgttttttgacgccgtcagtttgttgttgcagcaactgccacccgactcgcacgaacgtgaggatctgcgtgccgccaacgccacgtacgcgcagatggtcaagaaactggagcagatcgagaaagccggtaccggcgcatccgaaaaacgtttccaagcgttacgggagttggtttactttttccgtaatcatgaatatttctttcaacatatggtcggacgactgggcgtcggacctcaggtaacggaactctacgagcgatatcaacacgagatggaagaacagcacctggaacggctagaacgtgaatggcaagaagaggccggcaagctcacggtaacttctgtggaggacgtgcagcgtgtcttggcccgggcaccgagccatcgtgtcatgcatcaaatgcaacaaacgttaaccaccaagatgcaagactttttagacaaggagaaacgtaaacaggaagaacagcaacggcagctactggacggctaccaaaaaaaggtgcagcaggatttgcaacgcgtggtggacgccgttaagggcgagatgctctccaccatcccgcaccaaccactggaggccacactcgagctgctcttgggcctagatcaacgcgcccaaccgctactagacaagttcaaccaggacttgctgtcggcgctgcagcagctgagcaaaaaactagacgggcgaatcaacgagtgtctgcacggcgtgctgacgggtgatgtagagcggcgctgtcacccgcaccgagaagcggctatgcaaacccaagcctcgctaaaccacttggaccaaattttgggtccgcaacttctgatccatgagacgcagcaggccctgcaacacgccgtccatcaagcgcagttcatcgagaagtgtcaacagggcgatccaactacagccatcacgggcagcgagttcgagggcgactttgcacgctaccgcagcagtcaacagaagatggaggaacaattacaagagactagacaacagatgaccgagactagcgagcggctagatcgctcgctgcgccaggatcccgggagcagctccgtcacgcgtgtacccgagaaacccttcaagggtcaggagctggcgggtcggatcacgcccccgcccgccgacttccagcagcccgttttcaaaacgctgctagatcagcaggccgacgcggcccggaaagcgctcagcgacgaggccgatctgctgaatcagaaagtacagacgcagttgcgacaacgcgacgagcagctgagcacggcgcagaacctgtggactgatctggtcacgcgccacaaaatgagcggcggactggacgtgaccacccccgacgccaaggcgctgatggaaaagccgctggagacacttcgcgagctgttgggcaaagccacgcaacaactgccgtacctgtcggcggaacgcacagtgcgctggatgctggcctttctggaggaagcccttgcgcaaatcaccgcggaccctacgcacccgcatcacggaagcaggacccactaccggaacctgcaacagcaagctgtcgagagcgccgtgacgctagcgcatcaaatcgaacaaaacgcggcctgtgaaaattttattgcacagcatcaagaggcgactgccaacggcgcgtccacgccgcgggtcgacatggtccaggcggtggaagcggtctggcagcgactggaacccggacgcgtagccggcggcgccgcgcgtcatcaaaaagtgcaggaactgttgcagcgcttgggtcagacgctaggcgacctagaactgcaggaaacgttggcgacggaatactttgcgctgttacacggaatccagaccttcagctacgggctggactttcggtcgcagttggaaaagatccgcgatctgcggactcgttttgcggaactggccaagcgacgcggcacgcgtctctccaacgagggagtcctgcccaacccccggaaaccgcaggcgacgacttcactgggcgcctttacacgcgggttgaacgcgctggaacgacacgtccagctgggtcaccagtatctgctcaacaagctcaacggctcatcgctagtctataggctggaagacattcctagcgtgcttccggcaacacacgagaccgaccccgcgctgataatgcgcgaccgcctgcgtcgcctatgcttcgcgcgtcaccacgacaccttccttgaagtggtagacgtcttcggcatgcggcaaatcgtcacgcaggccggcgaacccattcacctggtcaccgattatggcaacgtagcctttaagtacttggcgctgcgagacgatggtcggcccctggcatggcggcgccgctgtagcggcggaggactcaagaacgtcgtcaccacacgttataaagccatcacggtagccgtggccgtctgtcagacattgcgcactttctggccacagatctcgcagtacgacctacgaccctacctcacgcagcatcagagccacacgcaccccgcggagactcacacgttgcataaccttaagctcttttgttatctggtgagcaccgcctggcaccagcgcatcgacacgcagcaggagctgacggccgccgatcgcgtaggcagcggcgagggtggtgacgtaggggaacagagaccgggccgcggtaccgtgctgcgcctgagtctgcaagagttttgtgtactcatagcggctctgtaccccgagtacatctacaccgtcctcaaatacccggtgcagatgtcactaccctccctcacagctcacctacatcaggatgtgatacacgcggtagtcaataacacacacaaaatgccccccgaccacctccccgaacaggtcaaggccttctgtatcacccccacccaatggcccgccatgcagctcaataaactgttttgggaaaataaactggtacagcaactgtgccaggtaggcccgcaaaaaagcacaccgcccttaggcaagctatggctctacgccatggccacgctggtctttccacaagacatgctgcagtgtctgtggctagaactgaaaccccagtacgccgagacatacgcctcggtgtccgaattggtacagacgttgtttcagattttcacgcaacaatgcgaaatggtgaccgaggggtacacgcaaccgcagctccccaccggagagccggtgcttcagatgatccgcgtgccacgtcaggacacaaccaccacagacacaaacacgaccacggagccgggacttttagatgtttttattcaaacagaaaccgccctagactacgcgctgggctcctggcttttcggcatacccgtgtgtctcggcgtgcatgtagccgacctgctgaaaggccaacgtatactagtagcgcgccacctcgaatacacgtcgcgagaccgcgacttcctccgcatccaacgctcccgggatctcaatctcagtcaactgctccaggacacgtggaccgaaacgccgctggagcactgctggctacaagcccaaatcagacggctacgcgattacctgcgtttccccacccgcttagagtttattcccctagtcatttacaacgcacaggaccacaccgtcgtacgcgtgctgcgaccgccctccacgttcgaacaggaccacagtcggctggtgttggacgaggccttccccaccttcccgctgtatgaccaagatgataactcatccgcggacaacatcgctgcgtctggcgccgctccaacaccgccggtacctttcaaccgcgtgccagtcaatattcagtttctgcgtgaaaacccgccacccatcgcgcgagttcagcagccgccgcgccgacatcgtcatcgagcggccgcggccgcagacgacgacggacagatagatcacgtacaagacgatacatcaaggacagccgactctgcattagtctctaccgcctttggcgggtccgtctttcaagaaaaccgattgggagaaacaccactatgccgagatgaacttgtggccgtggcgcccggcgccgccagcaccagtttcgcctcgccgcctatcacggtgcttacgcagaacgtcctcagtgctctagaaatactgcggctagtgcgattggacctgcgacaactggcgcaatccgtacaggacactattcaacacatgcggtttctctatcttttgtaaccgacactgacagtagcgggtaataaaaacaataggatttttatcgtttttttatgttacaaaacaacgtatcactttcacggtgatttattcttgctattccttttccccttgggctgtcagcgccgggtgcgcgacacggctaccatgcgcaacaggtccagcttaaaggcgcacttgtcattaaacaggctggacatgcgcgtgtacttgctcagcatggtggccaacaccgggtgggtggcctctgatatctcggtcggcagctccaaaacgacgttaacgacgtgacggtgtttttcgtcccgcttgttggccaccgtgggtcccggcgcggtgttagacatggggcaggccgtggggggaggacgaagaggaagccgctgctaaaccgccgcgcgcctgctgcacaatgtggccgccgacgtggcaggcggtctgtttaaccagcgcgcagccccgacacagcggggcgccgtcctcgctttccaaacagctgtcgcggtactcgcccgtctgacagcgcgcgcacagcaggccgtgcccgtgcgaagtgaggcgcaggagacgcgggaccgtcacgtcgcgtaccaccacagtggagtcgcaggtgcgtgccgcgcagggcagaatgacgtcgaaagccagccggtgatcgtacacggcacaagccgcgttgaggcccagcacggctttccagcccacgcgtacgcagcgctgtccaaagagcgtctcggagacgagctcgtagacgcgctgccgcaccacccgctgactgccgcagagcgagcagtgcacgagctcggcgtgcgtgttgaagatgacgctcttttcttgacggtcccgataatagaacatcgagttgagcggaaagttttgctggcagtgtagcttttccttacccaggttgaggcagtgtccgcactgccgacagaccacggccaccagcgagcgcgcgtccagatggcgctcgcacttgagtcgacacagacaccagagcggcaggtcgatgacgctgccgatgaggccgccgcgcagcgcggcgctgagtgcaaagaggacgatcttggtgggctctacgtgacgcgcctgctgtccggcgcccgcgtgtcctaccgccgcagctgccgccgtcgagcctcctccgcgcgtctcgtcgtgcagacccagtgcccgcaacggcaccaggtatcgcggacacgtgtcgcaaaacgtctgcaccgcttgtcgggccagtacgtagagcgggtttccgcagggtaccttcccggcgtaccggcgcaaggctgcgatgaggccccgcaactgcggcgaccgcggctgccgttggtgacaccactggttacggtggtatacggccaaatcagcgcgggcgtcgaagcgcttggcgcgtagtaatgctaggcacggcgagctggtggggtgaagcacgggcagccgaaggtccaccccgaaaaggaaacggtgaaggtcacctagcagcgaggcggtgacaccgtccaacaacgcgtgcagccgctcgggcgggtagagccgcagacggcgcagcaggtagtcggtgtcgtagcgttcgaaacgcagaaaggccatcgtgcggacggccacggtgtgcagacagtccatgctgtagacgtaagcgagaaacacaaagtagggcttggtcataaccatacgctgaaagagcgccgtcaccgcctcccgctcggcttgccgacacaccagccattcgcgcaggaagcgttggtagagacggtcgcccagctcgcgattcagaaagcgcttatccgtcacgaagagatgaaggacgcaagaacgtggcacgtgatgcaccagctgctgctggaggaccgccgacgtctgcgccgcaaactgcgccggtggctgcgacgtttctaccgccgcttcctccggctgcagcgcaccgcggccgatcaccagctgcacatggaaatggtcctcgtgaacgcagaggggcgcgaagagacggcgcagagcctggtggaactcatcagtcgcggtgtgcggagcgtgtcggagacgacgactggccatgaccgcgccacagcagagccagcaccagcagaagagccagcaccagcgggcccagagtcgcaaagcgcgcgggcagccacggcccagactgcggtcgcgatggcccggagcgcgctcgccaccacgatgacggtgcccaacgataaccagtccgctccaaggacggcgcgcacggcggagacggcggatgacggtgatgggtcgacacccctcgccgacgactcacgtgctcctccagaggccgacgcgcggaccctccgacgtcctggcccgccgctgccgctgccgccttcccttctcccgccagagccagcaactcctcctcctcttcatcagcgtctccctcgcttgcgcatccgcatcgtcccatacaggcctcacaacgacacagccgccacgaccccgccgccatgggtggcggcggcggccgaggcccggcagcggcgccgccagcggcgaccatggtgggagagcaactcggatgacgaggaggaggagggggagatgcggtccgagaggaccgctttcccgccgttcgcgtaagcgcggccgacatgcgggcgcgccacagggacggaccgctgccgctgtgactgcttacggtgacgtggttccggaccgccaacgacgtcgacgcggctttcttggcgtacagctcgcgcagcagattctcgtactcgccctcgttttcgggtccgaaggcgatgagctcgatgttgaagaccgacgccgaattggatttgcgcaccacgcacttcgtcagcactccgtaggccgagggcttgatctcctcgatgtccttgagcgtgacgatgagcgactcgttcaccttaagcacattgaactcacctacgtggcgcgccggcgaaacgagcttgacgggcgctcgtacaaaacagcagagggagacggcgcagccagtgtttttaaagataaaacaaggcacgtggtctgtgcggctctcccagtagctgagtagatactcgacacaatagaccgtgtctgtcttgagcatggcgtcgcacaccgagtaattggggtttttacagatgaggccggcatcggtgacgcgcagctcgctgggacccaacttgaggatacgccgcgtggcctgcaccagatcctgatggagaaccttgttcatctccatcgcaccgacgccaccgccgatttatttacccggcgccgactcgtcttttccctccaggattccgttaatgtccatgagcttgctgacgatcgccgttaatagttgcgtcttctcacggaggatctctccgtgactgcaggtcgcgcagtcgccgtgcacgtacttgaggaaggcggcgtacttctgacccgcgttcacgaaatttaagcgcgcgtccagagagggcagcaacagatcgtagacgcgcggcagcatcggctcgaactgtaatagcagatcgtcgtcaagatcgggtagcgcgtgtccgtcttcaccgtcctcgtcgtcaccacctcccccctcgagcccaccgctcgtaccagccgcgggctccgcgtcctcgtcgatcaccagcggtcgcgtcggcaccggagaatccacgtcatcctgcacgtcgttttcctcctctccgtcgtcatcgtccagaaacggcacccgctgcttagcccaggattccggttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcgactttctttccatcttcaagcatcccggtgtagtgcaccacacaggtctggccgcgcttggggaaggtgcgcccgtctcctggggagatggtttccacctgcactcccattcttttttccgcgtcctcaatcagcggcgccgatcgccatgaatccgagtacccacgtgagcagtaacggcccaacgactccccctcacgggccccacaccacgtttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtaaatcctcaacaccccgccaaccgttaccgtcattacgaattccagacgctcagcctcggcacctcggaggtagacgaactgctcaactgttgtgcggaagaaaccacgtgcggcggcacgcaatccaccgtactcaccaatgcgaccaacaccactagctgcggcggagccgtcgccggcagtagcaacgtaggacccgccggcgcttcggccgcctgcgacctagatgcagaactggccggcctcgaaacctcggcggccgactttgaacaactgcggcgactgtgcgcgccgctggccatcgacacgcgctgtaacctatgcgccatcatcagcatctgcctcaaacaggactgcgaccagagctggctcctcgagtacagcttgctgtgcttcaaatgcagttacgcgccccgtgcggcgctcagcacgctcatcatcatgtccgagtttacgcatctgctgcagcagcacttttccgatctgcgcatcgacgacctgttccgacaccacgttctcacggtcttcgatttccacctgcactttttcatcaatcgttgctttgaaaaacaagtgggcgacgcggttgataacgagaatgtcaccctgaaccatctggccgtggtgcgggccatggtcatgggtgaagacacggtgccttacaacaagcctcggcgccacccgcaacagaagcaaaaaaacaacccttatcacgtcgaagtgccgcaagaactgatcgacaactttctagaacacagctcacctagccgcgaccgcttcgtgcagctgcttttctatatgtgggccggcaccggcgtcatgagcaccacgccactcacggaactcacgcacactaagttcgcgcgactagacgcgttatccacggcctcggaaagagaagacgcaaggatgatgatagaagaagaggaggatgaagaaggaggagaaaaaggaggagacgatccgggccgtcacaacggcggtggcaccagcggggggttcagcgagagcacgctaaaaaaaaacgtgggtcccatttacctatgtcccgtacccgctttttttaccaagaaccaaaccagtaccgtgtgtctgctgtgcgaactcatggcctgctcctattacgataacgtcgtcctgcgcgagctgtaccgccgcgtcgtctcgtattgtcagaacaatgtgaagatggtggaccgcattcagctggtattggccgatctgttgcgcgaatgcacgtcgccgctcggcgcggcacacgaggacgtggcgcgctgtggactcgaagcacccacctcgcccggaggcgactcggactaccacggcctgagcggcgtcgacggcgcactggcgcgacccgacccggtattttgccacgtcctgcgtcaggcaggcgtcacgggcatctacaagcactttttctgcgacccgcagtgcgccggcaacatccgcgtcaccaacgaggccgtgctcttcggacgcctgcacccccaccacgtccaggaggtgaaactggccatctgtcacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctagcgtgagcggcgtgcgcacgccgcgcgaacgacgctcggccttgcgctccctgctccgcaagcgccgccaacgcgagctggccagcaaagtggcgtcgacggtgaacggcgctacgtcggccaacaaccacggcgaaccgccgtcgccggccgacgcgcgcccgcgcctcacgctgcacgacctgcacgacatcttccgcgagcaccccgaactggagctcaagtaccttaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttattacgggttctgtaagagcagcgagctactcaagctctccaccaaccagccgcccatcttccaaatttattacctgctgcacgccgccaaccacgacatcgtgccctttatgcacgccgaggacggccggttgcacatgcacgtcatcttcgaaaaccccgacgtgcacatcccctgcgactgcatcacgcagatgctcacggcggcgcgcgaagactacagcgtcacgctcaacatcgtgcgcgaccacgtcgttatcagcgtgctgtgtcacgccgtctcggccagcagcgtcaagatcgacgtgactattttgcaacgcaagattgacgagatggacattcccaacgacgtgagcgagtcctttgagcgctacaaagagctcattcaggagctgtgtcagtccagcggcaacaacctatacgaggaggccacgtcgtcctacgcgatacggtctcccttaaccgcgtcgccgttgcacgtagtttccaccaacggctgcggcccctcctcctcgtcccagtccacgccgcctcatctccacccgccgtcgcaggcgacgcagccccaccactactctcaccaccagtctcagtctcagcagcatcatcaccgtccccagtcaccaccgccgccgctgtttctcaacagcattcgtgcgccttgacactgtacggcagaaaagccggctccaagtgcaagcgccgcggcagcaccatgtgcaaaaacttgtccttgcgcgcggtttcgccgccgggaaagacgggcgacagcacgttagttacagccttgagaacctgctcaaagtacttgtcggcgtgaatgggcacgccgtgctcgcgcacgtagctcggatcttcggctacctcgtagttgcacacggccgacggtggtttccgcgccctcttctttgccggctctcctcctctcctgttgctctcctctaccccgccgccgtcagcgtcgtcgtccgtgccatcaatcgcgtccgaccgggaaaccacgccggcggttacagaatcaccgttgtcggaggaaccctgcggcgccgtccggacaccgggcgccgtcagaacgtaaaagacccgatccccgaccgagggtagctcctcagaacgggccgccaatcgcttaatgacggcaatgtgcggcaggttagattgacggtacagcgagatgtccttagagagcaccgacgaaagcaccaggtcctcgacacgcacacggtgcaggtacagatcgtcgcgggcctgcaccaagcggcgtaagatacgccagaaaccgcgtggcacgccgtacttcttgacttcatcgagtgagaggcgcgacaggcgcacggctgcttccgagacctcgcgatcctcaaagagcagcgagaggacgtcacgcgtgacgcccttgacgaactcgcaggccgtcttgcgcaccagatccacgcccttcatgctcagacccgaggcgccctccactttgccgatgtaacgtttcttgcagatcatcataagagagacgaagaccttttcaaactccagcttgacgggctccacaaaaagacaggccgtcacgtagtgcgccaggctgggcccacgcgccaccagagcctgcggcgtcaggccacgaaagcggacaaacacgctgtccgtgtccccgtagatgacccgcgcctccacccgccgttcgttcgagccccctgacgatgtttcgagcccctccggtaacgcgctgctctcctccgaatccccctcccgcgttcccactacatagtcttcctgattaaaaaaattgtgcaaaaaacacggctctgaaaagttgtctttgatgaaccgcgccgtgcgctctagcatgtcgcgaccgatgcgcgtgatgctggcggcgatgggcagacacggcatcataccgttgaccacgccggtaaaaccgtagaaagcgttgcacgttactttgagcgccatctgttccttgtcgagcagcatacggcgcacagggtcttgacactcgcgcatgcattcgcgcacggcacgccgctgcgaaacccacttgttgagcagttccgagagcaccgagacgcgcaccgaagcacgcacaaagcggtgggtcacgccgttctctagcgtgacgctgtatacgtcggcggggtccacagggtactcgccacccggcaccagcagggtggagtagcagaggttgtgggccatgatgatggaagggtagaggctggcaaagtcgaacacggccacggggtcgttgtagtaacccacctcgggctcaaacaccgtggcgccctggtacgaaaccgccgcagtaccgccggcgccgtgattgtcgttggaaacgccgacgccgccactactgccggagccgacgctgaaaacgccgacgctgctactactgttactgccggagccgggtgaaacgccgtcctgactggacggcgcagattgcaagggcggcgacatctgaaacatagccgccacagaacccgcgtcgccgggcacagcggcggtagagatgatagcagcgttaggtgacacagcaacgctattcgtttcgggcaccgtcgtacctttgctgtagtggttgggcaggataaaatcgcggcaggcgcactcgtccagcagcgaggtgtagatacggatctgctgtccgtcaaagatgacacgccgcaacggaattttagccagccgcgcgatggccccggcctcgtagtgaaaattaatggtgttgaacagatcgcgcaccaatacggcgtcctgcagacagtaacggcctacctgggcgcggccctcggcattagccacgaaacaacgcgggatgtccttgtaagacaggtcatccttgcgttgccgcaggtaaagctcggccatagtgttgagcttatagttgggcgagttagtcttggccatgcatacagggtacatgtcgataaccaccgaacccgcaatatacaccttggtggcggccgtgctggccggattgttgtgagaagccgagggaaaagcggcggcgtactgccgcttaaaacccacggcggggctgtgtaaaaagaaacggccgccctgcgccgtaggcaacttgcagaagcgctgcgagtccaccttatacaggtactcgagacgcgtgaggatgtacttcaagtcaaaagagttgatgttgtaaccggtcacaaaggccggcgcgtaccgttgaaagaaaagcataaagcccagcagcagctcgtattcggaagggaactcgtagacgtccacgtctgggcccacctgcccgcaggtgccgatcgtaaagagatgaagacccgagtgcccaaagatcacaccctccgaagtgcagccccgaccatcgttcccgtttgggatcccctgatccacggcggtgtttccccccgtctcgtagcacacgcacgagatctgaatgacaatgtcatcggacttctcggcgcagggaaaaccaccctcgccgctcatgcactcgatatcgaaggacaggcatcgatagcgcggccacgagctgtcgtcgggcacagccaccaggtcagagacatcgcagtctacctcgatatcacaagtcgacgcgcgaccctgctgccgccagtcgtaacgattcacggagcaccagccgaacgtggtgatccgccgatcgatgaccaaacgcgtcagcggatccacacggacctcgtacacgggaaaaccctgctccagcagatactcgccgatttttctggccatggtccagttgctgatagacacacactgcaaatcgggcacgggtcgcgtcccgtacccatagatggaggtcttggtggccggcgtgacagacacggcgtatggcgtccgcggttcgggcactagttcgcccacgctggcaatgacctcacgcagcctatcggtgtcgctgtactcacagtaaaagtagctgcgctgcccgaaaacgttgacgcagatactgtagccgtgttctgtggccccgaagaaacgcaacacgttccccgaaggcaccagatgctgacgatagcgcggcgacacgttttcgggcgagtcgaagaagagcacggcgtccgtctgatcgtaggtgtgaaaacgaataggtcccaccacgcgacccaccagggtctcgcgccaaggacacggccaaaccatgtcatgactcaacaaatgtttaatctctcgatagaacatgagaggcagccgtcccgtcttatgcttgatcaaccccgtctgaccgtcgaacatgacacctcgcggcacgatctgcaaaaactgtttctgtggcggccgcttgcccgagccctgcgcggagccgggctgcgaacgctgacgccggccacccgcgaccgcaccgccggtcacgccgccgctcagatacgggttgaaaaacatagcggaccgtgagaggctgacagcttacgaagcaaaatcacaaagaaaatacacatgcagcacctagatatccagtttaaccccgtatatcacaagtctctgtgtcaatattttttgtctagtttttttttcctcctggttcagacgttctcttcttcgtcggagtctttcaagtgtctgtagccgtttttgcgatgtcgcagccggtctagcaggttaggcttctgtcccttgtcctgcgtgccagtctgtccgtccaaagaatctgtaccgttctgctgcgctcgctgctctgcgtccagacgggccagggccagaagcatctggtaagcctgctcgttggtgtaaggcggagccgccgtggatgcatcagacgacggtggtcccggtcctttgcgaccagaattataaacactttcctcgtaggaaggcggagcctgtaacgacgtgtctttggtgctgcccgacgtcacggtggtcccgtcggcggacaccagatagggaaagaggttctgcagcggctgcgtgcacagacgccgctgtcgagtatagatcaaataagtgataatgactacggctatggccacgaggatgatggtgaaggctccgaaggggtttttgaggaaggtggcaacgccttcgaccacggaggccaccgcgccacccacggccccaatggctacgccaacggcctttcccgcggcgcccaggccgctcatgaggtcgtccagacccttgaggtagggcggtagcgggtcgactaccttgtcctccacgtactttacccgctgcttgtacgagttgaattcgcgcatgatctcttcgaggtcaaaaacgttgctggaacgcagctctttctgcgagtaaagttccagtaccctgaagtcggtattttccagcgggtcgatatccagggcgatcatgctgtcgacggtggagatactgctgaggtcaatcatgcgtttgaagaggtagtccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgattccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaaatgagttgtattattgtcactcgtacttcttctggtcctatgagtgatattcagactggatcgattggccaaacgttccaattccaccaaagatttttgcttgatgccttgccagaacaccaccagaccgccgctggtttcgaagacggacacgtttccgtatttttcatatgtttgattgtatgaagtattgaaaatctgctgtaacttatttatagcctcatcacgtacgcagtccagcgcggagtcggacatgttcacttcttgtttcttagacagaaaagttgcagtcattttggcagaagaaaagtggtacgagtcttcggcttcggaacggatagtacgttccgaggcttcccagaaggtgagctggcaggtgacattcttctcgtcctgtatatcccaagagatcaccgagtcggcacgttcgagaaaagccaccaacctatgggtttctggcgcagcgttgggtcttccaaagtcggaaacgatggtgtagttcgggaaaatgaaaaacttgtcggcgttttctccaaagtagctggcattgcgattggttccgttgtagaaaggagaaatgtaaaccacatcacccgtggaagttgcaaaaaaatgataaggatacttggagcgcgcagtagtgatggtcagcatacagttcagattacaggtctcacgatagagccaggtgctgccgcggctgtgccactgatccttgaccgtcacgtaacgggtactgtgggtgttggaataatcgtcgggaattaattgcatggttttgttttcataactgtccctatgatatgccacgaaaaccgtgcctcctataacgcggctgtaggaactgtagcattgagcaaacttgttgatgtgatgaatctcccacataggaggcgccacgtattccgtattgctgcccagcagataagtggtgtagatgtaagcgtagctacgacgaaacgtcaaaaccttttggtagacccgtaccttaaaggtgtgcgccacgatgttgcgcttgtagaccaccatgatgccctcatccaagtcttcattgataggcttcatcgaggtgcagatgatattacgttcaaagcgaataagatccgtaccctgggccatagaacacacgcgataggggtacttggtagtgttgactcccaccacatctccgtacttgagggtagtgttgtagatagtctcgttggctctatgactgacggcttcagaagacgttacgtgttgagaatagactgaccgggtttgagcagacgtcgtacgagaagtatggcttccattgtgagtagaagaagttgcatgggaagtactagaagaggaaaccgcagcacccagacagacgatacacaggttaacgcagactaccaggcaccagatcctggattccatgttcgtcgcgggccaaatccagcagcgatgaggcgcgtcgtggtctcttgcgtgtcgcgcggaccctccgggaaacacccgcagtcgaggaggagggatacggacttggcagccaaggtcggtccggctccctgaagacacccgagacggccgcggcggccgtcagggtggagggcttggccacgggagctgttggcacgtcgccactctcatccggtctggacagatgcctgtagaggaggagatatagatctttggacttataaagacttccttcgtgacgaagcagcagcggccactctttgttatacgtgagaatcacatctctgtccgggtgcagttcgtcgcgcaggcacgcgatcgagagttgtttcccgaaagtttcattatatagtgcgacggagagcacgagctcccgcacgtgcatccacatctccttctgcagcacgtttaggtcctgacagtccgaaaaattgaaaaaacccatatacttcaccaccatccactcactgggatacacggtaccttccgcgcatttgaccaaatcgtccttgacgtggggtagtacgcccgcgttgtcgcaggcataggccatgtccacattgtgagagaggggataacgatcggtgcagtgggtgaagaggggcccgttacacaactcgtagatctgctgacccagtagcgggagggattccacaggcagactcttgtggatcaggttattgaccacatacaggtgctcatcgtaggtgaactgatcacccacgtccaccacgtcttggtcctggtggtattggctgcggtacagaaacccattcatgagcttagagataaagtccagacacaagggccccactagattgacatcgatgagcttgctagtcagacgctcctgcgttttgatgcaacggatcaccttgccatagcccacctccgagaccttctgcaggtaggcgcgtttgcgcacgttcacctcgcgagtgacgttgtggatgcgggagcgcgcgtccaccaagtcgagagcctcgtgttcgtcgcagttgcgcacccgtaagccgttctcgctgccgtcgccgtcctgcccattcacccctccccctaccactttcttgcctcctccacgagcccggccgccgccaccgttattcctctgactgtgagtactgctgttgctgctgttgctggccgtcatcaaagtcgtacccgtccccgacatcgcctcccgtccacgcaggtgaatagcctcgccctcggggccgtcgccccccgtgccatctggcagcggacgtcgaatctcctcgagaatatgcttgattttggtgtacatctcgttgctttcgtggagcttgttgaacaccgggttgtcctcgaaagcttgaatgctgagggatgtgatgaggtcgatgatcctgttgggggcggcaaagaccgaccccacgaacatgcgctcctccccgtccaacgccttttccccgagcacgaagatgtcctccacgtcctccccgtacagatggcgactgatgccgttcatgagcgcccggcacagctggtgatacacatttagctgctggatggtgatgcccacccgcttgacgataacctccgaggtacgggaccagtaggtaaaatccgacaaggaatatattcgttccggtatatccgtaaacaggttgtactccctcagcgcctcctccgcctcctggatgtagctgtggtaggccgatgaagaagagaataggcttttgagggccgaaaggactccagccaagtgggggatgcgcgttgtcaggtccagcaggtcctgctccaccgtctggatattcacatcggactggcttgacggacggtggaccgctatatggttgcacagcaagccctgcagccgcttgttcagcgagcggccctgattcgggatgatggtcagctcctcgtagcattgggcgcatgtcgtcccttcgacgtacacttcctgacgcgccaccggcgagatgccgcataggcgacggaggagctccagcaactgcgcgcagacctccaggccggcctccggcgccaggatcccgtacacgtagttcattttgcacaggaagcgctcgatgtcgttgagtgtggccagactgacgctgaaacggacgttgtccgtaaactggagctccacggtgtgatggcgatcgcagcgatccaaacggaggacggtacggtagaaggccgcccggtccggctggcgcgagtaggccatcagcgcccgatccagcaaagccgtatcctcgtgcagcgccttcagcagcatctccaggtagagcgtcagcaacgaactctgcgtacgattctgcgccaccacctccgggtagatcttccggtacagatacactatagccgccgcgtttctcttgaacggcgtggactccgccagtaacacgttcggatcgcagtactttagacactccagctccatggcgtattcgttgcatttcgaacacactacgcatagtttctgtaacaaattcatctccatgactcgactcgctcacgtacgagacgctgtcgtccggtctggcgccggccagagacatggagtcggtgcacaaataactcgcgggccgctcgctatgccgactgacgttgacgttaatatataacgacgtcgtcgacgacgcgggttctgctcccgaagctgttgccgccgcttgcggcgcaacctcctccaccaccgccgccgccggctcctccgcctcgggcgacgggggctcggagatgaccggctgtgtctgacactcctccccttcctcaggcggcccgggcgccgacgcgaatgtcggagtttgccagcgcggcggcggtctctgtctctggtgccgcggcgctaaccttcggggctgttgctgctgttgatgatgcgacgccgtctgtcgccgctgttgcggcggtagctgatacggtgtcgcctggtgctgctgtgtcggtggctgctgttgctgctgttgttgcggtctgaaaagcggccacgggggctgcgactgttgttgctgttgttgcgatgctcgtggctgcggcggccgttgtcgcggcgtttgctggcggttacaaccggctgcgtttggccggcaataacccgctgcccccgccgcccccgctgctcccgccgacgccgccagcctcgtcttcgccggcgttcacgagaaagcagccacctcccgtctcgccgggcacgccgaagcaaatggagttgcccgcgacggactcgccgagaagaagaccgccacccccgacgccggacgccgcgccgacgccactgggcgcgaagagcgccgacaggtcgtgcacctcccccccggcggcgtccgttaatcgctgggcgtcggcgtccagcacgcgtcgcaagttctccagcgaaaagtcctccacgccctgctcctgcaacgcggcaaacttgtccatcagcgacgcggccagcgcctcgcagccatccacgaagaagagcacatcgtcggacgcggggatctcctcgcgcacgctcagaatctcgtacacggccatcacttcggggtcgcaatccaagttctcggcgtccagcgccagcatgacgcggttttttataagatccgcgtcaaaaagcacgttctcgcggcgcgagcgtttgatgagcacgtcggccagacgcgtagccaagaggtagcgctggcgcatgaaacgataatcttggccgctcatagagctcacgttaaggctgcgttccacaccgttgcccgaaaagtagccgatctgcccaaactgatagatctccttgctgttgttgatacccgcatatttttccacgctcacgggcacggtcaccaaggaacgatgctcaaaaacgctccgtaccaacgattcacgcgccacagtggcggccatgggcgccggcacgcctgcggtcttcaagcccttgacatgcaacgcaaattcggcgggcgacgagaaccgcggactagcacctaacacgtgaggaaactgcgcgtggttctgcgtcgttaagcgcgtcgtcaacccgtgcagcgagccgatgtagtctttgaagccataatagcagaggaatttgttatggaaacggctttccacgtaactcagcacacagtctggcgccacatccagcagatcgtgctcctgatagtcagccgtcacagccaccagaaatttgacgaaagcattgaactcgcccatgtcacctatgggcacattcttgggcaacgcgttggaacagaccttctgccaaaactgtaagcaggggagaccacattcaggaaagagtcgctcgtgatgtcgatacagcagaaatcccaagcagcccttagccggattacgacgcggaacgtgatcgcggcgaaaaaacacgctacccgcgttgcccttgcccgcgcggtagatgggtcggtttttcacccgcaccatgatcaacgtgggtaccgacagccgcgagagcttgatctccatgggcaccacggcgtacgtgccctgcgcgtacagcctaaagtccagcaggcggtcgtgatccgaattcttggacgacttgatctgcttggtgaagagaaagcccttgcgcgacgacgtggtggagaacgcgccgtgaatggattgaaaatgctgcgtcatccatttggataccaagttggtggtcaacggattgtccacaatgtatgaggtagcggtaataagcgccacgttctggatcacgtaaaagacggatctgaaataggcgtaggctagcagcggctggaaggccacggcgtagggattcagatccaggttgaaggcctgcgtggcgcccgccacctcgtcgcggctgctcttgaggcgcacctccgaaacgaaacccagggcctcgtcgtccacaaacttgttgagcgccgaaaagacggccacaaagtcgcttttgccgtgcgcgctaaaggtatcctcgcccgtcacggggtcgatgagccgcatcttgcggcagtaatccaagatgcgattgagccgataggtacggtccacgctagcgcccaacatgcgaccgccgcgccccatcattcccccggaatccccaccacccccaccaccacgaccgccacccagaccgtcgctcgggcccccgctcacgtctcgtccaccacccccgccagcaccgccgcccggaaccccgtcgtcacctttgccgtccaaacccccgtccttggcgtcgacgttgtaacgccgaccgaagctgcccaaaatatccacgtcgttgagaaaacgcgactgcacggtgatcacgcagggctccttcttgggctgcttgggcaccacgggcaagcgggtgcgcacccgcacgaaggccgtctgataacacgtgtggcaacaagtacccccacaggcctcgcacagccccgcggcgcagcccaccaggtgattcgtgagcgtcgacgaacccgacaagcccgtgttgtacaccgagacacgattcagataccagacgaagcccgaaactagctgcggacacgtgccacacaccaacgccaaatgctgcggcccatagcgttcgtccttgagcggcgcgccctgaaacttgagcaccttgcgcgcgtcgttgtagacgtcttcgcaggccgccgacaacccgttggtgaactgaatagccttgagcaacgtctcctgactggccgtaccgccggcgctgggatgccgcgccgacgactggagatacaccagcctgtgctggtagagcaccgaattagcgctgaagaccaaggcggccacgtgcgtcgagagatgcaacttgagctcggtcagcgcgcggatcagatcgcggtgatcggttgcgttggtcactaaaggccactcggaaaagagcatagattcggcaggttggtaagccgaatcgaaaaataccgaggcaaaactgaaggccaactcgcaaaccaccgcgtcactcagcatcagatgatccttttccagactgctgagtcgctggctcatgtaccccaagtagcgcttatgtggcgccagcttcaccgactgctgactgtcgtgcacaaactgccgcaacgccgcctcgatcagcacacgcggctccgagaagcgcagcgattgacaccatgacgtgtacacgtagtagaaaagcgtctcgcttacggccggcacgtagagccctcgcgcctccacaaaagcgctgcgcgcatccagcgagacctcgtcggcttcggcgtcaagctgcaacgaattaaagagcgtaggcgggtacaacggcacgcgcaccgcctcgccgccgtgcagtcgcaccgtggtcgcctcctccacgcatggaatcagctgaccggcaaagagaaactccttcaagccgttgcccaccaccacgtgcacagtcgtctcggacgcctgacagcccaccgccgcgcacaacgccgccagatcggtaggcacgcgatccgcctcgggcatgtaagcctccaacgcgtacttctggcgggcgtcctcgcacagccgatgcacgtctccgtgatcctcggtaaaagccacgatgccttgcgtatgatgaaagtagagcgcaaaaggacagaaggacgtgactttcgtgagcaccccgccgtcgtaacaaagcacaggcgtgcgcacagagacgccgaaatccgcctccaccgtgagccccgccaacaaaggagcgatcaccacgctcgaggaacggtcgcatagcgagagagtggccagaatctcctgcgtttctgcgttcaacctgctgaagtagagaaaagccgcgggccccaccggcgctagcgcggttagttcctcgtggctcatggtggatgaacggaagacaatggctacgccgccactgagtgaattttataccaaggaaaagttcagcacgtcatgtttgacgcacgacgtctgagacaccaccgtggccaccactgcggtctggctgcggttgcggaccaccaaaggcgacaaccgcaacgatcccagcaattcgtaagaaaagctaaccgctacggtcaggtagcctctcgcagccagaccgctagccgacgcacccgcccgcgaaaatagcgtgatgttcgggacggctttgcgtcaccgccaactaacgtcggtagtcgagcacgtcgtttatcctcagcacaccgtccgatcacaatccgttttcccactcagtcgcacaagcagcacataaaaaccccacacagggcacgtgaaaacaccgtccctagaaaacggcgttttctgtcctaccgtcacccgtatacacaggcaaatcccaatcccgatccccgaaaacaccgtacggtgtttgtgacctccaaaatcacatcagctaacaaaccgtgaaaagtcacgtttcacgaacacggtgtttttaaatcacaaagaaccgcctgacggtttacaagcagaaacaccgcaccacggtggtacaagcgcgatgaatctggtctcgcaacctcaatcgccgctatcaccaccgattttcgctgcgctccgccgacaaaacgccgtacaagctacacaccccaaaaacccgcgcgcctacgggcgccaaacctgtgtgttatctcaacgtcacaacacgacacaaaccgcgtaacgtggtttcccgaacacgtacgcggcacagacccccgacacgtactcgaagaccttacagtttacgagtcaataaaacaggaaaagatccgaactttaaaattgtgtgtttttattttcccatccccctctttttaccaaaaaacacatttttcgtcttgtaaaaagtaactttcgcccattgccatgaaacaccgtgatggggaacggtgttgtgtgtcgactgacgtcactacggcgatcagtatcgacgtcgtgtatacataacggtgcccggtgtttttattcggggcgttgtcgcgtcttgatgtaatgtaacctgaaaccgccgtgcccaagaatgcggaagccagcgtgtaatcataacggggttttgggtacaatctgacgacatctggcggcgagcgtacaccatcgaatgtggcgatcgccggctctacgtcacaatgacgcaaaaacacactgtaaaacccgcgtagacagctttcctggtcaacgagcgccatctggtgtcggcataagaacaggcatcaaccccgtggccggcgaggcggtgagcacttttgttggtcacgtgaccatcagcgcaggaagcgaggcccgtagaaccgcccaagaggcggtgccagatgccaacgtcataatcacaaggtgatttgttacgtcacgcgcgcgcacgcacgcgcgcggtagaatacagcgatccctagtgaagccacacccattacgtgtagccatatccgcttacgtatacagccacacccctaggtacgccaccttatctaccaatcacagaaacggatatacaatgacccctccctagactccaccccttgtacggaaatttcagataggtggaacccgttagggttccaccgtcctcggtgtacgtacaggcttctccgtctaccggaaatatacacctgctgacgtagacgctactcccggatacgcgtcataagctactggaccctaggggggagtgtctacagggctacgtgcacgcccccttacctagggtatccgcccccttcctctgttttggcctagtaaacttaacgccgccgcttctcacgtgacccctgacaagcctacgtcacactcgcgtgaccacacccactccggatatacgtcatcctgtggaattccggacatacggtgacgtagcgagcgtagcgagctacgtcacgtatgcgtgcgtcatctccggcggaaatcatctctgatgacgtagcgagcgaagcgagctacgtcatcagtccgttttacgtataccggatgctaggcgacgccccgtaggggcggagcctagcttccacccctaggatgcataccctatatagcataattcttctaacgaaacgttctacgaaaacggactggcggaacgggaaccaccgtaacccccccccctcacccccccccttctcctccggaaccggggggggcaaatttttaccaaatttgggcaaccatgatttccaatgggacggcgtttccgtgcgcatgcgcagtcggggcgagtttttggttgtcagggcgttgccacgcggattatgggatggtgactcgagtgcgcatgcgccggggatgccgcatggaaaacctatatataaggaggggtgaaccaggggccccggggcgcatgcgcgggccagggcccgcgggagggtcgccctgcgcatgcgccggtaaaattccactgtgtgtgtcgtgcgcatgcgccagtatttttccactagaggcggtcagtgcgcatgcgtcggtaaaattccactagatgtgcgccgtgcgcatgcgccggtatttttccactgggcggccgcacctagggagcgcgagccccgtgccgggcatgggccgcggcggtggaaaattaccgctccgcccacctaggcggggcatctgaaaacctataaaacccggcgtgcccgccgccccccggcgcagtccgcggcagggttccggccgtgctgcggtccgcacgctgcgcccgctcccgcctgcctcccgccctaccccccaccctccccggccgaggcccggcgccggtccgtccgcgggcccgtcccaccgccctggagcaccatccggggccgtgggccgggcaccgggcgcggcccgctccggacctcggccgggggtccctcccctccccccgctcgacccccccatccgacggcccggccgggctgggacccccgcaccggggtcccggttcccgtccgtggcccggggggacccgagcgggggcttcccacccccaccccgctcctccccgggctccggcccgggatccctcgctgctcccggcgacctccgccggcttcccggtccacccgccgcggaatggacgggacccggggtccgcgcccttcccctccccccacggggggctgggtcgcggaccccggttcctaggctcgttccgcggtgggcgaccggggatcccccacccagctccccttcccggcccgccttgctggcttttgggcccctgcgggctttttttttccggctgggggtcgcggcggtcggccgacgacgacggtaggtgggccgggtggacggtggtggggacgggcgacgccccggctcgacggcaatcggtcccggaaggttgggggctgggggcccggtcaggagctccgggagcggggtcgaccgcgacggcttccgggtctcgcggcggctccctctcggcggctccggttgggctcccctcccccctctcgagggtccggccgccagtcgtgaccgggggtccctcggcctagccgccggctctcggtccgccttatcctgggcgttggcctgtcccgtgacgctcccctcccccgctgctccccaaaaaaactccgcccgaaccgtcgcggcttgctggccctgggcgtggtcccccactcccctccccccatcggccgcccagccggggtcggcgcctcggaccccaccaggctgtggcgtgtgtgctggccgatgcggcggcgaggttgggtgtggccggaagcgctcggggtcgacggtgggccgccatgacacctcaattgtcgtcagtacgcccctccacaatcaccgtccccacacgatgggcccggcaggtcacccaacgttggttcaggcccagtcgggttttttccccggcacgaacgcacgtccccgtgggctccacgcgttttccaccctttcctggaggggtccggaacaccgtgaatccacggggagggtcccggcacgggccgaggagaccacgaccgtcccacccggcgtgtcgactcgtccgagacccgggaagggaacaggccccacctttttttcccttctccgattttgccgtggaaaacccgtgaaccgatacgggtgcagacggccgaaaaaaatcgagacgacaatatgacggcagggcgcgatcttctcccccatccgacaaaaccgtgtcccttaaaattccccacctttctctgttcaaatggccccgaaactgtaaaacaccgtttgaccgcaccccaaccggcgccatcttggtgaccttctcgacggttctctcgctcgtcatgccgttctgagctccgacatggcggacgagagaaaatggcgtcgagagcctaggagcgtttttgctccaggcgggtaaaaaaatagcacgataacttttctgtgcttttttttgagacgttttagaagagcttttttctgctcagagcgaaaaaatgatagccctgaaaatctcgacgagtctggccgagcggcgccatcttggaggaggggcgagtcgcgggcaccgcctcggtaccccctggccgaggcgagtccgcggtcgccgcctgttccgtgatgctacctagagggcgctgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatcgcgcctgaccccttttataaactgctcgaagaaaagaacaccttatgtgaaaaaatacagaatgatgacaagttcatccaacacaaccgctcaacaacgccatatctatcagtgtccaaaaactatcttctatcctttgaaactataaatgctgcctatatacatatttagtatccaagactcttaccacgtagacgaaaagaagtgatacaatgatcttgacgtgtatcgtctatatcgtgctagatatattcagataagacgcgcaaaccatagatttctcatcagtatcatgaaagacctatagctctatatacgaacctagtcattttaggacagccgccggagaagccgacgagggatcgggcgggtgcagccagaacctcacgcccgatcccgcctccggtaggcgatttgcatctgtttggtaaaaagctcataagtctgtatgtgacctatatatattatacgctatgtacaccgaactgtcgctgttgtataagaagaaaaaactctccatatttatatcgtctgaatttttgcttgatagacacgtgtttggaactctgtccccccacgttttcactgtgtataacaaaaatatgtgtttctcaaaagatcttgaggtgtttgaaaacgggggaaacctgcgtttgggtgctctaagccccggactgggacgtagccggcgtccggcacctatatttttctatttttttttacaaaatatatgatgaaccaagaataaaactctagctctcgtctatttttaatatgctctacttagaacctttttaatgacagaatgaactccatgttatacgctctttatatagtttctctgcactaacctttaaaaccgtatccttccctgttgtacaaatcatcttttgatacacaatgatgacctgatatccctccatatatatgatcggatattattccgttagacttgtcctccttttttttcctcatctcctgtatctggagatatatgttgaccaccaccgccatgaccaccaaaaagctagccgtcacgactagaaatgtgtaggattcggactttccgttcgagaagaaaaagagaccgcgtctctggacgctctttttgtcagtctgaatcgacccgggatacgtaagagagcggccctacatcggggggcgctcgagaccgacgacgttccatctgaccagaaaaaaaaaggcacccctcggtggcgacctctcaccatcgtttgcccgtccgcccgtccttcgtagccatcatcatctcaggctctatcggtaccatcgttgtcatctgaaaaaaaaactgcctcacccacctgcgtaaaaacaccatctttccggaggtgcggtaagacgggcaaatacggtcgtgccgaggcaaaaaaaacgcaccatcgacaccacaccctcatgagcaccacctgtcggtgttggtcgtcctccatcgttctctacgaacatctcgacgcccgggtgacggacgacggcaagacgtcccggagaagacggtgttctctcgggcggtacgctctctggatctataatatctatagtagctaaacgagactgtgagtacgacgaaccacatcatcttttttttatgttgcttctttagaaaatgacttatgtcgacgacactcggcatcagccatctcgtgaaacacgctcgcttttcgtctctccaaggaacactgggtccgctgaaagggaccgtgtaccgaccaaagcaaaaaacacacacgtagtaacatgatcaaccacgtctgaatgacacgaaaacacaatcgtataacgctctattcatggaacgaacttggaataaaaaaaccatcgcaggccagaggctaagccgaaaccgtccggggaagcgggcgcgagttttccgacttagcctttggtgctcgttgagcctcttttttttttctgattctctgaagaatcaccgtcacagccctatgacgcgaaatcaattgctagaacataaacgttctcaacaggtatgaaatgaacaaactagatgatgctataaccttatattgtgtgtatatagataggtgtgaaatttgtaggataaaaagtgtcgttgtatgatgcacaacgatcgtgaaactggagactgtagctctctaccgaatgcaaatacacaaatgacatcgattcccgtccccacataaagaaatgtgctttactgtgaaagaatgaagaagattcttgttcctcgtacgacggggccctcgctcgtcgtgcctcttcccccctccgggagaggggacgtcggggccctccgtcgcaccgggccgaagccagtgaaatgtttactacactgtcatcagaatatatgatgtatattatttcctccaaactcctcaccatagccaccaattcgcatcacttaagaaagtagtagcaaccgcggcggcggcgaccggccggtcgtcgtctcctcgtcctcaaatgttgtacatgtgcagaaaaatgtgtaaatacgtgttatttatcccatgcgtcttgtacatagatatatgtttttatatacgctatttatactttatatatccttttgcataaccatagacagtcaaggattttaatgatttgctcatccgcctttgagccatcgcttaggagttagttcctctatgttctcggcccaccttttcgactacagtagcaaacccttgtactaccaccccgataaaaaccacatcatcatcgtcaccacgacctggaaacgacacacgttcccccccaatcttgggcatgtgtatatataaaaagaatgggagggagaggacgtggggctcgagaagaaataaacgccaagctcgattcgaaccaaaaaaccacatgtgtattgtgctttgtttttttttttacggtgggggaaaaggagggggccgtcattaacggaaaccgtgtatggggtccggacacgaacagtacacagcttatggggaaaaaagctcacagagagaaaaaacaccaagctcaggcacgcgtacatcattattatcatcatcggatatctcaccacgggtcatagtagtaccaaggagtgtgtaacaccattttttcttttctttgtaacgggataagggacagcaatcatcacgcacaacacccttcactctctttttagtcatccatatcatcgctgtaacacagcatgtcctcgtaatcgggcgtctggcagcgcattaccaccgagtcgtcttcttgcggtaccggtggtggtggtggtggcggcggcggctgctgctgctgggttgccgtcgtactgtgattaccgttggcggactgcaccgggatgatgggctgcttgtggggaacctggggtggactgccgccgtgagaaggcgacggcgtcatcaagttaagctcaccacggtgactccggacaccggcgaggggcgccgggggactgggagggaccgcggtcgtcttgtagacgacggtgtccccgtgtcgatccgtggctcgtaccagatcttgactgctagcgtcgtcactgtcttcgtcctcttccagctcgccctcagagtagtgctgctgtggttgcgacggtggctgggcgggaggagcggcggcgatcattggagagggatgtcgatgactcccttctctgtcctttttatcgtaggctgtcagcgttgctgggtccgtcctgctttccatatttgcgtattgctcatcggtgggatgaatttggtctcctccccgctgttgtccgccggcagtggcgtggttgctggcggttgtcgttgtcgtaccggcaaagacggtgagatccaatagcgactgctcgtcgaagggacagtacgctatcatgaaacgatagggtgccaacgcgcgttggatgcgcagttcgcacatctcgttctgacactcgtggcactgcagggcgcctaggatcaggtccgagacagcgccgcagcggtaggtacccatggcgttgttagtatcgaactggtcaaaaaattggggcgtaccggtgacttgcaacgcgcgacggcgtagcgagacggccacgcgcgagaaagagcacacataggccatggcgcggtgcatgggttgcgagaaggtctcgggcggacgcttctgcagatcgcagacgtcgtcgcgtagccaggcgctcatttgaccgggcttcttgactagccgtttgagcgtgctgcaatggtcgccccagccgtcctggtggtccaggatgcagcccaggtccaggttgttgagtttgttgaagagtagctgacgcatgccgcccaccgtctccagatagggatcgtgcgggttgacgggtagcccgtgcaggtggtggtacttcatgtagctgagcgtttcgtcgatgatggccagcaacgtgtgcaagttgggagcgttgtacacggcgaagatcttttccaccaccagcttgcgcagcaacggttcctccagccaatcgaactgttgacgaatgtgcaacaggtagtcggtgtgcatgagctcgtcgtgtgacagcaggatgcgaccgcgcggctgatgatcttgcgggaaggcggtggggaccttgagatcggcggggtagggtgccagacgtagactctcggccgtgtagcgctgaaggtcgtaaacgggcgaggtagaactcggtgaggtacccgacgaggcggcgccgcgctgcagacgcgctctttttttcttttcgatcaaacggctgagttgctgtagttcgtcctcgtccatggcgtccagttcgtcgtcaataagcgccagcatctgttgttgttgcggtccggcggacgatccgtgatgattattggctgaggaggggtgagaagaaccgaaagtcgtaggacaactgggaactcggcgacgaagatgcgtcgaatcgccgccgtgatggtgcggttcgccgtcatcgttgtcgtaagacttaccgtagtgggggttaaggggcaccgaggcggacgcggccacgcgtcgcttgaaagaggaggacgccctatgtccgccacggaagcccgcggtgcccatgatgatgtgtccgccggtgcccccgagtgcgtggcgggaggagggtggaaggggaggaggatagtggtccggatcgccttcggtatcatcgtctttgctgtagcggggtcgtcgtgcggggacgcagggtcggtgatgatgcgaggcggcgccgacggtatcttccgcgagatggtgttcgctggcggctgctccgttccgtgtcgacggcgaggttggacttcgctcgcgtcggaacttccgtggcacgggttcgtaatccagacagaagcgccgtgcgcgacgggcgcggcgttcgcgctcgctcagggaagataacgacggagcgtcgtgacggccgcgtgagtgcagctccatggccgccgtcgctaggaaggtcacgttcgggcacgctgatgtatatatagatgagaccgctgccggggggcgggtcaccggcgccgtggaaagtgaggctcagacggcggtcgccggcggcacgggcgcgtcgggcggtctgattttgatggaaatgtggacgtttttggcgttggagtgacactttttggtgaaacagcggctccagaggctggcccagagcgcgtagctgtgctcggtgcgcaggtcgatgaacacctgcacggtctcttgcgggttgcggtgcgtgtagttgagacagcgaaaatcccgcgtgcgcgcgccgtcgcgccgcttgacggccacgcagcaggcgccgtggggctgaaagaggaggacgtggggcgcggtaaactgctcgctgacgtgcggttcgtagtgttgcgtgaggtgctcgagcagcggcggccacacgcgggtgacgacgagccgctgcaagtccgtgtcggaaatcgcagcggcagtggcgccgtcgccaccgtacaggtgataggcgagcacctcggtgagaccgcggcgtcgataacgcgtcacgttaagcgagcgcgtctcgataaagttggcttcggtcgaggggcagattttgtcgcgtacgctgagaatgacgcgtggcggcggcgacaggggcaacgcgggcaggtcgtgcggcgggtggtggtgaagcaggttacgcagatccagttgggcgcgcacaaagcctagcgggtgttcgcggtaggcgtcgggcacgatgaacagcggcaacagacggcgatgcatgaaatagccgtcgtcttggtccattttatacatgtagggcagacgtacagagcgtccatggtggtagatgcctgtgtctaggctgctctcgggatgcgagatggggtccagcagcgtgtgcagttcggcgtcgagacagacggcgtgattgagcacctgcgccacggcgcgtaaaacgctggggtgtacggcgacggtgcaggcggggaacggcgtgatgatgcgcagccccagtttgcccttgcagcggcagtaagggggtgacgtgtcaacggaggacgttgttttttggaaaacgccgttatccgggacgttatttttatcctctttcccgtcttcgtcttcctctgtgtcgcgctcgtcccggtaatcgagatagtcgtcgtcatcgaaaggcgcgccggccgcgtccacgggcacgctgttgggtgggcacgcgcttttgaagaaatagaccgggtgccggtcggggtgcgtgtagccaaagaggctcgcccatacggtcatccagacgcgtcgtagtccgcgacatagctcaaagacggtgtgtcgcgccagaccggagacgccgtcgcgcagccgtaaatcaaagtcggccacaaaattgaagacgggcagacgttcgttgaagacttcgtgtcgcgtgtagtagaactgtgtctcggggctggtgctggccacgtcgtcgtcgtgtagccacacggtctcggtcagggcctcgtccgagaaacggctgtcgggtacgtgacggagcaggtcacgcggaaagaggctgcgatgccaggtttcggaggccacggcgcagaagacgtgctggtcattgggcaggtgtacgcggtagacgggcagcggtcgctccagcagcggtgccagcgcgggctcgggtagcaggtagcgacgttgcgagtaacgcgttagcgtgccggtggtgtaagtctgggctgtgcgtagcgaggcgcatagacgtaacaagccggacagggagcgttccagcggggagaagacagactcggaaagcgtgttgatgcgttcgagctggcgcgccagctgcgtggaggtgccgaagaagcccgccaggtgcgtgccgtcgatgcggccgccgtagccggccagccccaagccgtgcgggctggtcgccgagtggggggattcgtcgagacgcagtaggtgcgtctccacgtagtcgtgtagaaagttgtcgagcgagaagtatttttgcatgacgtccagcagctcggtggaaagccggcggcccagaaaacccggttcgcgcgtgcactgcgcttcgggcgccgcgtcagcgtcgtaagccaccacgcgccggtactcgagcaaccgcgcgcgtgccagcgccgtgcggtaggccaggtagacgtagtgcacgcagaccgtgtcgggcagacgcgcacgttcgcggaacgcgttgatctgcgtgtccacctgctctagctcggtgtagtcgcggcggttgcgcgcgacggcgtacgccacgaaagcggacacgcgctgacggaagggcgagcccagtagcagacgcgcgaactcgcccatggaggcgtgcgtggggatgatggtgcccaggtcgcgcgtgcagaagctgcgcacgtactcctccacggtggagatggtgctgtactggccctcgaataggtagtaggccatggtcagcagcacctggccctcggtgtgcccgaagacgctgatgaaccacgagggcgaggtggggcagaggaagacctggttgagatgacgtagcacggccgcgtggtgaaagtacaccaggtgcttgaattcgcgcacctcgccgccgtgttcgggcgagagcacgggcgtgcggaaaagatgccggtagagcggttgcgtctcggcctcgtccagactggcgatgagcgccgagagggggatgggctggcgcgcggccaggtagcgcgagagctgcagcgtttcgttgttcacggcgaagacgggcgccacccgccgcgagtccgagcacttttgcgtctgtaggcagaagtaaacacgtcgcgagacctggtgtttgaccagcagggggaagacgcagtggtccgtcggtgtctgcgagagtacgttggcgactatatgagcagaatcatactctgttgcgaacagaacgagcgtcatcgtcgcgccggcacgatgcagctggcccagcgcctgtgcgagctgctgatgtgccgtcgcaaagccgcgcctgtggccgattacgtgctgctgcagcctagcgaggacgtggagctgcgcgagctgcaggcgtttctggacgagaactttaagcagctggagatcaccccggccgacctgcgaaccttttctcgcgacacggacgtggtgaaccacctgctgaagctgctgccgctctataggcaatgccagagcaagtgcgcgttcctcaagggctatctctcggagggctgtttgcctcacacgcggccggcggccgaggtggagtgcaagaaatcgcagcgtatcctagaggccctggacattctcatcctcaaactggtggtgggcgagtttgccatgtccgaggccgacagcctggagatgttgctggacaagttctccacggatcaggcctcgctggtggaggtgcagcgcgttatgggcctggtggacatggactgcgagaaaagcgcgtacatgctcgaggccggcgcggctgcgacggttgcgccactgacgccaccggcggtcgttcagggggaaagcggcgtccgcgaggacggggaaacggttgccgccgtgtcggcctttgcctgtccctcggtttcggactcgctgatccccgaggaaacgggggtcacgcgtcctatgatgagtttggctcacattaacaccgtctcctgtcctaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgctacaaggagtcggcgccgcaagagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactcaggggcgtgaagaaaaagaaacccacggcggcggccttgctgtcctccgcgtgaacagcctggcacgttttggaaaacgtacgtgatcacggacacgacgagtacggggtttctcatagacgtactttattaggtcagggatgacggggaggtttcgggccgacgtcaaaaataacgtcattcgtgttgacagggctttctgcgtcggagctcttttcatcttcttctgtctcgtcgacgtcatcgtctaccggcgagggtgtccgttgcagcaacgcgtgctcgggcgtgtgggtgaaaccgatgtcgggggtgggcggcacgatcatctgtcctagggggtgactgcccaccggcagataggtaaagcggtgggtggtaaaaaccgctttggctacggtggtgtgtggggagatgcagacggtggtgtgcgaagtgttgaccaccgtcacgccggccgcggtacccgggagccagatggtgggtcggatgatgagatccgattgactaaactggcgcacgcccactatgagggcgcagataccgggcgcgtgcacgtaggccgcgtcaaaatagacggtttgcgtgtgacccggaccgatcaccagcgtctgacgggtacgtaacgaaaagaaacggtgttcgttgggcggcggcaagttcatgagctgccagggttctggtacaaaacaggggaaaacgccgatatcgccttcgatggtgcccggaaagatggactgaaaagtgtcgttgaggttgacgacatccaactgcgggacttgcagcctggattccagcagctcgggcatgcaaacgaattgcgcgtccaggcatttgtaaaaggtaatgccgaaaaaaccttcggggatatagaggctgacgcccagcgaggtgggcactttgcgctcgcgtgatagccaaatgatgtgtttattgtaaaaggccagctgcgtgtggcattgtttgacgatgaaactggaaggcatccacttgtaaggaactttgagcggtgacggtaatggcgacgacgcttcatcctctcccggatgctgctctttgtcgtatttctcctcggtcgattggggcagcgtaaatgtggtttgaaaatcgctatcgctagcgaaacgcacgcagtaacgcatgttgacggatttctcggctaggatgatggagcctgatgacgatgcggactcttccttcattattaacgtaggggtctcccagaatcgctgaaaacgggagcgcggcagccgcgacagtaccagttgagagtcgattcggtcggtcaacatcgtaagcatcgtggcggtggtgtgatggagtggaacacactagtattaggtcttttagttttatcggtagtggcagagagttctggtaacaattcatccacgtcaacctctgcaactacatcaaagtcttctgctagcgtatcaactaccaaactaacaacagttgcaacaacttctgcaacaactacgacgactacgaccttatcgacaactagcactaaactcagttctaccacccacgatcctaatgtgatgagacgacatgcgaacgatgatttttacaaggcgcattgcacatcgcatatgtatgagctctcactgtccagctttgcggcctggtggactatgcttaatgctctaattctcatgggagctttttgtattgtactacgacattgctgcttccagaactttactgcaaccaccaccaaaggctattgagggtggacagatttacagcccggcggtgttccggcggggtaaggtttacatacgtgggtgaccggaggctaaagttacgaatctcatctagaaacagcagcgagtctagatagtcccacaggggatctataaatgttctctgaaaccccattgatggtgacgtaggtgtagttttgttactatcggaagctgttttgttttccacgaacatggtttcgttgtaatataaggagctcatgtcgagagtaccgtaaatagtgtacggcgtttcgttacggattagtacgtgcgtgtttttcataaattctgacacggcggttcggttgcggcttggttcacaaaaaggattttgccggtaacgtagagtggtatacacccacgttgctaggtcccttaactgtgtggccataatggacttcataaagctgctatcaggacgataagcaattgtagacgtggaaacccgccttgcggcggtagtaatactataagtcacgttagtagtgacgttgagagcggcagacgttgtataggaaaagtatggcgtagtagtactctgagttttcttagcttttttttcgaattgttccttaacgggcgcttgtttacgttttagttttcgcatagtgttttttaacttggtgccgttaatatacttggggacgcgaaatagattccggctcatggcgttaaccaggtagaaactgtgtgtacagttgcgttgtgcgtaacgtaaaagcagggcggttaaacctagaaaataaatcgtttgactatctacgttaaccttagtcggacccacgtacaatttggtgttccaacgcggtacattgaaaaacatggggttgaacgtggtgaaattaccgcaaccttgttcgccagtatcattacgtttggaaacgtttagcatttcggaaagacaagtcatggaaggcacagtaccacaaggtgggggtctgaatgttatcgttttagccgtatgattgtactgtgagtaaacgtattttgcgggttttctaagctgggtactataaaaatcaaaccacagataggttatactataattctgaatggggcccgctaaaatgtagtattgtggaaactctgtcatgttcatagtgagatttttaaccggttgtttacttacattgtattttgtagaaatagtcgtttctagttgtctcaaaatttctaacttaagctgatctaatttatatttgcctatcttagacagtaccaagcccctccaaggacgattataaagcgcttttgacataactttacagtttatgaaagaaacaagcaagaaagatatagatattagaaacaccatcttagggacgtctctcaccatcatctcttttctccccatgacagaggaggagaccccgcaccgtccgtctgccttgtggtttggcttgcctgcgtgtactcactgctgattctggtcgttttgctgctcatctaccgttgttgcatcggcttccaagacgacctagtctcccgcaccttggctgtgtaccaagcttgtatccagggcccgatatgtaaccagacccataacagtacctcgtaaataaagacgcacagacctcacgcatatagtaccatcacaccgtgtggcgtgtactttattacaacgagcaagagtgcccctaagtattggggcccgtaccgttttagaagattttgtgtgaatgtctttaacttttctgtcccttttctcataaactgtcaggttctacagtcagcatgtcttgagcatgcggtagagcagatagatgccgatgatggccgatagcgcgtagacggacatcatgaggagacgactgtcggtggcgtccacgacgacgtcagttacttctaggaccgtaccgtttttcaaaagcatgaggtagtgagttcgcggagatgagaccaccacttcgttgtagggatccagggcgaaaaggacgtcgtccgagtcgtgcatgtacatgatgttgatgacgccttgcgtgtcgtcgtattctagcagggcgctttggcaaaaggcgcagttttctagtgaaatgttgagcgccgctgtgatgctgtgtgtggtgtgcatgttgcgcgttagttcgcatttagtttgactgtccgtttgggtgatgatgaggctctggcctacgacggtggtggagacagggtaggagatacctttgatcaggtactggtttgttacgacataactgacgtgttcggagacggttagcgcggagaaggattcgccgagcggcagacaaaacaggtcggggaaggtttccagcgtgcttggttgcatggtagataggatggagagggcggcgggaacggtagcaggaacggtggcatcggggaagagacgcgtgaggcgttcgagcgagtgatcgcgtcgcccgctactggaacagggtgtgtacaggtcgctgaggtattcgtggtgcggatgagctagcaactgcgtaaagtgtgatagctcggccaatgaacagaggcccgtttctacgatgaagatttcgcgtctctccgtcgtatgtaccaacatggagtggacgaggctgcccatgaggtagagttcttggcgcgcgaaggctgaaagaaaagaggccaggtgcgttttgtgtaattgtagggcaaagtcggcgatctgtcgtagtgcccactggggaatgagatgttgctgattctgtttagaaagtatgtagaccaggcgtacgaggctggtgatgtcggtgatctggtccggcgtccagagggctcgtttggccaggtccacggctgtgggatatagcagcaatgtggtgcgtggtggtgtttgtgagaggcaggtgatcataaattcttgtatttgtaagagtgcggcctggcggtctagggctcgtgggatggagatttcggtgccggcctcttcttgtcgggctgccgcgaacagtgctaatgcgtaggcgaaggccatttctaccgtgcggcggtccaacatttgacatcgaccgcttttgagtacgtctacagcgtaacggtgaaagctgttacgtaacagtgcgctgaggtccaggtagttgaagtcgagtgcggcgtcgagaaagtccgagtctttgagataggagtgacggtttagttgagctttcttaactagtaccaggagctcgtgtttttcagtttgtcgtagtataaagttgtcgcgttgatagggcgctttgaagagtacgcgtggaagatgaccgaagataagcagcatgggtgtgtcgtcgtctatagataccgtaactacgaagaagtcctcggtcagtgtgattttaacgtaacgtagttcgtccatgaggtaaaagccctggtgcagacagggcgtaacggtgctgaaaagcagatcgtgtccatcaaagaggatacaggtctggttaaagtgtggccgatgtagtcccgaggtggtgtgcgatcccttccagtcgtgtggagtggtttggggtggcatccaaacgtgaggtattgacagatcaatgggcggtggcacggtggtgggctgctgacccaggctgtcttgtgccttcagctgctgcgaaaaagatcggtagctggccaggtctttggataccaatgcgtaggtgttaagtctctgttggtatctttctagggtttcggtcagatctacctggttcagaaactgctccgccagaggacccgcaaaaagacatcgaggcatatggaatacatagtattgattatagctttggaaaaagttgaaactgatggcgttttccctgacgaccgtgctgttacggaggctgctgttgtaggtgcactgggtggtgttttcacgcaggaagcggatgggtctcccgtaggtgttgagtagtaggtgaaatgcgtgagggtccagcgcttcggatgcggcgtccgcgccatatcgttgcgaaggtaggtgactgaggaggtagacggtgaagacagtgaggtagggggggaggccgggccgcatagcgcggctgcgccgctgggttcagcggcgtgatccaggtggtggttggcgttacacccgagagaaggagagaaaggatcccaggaaggggcacccgggtgtggcgctacgggttacaaaagtcgcgtctctgtctatttaatacgatgtcattggccgctgcgaaggaagaagaggggacacgcgggtaagccatgccgtccgggcgtggggacgacgctgattcgacggggaacgctctgcggagattgcctcacgtgcgtaagcggatcggtaagcgtaagcacctggacatctaccgtcgcctgttgcgggtctttccctcgtttgtggccctcaaccgcctgttgggaggccttttcccacccgagctgcaaaagtaccgtcgccgtcttttcatcgaagtacgattaagtcggcggattcccgactgcgtgttggtgtttttaccgccggactctgggtcgcgcggcatcgtgtattgctacgtgattgagttcaaaactacgtactcagacgccgacgatcagtccgtgcggtggcacgccacccacagcctgcagtacgccgagggcctgcgccagctcaagggcgccttggtggactttgattttctgcgtctgccgcgcggtggcggtcaagtctggagcgtagtgcccagtctggttttttttcagcaaaaggccgatcgcccatctttttaccgggcttttcgttcgggccgtttcgacttgtgtaccgattctgtcctggactatctgggacggcgtcaggatgagtctgttgcacaccttttggcggctacccgtcgccgtcttcttcgaaccgcacgaggaaaacgtgctgcgctgccccgagcgcgtgcttcggcggttgctggaggacgcggcggtgacaatgcgcggcgggggctggcgcgaggacgtgctcatggaccgggtgcgcaaacggtatctgcgtcaggagctcagggatctgggtcacagggtgcagacttactgcgaggatctcgaagggcgcgtgtccgaggcggaggcgctgttgaaccagcagtgcgagctcgacgaaggaccgtcgccgcggacgctgctacaaccaccgtgtcgtccgcgttcttcgtccccagggaccggcgtggcaggagcttctgccgtcccacacggtctttatagtcggcacgatgccatcacgggacccgccgccgccccgtctgacgtggtcgccccgtctgacgcggtcgccgcgtcagcggccgccggtgcttcttctacctggctggcgcagtgcgccgagcggccgttgcccgggaacgtacctagctactttggaatcacgcagaacgatccctttatccgctttcacaccgattttcgcggcgaggtggtcaacaccatgttcgagaatgcctctacttggactttctcctttggtatctggtactatcggctcaagcgggggttgtacacgcaaccacggtggaaacgagtgtaccatctggcgcagatggacaacttttccatttcgcaggagctgctgctcggcgtggtcaacgctttggaaaacgtgacggtgtatccgacgtacgactgtgtactctccgatttggaagccgccgcctgtctgctggccgcctacggacatgcgctttgggagggccgcgatccgccggactccgtggcgacggtgttgggtgagctccctcagctgttgccgcgtctggccgacgacgtgagtcgtgagattgccgcttgggaaggccccgtcgccgcgggtaacaactattacgcgtatcgcgactcgcccgatctacgctactacatgcccctaagcggtggtcgtcactatcacccgggcacttttgatcgtcacgtgctggtgcggcttttccacaaacgcggcgttattcagcatttgccgggctacgggacgataacggaggagctggtgcaagagcgtctgtcgggccaggtgcgcgacgacgtgctttctctctggagtcgacgtctgctggtcggcaagctgggtcgcgacgtgcccgtctttgtgcacgaacagcaatatctgcgttcgggcctgacctgcctggctggcctgctgttgttgtggaaggtgaccaacgcggatagcgtcttcgctccgcgcacgggcaaatttacgttggccgacctgctgggttcggatgccgtagccggcggcgggttgcccggggggcgcgcgggcggcgaagaggagggctacgggggacggcacgggcgggtacgtaactttgagtttctggtacggtactacatcgggccgtggtacgcgcgcgaccccgcggtcacgctgtcgcagctctttcccggcctggctctgttggccgtgaccgagagcgtgcgcagcggctgggatccctcacgtcgcgaggacagcgccggaggtggcgacggcggcggcgccgtgctcatgcagctcagcaagagcaaccccgtggccgactacatgttcgcgcagagctccaaacagtacggcgatttacgtcgcttagaggtacacgatgccctgctctttcactacgaacacgggctagggcggctgttgtcagtgaccctgccgcgtcaccgtgtgtccactctgggctcgtccctctttaacgtcaacgatatttacgaactgttgtactttttagtgttggggtttcttccgagcgtggcggtgttgtaatttccaccacgtgtcgctcgctgcataaagggcgaacgtcctcggagagggtatattcgttcggcgagagcgggcggcggtggtgggtatgtccccttctgtggaggagactacctcagtcaccgagtccatcatgttcgctattgtgagtttcaaacacatgggcccgttcgaaggctactctatgtcggccgatcgcgccgcctcggatctactcatcggcatgttcggctccgttagcctggtcaacctgctgactatcatcggttgcctctgggtgttgcgtgttacgcggccgcccgtgtccgtgatgatttttacttggaatctggtacttagtcagtttttttccatcctggccaccatgttgtccaagggtatcatgctgcgtggcgctctaaatctcagcctctgtcgcttagtgctctttgtcgacgacgtgggcctatattcgacggcgttgtttttcctctttctgatactggatcgtctgtcggccatatcttacggccgtgatctctggcatcatgagacgcgcgaaaacgccggcgtggcgctctacgcggtcgcctttgcctgggttctttccatcgtagccgctgtgcccaccgccgctacgggttcactggactaccgttggctaggctgtcagatccctatacagtatgccgcggtggacctcaccatcaagatgtggtttttgctgggggcgcccatgatcgccgtactggctaacgtggtagagttggcctacagcgatcggcgcgaccacgtctggtcctacgtgggtcgtgtctgcaccttctacgtgacgtgtctcatgctgtttgtgccctactactgcttcagagtcctacgcggtgtactgcagcccgctagcgcggccggcaccggtttcggcattatggattacgtggaattggctacgcgtacccttctcaccatgcgtcttggcattctgccgctctttatcattgcgttcttctcccgcgagcccaccaaggatctggatgactcctttgattatctggtcgagagatgtcagcaaagctgccacggtcatttcgtacgtcggttggtgcaggcgttgaagcgggctatgtatagcgtggagctggccgtgtgttacttttctacgtccgtccgagacgtcgccgaggcggtgaaaaagtcctccagccgttgttacgccgacgcgacgtcggcggccgttgtggtaacgacaaccacgtcggagaaagccacgttggtggagcacgcggaaggcatggcttccgaaatgtgtcctgggactacgatcgatgtttcggccgaaagttcctccgtcctctgcaccgacggcgaaaacaccgtcgcgtcggacgcgacggtgacggcattatgagcggcggcgctgtacggcagcggggagaaaagtggcagataaatcacgtcaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgcggacggtgcgggtgccgggacagcgtgccggcgaacgttctgtaatcttccataataaaagtaaaaatgcccgtctcgtgtcgactccgctggatctcgaaggcgtcgggggtaatgcgcatcttgccggtgccgatgagataaaagtaccacattttttgacagatgatgcgaatcaagggttcgtacgcttcggcaccccagtggcgcgtgaagaaggccgccagacgaaacaagcggtgtccgtagagcgtgcctagggagaagaggatgttgccgttgcgcgccaggtcttcggggaaaacgaccggcaggccggtgtggcgctgcacaaagcgcgtcagcagtccgccgctcaagcgcgggtgacacaggcgctggctgagacgggcggcgcgcgtttcatcgaacacggccgcctcaaagtccagccccgggaaggcctggcgcagttcgcggtacagatgaggccagtagggttgcggcgtcttgcgactaagcacggcgtggtccgagacacccaggttgttcatggtttcgcgcagtagcagcgtttcgagaccgcggtgaaagaggaggacgcagatgaggcgtacgatcttgagttcttccaaacgcagcgagctcagcggctgtccgcgcgacatcttctcgctaatctgtaatattagatgattggcgcaagtaaaggagaatttgcccgtgcggacccgcgggacggcggggttctcttcgtcgcgggccatcatcgttcgctcggtgagcgggtagcgacggtgacgacaatgacgatggacgagcagcagtcgcaggctgtggcgccggtctacgtgggcggctttctcgcccgctacgaccagtctccggacgaggccgaattgctgttgccgcgggacgtagtggagcactggttgcacgcgcagggccagggacagccttcgttgtcggtcgcgctcccgctcaacatcaaccacgacgacacggccgttgtaggacacgttgcggcgatgcagagcgtccgcgacggtcttttttgcctgggctgcgtcacttcgcccaggtttctggagattgtacgccgcgcttcggaaaagtccgagctggtttcgcgcgggcccgtcagtccgctgcagccagacaaggtggtggagtttctcagcggcagctacgccggcctctcgctctccagccggcgctgcgacgacgtggaggccgcgacgtcgctttcgggctcggaaaccacgccgttcaaacacgtggctttgtgcagcgtgggtcggcgtcgcggtacgttggccgtgtacgggcgcgatcccgagtgggtcacacagcggtttccagacctcacggcggccgaccgtgacgggctacgtgcacagtggcagcgctgcggcagcactgctgtcgacgcgtcgggcgatccctttcgctcagacagctacggcctgttgggcaacagcgtggacgcgctctacatccgtgagcgactgcccaagctgcgctacgacaagcaactagtcggcgtgacggagcgcgagtcatacgtcaaggcgagcgtttcgcctgaggcggcgtgcgatattaaagcggcgtccgccgagcgttcgggcgacagccgcagtcaggccgccacgccggcggctggggcgcgcgttccctcttcgtccccgtcgcctccagtcgaaccgccatctcctgtacagccgcctgcgcttccagcgtcgccgtccgttcttcccgcggaatcaccgccgtcgctttctccctcggagccggcagaggcggcgtccatgtcgcaccctctgagtgctgcggttcccgccgctacggctcctccaggtgctaccgtggcaggtgcgtcgccggctgtgtcgtctctagcgtggcctcacgacggagtttatttacccaaagacgcttttttctcgctacttggggccagtcgctcggcagtgcccgtcatgtatcccggcgccgtagcggcccctccttctgcttcgccagcaccgctgcctttgccgtcttatcccgcgtcctacggcgcccccgtcgtgggttacgaccagttggcggcacgtcactttgcggactacgtggatccccattatcccgggtggggtcggcgttacgagcccgcgccgtctttgcatccgtcttatcccgtgccgccgccaccatcaccggcctattaccgtcggcgcgactctccgggcggtatggatgaaccaccgtccggatgggagcgttacgacggtggtcaccgtggtcagtcgcagaagcagcaccgtcacgggggcagcggcggacacaacaaacgccgtaaggaaaccgcggcggcgtcgtcgtcgtcctcggacgaagacttgagtttcccaggcgaggccgagcacggccgggcacgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgctgcgcggcagagttctacgttactttcggcggctcttccctctgcggcctcttcctccccaactactactaccgtgtgtactcccaccggcgagctgacgagtggcggaggagaaacacccacggcacttctatccggaggtgccaaggtagctgagcgcgctcaggccggcgtggtgaacgccagttgccgcctcgctaccgcgtcgggttctgaggcggcaacggccgggccctcgacggcaggttcttcttcctgcccggctagtgtcgtgttagccgccgctgctgcccaagccgccgcagcttcccagagcccgcccaaagacatggtagatctgaatcggcggatttttgtggctgcgctcaataagctcgagtaagagagacgctatatttagggcttccctctcttttttttctacaccgtgataccctaataaagcacaccgcggttattatcaacgtctctgtgtttttattatttagaaataaatacagggaatgggaaaaacacgcgggggaaaaacaaagaagtctctctctagatgcggggtcgactgcgtggggtgctggaagtggaagcggtgctgatgggtgagggtcgtggcgcgggcacggaccgcaacgtgctgctgatgtctgccgcggtacgcacgtcgccgtccatgtcgctgcgcagataagaggtaggtcgtagtgcggcgtgctgcacgctcaccgttaatggtaccaagtcgtcaaggctcgcaaagacgtgccacgaggggatgacgagcgtgagagccccgttgttaccgcttcgacgtctttgtccggtcaggatcagtgccggggacagtccggcttgggtgtccgagtcctcgtcgccgctggcttcctcgaagccggcaaacatggcttcggacaggggggtcggcgtcggtgtggaggagaggtcatcttcgtcgtcctcttcctcttcttcctcctcttcctcggtgggtggtaatccgggggactgcgggagaaactcggagacggcgccgcgcatgacgttgctccgtggaaagagaccggcgcgcagctgcacctggggacgcttgattttgtccggtttaccgggtgtgagagtccaaaacccacggcggaaaaagtggatgcggcctagcggctgtcggtgttccaaatgaacggcctgatcgccggtcagcgtgacgcggagggtgattcgcacacgatcgggtagcgggccggcttctatggagacgcccgggatgttttccgggaaaaagatggtgtcgtgagtctgattggtctcgaaagcattctggatctgcacgatgtactcgggatgtatgcgcgtcagcgtaaaacttttgggaatcaacagctggaagccgttgtccggcaagcgtcgtaggtgcgggtacggattgtgtcgcgccaccacctcggcgcgatgcgtgtaaaccgaaaagtgcagaaacacgctggtcggcgggtgcggtgagtcgtgatgcagaaacagcatgatccattggcctcgttcgtccgtctccgttttgtggatgtacgtgttagggtccgaacaggccagctgctccagggcgtctaccagcgtcagcgggatggcgccggcgcgaaaggcgaactggctgacaaagatctgccctgcctccaaactgctgtcggttctgcggcgccagttcggcgttacggtcagtcgcacggcccagtggtgagccgtgcggcggatgatggcgcgcgcttccattcgcggccgattttcttcgccgccgcgccgctggctctgaaagaggtgcagtccgctaacgggcacgcggtccagcggcagcgcaaaggccagtaccgagaccgtgttgttttctgagcctggcgtcaggcgtcgtgggccaaagttgttgaggtccaccagcagtcggtcctgttcgcccaccacgcagcggcccttgatgtttaagtcggtcaggtctacggtgtcgtgcggagatttgttctcctgaaaacagcagagaaccgagggccggctcacctctatgttggtacgcaggtccaggagtcgcagacgaccggcttccagcgagccgccttccacgttggtgatgagccgaagcacctggcagtgcaggcgaccaaagcttccgctggcggcttcggcctcgctgatcgcggccgcttccgacgagggtccctcaccgggcgaggacgatgcctgagacattgcgaaggcgggatggggggagggtcaggggatgcgcaaaggtgaacgggtcttcgtgggaggtcgggaagggttccggcaactgtcgcaaatatagcagcggtgacaggtgtggcggccaaaagttgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaattcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgacgtgcacgccgtcgccgaggatgctgatttgcgtttgcggcccgcggaagtggaggcgcccgccatggcgccgccgccggtgacgcggggcgtcttgcgctcggtggttacgagttcttcgtcggagtccgatccgctggtccagacgtcgtcgtcgccctgggcggcaccctcgtcgtgccggtcccaggtgtgtcggtactcaagcttgccctggatgcgatactggctggtgaaggtggggtgctcgctgtactgaggcccgcgctgcagcagcaagtcgatatcgaaaaagaagagcgcagccacgggatcgtactgacgcagttccacggtctcgcgtatggcttgtacctccaggaagatctgctgcccgttcatcaacaggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcctggctggggcagatgcttcggcccgtggggttgtgcacgttgaccgacacgttctccacctcgctgcccgtaaagtacgtgtgctgcacctgcagctgattgtcgccgcggtggcatggcgtcgagtcgggcgtgtactgcgataccaagatcagcgagggctggctcacgcgtacgtggatacccgtctgcaggagtcgcgtctcgtgcggcagcaccggcgtatcgccgcgactaaacacggctttcagcacgtgccccgaaatgggacccagtacggatatcatttcgggacaacggcgaccgcgcgactccatgctgcctgcgcgtacgggtgtaggcgactgagcggcgcgccctctgcggccgccgccttacataggcaggcgaccaaacgcggaacccgaaataaaaacgttctacacagagacaaccgcggattattgagtgtctttttttattacaaaaaaaagaggcgaagccccaccgtcaccacaccccatcacacaccaccaccgatttttttttgttttaatcccgtatggcgcggacgcctagtgtccgtttcccattatcagggtcctctgtttagagatcgccgcagaccatggctaaagtgacaggactcgtcttctctgtcgtattttccgtaagcttacagtcttgcggttccgtctccggggacgccagtcgcatgggcagcaggtcctccagcgcgatggaagcgcccagcaccgagagctgctgttgcgacggcgaatgggatgtggaccgcgagtgtagcgtggatttgacttggtgcgtcattgctgacaggcaaccccgattcagcgtatgctttgacgagataaaatagaggcgccccaggagcgcgtcccgtgggaacgtggcgccgttctcgtcgctcaccagtacggttaattccaaccaggagcgcggtagccagaccgtaacgggcattttgagtccctgacggttgtgtggtacaaaaacacccagataaggcccgtaaaagcggcggtagatacgtaacgtgtgcgagttcttcagcgtcaattcgtaagggacgcgcacctccagtccctcgtccgccgcgccggagcgtggcggtacaaagtaaggcagtggcgcgtccgaaaagaagggtcgtcgcaccgtttcgcgtcgcagccgcaggcgaaacgccactgggtcggctggcgcctcggtgcggtcgcaggtcacgttgaaacgtaatatgccgtcttggtatagcgtgagtgacgacagcgtcaggtccggcggtgattcgttcgggtctagctccaatcgtccaaagacggagggtcccaatgtcttggccgtggtttccgagaggcgcgccgagatacggctggtgagtccacgcggccccgagatgccgccttccactcgatgccagcacagcgcgtgtcgtacgcgcaccgtcagcgtgggcgtcagatccgcgtccgttgattccgcggtatcagcgacggaagccgcgttctccgttacgttgtttatatccagcgtcggctcgaacgtgagttctggcagatgcagcgccagacagtcgtgtaacgccgtgtgatgcgcggctttacgtcgtagcggtagccgtttcaacagcggcgtgatgatacggagcgcgaagagattgagtgataggcgcacgatggccatgcgcgtcagttgttggtcaattaccgagcgcaggatatggcagcctgggcgtgcgggaaagagagagaaggccgggcgcacgtcagaatcctcgttagagaccacgcatagaatgccgcgttcacgatcgtcgttgcggtcatcctcgtcctcttctttcttctcttgtttttccttttttttctcgggctcgtgggaagccgccgtttcttcttcttgcaacgtcgcgggggcggtttgagactcgtcgttcgcttcccccaattgcagcggcgtagagagcagaatctggaagggatcccgcaattcttcgggtcggaggtcgaggtgcaactggatcagatggtaggtgccgcggtgcacccgaggctgacggatgtcgtgtttatccgtcagtgtgaggatggtctgcggcgagccgctgtacttgtccagctcgtccggcgttttcaggaggagactgtcgtcgtcggtactggcgacgcccatcatggtcgtggtggtagtggtggcgaggaaagtgagcggcggcgccgacagagctcggcgttggcggcggcattttccgctgtgtcggctgctattgctgccaacgccaccgccgccgcctcgtctggctcgtggccggcgggcccgattccgaaggttggggtcgacgcgtggcatgcttggtgtctgcgggcgcgagagggccggctcagcctttaaatatgcaggtcgcggatttgttatcgggtgaaacgtcacacaccgtgaagacgacctgttcgcggatgaggtcatccagctgtcgcagcatgacgaaaagcgccgacagccgcgcgatctcgtcgtcgggcgacacgtgctgcggccgcgcgggcgtgcgcggctcgccgacgctgcgctcgcggtccagccgcatcagcagctcctggcacttgacgagcagcatggagctgtcctctagcgccaacttgcgcacgtaggtcatggtcagctccgaggctaggttggccaccatggacatggagaggcaggcggtcttcatgtcgatcagcaggtgctggtcgatgaccggatcggggatggtgaaggtggcgtcgcgaaaagtaatggtctgcagctgctgcacggcagcctttacctcctcgtacgaacggtcgagcgagaagaggcccatgatgagtagtcgctggttgatttccagcgccagtggcatgggtacgatccagggcagcaccagctcccactggcccagcgtcagcaggttctcgcgcgccagcggtccgtggaagagcggcggcagcacgcatagcgcgtcgcccttctcccaagtcacgggtcccgtgttgaggacggtgtagagcagtccgtgcgtgggtacgtgtaggaggatctggttgccttctacgcgccgcatcaacgtcagcgtcatattgcgcagcaggccgcgcagtcgtacgtagccgcgggtgtgatctacgaactggtgtaggcccagctggtagtgcttgatgagatgtagacgttgcggaatgggcacaacggccgctactagcttggtcagtttgcctacgtcggcgatgctgagcttgtggtcgaaagtgcagaagatgttggcctccatggccgccatagcggcggtgaaatcctggccgcgacggaggagaggcagagacgaacaacgtctgcaccgggcgcggcgtcagagcgagcgtggcgcgtccgggcccgcgtttgcgtctaggtgactcgccgctaacctgcggtcgtcgccgtcctcctcaccggacggcctcacgagttaaataacatggattgctgcagcgggatgatttcgcctacgacgtagttaccaaagtgcgtttcggacgtagcaaaagccccggcgccacccttgagtttggtctccatcagcgccagcgtggtggtgctgaggatcggtagcgcttcctgcgtcagacggcacgggttttcgatgagttgttccgtgccttcgacgcagacgtactgcgtgtccgtgtcgccgcggatgcagtccttggcgcgtagcaggtactcgtcgatggttttgaagagcgttttgttggccgcgataatctcttctgtgttaaagtactgcgcgcaagggctgtagaatttggagttgtagcctagacgttcgcgatgtcgggtgttgtagagtacgtcgctcagacagccggcttgcgaggcccaggggttgtgtgtggccgcgaaagtctgtgcgtccgcttcgcgatggtcgtagatggccttggtggcggcctccgtgtcgtacggatcgacggccagcatgcaggaggcacgcccgcgcgggttgttggggatcttaaagtaattaacgtccatcgtcaccggcgtaaggattagttcgcacgcggccttttgtccgtgcaccgtggcggcggcattgcgctcggacatgctgccgaacgtcagcatagagatggtctccgtgtctaacagttgcggccgttctacgccggccgcgtgccggatccagcggtccacctcgtcgtgccggtacacgttcatagggaagacgcgaaagaggtcctgcacgcggacgcccatgtcggttcgcacgcggtttacgtaggctacgcaggtatttgacgtgtaacccagacccatgtctacggtgttaatgttctgcgtgacgtggtacgtagtgctgatgtcgcgttcctccttggtcacgatagggttgttgatgataactgacgtgcatgatttgccgctgtagagcagcatgtccacctcgaaggtgtcggtgcgtacggccgtgagtgcgaatcccgggtggatgtgcgccttggtctgcagcaccagtgaaactggtgagattttgtataacatggcggccagcgtcatgactgagtgcaacacgttgggacaggtggccgagtaacgcgaaaagggcgagcgcagccagttgtggtactcgtgcgcgaaggctgtgggtagcgggaaaccaccgtcgtgacggtgatagtgcgggaactcggtcacgtagcgtttaatgtcgtcgctcaacgccgcgcagatggtggggtttgagtagaaacggtggaaaggtacgggtaggctgtactcgatcaacgtcttaggcgccgtcacgacgcagcagccgttgtaaagcacgtgctgacgtgagataaagtccggcaggccctgacgctgcgcgtggtccagaggcgcgcgcacttcgagcaccttgacgtgctcgcccacgaattgcacggccaaaaacagttcacgacaggcctgcagcagcggcgtatgtgcgtcggtggcgacgtcctccaccagctcggtcagcatctcgcctacggcttgacgttgcgccgctatcgagtcttcgggggtgacaccgcttgtgctctctttcgacgtcgtacctgacgtggagaccgcggtggcggccggcatcaggagaaacgccggtcggtaaaagaggtctactagcagcgtcttgaggttgagtcccaggccgcaggcccggttgttggtcatggcgggcatgaggcagagataaaagaccttttgtaacgtccattcgtcgtcggtggcacggtaatcgtccacaaacagcggctcgtcggcatccatggcgcccaaacgcggtacgtccgaaacgccgtggtgtcgcgcctcgatgttggccgggttcaacggttgccggtcggccactacctgtacgccttccatgttacgcggcaggtgcgtaacgaaggggggccacagccggtggtcgtgcagcgcgttcacgtaagccgatagcggttcctcagccagttgaccgttgttaagtcccggcagcgctgagatgcgcgttaccagacgcagcacggcgaccagattgcggtagtgaaagagcaactgcggtggtagggcgccatcagccaggtgttcggcgatcaacgtcaccagcgcgtagctgtgcgcaaaaaccagcagctgacgtgtgtgaaacatgttgacgatacaacgtgctacgaaagtgcggattagcaaaaaagcgtcgacgttgccgtgtaccagcacgtcgaccaggtagcaaagctcggggtaattggggcttgtcacggtggttttgaaaagtcgcaacgtctcttcgtagtcgggtggtggccgcagtcgcatgtgttccatgatctcccaggtgcgcagttcgtggaaggggcccggtgccagtccatctggcaaattaccgatgacgatacgcggtgtacacagcgccaccgtttcgctgttttcctggcagtgcgtaaagtcgaagaaggggtgcagctcggtgtagagcgtgatgttgcccaccttgtagaagtcggtgaccacaaagtcctgcttcatttcgttcaccgtgcgcgggacctcgcgtcgtacgcggtaaaaatgcggtatgcggcgcgccgcaccgcccatgggttcctgctgaaaacgacactcgagcagtcgttgcatggcgggttccgagggcggtccgcgttccgtgaaggtctgtagacagggcgcgggctcgtgcagcaccgggtggcacagcgtcttgagcgcgtccacaaagtctattttttgtacggcacggtcccggtttagcaggtaggccgtggtgggcaacgcgttgcgaacggtgtcgttaagcttaactttgctttccaccgtggtgtaaccgcgatcctcgggcagatacagccctacggggaagaaaaacgtcaggtccacgttacgttctagcggatctttggtatcggtgtttttgtagacgcgccgcaagttttccataatcaccgttttttcgcccagtcggatcacgtccatgctcagcggcgttaagctgtgcgccccggcctgcgaaagcgagtcgttgggcaaatgcggttggcccgaagtcagatgagccttgtacgagttgaaatcggccaggatcgagtgataggatatggcagtgacggcattttcgggactgagtacaaaattgccgtaggtggccggcgccgagaccgtttctttggtgatgtggcttgagagcagcgacatgatgatctgcataacgttggccgtgcttaccatcacgccgctgatcttggcccccgagctcgtggtgtacgtggtggggttgtctaggatgctatcggtggccgcttcggccagacgcgtgaggaacttgagcacatagtcgcgatcgcgcgtgcgattcagcaaaaagagcgtggccagcattttggccttgaagctctgcaagatgttgcttcgctggatgcggttcagtgcctgtcgcgccagtgtggcgttttctaccagcgtctgcaccacaaagtacggcggcgccttgcgtagcagtgtctgtaaaaagctgtgaatcaagccgcgctccatggcgtcggccgtgtttttaagcgcgcgcagcaccgtgtgcatggcttccacgttgaggatcttgtccaagatggtgccctcgaatgtctcgcgcagatacgtgaggcaggctgcgctgagctcgaaggggatggtgatgggggatttttcactgtatttggtgaccataatggtggtctgacgactagtgggcaaaccggcgccgctggccacacgcggcacctgcacgtggaacagcattttgcccgtagtcagtttattgaggtcgtggaacttgatggcgtgcgccgccgcggccaagccgctggtcaaaaaataaacccattccaggcgattgcagaaggtgccgaagatggcttcgaagtgaatattgtaacgctcggggtcatcgccgtagtagatgcgtaaggcctcaaacatctcctcgccggcgctggtcttgacgtgcgtcagaaagtcagtgggaatgcctactttaggcaggagctcgagcgccgaccagttctccatcgcggcggcggcgtgagcgcgaggcgtcggagctcggggaaagcagcgcgacccggagaatggccggcgctgcgccgcgccgcctcggctgtgacgctctaatagtcgttggcggctccgctatgccgcgccgggttttacacgtccccgtgcacgttcgcgcctgcaacctcacccaagagctatcgacgggcgaggacgcccgcttttgtcgtccgcgacccgttaacgtcgaacgggtgcgcgctgtttttgcggctctctaccgtgcctgtccgatacacgtgaggaccgagcccgagcgtgtcaagctggtactgggtcgtctgttactgggacccgtggccgtaccctgtttttgcgacggtgaagtggagggccacggtgaacatctggtacctacgacgcagttttgtcgcgggccgctgctctacgtgcaccgacgttgttgttgcggatccgtgaccgccgggcgcgcgctgtcctaccacgttctcgaaaaccacgtggccacgcatgtgctacgcggattgctctcgctgacggaatggaatcgagaattgccgagcctcttttgcgactgtcctggcggcggtggcgcctcgggaaccgaggaacgctacgctatggcctgcctgccgcgcgacctcagcctgcacctggacgactatccttacctgatggtggaaatcggacgcgtactcagtgtcagcgaggtagacgactacgtaaccgccgtctccggctacctgggcgaggccgcggcgccgcgcatccaggttcactacaagctgctctttggactcaacgtgcgtccgcaagcgccgtgcgcgttggacgctacacgcgacttttttctgctggagctgcaaaagctttggctgggcgttgaatatcaccacgaagtcacgtcggagtttttcggtcgcgtactggctcagctgcatcgcgaccgcgcccgcgtcatgatggcgcttcgcttgcccgagcagacggtgtgccacctgagcaccttcgttctcagtcgcttcaagcgacaggtactgtacttcaagctacaggtgagctacggcaagtgccggactggtcacgctgacagaagtgggggaggggggaacggtggaaatcagggacaccacaacctactgtgttatcgacgccttagcgtcacatttgccgacacagacacggtgtggagaaaccttttctacgtttattacgaactagctcgggatctggggtcccatgggacggaggaccgacccgtaagccgcggttacggtgtttcttgcgcttcgaggacgtcgcgactgtcaccgtcagaatcgacggtggtttcggcgaacggacacgcgctgtcttccaccgcgctcccgacgacgagcgcgggtcacaagctgtcactgccgcgcgacccggccgcagatcgcgttcgacgttacgtatgcattatctcgcgtctcatgtacgctcggtacggggagagatggcgtaaacactgtcaacggcggtcggagacgggagaagaggaggaggaagagacgctggaatcgggggagactgacgccacgccgccatttgactttacggggcagcagctgcgccgggcctatcaggaacaccgacgtcgtaaacatctagccgtgcagcgttacgcgccgtgccgtcgtaagctcatcggcgggatggagtttgccgaggtgacgggcgtgagtctagaccgcatcgccgtcaacgctttcaacaccaaccgcgttatcaatatgaaggctgcgctctcgtccatcgccgcgtcgggtctcggcgtacgcgcgccgcggcttcccaagaacatgacccacagttttgtgatgtacaagcacacctttaaggagcccgcttgcaccgtcagcacttttgtttccaacgacgccgtctacatcaactcgctcaacgtcaatattcgcggttcctaccccgagtttctgtactcgctgggcgtgtaccggctgcacgttaatatcgatcacttttttctgccggccgtggtgtgcaacagcaactcctcgctggacgtgcatgggctggaggaccaggcggtgattcgctcggagcgcagcaaggtgtactggaccaccaactttccgtgcatgatctcgcatactaacaacgtcaacgtgggctggttcaaagcggctacggccattgtgccgcgcgtctcgggcgccgacctggaagccattctgctcaaagaactctcgtgcatcaagaacatgcgcgacgtgtgcatcgattacggtctgcaccgtgttttcacgcaactagagctgcgcaattcgtaccagatccccttcctggccaagcagttagtgctgtttctgcgtgcttgcctgctcaagctgcacggtcgagagaagcggctgcagttggaccgcctagtatttgaggcggcacagcggggtctctttgactacagcaagaacctcacggcgcacaccaagatcaagcacacttgtgcgctcatcggcagtcgtctagccaacaacgtgcccaagatcctggcccggaacaaaaaagtcaaattggatcacctgggccggaacgccaacgtgctgacggtgtgtcggcacgtggaagcccacaagatccctcgcacgcgcctcaaagtgttagtcgaggtgctgggcgcgttgcagagtatcagcggtacgccgcacacgcgcgaagtgatccaccagacgttgtttcgattgtgctcggcggccgcagccacatcgggcctgtgttcatcccctcccccattgtgtgtgtcctcatcttcctccgtcccttctgtcccaacctccgtcagcgttgacggcagttctgaacccacgtcgccgcgagcgcggtttgcatcacgatgatggaagccgcggccgctgccgccgcggcgtttcgtccggaggagcgtccgacgccgggttggcacgacgcggcgttgttaatggacgacggtacggtgcgcgagcacgcgtttcgcaacggaccgctgtcgcaactgattcgccgtgtgttaccgccgccgcccgacgccgaagacgacgtggtttttgcttccgagctgtgtttttattgcagcggtcgttttaaccgcaggtcgtccgtcttctccatctattggcagaagcatagcgatctggtgtacgcgcttacgggcattacccattgcgccaagttggtggtggaatgcggtcagttggggagtagtaggctacggtggcgcgacggtgatgcgagtggtgaggagcgccggggagacgacgacagcagggacgagctgtacgacgtgccgggcatttatatgattcgcgtcaacgacggcggcagcaccggccccagacacgttatttggccgggtaccagcgtgctttgggcgccggacgttgtgatcactacggtgcagcgacgaatctcggcggcgcgcgccctggtgaacacgttccgccaatatttttttttgctggaacggcgctcgcacgaggagctggttctttgtccgcccgagatggaggagcgtctagcgccgctgttgcagagtgccacgcgcggtgattcggacatgtttgacggtgtggtggccagcgcttatcaccgtttgcgaatgagtaatattccgcgttcatccgcccgtctgctggaacactgcgtggggctggcgggtgctaagaagctgctcttgctcgacgtgccgcgtctggagaactattttctttgtcaagtctgtctttacgagctggacgaggacgagatgggcgaggagatgctgggcatgttggccggaaagcccgaggatgccgccgtctcgggcgcaagcggcggttttctgctacatcgcaagacgatgaagctggccgcctgtctgtgtttgttgctcaattcgctgcatttgcaccaggaggcgctggaggccttggatcctccgccgccgcgcgtcgaggagaacgaccttgtcaacgtggtgctgcgccgttattatcgcagtcacggcggcgtgcaggcgcggacgctggcggcggcccgggctttgttagccgactacgccgaaacgttttcgcccttggggagttttacgcgcctgggttacgatcgtctcgtttctgccgatgccggcgtcagtcgccggcacctggtggctctgctgcgtgcctagctgaccctgaaacggatggcgtgtatatcgtcacacaggtaggtggccatgatgacggcgatgataagatcgtccgagatacgattctggcgcttggccgagtaacgcgccgtcgtgccttcggccagcgtgacgcggtgcaggttctgaatctgctccagaagatactcgatggggtcgtggctcagcttgatggtgtaggagacgagctcttgcgaggctttgatgtagcccgagttgaaacgcgagatgaactgttccacggccagcgccttgtcgcggcccatgaggtagaagggctgttcgatgtggttctggtcgggcgtgtggtagaagagcacgcggatgagcgtgctgctctgcacgctctgtcggatgaggcaggcgatgcgcacggccgccgcctggttggtgttgccctccacggcgatgcgcagttcgtccaggtaagggtgcaggctcagcaccgagatgatcatgtgcgccgcgcactcggcgatggctacctcagaactctcggagaggtcgcgcaaaaagaaatgctctaggccgtaaatgagaaactggtgtcggtaggcgcctacggccgccacgcccgtgcccgaggccttgcggttggtggtgaaggccgggtccagatacacgtaaagcgtcttgccgaaataatcgtaggcgttggtgttgagcgtgctgtaacgcaaaatatcgaactcttcgcggctctggtccgtgatgagcacggtgttctgcgagattttattggtaccgccgatgatctcgtccatgaaagcgcccggcataaacatgttggccgtcttgcgcacttgcgagttgaggctgatgaaggtgggcttgtgcagtcggtagcaaggacacgccgtggcgtcgcccttctccgtgaagctgtgcaggtgctcttcgcacacgtaagagaccacgttgagcatgtcaaagggcgcattgttaaggcgcgtcaagaaacacgtggagtcactggtagtgttggtggacgatatgaagatgatcttggtggtattctgggccaggaaccccagaatggtgttgaaggcctctttcttgatgaagtgcgcctcgtccaccagcagcaagtggaagttttgtcctcggatgctctgtgtagagaggagacagaaaagggactcttatgattacgcacgctcgactggaagcctacagagtcggggtggggccggacaggtgagccaggtgagccgccaggtgaggcgggatcgccgtgtgccaaccgggctgcgacctgaaaaccggaaccaatccgccgacaccggcgccgcgtgacgcgcgcccataaaaacgaaagtgtcgtcgtcgcgacccgccacagccgccatgaactcgttgctggcggaactcaaccgactaggggtcgcgcacgccactacggaggatgtttttatctttgtcgaccgcctctttcaacacttttccttccttttccaggccgaggagtcaggcccgcgccgcttggaactggtcgcgtccgtgttcgagcacctgacggtggagtgcgttaacgacatcctggacgcctgcagtcacccggacgtgaacgtcgcggagacaagcaacacctgtcgtccctgcccttctcctgttccctccgcccccaaaactgtcagcggcgctcagacgtcatgtgcgacgcctcgggcgcctgtgacatgaggcacgtccagaacgcgtttaccgaggagatccagttacactcgctctacgcgtgcacgcgctgctttcgcacgcacctgtgtgatctgggcagcggctgcgcgctcgtctccacgctcgagggctccgtctgcgtcaagacgggcctggtatacgaggctctctatccggtggcgcgtagccacctgttggaacccatcgaggaggccgcactggacgacgtcaacatcatcagcgccgtgctcagcggcgtgtacagctacctcatgacgcacgccggccgttacgccgacgtgatccaggaggtggtcgagcgcgaccgcctcaaaaagcaggtggaggacagtatttacttcacctttaataaggttttccgttctatgcataacgtcaaccgtatttcggtgcccgtcatcagccaactttttattcagcttatcatcggtatctactcaaagcagaccaagtacgacgcgtgtgtcatcaaggttagtcgtaagaagcgcgaggacgcgcttctgaaacagatgcgttccgaatatggaaacgcacctgtattcggatctggcgtttgaagcgcggttcgctgacgatgagcaattgcctctacacttggtgctcgaccaggaggtgttgagtaacgaggaggccgagacgctgcgctacgtctactatcgtaatgtagacagcgctggccgatccacgggccgcgctccaggcggagatgaggacgacgcaccggcctccgacgacgccgaggacgccgtgggcggcgatcgcgcttttgaccgcgagcggcggacttggcagcgggcctgttttcgtgtactaccgcgcccactggagttgctcgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcgaccggcagcaccagttggccacgaccacgaatcaccgtcggcggggcggactgcgtaataacttagacaatgggtcggatcgccgtttgcccgaagcggctgtggcttctctggagacggccgtcagtactccattttttgaaattccgaacggagcaggaacctcctccgcgaacggcgacggcagattcagtaacctggagcagcgggtagcgcgtttgttgcgcggcgacgaggaattcatctatcacgcgggtccattggagccgccttccaagatacgcggtcatgagttggtgcagctgcgcctggacgtaaatccagacctcatgtacgccaccgatccgcacgaccgcgacgaggtcgcgcgtacggacgagtggaagggtgccggtgtctcgcgtcttcgcgaggtctgggatgtgcagcatcgcgtgcgcctccgtgtgctgtggtacgtcaattccttttggcgcagtcgcgagctgagctacgatgaccacgaagtcgaactataccgggcgttggacgcttatcgggcgcgcatcgccgtcgagtacgtgctgattcgcgccgtgcgcgacgagatctacgctgtactacgacgggacggcggcgcgttgccacagcgtttcgcctgccacgtgtcacggaacatgtcctggcgcgttgtttgggaactttgccgtcatgccttggcgctctggatggattgggcggacgtgcgtagctgtattattaaggcgctaacgcctcgtctgagccggggtgccgccgctgccgctcagcgagctcgtcgccagcgcgagcgctcggcgcccaaaccgcaggagctgcttttcgggccgcggaacgagagcggtccgcccgccgaacagacttggtacgctgacgtggtgcgctgcgttcgcgcgcaagtggatttgggcgtggaagtgcgcgcggcgcgttgtcctcgcaccgggctttggatcgtccgtgatcgccgcggacgcctgcgacgttggctctcgcagcccgaggtgtgcgtgctgtacgtcacgccagacttggacttttactgggtgctgccgggcggctttgccgtctcttcgcgcgtcactcttcatggcttggcgcagcgggctttgcgagaccgattccagaactttgaagcagttcttgcaagaggaatgcatgtggaagctggtcggcaagagccggaaacaccgcgagtatcgggccgtcgcttgccgttcgacgatctttagtccggaggacgacagctcgtgtatcttatgccagttgctgttgctctaccgcgacggcgaatggatcatctgtttttgctgcaacggccgttatcaaggccactatggcgtgaaccacgtacatcggcgtcgtcgacgcatctgtcatctacctaccttgtaccaactgagcttcggaggtcctttgggtccagccagcatcgatttcttgccaagctttagccaggtgaccagcagtatgacgtgcgatggtattacgcccgacgtgatttacgaggtctgcatgttggtgccccaggatgaagccaagcgtatcctggtcaagggtcacggtgccatggacctgacctgtcagaaggcagtgacgctaggcggcgccggcgcctggttgctgccgcgtcccgaaggctacacgcttttcttttacattctgtgttacgacctgtttacctcatgcggcaatcggtgcgatatcccttccatgacgcgcctcatggcggcggccacggcctgcgggcaggcgggttgcagcttttgcacggatcacgagggacacgtagatcccactggcaattacgtgggttgcacccccgatatgggccgctgtctttgttacgtgccctgtgggcccatgacgcagtcgctcatccacaacgaggaacccgcgacttttttctgtgagagcgatgacgccaagtacctatgcgccgtaggttctaagaccgcggcgcaggtcacactgggagacggcctggattatcacatcggtgttaaggattctgagggccgatggctgcccgtcaagaccgatgtgtgggacctggtcaaggtagaggaacctgtgtcacgtatgatagtgtgttcctgtccggtgcttaagaacctagtgcactaacggggtctgacagttcacggggagaagaaacaagaaacaacaaaaaaaaggaggacatggactcgccacggtttgtggcaaggcgtatgttatcatcatggagctactcacgttggtgttgtagcaactggcaaaaagcgccgtgctcttggcgccgcggtggtcgatgctgatcacgttgtccttgttctcgaccacgtagtcgcgcgcgaaggtgtggcggcagcggaactcgacctctttgagcacaaactgcgacacgtgcttttggtgcgccacgtagccgatgctgatgccgatcatgtgcttaagcagaaacgagataatggggatgatgaaccaagtcttgccgtgacgtcgcggcaccaggaacacggtggctttctgcttaaagatgtcgatggaggtctgcgagaggaagtcgatctggaaggcgtggatgaggtactgcagcacgcgattggccagcacggggatcttggtcacggctataaaaaagatgacgtgtatcaataaattcttttgaaacggttcgagtcggatggcttttgcgtcgccctcgacggcggtactgaagccgccgtcgagccactttttaaagtcggtcatgaagttgttgatctgctgaaactgcggatcgcggtagagctcggtcaacgcgtccagcttctggtaggaggcgcgctgctcctcggagcacgggcgaaacgtcagtttatcgagcgcgctcttgaggcgctcgtgaaacagcagctcgcgctggctttcctcgggcgagttgtagtcgcggtggcggccgcagaaggccatgagcggcaggaaggcctcgttgcacgagtgggccagcccgagttcggggtgcatcatctggtagcgcttgcggcacagcgtcgccacattggtgaaggccgtggagatgcaggaggtggggtggctcttgcgcttctgcagctccgcgtagcgctcctggatcttggcggccgagtctccgcgcaacatgatggcggcggcggtggtgcgagcggaggttaggcggcagcggcgagaggagaggaaaaagatggcggccgcgaggacgacggaggatccacccgaaaaccacgttgttgcggacgtggctcgtgggacgggcgccgtcactcgttcgtcttcgtcgtccctagtggtgtcgtcttcctcggcgtcaggctcggacgaatcttcctccgcctctcctctcagtttccccgtctcctccccctcaactgccgtcaggtctccggggtccgccggggtttcaacgtccctgtgctcggtggaacggatggtcgagctgtcggcgcagtctccggccgccgatttctcggtctccgaggcttggcgcttcgaggaggccgtaaatatggcgctggtggcctgcgaggccgtgtcaccttacgatcgctttcgcctaattgaaacgcccgacgagaatttcttgttggtcaccaacgtaattccgcgcgaatcggccgaggtgccggtgttggatagcagtagcagcggtggcgatagcgggccggaggacaaaaagaaaaacgtcgggaataaaaccgcgggggaaaagaacggcggtgggtctcgggccaaacgccgtcgtagacgacgcgctccgaaaaacgacgccgccacgccgtcttttctacgtcgacacgacgtgctggagcgtttcgcggccgcggctaagcctttgccgtcgctttgtgtgcgtgattatgcgttacgcaatgctgaccgtgttacctacgacggcgaattaatctacggcagttacctgttgtatcgcaaggctcacgtggagctgtcactctccagcaacaaggtgcaacacgtggaagccgtgctgcgacaggtgtacacgccgggcttgttagatcatcacaacgtgtgcgacgtggaggccctgctgtggctgctgtactgtggaccgcgcagcttttgcgcgcgtgacacttgtttcggtcgcgaaaagaacggttgtcctttccccgcgttgttgcccaaactcttttacgaacccgtgcgggactatatgacctacatgaatctggctgagctgtacgtctttgtttggtatcgcggctacgaattccctgcgccgacgccgcaggcgacgacggcgggtggtggtggtggtagtggtggcggcggcggggccggcgcttgtgcggtcgagacgagcgcgtcagcaggccgggtcgatgacgccggcgacgaggtgcatttgcctttaaagcccgtctcgctggaccgtctcagagaggtgttgcaggcggtgcgcggccgcttctcggggcgcgaggtgcccgcctggccggcctcgtcgcgcacctgtttgttgtgcgcgctctacagtcagaaccgtctctgtttagatctcgcgcgtgacgaggcgcggaccgtgagttatagccccatcgttatccaagactgcgccgcggctgtcaccgacgtcactttgagccacatcttgcccggccagagcaccgtctcgcttttccccgtctaccacgtcggcaagttgctggacgctctctcgctgaacgacgcgggtctcatcacgttgaatctatgacgtcggtcaacaaacagctcttaaaggacgtgatgcgcgtcgaccttgagcgacagcagcatcagtttctgcggcgtacctacggaccgcagcaccggctcaccacgcagcaggctttgacggtgatgcgtgtggccgctcgggaacagacccgatacagtcagcgaacgacgcagtgcgtggccgcacacctgttggagcaacgggcggccgtgcagcaagagttgcaacgcgcccgacagctgcaatccggtaacgtggacgacgcgctggactctttaaccgagctgaaggacacggtagacgatgtgagagccaccttggtggactcggtttcggcgacgtgcgatttggacctggaggtcgacgacgccgtctaacaggtatagcaatctccgtcacgcctctgttcagattttattaaaaaaaaaacacaacataacgacagtgtcggtgtggtagctagtgcagccttaggaacagggaagactgtcgccactatgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaaccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctgtttctcatgaacacctttttgctgcaccaggagggcttccgtaatctgccctttacggtgctgcgtctgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcttcgcgcgccgagatcgccgaggccctggagcgcgtggccgagcggtgcgacgaccggcacggcggctcggacgactacgtgtggctcagccggttgctggatttagcgcccaactatcggcaggtcgagctcttccagttgctggaaaaggaatcgcgcggacagtcgcgcaactcggtgtggcatctgttgcgtatggacacggtctcggccaccaagttctacgaggccttcgtcagcggctgtctgccgggcgccgcggcggcggacggttcgggtggcggcggctcgcactacacgggttcgcgcgccggcgtctcgccgggcatccagttcggtatcaaacacgagggcttagtcaaaacgctggtggaatgttacgtgatgcacggacgcgagccggtgcgcgacggcctcggtctgctcatcgaccccacgtcggggctgctgggcgcttccatggacctgtgcttcggcgtgctcaagcagggtagcggtcgcaccttgctggtggaaccgtgtgcgcgcgtctacgagatcaagtgccgctacaaatatttgcgcaaaaaggaggacccctttgtgcagaacgtgctgcggaggcacgacgcggcggccgtggcctcgctgttgcagtcacacccggtgccgggcgtggagtttcgcggtgaacgcgagaccccgtcggcacgcgagtttctgctttcgcacgacgcggcgctcttcagggccacgctcaagcgcgcgcgcccgctcaagccgcccgaaccgctgcgcgagtacctggccgatctgctgtatctcaataaggccgagtgttcggaagtgatcgtgtttgacgccaagcacctgagtgacgacaacagcgacggggacgccacgatcactattaacgcgagtctcggcctagccgcgggcgacggcgctggcggcggcgctgatcaccacctgcggggcagcccgggcgattcgccgccgccgatacctttcgaggacgaaaacacgcccgagctgctgggccggctcaacgtgtacgaggtagcgcgcttttcactgccggcttttgtcaatccgcgtcaccagtattactttcagatgctcattcagcagtacgtgctcagccaatactatataaagaagcatccggacccggagcggatcgatttccgcgacctgcctaccgtctacctggtctcggccatcttccgcgagcgcgaggaaagcgaactgggctgcgagttgctggccggcggtcgcgttttccactgcgaccacatcccgctcctgctcatcgtcacgcccgtggtctttgaccctcagtttacgcgccatgccgtctctaccgtgctagaccgttggagtcgcgacctgtcccgcaagacgaacctaccgatatgggtgccgaactctgcaaacgaatatgttgtgagttcggtaccacgcccggtgagcccctgaaagatgctctgggtcgccaggtgtctctacgctcctacgacaacatccctccgacttcctcctcggacgaaggggaggacgatgacgacggggaggatgacgataacgaggagcggcaacagaagctgcggctctgcggtagtggctgcgggggaaacgacagtagtagcggcagccaccgcgaggccacccacgacggctccaagaaaaacgcggtgcgctcgacgtttcgcgaggacaaggctccgaaaccgagcaagcagtcaaaaaagaaaaagaaaccctcaaaacatcaccaccatcagcaaagctccattatgcaggagacggacgacctagacgaagaggacacctcaatttacctgtccccgcccccggtcccccccgtccaggtggtggctaagcgactgccgcggcccgacacacccaggactccgcgccaaaagaagatttcacaacgtccacccacccccgggacaaaaaagcccgccgcctccttgcccttttaactcataaactttcaggtctcgcgtacgattcgcgagtcgggaatgggacacccgtgggtgtttctccgtgtgtatattattttttttttttgtgtgtgtttgcgcccccgtgtgtctaatgtgctgtttgaaacacgtaaagtagctggtggaagaacagataaacctttaataaaaaaaaaagtatgtgctcccgacccacggtctgcgtgtctcttttttatgtccatgtctccaagtctggtgcgggtggcggcggggttaagcgtcctcgaagtcttcatcatcgtcgtcgtcctcttcttcgcggaggcgacggctttccaagctgtcgtggtgactgagcacagcgacttcttcgccggaggctgtggccagcgcctggtacttgacactgccgctaccgcgtccgcgaaagtagcggacggcgcgacacgtcgtaaacatggcccatatgaaaaagagcatgccgaacgaccagctgatgccggtgcggtattcgttgctgaggaaggtatcgtactgcacgatggggtagatgaggccgcagagtccaaagaaggcgcccaggtggtagccgaattgcaccttgacgtattgaaaaaagacggcctcgatcagtaaaaagtagatgatggagatgatagcgtagaccacgaagacggctaacaccatgtggcctgtacgcacgaaaaagttgtttccgaagccgtagcacagggccatggctaccacggtggtgttgaaaccaagcgctacctctaccaggttgacgatgagcgtgcggaactgcaccgtacctttgagcttggggtgcagacgcgagaagaaaaagagtgagcgtttgtagctgcggtactgcgtgaccatgctcacgttgaaaatggtcaggcagaaaaagtgcacggcggccatgaaggcgatcatgctgggcagccgaaatgacatggtcagtgtgaatagttggaacgtgtccatgctgagaatgaagaggaaggctgtgaggctgtcgcccatgtacgaaatgtcgcgtgtcgactggtttaggctcatgcctttgtccttgcgcatgctgatcttgatccagcataccaggtagtagatggtcacggctaaaaagacgagctgcatgaacacggcgtagcacaccaactgcaccgagtctaagaaaagcataggcgtgtgcaggtgcattacgttgtaggccgacatgttgagcctttcaaagtccacgacgtgatagtagacgcaggggtagcccaggtgcggaaaattgctcagcactagatgcacgctgacgttgacaaaagtcagcaccatgaaaacgatagaagcgctccatgtccgtgtattcaccttatccacgtgcgagggggccatggcgatagcggcggcccgctcgctcgggaggcgatgggggcgcgccgatgacgacaggctcgcgggtcgttaaatactacgatgggagccgccgcggctcacgacgcggtttgagcacgtccgggcggtcggtgaaaaaagaccccgcgggccttcgcgactctcttctgtccgaggatgaccgctcagccgccgctgcaccaccgccaccacccgtacaccctgttcgggaccagctgtcatctcagctggtacggccttctagaggcctcggtgcctatcgtacaatgtctgtttttggatctgggtggcggccgtgccgagccgcggcttcacacgttcgtggtgcgcggtgaccgtctaccgccggctgaggtgcgtgctgtgcatcgcgccagctacgctgcgctggcctcggccgtgactacggacgccgatgagcgtcggcgcggcctagagcagcgtagcgccgtgttggcgcgcgtgttgctagaaggcagcgcgttaatccgcgtgttggcgcgcaccttcacgccggtgcagattcagacggacgctagtggcgtggagattttggaggccgcaccggcactgggcgtggaaaccgcagcgctgtcgaacgcgcttagtcttttccacgtagccaagctagtggtcatcggctcgtatcccgaagtgcacgagccgcgtgtggtcacgcataccgcggaacgcgtctccgaagagtatggcacccacgcgcacaaaaaattgcgtcgcggttactacgcctacgatttggccatgtcgtttcgcgtcggcactcacaagtatgtgctggagcgcgacgacgaggccgtcctggcacgcctctttgaggtgcgcgaggtgtgttttttgcgcacctgtctgcgtctggtcacgcctgtcggtttcgtggccgtggcagtgaccgacgagcagtgttgtttattgctgcagtcggcctggactcacctttacgacgtgcttttccgtggtttcgctgggcagccgccgctacgcgactacctggggccggacctctttgagacgggcgccgcccgttctttcttttttcccggtttcccgcccgtgcccgtctacgcggtccacggtctgcacacgttaatgcgcgagacggcgttggacgcggcggctgaggtgctctcgtggtgcggcctgcccgacatcgtgggctcggccggcaagctggaggtggaaccctgcgcgctctcgctcggcgtgcccgaggatgagtggcaggtcttcggtaccgaggccggcggcggcgccgtgcgtctcaatgccacggcttttcgcgagcgaccggccggcggcgatcgtcgctggctgttgccgccgctgccacgtgacgacggcgacggtgaaaacaacgtcgtggaagtcagcagcagcaccggcggtgcgcacccgccgagcgacgacgccactttcaccgtgcacgttcgcgacgccacgctacatcgagtgctcatcgtggatttggtcgagcgcgtgctggccaagtgtgtacgcgcgcgcgacttcaatccctacgtgcgttatagtcatcgactccacacttatgcggtttgtgaaaagtttattgagaatctgcgttttcgctcgcgacgcgctttctggcagatccagagtctgctgggctacatctccgagcacgttacgtcagcctgcgcttcggccggccttttgtgggttctgtcgcgcggccaccgcgagttttatgtctacgacggctattcgggtcacggacccgtctcggccgaagtgtgcgtgcggactgtggtcgactgttattggcgcaaactttttggcggcgacgatccgggtcccacctgtcgtgttcaagagagcgcgcccggcgtgctgttggtctggggcgacgagcggttggtgggtcccttcaacttcttctacggcaacggcggcgccggtggtagtccgctccacggggtggtgggtggtttcgcggcgggacattgcggtggcgcttgttgcgcgggctgcgtcgtcactcaccgccattctagcggcggcggtggtagtggcgtgggcgacgcggaccacgcgagtggcggcggtctagatgccgctgccgggagtggtcataacggcggtagtgatcgggtttctccctccacgccgcccgcggcgttaggtggctgttgctgcgcagccggtggcgactggctctcggccgtgggtcatgtcctgggccggctgccggcgctgttacgggagcgcgtgagcgtgtccgagctggaagccgtgtaccgcgagatcctctttcgtttcgtggctcgccgcaacgacgtggacttttggttactgcgcttccagcccggtgaaaacgaagtaaggccgcacgctggggtgattgactgcgcgcccttccacggcgtgtgggccgagcagggccagatcatcgtacagtcacgcgatacggcgttggcggccgatatcggctacggcgtctatgtggacaaggcctttgccatgctcacggcttgcgtggaggtctgggcgcgagagttattgtcgtcctccaccgcttccaccaccgcttgttcttcttcttccgttctctcctccgccttgccgtccgtcacttcgtcctcttcgggcacggcgacggtgtctcctccgtcttgttcttcttcgtcggcgacttggctcgaggagcgcgacgagtgggtgcgctcgctggcggttgacgcgcaacacgctgctaagcgggtggcttccgagggcctgcggtttttccggctcaacgcttaacgagtcacgtaggggaactacgtgggtaagtgacgtggatactagtaaaaaaagtgcgtcaaagctcttagcgtgtgacgtggatactagtaaaagggacgtcaaagctcactacgtgttgcgtgttttttttttttctatgatatgcgtgtctagttcgcttctcactcttcctctcctcgttcccagcgcggcggcagcttggggggtgagggcaaattggggtagttggcgttgagcacgtctagcaggcccaggcccacgggccaaccgtccacggtcttgcgctcggtcagcttgaggctgaacgagtgtgcctcgtcctgaccggtaaggcggaaaaagaagcgtgctaccagctgcaggcaggtatgccgcgtctgctggaagagcacgaaggtagcgggcacgtactgcacaatgtgcggctctttttcctcaaagagcaggtagagcgcgctgcagatcagccgcctggcgctgtggtgcagcagccggccgaagctttcgcgcacgttcaccgcgtccaggtactggagcaggtcgtgcaggcacttgcgcgttaagttgcaattttccacgcacgaaataacggtacagagcgcgaagtgcagcaggttgtcggctttgacgatgccgcagcggtgtttgagccgcagatccgagagcctcacctgcgtgacggcgtcttcggtctcgagcaaaaacacggcggagtagcctagaaaggccgaggtgcacagcaactcgctgcggtactcggccatggagaccagcagcccgtgctccgtgtgcagccacagcttgtcgccgcgcaccgtaaagtcgagcacttgcggctccatgatcatcacattctgtctagtgaaatccgtatggacctccagcacgccgcggatcatcagggcctccatttcgaaatcggccgacacgctctgggccgcgccgctcctcgtctgccgtgatcaagcggcgcggcgcggacctttcaagtgttcctgggccgccgctcgaggcagttcccctttctggcactccgcccgccgcttcgcggctcatttggcgccgacgcgccttctcgcggctgcaaatcagctccacgtatcggcaaaacttgctgtcgtcgtaggcggcggccacgatctcgccgaaggagagctgcaggtaggcctcgggtacggggtccagcgtgcccagcgccaggatgtgacacagatagggcagggtcacgcgctctaccgtgtaattggagtagacgatggcctcttcggccccttgatgcgtgaccagacgccgtaggcgaaaggtacggaaatactcgttttcccacaactgcgtgaggaagcgttccagcgactcggtgccgggcacgaactgcgagaagaagctgttggccaccaggcggttgtcttccaccgccagcggacggaagggcgccgcgtcgcgcgccttgcgcacggcctccaacacgggcaggtggtagagttcggcgtcgcgcgcgcccaggctcatggagtcctcgcgccgcgaggcgtagcgcgtgagcaggtcgcgcagttcgcgcacgcgattctcccaggtctggttaagcgtgcgcaggtcctggatctcgtccacctgcgactggatctgctcctccaggcacttgataacctgcttcttaaacaggtcgcggatgtcccgctcgggcgccgccgggccgggtggcggcggcagcagcccgacgtggcccgcgggtcctcccaccacggcgccgccgggtcccaccacgccgggtccgcccggaccacgcgcgggtagtagacggttttggtccaccagcgagggggtcaggtcctgcagaaaggactcgacgctgtcctcgatgccgatgcgcgatttgctgtccgagacgttaagcaaaaacttcataatggactttttggcgtcgctgccccggtcgtgctgctccatcatctccaccagcttcttgcagttgagctcgtggcggctggcggtcaccactttcacaggaaaggtattgagcaactggcagatcttttggtggcggcagagcccgtcgtagcgcagaatctcctcgtgcaggtgtgccaccggcgtggtgaacagcagcttgtcgcgctcataagccagcggttcggccgccacgtacaagcggatgtgcttgccgcgcagctgcgcctccagccgctccgagcgcaccttcttgaagacgcgtacctcgggcgcgttggctacgcgcacggcgcccaggcgctcggccacctgcagcagcagcgccaggttagcctgcagcaggtcctgcgccagcgggtgtgtctcggtggcccgctgcacggccgcgcgtacaaattgcgcccgctcggccgcctcgctcggcttggtcttcacgtccagcagcggtaccagtcccaccgttacgcaccaatccacgtagagaccatagtcgtcgttatcggcgtactgatataaaatgtcgcggagcgcgcccagcacgcccgtttgcacgctctggcgcaacgaggcgctccacaccaacagatactgctccaggtcctcttcgtccagcgcgcggtagggaaatagcgccgcgtgcaacttccactcctcggccacgcgccgcaccgtgatggtgtcaaagagcgttttgcacactccgtagagcagctgcttgcgcagcacgcacgggtcgcgcagcacctggtgcatgctttggccgcgacacgtccccagaaagccgtgcagcaaccgcaggaagctcatcgtctgccccgtggggaaaatgtcgatgacggcctcgtcatccacgccgcggcccacgcccaagtacgacgacgccttgatcctcaacctctcgtcggccgccaagatcgaacggatcgtcgacaaggtcaagtccctctcgcgcgagcgctttgcgcccgaggatttttcgttccagtggtttcgctccatcagtcgcgttgaacgaacgacagataacaacccctctgccgcaactaccgccgcggcaacgacgaccgttcactcctccgcctcctcttctgccgccgctgccgcttcgtccgaggccggcggcacgcgcgtgccctgcgtcgaccgttggcccttctttcccttccgcgcgctgctcgtcaccggcacggcgggcgccggcaagacttccagcatccaggtgctggcggccaatctagattgcgtgatcaccggtaccacggtgatcgccgcgcagaacctcagcgcgatcctcaaccgcactcgctcggcgcaggtcaagaccatctaccgcgtcttcggcttcgtcagcaagcacgtgccgctggctgacagcgccgttagccacgagacgctggaacgctaccgcgtgtgcgagccgcacgaggagaccaccatccagcgcctgcagatcaacgatctgctcgcctactggccggtcatcgccgacatcgtggacaaatgcttaaatatgtgggagcgcaaggccgcttcggcctccgccgcggccgcagccgccgcctgcgaggacctctcggagctgtgcgagagcaatatcatcgtcatcgacgagtgcggccttatgctgcgctacatgctgcaggtggtggtgtttttttactacttttacaacgccctgggcgacacgcgactttaccgcgaacgccgcgtgccctgcatcatctgcgtcggttcgcccacgcagaccgaggcgctggagagccgctacgaccactacacgcaaaacaagagcgtgcgcaagggcgttgacgtgctctcggcgctgattcagaacgaggtgctcatcaactactgcgacatcgccgacaactgggtcatgtttattcacaacaagcgttgcaccgacctggactttggcgacctgctcaagtacatggagttcggtatcccgctcaaggaggagcacgtggcctacgtggatcgcttcgtgcggccgcccagctccatccgcaacccctcgtacgccgccgagatgacgcggctttttctctcacacgtcgaggtgcaggcttacttcaagcggctgcacgagcagatccgcctgagcgagcgccaccgtctctttgatctgcccgtctactgcgtggtcaacaaccgcgcgtaccaggagctctgcgagctggccgacccgctgggcgactcgccgcagcccgtcgagctctggttccgccagaacttggcgcgcatcattaactactcgcagtttgtcgaccacaacctctccagcgagatcaccaaggaggcgctgcgccccgcggccgacgtcgttgccaccaacaactcctccgtccaggctcacggagggggaggatctgtaatcgggagcaccggcggcaacgacgagacggcgtttttccaggacgatgataccaccactgcgcccgatagccgtgagacgctgctcaccttgcgcattacctacatcaagggcagttcggtgggagtcaactctaaggtgcgggcctgtgttatcggataccagggcacggtcgaacgtttcgtggacatcttgcaaaaggacacgtttatcgaacgcacgccctgcgagcaggcggcctacgcctactcgttagtttcgggcctgctcttctcggccatgtactacttctacgtgtcgccctacacgaccgaggagatgttgcgtgagctggcgcgcgttgagctgcccgacgtgagttcgctctgcgccgctgccgccgccacggccgccgctcccgcttggagcgggggagagaatccgataaataatcacgtcgacgcggattcttctcagggcggccagagcgtgccggtatctcaacggatggaacatggccaagaggagacccacgacatcccctgcctgtccaaccaccatgacgactcggacgccatcacggacgccgaactcatggatcacaccagtctgtacgcggatcccttttttctcaaatacgtcaagccacctagcctggcgctgctttctttcgaggagacggtgcacatgtacactaccttccgcgacatttttctcaagcgctaccagctcatgcagcgtctcacgggcggtcgcttcgccacgttgccgctcgttacctacaatcgccgtaacgtggtgttcaaggccaactgtcagatcagctcgcagaccggctccttcgtgggcatgctttcgcatgtgtcgccggcgcagacgtacacgctcgagggctacaccagcgacaacgtgctcagtctgcccagtgaccgccaccgcatccaccccgaggtggtgcagcgcggcctttcgcggctggtgctacgcgatgcgcttgggttcctctttgtgctcgacgttaacgtttcgcgcttcgtcgagtcggcgcagggcaagagtctgcacgtgtgcaccaccgtggactacggcctcacttcgcgcacggccatgaccatcgccaagagtcagggcctgtcgctcgagaaggtggccgtggactttggggaccatcccaagaacctcaagatgagccacatctacgtggccatgtcgcgagtcacggaccccgaacacctcatgatgaacgttaacccgttgcgactgccctatgagaagaacaccgctatcaccccctatatctgtcgcgcgctcaaagacaaacgcaccacgcttattttttgacacaacaccgtgtaaggaaaacgtgactttattgagcagggtaaaaaccacgtacaagaaccacgttgtctatccccaaaaaaacacacaccgtcagggaacacatcgcctatagatagcggcactttacataaaaccaccgtacctgcatcacggtggctcgatacactggaaattcaataaaaaccaccgtgtctccgtgacggtacttatcgggtcagcgtcttttgagatttctgttcgtaaacttatccgtttccccggtccgcggtgtctcctcgcgaggctgacagtcgacgggtggtacctgcaagagaagaaacccgggtgggagcgacgccgtcgctgggtatcaaccccgcggctgaccgtcgtccggtaaaggaacaacccgtcgtcgcaagccgggttcgaccaagagaaaaaacccgggtgcggggggagacgggtcgtcctttggttgttcgcggacggcgtacatgccgcgtgggtcagtcgacggcgtcgctccgtgcggtcggtcatcattctgcttcacatatatgggttgtttgtgtttttttttataatgaatacgcactgatcctatccgtgactgcgcgtgtggcagagaggatgccttataacatgtattttgaaaaattgccaacagctataatttctctcatgtagcagaatagagaccttttgtcgtctttttgtttgtcattacttgttttccagggaattagagagagggaaccgcgcctccggcggcggtgcccgcggaccccggccccttctcgcgtgcgcggtgtgactggttgagcgaatgagcagctaggcttggtggtgctccgcgtgcgggggagaagacgattaacaacaaaaaataagtggaagtggccggtgggtctttgtccgcgtgcgcgcccatccgtcgccgggaccgagcagaaagtgatgtggtggtacattgattttttccttgacaggaaagaaaaaaaagagttttgttttcctatgtgagaggagaaaggtatgtgaggagatgttcgatgatcgtatgttacagttatgctgtaaggaagcttttatcgtgcgtcctgtttttcatttgatgtatatgacacaattgaaacctatcgataggcgtatatcgaggattcatcaattcttagaatcgtcgtctttttggctaattggactttgcccatgttggttgtcattcgtggcctgaggtcatcgtcgtccacgacgacgtgtctatagcgtgcggtgtgatcattgtgtcgagccagagaaagcgcgcctcgcacgacgtttgcggatcggctcgcgggtgtgtggaattcctaagaacataatcagctggtcgtctttctttgatgtgttgttgtcgtcgaggtcttgcttcgttttcttttttctttttagtcgatggaacttttcttcggtacgggttcttgttatggaagcttgtgttttcgaacatgaattcgaaaaaataaaaaggcctatcttcgtttcaaaaaaaggacagatatcaatcttcttaacttatatcatggtaaattcagaatcctatggtgtcttattatctctaaagtagtcaacattatggtctaacttgtatttccctgacgagatatatatgatccttataacctggctactatcatgaacaacaatatccttacttacagtcatcttcgtgagttaatgaagtataatatcggtcatctatcaacttatctgctatgtaacgtacccttttaggtattttgcgtttcttaacgagtgtacccgcctgtgtgaggcgaaactctgagaagtctaccgagtcgagttacaagtcactaaaacacttacacgagttatctatactaaaatcactatctatgttgtttgcttacctaattattatcctacatgacgaagctacctcccaacgtaaggtagggggagaggagacagaacaataaaaagtaactaatgtttcttagaacttacccgctaaggacttaccaaactatattcaccaaaaaacaacagctacgtgtttcatttgttttaatctaccgaagtaaaaaaaaaaagatgattagctatccagaacctacttacttcttaatgttttaactaaggatgcctatgggattggaaaaaaaatcacagcaacttgctactaatcagttgacagcgaagagactcataacaaagatttctgggtaatacggttataataatgcttatggactaaaggatacttggaaaaaaagaacgggctatgactatagagattcgtcgagatatcaaacttcaaataggcggctatcattcatggttgtggtgactatatcgtggagaaaaaatgtgatcgttagttagctaggtgagacttacagctatccatccgtctagtttttcgttgtaatgatgatagtacgtctatggtggtgatcgattttggttaacaatttgttcgtttaaaggcttaatgtacttatgctacatgatgtattattctttgattcatcgttcctcctaagggggtgtatgtatgtatgtactagtcgtatagtgttcctaacatcatgactattcagactatggcttcatctatcgtgtctaaagttcacttattctactattactatatatatgcactactatgtaactaggatatggtcctataaggtgtcttctatcacggtggcttgtttatcgcttggcggttacgagcaagagttcatcacggaccagccgtgaggcagggcacacgcgggtcggcggcgatgatgtcccccgcgaaggggacaacgaaaacaagaggccgccggccgcggccacggatgcgtagcggttacacaatgtttggttgagcgttttgtttcatcgtcgtggtggttttgttgttctctgtatatatcgtgtggtggctttatcgtcatcattattatcatcattcttgtttccatcatcacgatgagttttctccgttttcctctcctccagtggtagtcgtgtatcatcatcaatcatcgtagtgacgtcgttgctgctgctgctcttgccttcatggcggtatttctcttcctcccccctaaccccatattaactcgtgagtgtgatggttagagtggctgcttgttttttttttcttttctctttggaacaacaaaagaggataaagatggtcggtgaatgtattattattatcatcattatgatacggtcgcggtcttcttctccgatgacgaaacctgcgcacatcgaagaaaagacgagcgcgcgaaccgatagccgtccgtctgggacgaaggagaagatgatggggagaggaggagagccccagaagccagagcgagaagggagacgacagacatacgtcgtcaccgtcctctggaggaggcacggcggcgctgtttgttgtttggatgcttgattatatcctgttctatggggtagattattatcaataggcttggttttcaaaggtcagcctgtgtattgtcgtgtctttttttttcgttctcatgatcgcggagaccacacagacgtgcgcgtctcccaatggctaggcgttctttttaggtagtaattttttgatcttttttttttcttaacaagtctggcttgatttcttttatctatgatcgattcttctttttctcgggggttgcatcttccgtgaaagtaaagtgacactactctaaatggtaaccatattatctgttgattaggagaaaaaataattttttcgcacgaaatcgatcctaagtgaggtgatttacttgctatcacacgaaatgattatcttttgctgctaacgtactgaattttttaacagaattgcttctccgtaactatttccgcagattcagacagattgtcaaaaaagaatacggcacagaaatagtgggtctgtggcttttggttcgtgtacattcgcgtttgcgtgtcgagatttctacggtatgtttattcttcctgcgatgatgtagggtccttggtgtaagtaggatttcgagtatctctcttagagcgaacaaaataatcaaaaaacaacagctaggaaatcgagggttactctacgataaagtgtctctacaaagtgaagaatgttacgttgtggtggaataataagactcgcgtgatcgatgagtgatcgagagcggctcgaaccttctttaagagctttgtttagtgcaactttaaattacaaggagtagaaagctgaaatgaatctatgaaggtgctattctttgaatatcttactttgtacgcttcacattcgttatttggatagagagttgtctagagaaaatctgtgattctctatgagtgttatttttattatccttttggggactacgatttttcttcttgttctacataccactactactcgtaatcacatacatggacgaaaaaaaaattcgtcaggcagtagataccagattctccgacgttacggcgtcttttttttcttttgagagagtatctgctgagattgtccgtggtgtatctagtcgctatttttgttgttactagtagttttgcacacagtttattcagtatagtttttcttcttgccatgatcaattaagcccaccacctttttttttagagaggaggaatttcgtcttgatctccagccggagacaacggcggtggtggtggtggcgggagagacttcaaggcaatgaaaaaaaaaatttcgttttgccatcaagtggtgacgataacccgtcagattgataattggttcctacagaaactattctaaccgcggaagaaagaaattgaaaaaaaaaattgacaaaaacatcataacataaaggaccacctacctgggacgcgcagttgggcggcggactgggacggcatgctgcggcgatgctgtcggtgatggtctcttcctctctggtcctgatcgtcttttttctaggcgcttccgaggaggcgaagccggcgacgacgacgataaagaatacaaagccgcagtgtcgtccagaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgtttcccgcaggcgaccacgtctatcccggactcaagacggaattgcatagtatgcgctcgacgctagaatccatctacaaagacatgcggcaatgtgtaagtgtctctgtggcggcgctgtccgcacagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtagcgttgcgatttgcagtccgctccggtgtcgttcacccagttactttaataaacgtactgtttaaccacgttgcgtcgtgacgttgtttgtgggtgttgctaggcgggctggaaagatgatgtataaatagagtctgcgacggggttcggcgctctgccggctgcggcggcactcgctccacggcctccgacgagcgttgcgctcgcgctttgcgccgccgcgtcatggatctccctactaccgtcgtgcgaaaatactggacttttgcgaatcctaatcgcatcctgcatcaaagcgtcaatcagactttcgacgtgcgccagttcgtcttcgataccgcgcgtctggtcaactgcgtggacggcgatggcaaggtgctgcacctcaacaagggctggctctgcgctaccattatgcagcacggcgaggcttcggccggcgccaagacgcagcagggcttcatgtctattgacattacgggcgacggggagctgcaggagcacctctttgtacgcggcggtatcgtcttcaacaaatccgtctcctcggtggtgggctccagcggacccaatgagagcgcgctgctcaccatgatttccgagaacggtaatttgcaagtgacttacgtgcggcattacctgaaaaaccacggcgaatcctccagcggaggcggtggttgcggcgccgcgtctaccgcttccgccgtctgcgtgtcctcgctgggtggcagcggcgggactcgcgacggcccttctgcggaggaacagcaacggcgaaggcaggaacagcgtcacgaagaacggcgcaaaaagtcgtcctcgtctgccggtggtggtggaggcggcggcgctggtggtggcggtggcggcggcgggagcggcggtcagcactcctcggactccgccaacggactgctgcgggatccccggttgatgaaccggcagaaggagcggcggccgcctccctcctccgagaacgacggtgagtcccggccctcctcgcgtcacggtgctttccgagtggactcgtgagcctcccgtagcgcacgagcgagcaggcgagcggtgttggtgcgctggtggttgtgtggatgataaccatgtgctttttcgtgcgctatgtgtcgtcccgtctgtaggctctcctcccctccgggaggcgaagagacaaaagaccaccgcacagcacgaaggccatggcggcggcggcaagaacgagacggagcagcagtccggtggtgctggcggtggtggtggcggcggcagcggccgcatgtcgctgccgctggacacgtctgaagcggtggcctttctcaattactcgtcctcatcctccgcggtctcttcttcctccaacaaccaccaccaccatcatcaccaccataacgccgtgacggacgtggccgccggcaccgacggtgcgttacttctacccattgagcgcggagcggtggtttcgtcgccgtcgtcgacgtcgccgtcgtcacttctttcgctccctcgacccagcagcgcccacagcgcgggcgagacggtgcaggagtccgaggcggcggcgacggcggcggctgcggggttaatgatgatgaggaggatgaggagggctccggctgaggcggcggaggcaccaccgcagtcggaggaggagaatgattccaccactccagtctctaactgccgtgttcctccgaattcgcaggaatccgcggcgcctcagcctcctcgcagtccgcgttttgatgacattatacagtcattgaccaaaatgctcaatgattgtaaggagaaaagattgtgcgatctccccctggtttccagcagactcttgccagagacgtcgggcgggactgtcgtcgtcaaccacagcagcgtcgcgaggaccgccgcagctgtctccgcagccggcgttggccccccagcagccgcatgtccgccactcgtcaccaccggtgttgtaccctcaggttccgtcgccggtgtcgcgcccgttgccgccgcaatcgaaacaccagctgctcctccccggcccgtgtgtgaaatcaagccctacgtggtaaaccccgttgtcgccaccgccgcggctgccagtaactcttcctcgtcttcttcggctccactgccgccgccgccaccaccgtcgggcggacgtcggggtcgggcccggaacaatactcgaggaggcggcggtggtggcggtggtagaaacagccggcggcaggctgcatcgtcgtcgtcctcctcctctcggagatcgcgacggagaaacaaccgccacgaggacgaggaggacaacgaccctctgctccggttgtcgcaagttgccggcaacggccgccggcgagggccctcgttcctcgaggacggactcgaaattatcgatcccagcgaggaggctgcgatcgccgccgcctcgatcgcggcgtttttcgacgattaaaaaaccgagccgagaccggaaaaaatatgaaacaggacgcgcttggacatttgggtttccaccccttttggtgtgtgtctatatatattggtcactgattttttttacaataaagagatagacatcacagttcaccaccttgtctccccggtgtgtctattatcatcaatcacccacagagtcgccagtccatggtctctcggtaatgcgtgtccagatacgcgttggccagtataaaatggtcgttgcccacgaaggcgcgggtggtgttgcgcggcgacgggtggcaggacttgagtaccaagtgccgccgtcggtcgatcaggtactcgcaggtgtgcgcgtcggcgccccacagcatgaacaccagatgctcccggcgctctgacagcctccggatcacatggttactcagcgtctgccagcctaagtgacggtgagatccaggctgtccgtgcaccacggtgaacacggtgttgagcagcagcacgccgcgtcgcgcccaggcgtccaggcaacccgaggccggacgctgaaacccgtccaccgtacgcgccagttcgcgaaacacgttgttgagggagggcggcggcggtcggcccgccagcgtgccgaaggccaggccgctggcgctgccgtcgcagtacgggtcctggcccacgatcaccacgcgcacctgctcgggcggacacagatagctccagcggtgtacgtgctcgggtgccgggtacaccatctcgagttgccgcgcgccctccaccgccgccaccgtgtcgcgcagcagcaccgtgtcgtggtcgggcaagctgaggaagcggatccagtcggcgctcagacaaaacacgcgagcctgctcgtcgggggttaacagagagcctttattatcagcaatgttagcgagcatccactgcttgagggccatagcgcgagtgagccggcaggttgacgcgcgtctgcttcagctcgggcggcagtccggcgtagtatttatctaggtggcgtagtagcggcgggtccagctggtgacgcaggcagaattccttcactgcgttgtacaggccgtaaaagagtgtgatgccctcgggcgcggcagcggtgctcacgggcagacgcacggcgcggttggtacgcgtggcttcgttgcgtatggccaccaccacgttaaagagagacggtggcaccagctcgaagcctaacacgtgttccgtgaagatgctgcgcccgtatgacagtcgcgtgaggtcgtagccgcggcacaggtcgtccacgcacgtgtacacggccggcgagccatcgccgcactcgctgtagccgcgcatcaccgtcatccagcgcggcgctgtgtccgagctcaacagcgtcagcagggcccgcaattgatccggattgttgtacagcagggccagagtgtccaggaaagcatcgtccaacagcacggagttggcggcctccggcgtaacgggacggtaacgaataagttgcgatagcgggccatcgcgtctggtaacattcaccaacgggcgcagccaactttcatacttgtcaccctgaaacacctcacccaacaggcatcgacgcgttagttcggggcactccgcggggactttctcggcggcggtaggagcgacgctgacggcgactgaggaaacaatgggcagcagaaggcaacaccacagcagtaccaccggtccaggtgagaaagagaagccgcaatccgggcggcggcacatcaagtctgcggcacgatgagagtgtgacggtaaggagccagttggcgccgaaagttggcgctcaggtcttcgatccctaaaacgttatatattgcatccagcaggtgagccaggctaaacggattcacgtaccaggtttggttacccgcgacgatgacggccagaccgtgggcgctacagttggagaggttcctgggtacgaaggtaactgagtcgatgtcgcgccacggggggaatgagacagacgactggcgcacgctgtaatcacaactgtgattgacgtgtagcgtgtaatttcggttgcactcagcctcgaagtagagggggaaccacagttcgtcgtactcgtcgtcgtcctccagttctggctcttcttcatccaccgcaatgtctacgctgctctgagattcctcttcgtacaggatgattgacaggttatggctacacaggtcctgggcgggaggacgcgtgggagcgcgggtggtggtaatgttttccagatcgtcaaaagtcggagtgtagtctgacgccgtgacgacaccgtcgacggagatggtagaagttgcggccggtgtcacggtggtaagtatggatacagaaggggagggggaagtagcgttcgtaccgatggttgtggtattattattccctgtgtttcttgttccagaaaccgttgacgttgagatgggaatcgacgtggtgctggacgtcagattgctgaccgaggaaaccgtggtgggagtggtgacggtgttactcgtggttgaagtgacgttaggggaggtagtagtggtaccggtggtggcgacggtagtgtttgtcgtggcggcggcagcggtggtactggtaacggtggtcgcgttggtttccaccgcttcacacagtaagcaaaagcacagggccgggaaaagcaaccagccccgccatcgccgccgccgcttcatgaggtgggcaggcgaaagctggtgaattcgttgtacagcggcaagtggggcgccgcgatcgaagggtacgtcaacaagctgacgttgatattaaatacgtctggctgcttttctacgatggaagcgcacagggttacggcgtcaaacaggtctttcttggtggcgcccgagacccacatctggtatacacccgtctcgtggtacgaagtagagcgcggcaccaccggacggatgcagtccagaacgcggttgggatcctggtgaaagaatttgaacgtggctacggcctgtggcgtgtgcggcatcgtctgcgtgatgagctgctggcccgctaacacggtgacgttgtgcaacttgagcagggcactcttgagggcctggaaagcgttgccgcacgaggcgctgatctgcagctgcacggccgtggagtcgtgcagccgcatgagacgtgatacctcttcgaagacgtacttgtatttgctggcaaaaagtggcgcgtaccgacagtcggccggcaaaatgtaggtggcgttaccgccgttggtggccacggcgggcgcagcggccgcggaggccggcgtaaacagcgtcagcggccggtggtggctggtaaggtcgatcatgggcggcgtggtgaccgtggcggtggcgggcatgacggggtttgcggcgacgggcactccggccacagcggcggccgcggcggccacggcggcacttgccgagcccacacccgccggcagtcctccgccacccatgacgccgccgggcagagcgtcgcccagacagacttccgcagtggcgggcgcgctctcagcggtcagtacggtttgccgatcgacctcgcgacgaaagctggtgaggaactcactgtgatccatggccgcagggcccgagatcccgggattctgcgggtgctgaccgagtgcggggcgagttatatggaagacgattagcttggagcggagttttgcgtccctagctgacctgcggatcagcgacgtgccatagggatagactgtgagcggcggccgcaacggcggggtcggccgccgctcgtcgtcacggggcggcgcgagggaggaggaggtggtgggtacgatcttgacgtggttgacgtcctgcccgtccgggggaatacgcaaaaaaccccgtcgcggcgctaccacgatggtgcgatgggtctttctcttgttggccggggccagggacttgcagatgcgtgtggagccgtagacgatctggacgtggtcctgggagaacatgaccatcgccgccaacgctcagcggggggacgtgttgggaacacagaggctgagggaaaactccgtagaagtcagcgaaataaagacaacacagcagccactcctctcgtctcgggccctaccactgcttgaagtagggcaccgggtgtttcttttcctcaacgggctcctccagtctcttataggaccagtcccgccggcgcgccagcatgtaggtcacgtacaaaagaataatcaccatgaacaccaggaaagccagcacgccgtaggccagcagccggtcctcgaacagcgggtcgctcttgataaacacgtaggtggtggtaaaacttcggcccgcaatctgaacgtggagacgcacgacagtatacgtgccgttgaggtagaagacaaactcgcgtaaccgttgtccgttatacgtcacgttactaatattccacggcggaatgagctggttgccctgatgcagatgcacggtgctgttggggtgatagaggctgctaccgttgagcaagcagtgttcgtgttcctgaagcagcacgcggacccgcatcgtggtggcgttcaggcgagtcccgtacacggcgtagatgggataggtgaaaaggtcccaagtggcgttgtgatggcggccccagctgaagaaagagcacgtgtactcagtggtctcctgcggcctgagtcccgagataagcagctcttgagcagtagcgttgtaggagagatgtagttttcctgtggataaaattcataagttgtttattttgttggcaggttggcgggggaggaaaaggggttgaacagaaaggtaggtgctacttaccttcattatcgggggggaaggcgctaagataccccacctgagtgaagggacccttgcagtctgtccgtgcataacaagtaactgataaaatgtctggatttttggtgttattcaacaggataactttgcaggtggcgtttagagacacttggtcgtggctgtagctggcttcgcaattcacagtatacaggtgcccctctttctgcgtcgtggctatcacggaagtggaggcggacgaggtagaggtttgtaccgtggtggtgacagcagaagtgacgttgttagaggtacttattgacgtagtagacgtgacggtggtattactaggggaagtgacggcgcttgtggtgctacttttcacccccgggtgcatgtcgcccaagagcgcaactacgagcgcgatcgccagtacggaacacatgttgccgtgtgacgagacggcgtgtggacgagctatatgtggcaggaggtcgcgtcacctcttgtgacgcctaaacgtccagctccagataaaagaggcgttaataatgaagaccacaaaaaccacttgcgtcagtatgacaatcataaaggctcggtgattgctacgcctaaagtacgcgggattatccaccagttcatcctgctgaacaaagtggatgattgacgtattggtgttactatccgtgttgttgaccatggatttgactaagaaagtcttggcgccaaaagtcccgttagagccccagcaggtgacgctgctattcacataagttcccggtgcccccgctagcatgtatttcagttggtgggtataattttttctgtcgttatccatgtcattgctgtagttgaccttgctggtgagaaagcgtgttttcaacggggcacttatcatcatccctgaggccaaaaagggcgaattgcaagctgtagtgttacaaaaaatagtcaagttagtgtcattgtattgataaatgtaagccatgcttacttcaggctcataccacccgattccaatcgcggccgccatcgttaccacgtcccatcttcccagacttaccaccgccaccactaacagcgtcacccccgcacggtacatagttaccctctcgacgtcgccggctgtcaatgacgtgcctgcgtcagtggctatgatttatagcttttgggcccaaccgcaacggatctgtcgtaatctaccttccacagggccgccgcgacgatgctgaacgacaggatcagacagacggcgtacaaaagtcctaggtcggcgtcgacgcggcaggtgcggatgtctcgcagggtgggtagatgggcgatgcacaactctttctccccccgcccgtacatcccatcttgtatcagcagccgtagcgtggcattgatggtcagcggggtaaccaaagaaatcacatagggatgtgtacaggaagtgcagtgacgggtatccgtgagatgtaagtcaccaccctcctcaccgtcatcatgaaagaccaggactcgggtgagacgacccgatgaatactggatctcccaccacagtctttggtccaacaccgagagggcgcaagagattctaagtctccctgggttgggggagcagatgtaagtcccgtatgtgcctctcgccatcagggccatacacatgagggggagaaggacaattatccgggaccacccgcacccccacatcacgagaccagagacggagatgtataaaaaaagctacttttattaaacagcattctcaccacacgttaatactgtcacggggaatcactatgtacaagagtccatgtctctctttccagtttttcacttactgagacttgttcctcaggtcctggatggctgcctcgatggccaggctcagggtgtccaggtcttcgggaggggtctcggtgggctgctcaaactgccccacggcgtaggccttcgcggccgtctcgtagataggcagcatgaacccaccctggttggtggagaagatgcgcaccatgacctgtttgggaaacttttgcatcaggggcaggcacaggttgagagcgcccaacaggtccacgggggtggcagcgtggatgatcatgttgcggtaatcggaggaacgggggcataattggtgggtgtgcaattctttgaggctccacgcggccttgacgccttcgttacaagcatcggctgtgcgctgcgccacttcgggtggatgtgtcacgggcatggtgtgctccatgaggaagggagtggagagggccaggttgcacatggtgcccaggcgacaccgcaccgcatccacctcactcttcacctcatgattgcgggtgtagataatctggatgcccttgttgttcacctgcatggttttgcaggctttgatggcctcatctaacacctggtgcatactgggaatcgtgaagggcaggttcttgtactcaagagagcgattggtgttgcggaacatgcggctcacctcgtcaatcttgacgcgaccccgccgagtctgcacgttgggtgtgcagaagggggtgttcttatctttcatgatattgcgcaccttctcgttgtccaactcggagatgcgtttgctcttcttcttgcggggtccggtgctcgccccgccgctgctctgatggccgcagctcagcagagaggaggaggccgcgccaccaaaaccgccgcgcccatggtggctcgaggtcacggatgctcctccgccactgctgcatttcatctcctcggactcactctccgagtccgaagccgaactgcaggaggaggaagacgaagaggaactatcttcatcgggccggcccaagggatcgggaagaggagggtggttcatctgggagagcgggtgcgtgggagaggtcactcgcggcgtgccgctgccggtggaaggggaagacgcggtagcaccgcgggtttcgacttcttcaccctgttcttcctcgctatcagagatcacgatacagccggcggtatcgataatcttgttgcggtactggatggtaaagtcgggctcgggcttgatgtcttcctgtttgatgaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaagagtgggttgtcagcgtggggccggtgctactggaatcgataccggcatgattgacagcctgggcgaggatgtcacctgatggtgataagaagacacgggagacttagtacggtttcacaggcgtgacacgtttattgagtaggattacagagtataacatagagtataatatagagtatacaatagtgacgtgggatccataacagtaactgatatatatatacaatagtttactggtcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgactgtagaggaggataacaacacatataagtatccgtcctcctgactcatttatcgctatctcgatgccccgctcacatgcaagagttaatctttactctatctgacatacacaagtaaatccacgtcccatgcaggttagtatacatcacatacatgtcaacagacttaccgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggcctaaacacatgagaaatagagtcataagcacatgggtcacatacaggagatatgtatataacattaatacaattttattaaaaaaaaagggggggcacaaaccccgacacgtaccgtggcaccttggaggaagggccctcgtcaggattatcagggtccatctttctcttggcagaggactccatcgtgtcaaggacggtgactgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccaatagccaatattgatttatgctatataaccaatgaataatatggctaatggccaatattgattcaatgtatagatcgatatgcattggccatgtgccagcttgatgtcgcctctatcggcgatatagcctcatatcgtctgtcacctatatcgaaactgcgatatttgcgacacacagaatcgcccaagtcaccaaagtcgtctatcgccatcccccgtaaacgatataagcgctatcgccagatatcgcgtatgcccaaaaatcacttttggaaaaatggcgatatcagttacacagaaactcacatcggcgacattttcaatatgccatattttcaaatatcgatttttccaatatcgccatctctatcggcgataaacaccactatcgcgcgacatgaatttagtcggcgacagaaatctcaaaacgcgtatttcggacaaacacacattttattattcactgcagcatatagcccattttagcgcggcacacatccagccgtttgtgttttttaacgctctccaggtactgatccaggcccacgatccgggttatcttgtcgtattccaggttgatccatcgatagggaacgctgccagcggcgcccagcaggtactgcgccttgtcgttcactttgccgcagcgtattcgcccgtcagcttcgaggtataacctacaacacggaggggaagggggggtacaaaacgtgaaattagactttttttttaatgatgttttgtccctctgtcttactttcccataggctgtaaggccctcgaggaagagacttacggattgtagttgcagctcgtcagtttgttgtgtacgacctggcgtgtcaatgaatgggtcatggtggtgacgatcccgcgaatctcagccgttttctcgggactgtagcagacttcgccgtccggacaccgcagcctgtggattcatgaaaatctactctggcattcccgaggatcgtcgatggaacatggctatcagaaacgtcgagagacaaatccagacgcaccacagaacgcagacaatcataaaaatacgtacgcgacggtgaagcgattgcacattttgaaatcgtaacagcgttccggcgggtggttgacgtttatgaattcgcaacattcttctgcgcgcacccgcggcacgcggctgtgacccaatagcagccacaacgtcgtcaagaacggcgtcaggtctttgggactcatgacgcgcggttttcaaaattccctgcgcgcgcgacgggctcaaacgatgagattgggatgggtacagaaggtgtaagtctggttattggcctcggtgaacgtcaatcgcacctgaaaagacacgctgtagtcccggaagacgtgggcccagctctccagcttcatcacacacatctgataacgcgtgccatcgttgacgacgaagcgtagcagcttggtctgcttgggcaccatgtgcgctccaaaaatcttggcgtcttccacgctgatctgcacgtttccgtcgctcggtttcgaagccgtttggggcatccgttggaggatggtctggttgcgaccgctcagataccagatcacctttttcacccaggtggagcttctctccaccaaggtctggccttcccggttgtacagcagatacagggtctcgttgcgacactcgggacccgttgatacctgctggaaccccgagaattgcaagggggaccgtgggggcgagggatagagaaaaggacagtaaaacgtcgccgcgtcatgcggtttggaatacgtcagtttagaccatggcggggacggattctggtttgccgttagcgtcgaccacggagacgccagacagggcgttgcccaaaccgcgcacagaagcaggcagtgaaagtggtgacgaagcagaagccgcagcatattatttcccgtgacgcaggctagttggcaaagagccgcacgctgaactcgaggctccgggcgtgtggcgccagcgaaccggcggcgttgaacgtggtccttttgttggtgccgccgcgacggttctgacgtctaaagtcgctgatgagcaacgacacctcggtcacgttgattctgcaagcacaggttccaaacgtcatttcataccccatgcggttacttagccgttacccgttcgcccttaccttcccgttgtcatgcacctttagcgcgtaccctcacctcttgagcacgtcaaagttgtccaagccgtggctcgcatcgtagtggtagttcaacgtgaggtccacgagctgttccacatacttgtaacgggtttggtcgggcagcgcgcgagagcacgcgtcccagtaatgcggtactcggtaataatcgtttttttccgcggtttcccgctggcactgacccagcaccacggcgcacagacaaacagacagccacacccgacacagccgcatgttgcagactgagaaagaaagctttattatgagacatcatacacatagtataggcgaggtgatggggcggggaaagagttggaaccgaaagacaaaaaaaaaagcctagtcgtactcgggatctctgagcgagacgggttgcatggcaactttcattagtttgggaatctgccagctggtgctgttcgaaggttcttccatttccgaggcggtcagttcatcgtacaccgaaacgtagtacctgatggggtcctcctcattgtccgagaggtgagattcgatggtcaaaggcgagcctctcccataattgggattcacgaacgacgtgtccaagttgccatcctttctgaaatagatgacgttctcaggatcatgtttcatgcgctcgcgggccgcggacgcctcctcctcctcgtcccagtcccgagtttccaaccgctgataagggctcgaggaacaaaatccggcggggatctgagaacctcgtcgggaaccgctgccaaacgggctgctgccgccactgtcgtccgtgtcgtccaacaggttgacggcctcttcgtcggcgaaacgaaagcggcccgggtgcttgcaacacgaggagtaaactaccgcgatcagtaccgctatgaagctgaaaatggaggtgcctgtcacgatgtagaagaggatagccagcactttcatgatttcgtcattgcgcgcgtcgtgaacggaagattcgcgggcagtggtcatgttggtttcggttgtaggttcgctactcgtggtgctctcgacggtatttctgctgctggtgctagtagggacgtttgtgctgctggtcatatttgtagcgtcgctgaagtcgatgtgaagcagcaacccgaacgcgaccaggaccaggaatgttgcgcgaaggagaccccgcggggccggcattcttgagacgtggcgacgtggatttcttgttatgtccgcgaacgacgtgtaacgaggacgtggtttccgcaagcctctaccgacgccgcgacaccaggtaggttatcaaaacgcgagcccatatcgccgccatcattgtaatcagcaatgtgttgaggtactgcacgatgaatctgtctagtgacaccagccaaccctctgcttttgcgggcaagcgcgctttcggtgacagggtgtatcgtacgtagccgcgggtcaggcgcgcgttgtagcggtacacgcagaaatctatccacaggccaacgcccggctgtagcttcggatggtggataatagcgcggtgacgtacgccgcgtggctttagaatctccacctgtaaggccatctcctccaggtagtgggtctgactgcgacgcagcgtccagttcatgtaaaagtcggtctcgccgtgtccggccacgaagaggctgcttactaatccagtctacattgtgccatttctcagtctgattgcattttttagagttatgttgccaccaacgtatcctgttacgttgattacctcgtaactgcggtcgcatcttttatggactgtataattgaaaccatcacaagtaacggtcgtggtggtgttggtacatgtggtagtctcaacgtttgtatttgtcgttgtggatatctgtgtggttgttttcgacggttttgtagaaacggtggttgctggtgcagttgcagtagagcaatttatagattctgaagtgcttttattgctgataactgttgtactgtatgttgatgtggctgtctcagtactagtggaatagttaacggtagtactacagggacattgacaggaacaggtttgattgcagctttctgataacgcggatattagtatcgtccacataaccgtaaatcgccagtccattgcaatattagttctcgctcaatgggcattaatattcctttgaacgctgagccttacagaatgttttagtttattgttcagcttcataagatgtctgcccggaaacgtagctcaatcttcatgttctgtgtgatatcgaacaatgaattctgattcactgacggtgtcttgcaacatatggtactttttgttaaaggctcgtcgtgcaaaaaacagaactatgcaggccatacaaaccaacacgatggtccatacggtgcggcttctttgcgagctatgatagagattacgtttgtggtgatgtgtattgttagtatgttgacttcctttctctctatcttcattttcgatatcggtgttgtatctagggcaaacgaaagtagcattaatagcttcagtatgattttttggtgttactaataggtagaaattttcatcttcgtgatgtcctgtgaagtaattttctttaaaacaacgtctgctgtacctgccggaattggtgatatttagatcgtacagatgtagttctgtgttgttgcacgaacgacataagtcatggtacagtgaaggtctttcatgttgagaatgacatacagtatgagaggtaaggatgcgtaaccaataccaagattgggtatgtgcgttcttacgatgacctagatgatgtccgtgtgtggatcgattgtaatgtcgtatccaggcgactgaaagacaatcccacgtagaattaccttttatggtgacattactcccttctattcctgttgtattagtttctttgaaacgtatgattgttgtctctgtgtgacaagcgttggaagagttagtacggttgtacgtggtgtacgttgtggcgctgcaatttgtaagccatggcgtgcttataagtgcagtattagtggatacgttgtgcgaagtttcatctgacgtgatagttacggtgattgttgtgttataagatgatgtagcgtttgctgttacgttggtaaaagataatatagtgttggtatttgttgaaatcaattctgtagtggcagccgtattggatatattagcatatgatgtattgaatgtagaatatacggttgtgaaagtactcaagtcggaggtaacgttggtgatgttgccaatggttgacgcttgtgaggtgacagatgtgtgtggcgtcgttgatgtgttgttattcggagtagaaaatacgctggtcacaaaggtggtagaagcagtgttgggtgatgtgatggatgcagtattggtagtagtactgttgcatgtaactctatgcagaatatagaatattatgattgtatacgccgtatgcctgtacgtgagatggtgaggtcttcggcaggcgacacgcatcttttactgtaaatccccgtccaccgtcaacaacaaaggttccgtatctaggtccgtccgcagatgttcagcgtcctgttccccgattcgttgcgatcgcaggaagcagatgaccagcgcgccaacaaagatcatcattcccgaaacccaggcgcaatggagtgagaggccggaccactggcgttttaaatccgagataattgcccggtctgcctcttgggaatccgtaaccacaactctccctggtcccggataaaagcatcgacgcgtttccaaggctcggcagaagctacgtgggtggatgatgaggtagaaagcctcgacatcgccggtatactgatcctgcaggaggtagactcccgtatctttaaccgtgagattgtacagcgtcagattttggcgcgtgcacgcgaacgccgcaccgccctgacgcgtggtttctttataggcgtctgtaatgatacaaagtggcggcatacgacgcatgtatctgctgtagatatcataacgctgccagactacgctgtgatggctagtgttaagcctggtaaccagcgtgcgtgtacggtcctcgcaggtggcacggtagttggcgagctttaggggttttttggttggttcgacggcgttcgatgaacttccctgagttgtgaacaaaaacagcgacgtgactatgacaagcgtgaggggggtgctgtaggtctgcatggtgcaaaacacgttctcgccttccttatcagacgttgtcgtcctcgtcctcttcgtcgtctgtgcccgtcggttcgatcaacggggagttatctttctgtctggagggtcggtatggaatccgttcgtagatgttctgctttttagccgcgtgttgttccagctttttgcgtgtcaggctccgataggccagacattgatctacctcggtgcccgtgttgtttttctcctcctcgcgcgcgtaaattacaaagaagaccaccagcaggactatcagcgtagccacgaacgagcccgcgccccaggccgagtatgcgcctagcatggtaatgggttctgtgatccggcatttgcacatcgcgtggcacttgctgccattgccggtattagatgatgtgttattcggactgcacttgcacgtcaaatgggtattttctgatttcacgagacagttggtggcgactttggtttcggcgcagacggccacatagcttaccaagctgagtgccagaaagcacaccgcgtgcattacacgcggatacatattaaaacaccgtgttccacaagcaccgcacacgtcaatcctccccgcacggtcttcagcccgcccatgacatgatctccctcacgttacccttcaacaccctgtagtactctgtctcggcttccggtccccatgtcctaattataacaaaacaccgtgacactgtccatctccctgtctttttgcgccgccggtcccccccaaatcatgtctctagatgccgccggccaccaaccggaggcacggcggctattggattcggcattggtgcgccgcgtcttggcctgcatgatcatcgtcatcatgattgccattagcatctggatcctgacctacgtgctgtttctctaataagaaccccggcccctgacggtaattttcctttcttctccgtttctcctcagctgccgtacgtgatgcctcacggccatctccgacaggccctctccccgacctcctggacatgtgagggcttgttgctcctcctgggattgctggtgctcttctttcaccaccacaaccagtcggccgtggagaggcgtcgccgcgtctcgttcgtcgaggccgatcgactgccgcatgagagcgggtggtattcttccgatgacgacggagaccgggacggtgatgaggaaactggagagagccacaacagaaacagcgtgggactgtccgctgtttttagctgactggcgtgcgacctgtaaaccgttactcgggtctcaagatggtttggaagttgtgactcatcttcctgtgggtgatacccaaccggacgcgagtgttccataaaagccgggcgctccggcgagaccatgccatcctcgccttcggacgccccgctcctcttctctctcctctcctccccgctgccgcggccattgccgccgccgcccataccatcggcatgtcggccgacaaatcgcagctgtcttcgccgccgcagctgtagcagttaacgtcgccggcctccaggaggagatggcgctgtgcgtcgtctcttcgtcccgtctccctctgtggtcgtgggtggtgcgagagtacacgatgggtggctctcgtctcgggggaccacagggggaggggggtaatttattattcgtattactgtaattttgtatcgcttaatttgtttagagccgcacgcttgacaacgccttgtatagccttatttatcccgatgacttttttctccgtacaagaaatggacgtcacttgagcagacacagtttcatcgaccacgacagtctcatgatctgactacctctgacccgccaacgagaaaaccgaaaagtaaaagatgaccgcgccctcggagtccttttttccttttcaatcatgaaagcaagaggcagccgagagaatgccagtaagagacgaccatcgcagacacagtacgatactcatcttagaacgaaccagcgaataaccatcacacgtacagcagaatctcatgaactagtcaaccaacgtcataaaatcttcacacaatcgtttttgcgaacttttaggaaccagcaagtcaacaaaagactaacaaagaaaaaccatcttggaattaaaaaaagtagcatcgttaccttatgaaccagcagcattcagtatatacaccagatataatatatttattaatgtatcctctctttctcctgatgtaattttgtttttgtaaattcaattgttgaaagtctctccctgggggaattgcatatcttattgatgaagaagaaatccctgccatatgtgttgtcaaactatcattatttctctatatgggtattttttttctaagaagcaaaagactagcagcagccaaaataaacctgatgaaatctttaactgaactcccagtggtctgtgtgtatatttctgttggtggtcggttgtctgaacccgggtgggttgttcggaaacggcgggacggggaaacggatggaaacagcgtcgctatatacgtgacttttgatctaaacggacgtcgctaggctgacagtttacgaattgctaaacaagataggaacaaaacaagcggggctttgcctggtaggatttcctgtggaaacaataaccggatgtgattgtggctggtacataagctggttctggctgcaagcgcttttcactgcattaggtttggcgtttgcttttgcctgggaacgctatggctataacgggaaagaaccggtttggcaacattccattgtggggggggggtacttatagcgtgcctagctatgacgttgatatatgtggatgcggataatactcgtaatgagctaaaagcgacgactggtagtaattttaccattacgcataggaaagatccgttgacaactaagtggaaaaccgtttttggtaacaatggtgatcagtggttgtgcaacgttacgggtataggtaatgctactgtgaatagtaacgcaactatttgtgtgtcgagctgtggtcataatacgttggatttatgtaatttaaagtcgggagattctggcttcttcgatctgtctcgttggttcggtgaaaacatggatgaatacagtggtgatgtgtggcacttggaagtcagctaaatgttgtatcgcttagtgaattggtgttcttacagttttcatgtaataaactacgtgtaattcgttaaatttgtgtgtttttttgttagtattctgcgtaacggtggaataaaattgcgttgacctagttagatttcctgtgtagaacaatgaccggacgtgcttggactggtacatacgcaggggctggacgtggttaccggtcactggactcggtttcgctgtagctgtggttcaacctgaacatggctcccagagctgctaggaaccggtccagtcacattttttggtgggtggggggtactaaaaaagtgtttaatatttgggtttaatgataaaatccaggttatggatatgaggaaactgaatacctcgcagggtcgaaatcttaccacagttgatgatagaagacggttttccatcgggtgggaaacatgggataacggtggtgactaataatggtacaacggtcgtcaatacaacagcctgtgtttcaagttgttcgcatacgtcgcttgtgctttgcaatatgacgcagcagactgattcgttgtacggagtgggtcatcggttgaatgacgaagaagatggtgaactgtggagagtttcggtttcttaataatcccatacgacatgtgttcatttatatctgaattttaggatgatgactatagtataactctggggaacaaatatcatacgttaatcactttaagttacgccgttaggaaaagaaaatcagtccgaatgaagcatagtcagccgaatgatacagcaatagcttgtttacaacgtgttcttttttacattatgaacgtgccttgctttttatacacacatggagacagaggtccctcagcccttgtcacgacaactccctttttctaaaccgtatgtgctccaaaccgtatctcctcatcgtcacgtgaaataccatgggaccccttttcgtcacacacgtctttccgcttacccaacgcgtcagcccgcgctcggcagagctaccatataaaaacgcaggggtttagcagcttccccagatcgctgctgccccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatcttgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccgttcctcgtaggctggtcctggggaacgggtcggcggccggtcggcttctgttttattatttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtataggtagatgaaattatcccgtgtccggtcctgattccccgcatgccctgcacatcctgacgcgtcggtcagcagccaaacaatcataggaaatgaaccagaagaacaaaaagatcatctctctcggtgtatagcaacaccaacaacaaccgcatcgcaacatcttcatccgcaagacggaaagaaaacaacaataatgagaatgaaatcaccacaaccaagccagatttcacgtccatgagtttttattatattattatcaaaacgaaaaacagaaaaactgtcatagataaatataaaaaaaaatagaaaccacaaacgactactagtactccaatcttagatgtatatgctcctagataagatttagtattaccataatcatcgaagaatgaaagacgacgatgattccttaccgctcctgccacccggtctgtatgtagagagagaagagagaaaacggtgaatccaagatccccgggtcggcgtcggcatgccgctgatcgcagtggccccacctcggcatgccggcgccgggcgaggaattgctcatgaaaaaaagtatctttctgtaaaaaaagaaaacaatacatgattaaccgaaaagaaaccaacaaaaagaacccgagatcagtcgatttcgatcactacgataaacacatggaagatttcttgaaaaaagaaaagagaaagagaccaccttcccggcggcggacacgctcctctccgtcgccgttctgcaccatgattcgatcaataacaacatcatcatcggagaccatcttttaatcaatcagcgttgcagtagtcgactccctggacacgaaggagtcatccatttttatcctcgcacttcttcgctctcaaagccgcctttaaagttgaaatgaaaggatggaaacatggaatacagttttaattgcacgtatcaccattttactacaaaaagaaaaaaaaacaacttacacatagtattaccttaggtttacggataagtagagtgtaggcgtttttgaaacagttcagccaatgcaatcttgtctcggcataatcactctttctgcatataatagtagtagtagatttattcacatcaacacagcgaaaaactccagcatcaaagtacacctagagacagcccttaaaatatagtttgcagcttttagatgtacttacaccaaagaagattaccgtccttacgagaaaacagatactcggatataggaatcaagacagctctgcactgaaaacacactctcctgtcacgacaccgcgccacaccagaggcgtacgcgtgacttcatcgcaacgatccatcgtgatgtccctcgcagaacctaaaaagaccaaaaaaaaatcttggaccacagttgtcgatacttgaagacaatattctcgtgagaactttgagattcgcacttgaaacctcttaggatccacaaaaacaacaacctctgtatggaaaatgcgctattttatctcagcttttctcccaaacctcggtttcttcctattcttatgttttccctagtatatttgcctccttataagaaaagaagcacaagctcggtcgcacggattattccttctgctaatctattattttgttccttttttttttctttgccttcaccctcttcactccctgtagcaacacagagtagtagacacaataaatgagaagtttgcatgcatttgtcgtgtccgtggtttgttatggcgtgtggagtgctcgggatgggtggacgtggggacggattcttgaggctacaaagatacgcggagacgtcgtggcgaggggatgggtttattggatatcggtgaagcagcgtggcggcgaaagacgcgatccctgggctggtagatccccctaccccgtctaccagggacgtttatcctttggacacgtaaatgtctcggccggcatccacgcgccacgttcaccgcgttgtgcccagcgccatgtgcgggtcgtttcggcgtgaagttggacggcgtagtttcggggattgtgaaccgtggctgagggtgtagatgggacaggaaaaagcgtgtgatctgaccgaggcgaagcatgtgggtggtgcgatgcggtggatgtggcggggtgcggcggtttccgacgtggagatgtggagatgggggtgatccggatgcgtggcaagaggcctcgagcttgggcttctcccgcggatggacgttctaactgtacacggcggccgtggcctccgagtaaaaaaaccaggtgctgacgccagacagagacgccgtcctcggaatcgtgtgcgcgaaagcctgtgccgcggcagcgtacgacgttccagtcagcgaggccgtcgcgttggcgcgccaacagtaaggtgacgacaggttggcggcccatggttccgaagcgtccccacatgcaccagcagtcggcgtcaaagtcgcttgcgctgtcggcccagtcgccaccgccgcggcggatttccgcgcgggggacggggtagccgagtgctgcgccctcgccaatgttgtgaagtggatgcgtgagttgatgttgattctctgtgggaaaatgagcgctgtcctgtgggttggtgttggggtatgcgagtagtaggggttgtgtttgatcgtagaggtgttggcgggcctgtgcgcaagcagcgtagtctgcggcgtcgagctccatctgtgtgcggtgttcttcgtcggcgtgtttgtccgaggtttggacatgcggttgtgtgttgctgtggtgtaagggtaacgtgtgttgggcgtctgggtgaagcggcgtggtgtgggtgctgtttgtgtctgtggctggcatgattgtgcggcatgtgtgtgttgtagtgggtggaggttaaataggtgaggtgggttccctggtccgcgccgcaaactgtccccgtccccaacgtaacctcccctacgcggcgcgaacagccccggccccagcgcaacccccgtccccggccccaacaccgtcccgcacaccccccgtctccgcaacaccccggcatcgccggcggccagaacgctcgaaaacccccgacaagcgcagcgccgaaacgacacaggcaaggaccgtggaacgcaccggcagcgcgccgaaacaccgtcccgaagcccggtgccgacaacaaataccgtgggacgacacgcaccggcagtgcgcaggcagcgtcggacacaacacgcttacggccctcaacactccctcgaggacccaccacgcggccccgcaccggcggtgttttgggtgtgtcggggcgcggccgggtgggtgtgtgccgggtgtgtcgcgggcgtgtgttgggtgtgtcgggggtgtgttggcagggtgtgtcagggtgtgtcgcgggcgtgtgccgggtgtgtcgtgccgggtgtgtcgcgggcgtgtggcgggtgtgccggcggggtgtggtggcggggtgtgtcggcggtgtgcgcggcctcggggtgtgcggcttcgcaggaacgagtgtgtggcctcgcggccgttatttcccccgcggtccccagggccgtcgtccctcgcccccgggcgttgcttttcgtgtgtccccagggacccatgctgccgtcccccgggaacttcctcttttccccggggaatcacacagacacagacacgcgtcttcttttcgccgtgcgcgccgcacgtcgcttttattcgccgtcgccgtcctccgcaccacacgcaactagtcgccgtccacacacgcaactccaagtttcacccccccgctaaaaacacccccccgcccctcgaggacccaccacgcggcccggaatggatgtcgggcgtccacctagatgggtgcgcgcccgggaggcggctgtgcgctccagtggtacgcgcctgccgcgcgtcttccttcgggtagctgcctttcccagtccacggccttccagactgcgtggcgccaaggcggcgccagcacgcgccgtgcacgtcgctgcctataaaagccagctgcgtgtcgcccgcggcacacgggcgacgaaggcgtccgcgtgtctaaaccgcgtgctcgctgacgcgggtttgcttcctatatagtggacgtcggaggtgtccggcgcccatggcccagcgcaacggcatgtcgccgcgccccccgccccttggtcgcggccgcggggccggagggccttcgggggttggttcctctcctccttcttcttgtgtgccgatgggagcgccgtcaacagcgggcactggtgcgagtgctgcggctacgacgacgccgggccacggcgtccaccgggtagaaccccgcgggccgccgggcgcccctccgagtagcggcaacaatagcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgcgacgcgggcacgctgctgccgctcggacggccgtacggattctacgcgcgggtgacgccgcgcagccagatgaacggcgtgggcgcgacggacctgcgtcaactgtcgccgcgggacgcgtggatcgtactggtggctaccgtggtgcacgaggtggaccccgcagccgacccgacggtgggcgacaaggccggccatcccgagggtctgtgcgcgcaggacggactgtacctggcgctgggcgccgggttccgcgtgttcgtgtacgacctggcaaacaacacgctgatcctagcggcgcgcgacgcggacgagtggtttcggcacggcgcgggcgaggtggtgcggctgtaccgctgcaaccggctgggcgtgggcaccccgcgcgcgacgctgctgcctcagccggcgctccgacagacgttgctgcgcgccgaggaggcgacggcgctcggacgggagctgcgccggcggtgggccggcacgacggtggcgctgcagacgccgggcaggcgactgcagccgatggtactgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgcccgcgcggtggctgggctgcgcggcggggccggcgacggggacggcggcggggacgacgtcgccgccagcggcgagcggcacggagacggaggccgccggcggggacgcgccgtgcgcgatagcgggagccgtggggtccgctgtacctgtgcctccgcagccgtacggcgccgccggcgggggcgcgatttgcgtgcctaacgcggacgcgcacgcggtggtcggggcggacgcggcagcagcagcggcgccgacggtgatggtgggttcgacagcgatggcgggtccggcggcgtcggggaccgtgccgcgcgccatgctggtggtgctgctggacgagctgggcgccgtgttcgggtactgcccgctggacgggcacgtgtacccgctggcggcggagctgtcgcactttctgcgcgcgggcgtgctgggcgcgctggcgctgggacgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccctcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaacgatgcccccaccgctcagtgctcaggcaagcgtcagctacgcgctccgatacgatgacgagtcctggcgcccgctcagcacagttgacgaccacaaagcctggttggatctcgacgaatcacattgggtcctcggggacagccgacccgacgatataaaacaacgcagactgctgaaggccactcaacgacgaggcgccgaaatcgacagacccatgcctgtcgtgcctgaagaatgttacgaccaacgcttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaacgggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgggacatgtacctcgatatgtgtacatcttcgggccacaggccacggccgccagcacctccgcggccgaaaaaatgtcaaacacacgcccctcaccacgttcatcattgaaagtctctccagtccatatgttgtcaggacgtgctgtcgttctccgcttgctgcgaagcccgttcttccgagtcgtgtcgctgcgtccagcgtcgcgcccaagatgggaatttgggtcttttcacgcgtagcctcctccaccacggctgctgatcgccgtcactaaggaccgacacggaggatgacgaggagcttctccccgactccgcggtccgcgaccggctacgtagcgcgtgtccctgccagtctccgcagttacaccacacgtcgtgagcagcgtgcacctgctgccgccactgggcctcggcgtgctcaggccacccgccggagcccggtctgagctccgacgcaggatgcgcgtactcaacgtgcgccttccagtccatacagcaacaccataggtcgtgcgagtcgtcggctacccgccgccaggccagttcccgcatgggaaggctggacacgccgaccgagaggtcaccgagcccggacgccatctcttcttcctctccgtcgctgtcattaagcagccaggtcacctcctccgctccgcggtccgccggtctcgacggaccgcgccgccgtcggcaacacggaaaacagcacgccagcccgagccgctaaggccgcatgcccctgccgcccaactgaacacgcataccccgctcaactgcgttttgccacccctgtcagtgctctcgctcgagcaccaccccgcatctcccaacctttttccaataaacgaaaccgacatgacacacgtaatgggtactcgtggctagatttattgaaataaaccgcgatcccgggcgtctcagcacacgaaaaaccgcatccacatcatagacaagttacagtccacagtcacatacacgataaacaataccaacagggtaatgtttatggagtaaaacactattgtccaggccacatgcgtgtatgacttccgcaccatcccgtactgcatgttccacatgtacgcgctagacgtgtaatccactcgcagttcggggacgcaacgcagccagatcacatccccttgcagtaccagacgcagggctagcgtctcgaagatcggcatcacatctaagttccgcacgttccactttaacgactccccgggaacgaactccacgtcgtcggcgtgtacgtacaggttctctcccacgccgccataatcggccttcggatcgaagacgaaccgactcatgttgcccacgatgctcccccgagcaaacaacttgccgttgtcaatgtagcaccggttgtcctcgatttgaaaccagggatgcttggccgtggacttccagggccggagcgcgtcttccccggctttagtgattccatcgggcaggcggatcaagggacccatggaggtccaaagacccacccaggctttccagagattgttcatggtgaaacagcgtgtggactgtacgctctttcccaatttatatcccagagtagtgacgtgagcccagccacctcccagattcctgacgttttggttgtctttcctgccaattcctcccgtaaacttatgattatcctagcccattcccgataaaaatacacggagacagtagatagagttacgaataaaccggtttatttattcaagtgtctcaggagattattgaacgagcgtggataccacgccgtcgtcagttcatggtggcattgagcagccatagcaccagagtcccggcgcccggtatcagacacgctgacctaccgggcgccttcgagtccgtaccccgcggcctgggtgttagagtccgtaccttgcagcccaggtaggtttcaggtaccagctggttcgtacctgttaaataaatcgcagacgggcgctcacccctacggtcaggagcacaagaacaaccagagagaacagatatacgagcagggttctgaacagcagaccccaattgtcgtctctcatgcttcgctgaaggtaccagttgatggtctgagagctatagtccatcctcacctgaggaacacacgcggcatatttcttggggtctccccacctcgtagacaacgtgatgtccaccatatccacggtgtgcgtcaccgggtgcccaccgatgttccactcgaaataggctccgcgctcatcatggtggtactgctcaccggacacctgcagtctgtccatgtaagattgagagacgatacccacgttcacaaagtgtttctcggtgaagttgcccgacatcctccccttgaagtacagcatgcccatatggaaccagcattggttctcctccactcgaaagtgggccgatctgatctccgataccaccacatccaggggccggggcaccgagtccgcgagtctcaggaacaagacggccaggatcgcgagcaccaacaccggcttcatggctccgaaggtccgctgctcggctccgctcaccgctccggtctggctgcagcagtgcttcgctgagaagtagcgtgtggactgaacggtgtttttgaatatatagcgtttcttggtgacgttgtttcccctacgtagtaggcaactacgtgccaaaagaggcgttacggtactttccgtactgggatttccaaaccgggactttccacacggcggtttcaacaccgggacttttcacacggtgatttcggcaccgggactttccgcacggcggtttcgccaccgctgacgttctcatcgccgcccacgtcaacggtggcgacaccgtactttcccatgcggtttataaacgtcaagagtcacgtcagtcgcccacccccattacacggcgatatcccgatagggcatgaggggacccgggtgtcgcgacatgtcgacgacaggtgcggattagtggtcgtgtcgcgacatggacgtgcagggggatgtctgtcgcgatagagttgatgtgacagcccgctacacctctctgtcgcgacatgcatacacaacgggccggcttgtcggcgattgtcgcgacatatcgttatcagttagcgaccggagttgtctatcgcgacatatcgtcgactatcgcgacagaaaaaataccgttcgtagagaatgccgtgttgaaggaacgcgcttttattgagacgataaaacagcatcaggagccacaacgtcgaatcccacgtccagtcgattcgtatgttatgctgcacagcaatgctagaataacaaccagcagggtaatcccgcaacataaatacaaagtcacagcgaagaatccgtgtcgttctatcaagcgaaacgcgttccaaacggccccgtcacagacgcagttattcataagcgttaacaaccggtggctaggatgaatatccaaatcacagggcagtagccgacggactcgttgacaggtcagcctaccctcaaggttcctatcgttcggacgggatttgtgcgttttaggcctctttttcgccgcctgcaagcattggtgcgcaaagtcctcacccagctgtttccagctatcatctgcatctgtgcagtcccctgtatcgttgtaacaaacgggtctgtgcgacttcgttctcggaacacaagcttgttgtcgcggagacagagagagaagggttttcgggtcacgcgaagaccgctcaccgggggtcggcaacgcacacatcaacagaaaaccgagacgaatcaagagatccatagtgaaggagtgatatcgacgtgcttacgaaacggcgattatatatgttctcaacaataccgccctacgttgtatgatgtaacgtgtgacgtgagtctgatccaacactgaacgctttcgtcgtgtttttcatgcagcttttacagaccatgacaagcctgacgagagcgttcatcggggcatgaagtacgcattacacaaactccatatatttgttacgatagaatacggaacggaggaggctttcgccacacctatcctgaaagcgttgcattctttatgataggtgtgacgatgtctttaccattcccacggctgctttgcgtgatgatgacattcatcatgtatttccattcacacataccttttgtgcatacggtttatatatgaccatccacgcttataacgaacctaacagtttattagcccttgacaggataggtcaaaagattatatgtaggttttccggtaaaccgaattgtgatatttctctgcaggaaatagaacagcctggtacctataaaacggacaatgcagtactgtagcagcgtaaccaagtaggtccacatgaacacgtacaaaattatggtaagccatcgtttttcataccacagcctgtagctgtcgtacatgaatgaggacggtcgaggaacccagggtagttgtaattgggggcgacattcgtactgtccagaagacaattgcacgggtttcagtgagatgagtactttagcgatgtcggcgggggcgctacgtttcaccgtgacggtgagaacttgaccgtcgttttgtatttcatgaggcacgttatacaagccactggtatcatgaaggatgacctctgatgcgatgtgaggattaaattgtccctcaaaccgccaaacgctggtcatgtttccaccgtcaattacgcagctgacggtgtgagataccacgatgttggacttaggtttgggggctaattgcctttttacaaattcccttctgtattgcaggtcctgctgccactgcttttccgtgcggaaagtcgccatgtcttccacacgtgtggcgacgatagacgccaccaaggtagctaccagaagcagctggatccgcatggcattaccgtatgtcaattagaaagttgagcggacacggttatcgttcctggcggatataagtatataaacgcgagttagcctttcccgtccgttttgtacacccgttccccacacaaatgacgaatacgacctttttttttataaaaataaaccacgtgtattatataaaaacatttacatagaaaagagacacacggatcaacataaggacttttcacacttttggggtacacaggcgtgccaccgcagatagtaagcgctggatacacggtacacagtcctggccagcacgtatcccaacagcagcaccatcgccatacagatggcgatcacgaccccgagctctaagtgtctgtattcatagtgtagtcgccgcaggttatccactgaattcccgtaactgaaataacgtatatggtaccgaggctggcaccacatgggtttgcatttggtgcacggcaccaaatgcagagtgagatggtccaagtccgtgggcacccactggcgcaaacggaatacggcttcggtggtctccacgaggcactccggggcgtgcagacggccccactttcgtccgcgacggcccgaccagccgacccgagccactatccctttctcgggatagaacgtaccctgtacacgccacacagcgtccaacacgccgtccttgacgacgcagctggcctgatagctggacacgttgttaagcggcggaaagcgaaactgacgtgccggcggagccacatagttcggttcaccgtgttgtcgcggttcgtcctccctatagtaatagtagtcgtcgtcctcataggggttgccggcgtgagccagcgttacccaacagcagcccaggccgacgaggaggcgcagccaccgcctcatggcggcttcgccagtcaatcgtctttagcctcttcttcccgtgaggtccttccggtggcgcggtgccgacctcggacccagggacgtatccacctcaggtacacacagcaggctacctggacaccgaagctgaacaaggctacgtgtttcacaaactgcaccagtaccacatagaggaatgtcaggtagcgtctctccgcaaacagccgttccaagtctgagggcgttacccgcagcggcaaccagggcagcctggacgccggccggcaatggagcacgctccggttacaggcactgcaggggtaaacggttaacatcacgtaagagagtcgtgcgtccacctgtgggagctcagtttcgtaacgtagagccccgtcattttccagctggggtgcgccgaccttgaaatgggtcgcgctccgctcgttaccccaggtgccgtaggctctcggggccgtatcggagaagttgccacgcacaagccaggcggccacgagtaccccgtgctggacgtaacattcggacacggaactggagacacggtagccggacacgtccccaaacccgcgagggtactggggcagacggacggacttgctatttgacaacggacagatacgagacgacgaggacgcagacgactcgtcgctggaccacgacaaccggagcgactccttggagcggctcgagagtacacttactgcgatcagacaccagtgccagaagaaggaacaggtggacggggaccacaggatcatagccgccggcaccgcggccggccgcaggaagccgcccggcgcgtcgtctgtgtgcgggagccgaaacaccgtgcctctttatatcgtcccgacgtgacgcgagtattacgtgtcaggggaaacccccgtcacgacgaacgtgatttgtaagtgacgcggggtgctgacggggttcggcccgagaggtgacggagcgcctcacgtcagtatgatgtccgatccgcgtcagccccgacgtggttgtggtcaccgaaacccacgtttatatggacgttgagagcagcgcctgaccacatgattcatcataccatttctcggaatcgggcccatgccgggaaagcacattccttttcagtaaacaacaatgacatcataacaaatcattttattcgcgaggtggataataaccgcatatcaggaggagggatcgggtgatgacgcaggccccgcagaacagtccgaaataaatttttagtattgccccatagtcgcctagataccagaggtacgttaagttcatcaaaacgcccatcggcgtcccggaatcgtataccgggcacacgaagcgttcataacaatcccgggaggcgagtgttagggtagcagagtagtttcggggtcggtttccttccggcgacgacagttccgtgggcagcagaatgtacagcgcctcggtagctgtcgcggtgccttccacgaggatgggctgccggtgcctttcgtgattttccccgtcgtgtagccaagccgaggcccgcaaagtcttaggcgaggggaattgtccatagagtttcaccgcacccttcagtacatggttctgaataacacagccgcacgtgaagtaggtaggttctctcgtctcctccgtggctgccgccaccactcccagccaccacaacaggcagatcgccagagggttccggaggcttccccggcgtagcatggttttgggttaaagcaaaaagtctggtgagtcgtttccgagcgactcgagatgcactccgcttcagtctatatatcaccactggtccgaaaacatccagggaaaatgtcggtgcagccaacctttcacatacagcccccaaaacacttgaatcactgccaccatcatcagcgtatactgcgccgacttaatcgtgagcgcgtagtacgccattagacggcgatcttcgaacaatagtcgttcgatgtcctctaacgagctccacaggggaacccaaggcacgaggcaccggggttcgcactctacataataagtttggcattggtggcagggggaaaagtagaacaacacgagttttgtgcgttggggaacacgatagtcccggagccagtagcgttttgcgacgaggctttcggagacgtcctccaccggcgtcggcactcgatccgcgtagccctccagcgtctggtagtacacccggggtgtcggcgtgggcacggacaggttcccgcgcagggtccacagagcctccagtcgaccgcccgatcggagcacgcagcgcgcctcggaatactctactcggtactccgaaacatcggacagaggcggtaacggctccgtctccaccaagggcggaggttcatcgaaaagagtcaaggataattcaggcatactacccgcgaccggggcccagagggctagaataagcattacaaggttcattctgtcttacaagggaaggctgttaccctgtctagactcaaaagctgtaaggctgtcttatagcatgtagtcttgcacgtcacggggaacagggtggtgatctagtgacgtcgggagaacacggtgttttagggtgcgggggacaaaggacagtacgacagattaggtgatagaaacgtttttttttatttatgaaaaagccagtgtgccgtgcggcctagggccccggcgtagtttggataccagatgggggccgtcaggggtactaccacgagcagaaacataatgacttggtccatgtatagcagcatagcggtgcgcagcaggtcgccgtccgtgtagcaatttgacggtgagcgataaagcaccgttaatgtgtcgcggataagcacgatcttgaggccgtagatgaagctcacagtcagtgctaaaatgatgcgttggtatggttcccaggactgcacggcgatgaagagccagagtatgggaagcatgaagcttagcaaacagaggatggctaaccgtcgttgcatgttccaggccatgagccaggctaggcccgtacaccagacgcagagcatggatgacaggacataggcctggattaccacggtgcgatcgaaacacagcccgatggtggacacggatatcgtagtgagggtggtatataccatgaccagcatcagggtcccgggtcggcgccgacgttccagccagtacgcgtggcaacgcagagcgcagggtagcagtgtgctccagaagggcaatgtatcgcgcaggtagggggccgtcacgcgccacggtatgagcatgaaaaggatggtagtggctatggtggcgctggtctggaacacgacagtgccgtagagacgtaccatccagagaaagtgttgaacgctccgcagggtgtcttcatctttggtgattacggtgactcgacggatcggcggtggtgacggcggcgacacgggtgggggtttctctttcttatggccgagtggctcgccttggtgaaactggatctgtaccatgacgggtgctcgacgaacagtcgtgggggctttaggtacccggcaagttttatagagaaagggggacgatgggtggtggctacgagccaccgccaccttcgcaatacgaggatctgaaggcggcaaagacggtcgtccagggcaggcgccagaggttgggactgagcacgatcagcgtgattttaaacatggtcaccagtcctacgtagatcagcagcgagccgcgtaacgtctgagcagccggcagttcgtcgcggatgtaacgcgtgccgtagaaagtcacggtcatcataaggaagacgatggcgccgtagccgtagagtagaatacgctgatgatggaacacggtctggtcgccgataacccagagcgtgatgaaaaaaacgctggtgagtacccgtgagcatatgagctcccaacgcttagcgcgaaagctgtccccaaccatgacagcgccggtgcaagctatccacagcgtgaggaccagtgtgtagtcgatgaggatggcgggcaggtcggagcaccaggtgtagaaaaccgtggtaacggagaggaggcctacgtagcccatggtcaataccacgtcgtcggggtgcctttcgccctgtatcaagaccaaacaccagagaagggagggggcaaaaaccagcagcagaggggaagattcatgttgacatatgttgtgggaatcggggatacccagccaaatcattccgcagaaagccgtactgatggcgatgtgaaagaccactagggcgtagacccggacgaggacagcaaaacggcgcagccacataaggccgtggtgcagctgcaggagggaagcccattgcggcgaatgtagcgacggtagcggcgggtccatgaggcgggtgatgcgcccgagtgaacgggtgagcgtctcggtggagtcttcttataaaccagcggagctcaggcagccttgctctggaacgtcgcagtggtggtgttgaggatgacgctgagcgtgccgttgtcaatcaggtaatgatgataggtgccgagcttggccaggtagctgaacatttggtcccagcgtgccgaccacaccacgggcgtgagcatcaggagtgtggtgtgatagattagtgtttcggtggcgtaaagtatcagcgagctgcggatgacgtggctcacgggcattttggtggcgatgtagcgcacgtcttggaaaaggacggccaggatgcagcccacgaacacggtgtagagacacagcaaagtcttatgtaaccaggtgtaagtagaagccaggacgctgaccatcaccgtcaaaagtgtggaggtaaaaagcgcgtcacgccacacggagctgagacggtgctcccaagccacgccgttgcaggccacgaacaacgtccacgttaggatgaggctagaaatgccgatgggcgctgtggcgcacaggttgagcccggcggtggtgaacgagagaagcgccacatacagcgcaaacaccaggccgttgctggggtgtctgtgatcggtgagctccagcgcgcccagaaccaatactggtgtgcagctaagcaatagcggcgagggatcgtcgctgcacttgtagcccagcgaggggtaacccagccaaaccagcgcgctaatgagtacgctgaaagcggtttccagcgtcagcaatccgtagacacgcatgacaatcgcggtccgccgtagccaacacacggcatcttcggaaactgtggacgctgtttccgaataccgggaggagatcgtgcttccctcttccaaggatcggaaagtagcgtccgtcgtttccgcggacgcggcttccctggtacgctccgtttccgacgacgcggtttcccgctgcgtggaaactgtctccatgtcgggaccgcagcgcccggcggcgtatccgcaaggtctcgaagctacagcttgtcagaggaaaagtaggtttgcaaaaaggtgcgcagggtcatgattctcagcaccatcagcagagtgaaaaccagactgagaaacaccttgacggccgccaaaagcgcgcgttccagcggcgtctcgtagcgtacagccagggccgcttcgtggaaatgcgagacggctagacaggtaatgagcacgctgaaggacaagacgatcttaaagcaccaggaccaaccacgcctcaagatgaccaccacgattgccgtgaaggtcaacgtgatcaaagcatggacgaccacgatctgacggcggacggtacgttcgggagccaacaacgctacgccggtgcagctgagaaaggccagtaaggtgaacaacgcggccgagatgaccaacgtaccgtccaggcagagacatatcacgatcaacggcggcacgtgaagcagcgtgtaaaagagcagaacgccgatattgctgggatgcgatgtttcgtaacagtgaatgaagatcactgacgtgacgggtatgacaaagacgaggctgggcgaggactccgtgagacacagacgagaatggtgaaaccacgtcgcgggcgccgcgtagcagaaggcgctcaacaacgcggtcaagccggccagctgccaacccacggcgccataggtgtgcagcgccacgcggcaacagtcgacccaagccagactgcgggtcgccagccgggtctcttggatcccggggggcacgtagatgaccgtgccatcggtgggtacttgaaaccctttttctcttctcatggtgcgctgcgttctctggaaacggctgctctgtccgaaaaccagttccgaacgaaaatctagggcgagagggtggacaacggcgtcgacgacgaagcatgggacaggtcgttcggcgttaacgtcatcgcgtcggacgacggtagttctaagagacgtagatcgctcagcaggtcctgacagttgcggattcgcaagatcagaaaaaaaagggaaatgaacgtaataaagagctgtagcgacgtatgcgccacatcgcgtggcataagaacgtgacggacgaaaaggacctgctgcgaaaagtgaccggcgaagataaggcccaccgtgctgtagaagcccaaaagcagccgcaggggccaagtccagggccgcgtgaagacgatgagaacgttgaccagaaagaccacgacccagacgccgttgatgagggtaaattgatcggacagggtgcagttgtcgcgacagatgaagactacttccgcgcagagcaaggtgatgaccaacgtgagcacaaacgacgtcaacacctcgcggggctcctggcaggcacacgtgacacctagcgccgggatgtgcgccaggaggccggcgagtaatagcaccagctgtcggaacggacgacggcagcgcgggtgccggtttcgctgagcgagaaccggtcgctcatagcggaaatacacgaagagcgcggaggccacaggcaccaggaggagcacctcgggcgcccagacaacgtgacaaggaaagcccggacgcgacttgagagtcgctgtagggaagaccagagagaagctacccaagacggccaccgccgcggagatttggaagaggagcaagccggcgattcggacgacaacctcgaagcgatgcacccagcccagcacggccaccacggccgcttcatcatagtcgtcgttgttgccgctgtcgaacagccgccgaaacacgatctgtcgctgggtcgcggtgggaaagcgcagacccatgacagccggaggctatatgaccgcgcgtctaagacgcgagatccgtggggggacttttagatgtttgggcggcccgcggttctaacaggcttgattggtggagacggccggcgcggcgggtgggggaaacgacgagtttttccgttacgccatggttcgcgtgaggtttctctgtacctcccgcaaaaggtcacagcccgaaatggaggccgcgttggtggccccggtggcgcgtgacgataaccaggtcatccaagcgatgagtttgtctaatgagtcctcggtggtgaagaggatgagaatgagcaggtacaggtacaccaggttctcatagagacacaaggtgagcaggtcagcctcggaccacgcgatctcaaacaggcgcgtggtgtcaaagaccgtgacgaccagcatgaagctgagcgccatggcgtaatagcccaaaaaaagtttgtgccccaacggtacgggctgcaggtaaagtgcgatcaagaacgcgataacgccgatcacaaacagcgtgacgatgacctgccatcgacggtgattatggccggctagacccgtgacgcagctgcagaggctaaaaagcacgcaagccaagaggcccgagaaggtcactagcgtagaggaggagcaggcgctggccacgatcaccgaaagcgtcgtgagcacgctataaatggtgagcaggccagggctcggtggcgacgtgaacgatccttcatcgcgtttgccgtgcagcagggccaaacagatggtgggcaccatcaaacttaagggcggcataaagccggtgcaacagagaaagacggtgcctttaagatgcggaaaagccagcaccaggcccagacagagcaagaaggtgcaggtgccctgcacggccacggtgctgtagacccgcatacaaagtaaaaagcgacgtacgtcgttcgtcgacacggaggaaatcataatgactccgcgcgagggtcgcgggggtgggggcgcccaggccgtcccggtggcctctgagttcggagacatgacggcggtggcgatcaaaaggcgcgtatgagaaaccgtttatagagtgtaatagaatcaccgtcattcccacacggcgttcccccataaagtcacgtaacactcgagtaagcgtgaaaaagctttattgttgaataaaaaacacgagtacaacaccgagttgcggtgtcctgtctgtctactgggtggggaaggttcatcgtctgtctctagagggaaggtggggaatgtctaagcgagcgggagcgtgtcatctcccccatctttttacaacaagctgaggagactcacgccgtcgatgcgtccgccgtgtttctcggcgtactgctgcacccagacgtggccgctaaatatggcgacgctcatgtttaggagactcatgacgatggtgtacaacacgacgctgacacagacgctgtttttagacaacgttccacgctggtagatgagatccagggtctcgtaaataagcacggccgaagcggcggtcaccaccaggacgtagagtccgctgtagatcttgctgacccacagcacgggcgaaaagtaaagcaataggtaaaagacgatgacggaccagccgtagccaatcccgatgactttccagcgcgtgggattgttgccggccaggtaggtgagaccgctgcagagaacgaaaaagaccatcaccagggcaaacgacagaccgatgacgcgcctttctccgcaaaagcccgtgcacacggtgatgccggtgttgatcagcaagcacgccaccgtgagatgagcaaaattggtggtgtgtgggcgaaactcggcgaaaccgcgtagcatagccagcgtggacacgggtacgatggaggatagggctggcactatgccgttggcgcactgtccctgcacatcggggaaggcgagccaagccagcaagcagaccgtgagggtacaagccagctgccacacgagcccgtgatagacctccatgagcagcttaaagcgtttcaaccattggaagagctgctgttcggccaccagcgcgtggctgcgatggagcggcacgatggtgaccgtcggcgactcatggtgttcggaaaccgaggcggtgtcgcccatgctgccgcttacgaccgctgtcggtctaaggtaggcgtcgatgaaacagtccgtcttatcagcacccggttaccgcggatttgattgacgtcacgagtgtggtcaaaccgtggcggcaccctgtatccgacccgtcgtcatgggctccacaaccagagcctcagaagatggtacatgccgatgaataaagccacattttcgacatagaggcgtagcgagggctgaaaactctccgggaaagaactctgacaggtgatcagggacagatcgtgaattagcatcagcgtcaccgtcaacagcgtcgtcgcgtgtaaaccgagaaagaacggggccgcggcccgcagcagccaaagtcccagcgccgtagcgcagagcagagacaggaccgacggtagccacagccgccggagagacgcgccaggatcgcaacccaaaagcgaggcccccaggcagctgagatctaccgccagggcgagaagagccgcgccgacaaaggcctgcggcgacggctggcacatcagcaaggtcagaaaggctagcgcgtgcggcaggcagtaagccaacaggagtgggagtttgcggggacaacggtcgatcgacggaccgcgtagcagcaggaacaggcagccgacgggcacgacgaggctgagatgagaaagcggcggtgggtcgtcgtcccgtccccgctcgcatagctcggccaccggtggcggcatgagccaccagctgagcacgctgagggcgacggtggcggtaagctggaaggcgacgaggacggaggcgcgcagccataccgccagcctctctaggtaggggactacctcctcgacggtccattctagcgggacgacatgaagcatggcgacaagcgcggctgctgtgaaaacgggcacggttttataggcattaggacttccccgtcgtactggcggctgtcaaagtcccgttgtccaaaggcgcgccgtccgaaagactaatccaacggggacccgagagcatgagcaacaacgtgagaaagatggccatgctgtccaggtagagacagacggcgtgacggatgcattggttaggtgggcagaaaaagatgaccataagactgtcgtaggccagaatacccaaaaagaagctgatagagaaggcgcacaacgtcaccactatcttctgcagccaatcggcgtcgcttagcagagcgagcgtgaggaacgaaagcagcattaccacgtagacgcagctgatgcatttccagcgacgtcggtcacggccacctagaaacgccagccccgtaaaggagataaacaacgccagggtcatcacgtaggaacctactagtacgcggctttcagagcacatttggaagatggccgccgtcaggctgttggccaacagatagatgaaaagcaccgtggcgttactagggtgttcgttgcccaacgtgtacgtgatgaacatgcagacgatgggcacgagcacggtgagaaagaagctgtagttctcgacgcaaaagttgcggttttgtgggaaccccaaccaaaaaacgcttcccaagccgaagctgaaagccagctgaaagatgaagatggcgtacacgcgcagccatacggtgaactttttgaaccactcgagagcctccatgcgggagagcagcagcgcgttagcctcctgcgcctgcatggtggcgacggtctcggcacaaagccgctgcggcgcacctacccttctcttatacacaagcgagcgagtggggcacggtgacgtggtcacgccgcggacacgtcgattaggagacgaactggggcgacgccgctgctgtggcagcgaccgtcgtctgagcagtgtgggcgctgccgggctcggagggcatgaagtagagcacggagacaaagaggtacatgaggtccatgtacaagcagagcgcgcccgggatataactctcatactcgatgtcgtgcaggatgtcctgcgtatcgcacaccaccgaggtcacgatgacggccaaaccggctatcatcaccaggatctcacttaccgcctcgggaaaaagagaaaatacggcgaacagtaagagaatcagcgtggatgcgcccgtcaatagggaacgctgtaattccacgtcgcgggcaaacagatacgtagcgagcgtgaggaaacaaaatagcgtcactgtggccaccatggcataaatgactgaacgatgactaaagtggaagcctgacgccgtgacagccacgctggtaagcaacgtgtacgtcagtaagatccatacgtttttgggaaagttgggctcggcccaacgcaacagacctaggcacacgatggagatcattaagcaagacagcgtcagacgcacgctggaaaagagctgctccaaccggtgcggcaacaccagccagcaaaaggcgcagacgctcataaggatgaggcattgcacccagataaggatgtagatgcgcagcaggaagaccgaccgggctatctggacctgaccgcggagcgacatggcggcaacgccggcggttatcgccgagattcgtctaaatacacgaagcgaactagaaaacgcacacacgtgatttgcaaaaagaaagcagctgccggcttattattttattaaaaatttatctgtgcagaatcataagtttatgatgaataaaaacggggaaagggaatctgcttttagggacccgggtctggtccgtcgtctcccatctggtcgggttcggggatggggacctgtttcagcgtgtgtccgcgggcgtgcatggcttttgctcgccggccgcgctgtaaccaggcctctttctctgtggtcggcgagtcttccgacgggtagggagcctgggagtccatcgcttcaggcccaccgctcgttccctcgaccgtcgtgtcgtcctcgttttcgctattacacggggtttctggagtatcgcctatacggttggcgattctccgggggcggccgctctcgtcctcgtcgctgctatcgccgcccggtaattcgacgccgcattcgttgtacggaacgcggcacatgggcggcggaaagaacttgggcatgcgaaagcagcgttgtccatccacggtctgcgtggtttcatcgttatcctcccataatcccccctgtagcgccggcagcgtttcgacgctgtgagaggggaaggcccagttctggttgtcttgcagcgcgcccgtgggcagtaggtccgtgcggccccatgcgctgctgttgttgggtaccttgtcagtgccgcgagtaggtcgcagaaaccagtccagagcgctctctagctgcgagcgtgtgatggtgcccagtgcgccgtgccagcgcagcacgtctcttttcagcgtgtggtgacagacgggcagctcctccaaccgacactcgccgcgcaatccgcggtcgaagcggcagagaccacgcagtttaagcagaccgcacttgagaaacatgtgaaaattatcggcaatgcgatataggtccgagtcctcgatcttgtgtaggtagaccacgccaaacttgtcgagcagcaccaggccgctgggcacaaaaggcccgtaggccaggtaatagcccacgaggccgacgacgtaccactcgcagcacaagcgttgacgaataaagttcagaagatcgcgaaagtccgcggccggcatgtggtcaaaaggccggcaggcgcgcaggccctcgatggagcccagcatgagcaacggctccacctcggtgcgacccggcgtgcggatgaccaggttgagaccgctcatttcgcgggccgtcttggccacggccgcagcgtcagtggggtcggtgcagaggaatttttgcacatgatagcgcggttcggtggtggcgaacggcgtttgtgggtgccgatacacatattcgcaccagagtaggccgttcttggaaaaggctttgatatcactggccacctcgtagagcccgtcggtctcccagtcgtagacgtagacggtgccgtaatgacttagcatgagcacgcagggcagttcctgcgcctgcttggtgtttcgtgttagatcgctgtcgggtggacgcacggctagtacaccgacggcttccagggtgtcatcgcagcagagatagtcggcggccagagaacgtgcgtaaatctgcgggatggcggcctgttcgcgcatcactaggaaccagttggcggggttgcgcagtgctacggtggttccttggtggcgttgcacgtaggttctcagcgccggaggatcgtactggcgcagatagaggccttgcagcatcgataacgtcttttgaaagacggtgtttctaaattgaaaaacgccgtagtcgcagcggatagcatcttcgcagcgctcgtcgcgctgtcggagataggtgccccaggcttcggcggcggctttggtgagtagggacatgccggcggagccgtctcgacagcgagtcggataaagcgcgctgcgcgaaagcttaatataggagcagcgtcagacgaatcgcggctggtggcccggggggtgggacgcgccgcctacacaaagtgctcccgaaaatcgaaactcttgacccactccggagacaaatccgtattcagattgatgcgtcgagcttccacttcggcttccgaaacctcggcctccgtccggtaggcgttaacaatacgctgacccaggtgccaacgctctttctctgccaaacgccgttgctcaaaccattcgtctacgtccttgaggtcaaagacagtgtcctcctcaaggtcaaagcctaggtcttcccactcgtcgtcatcgctctcgtggccggcggccatacgcgcggcaaccgcgtcttcccctcctcttctttcaacgttgggtaccacgttgttttcttcgggttccataggttctgcgccgctgtcgtcatcatcctctccctgctcctcatcgtccgccaaggcgtcgtggattacctccaggttctgattgtcgggtacgacgtggttatcttcgtcgtcgtcgcgtggcatgggcggcggccgacggcggacgaccggcatggcgcggccgtcgtttccttcgtcttcctcttcaccgtctcccaaggaacgcggtcgacgacgttccgcgaagtcgccgcggaccacgcgcgcctgccaaatggtaaacgcgtcccaaccgtcccagttattgagcatttcggcgcgaaaacggtcgcctcgacagagccagcgaaactgccgcgcgtagtcgcggtctacgccgctgtcgaacatggtaaagtgcagacgcgccgcctcgcccatgtgtacgcagcctccgttgcgttccagcctggccgcgcgccgtagaccgtgttcgtagcggcgacgcacgtacaccttcatgaggccggcgcgaaaaagttcctctaggctgtcggccagccggtagatttcaccggctagacgctgcaggggcggcgagcggtccagatgcgacttgacaatcaccacgtaaaaacgacagaaacggtcgaagatgatgaggaaggacgtgtcaaaaaaaccaccggcgcggtaagagcccacggcacccagcaggtaccagcggcaacgcagttgcagcgtgacgtacatttcgcactcggccaagcgggcggctggcgctacctcgaagggccagcagtccgtcaagcagccgaaactggtcaggagtttcaacgttttggcatggcgtccaggtgtatgaaagttcacgtcgcgtccgtggtgttcgccaacgcaggcggccaacgcgtcggcgtcatgaccgtgacgcagcagcatcgctaccacgtcgtgcggtacccgcgtagcaaatggcgtctgtggctgacggtatacggcttcggtgtacatcataccgtaacgcgccagctcgtccagatgacgcgcgcacagcagcagaatctcttgcgagggttcgtagatgtagaggcgcgtaccgccacccatgcagagcaccagctccgtctcttcgtagtgatcttccaccatgatcacgcacttgcctagcacgataaggcgttcggggcaacaaatcacgtcgtccagcagctggtcgcgtagctccggcatggtgctgccgggccgtacctgcaggaaccagttgtgcggaatgccgagcgacagcacctggtcgacgtggttacggacccagtcgcgaagcacgtcggcgctgtactggcactcgaagatgccctgaaagtcgcccatgacccgcagaaaagtttcgtagcgcgtgtggcaatagaggaattcatcgtttcgcgtaaacgtgggagctccgtcttcccaacgtgtacgccacatgtcaaaagaggccgccagctagacaccccagaaaagaagcagagaaagagacttctttgtgcgacacgttttattctgcgtcctccgctcgacgttcaaatctggatgtactcgcgcacacccgtcaggctctttaagggaaaagggtccgagtacgtcactaaccgcgactgatgcaccagggcggtaatcacccgctccgcgccctcgcgcgtcgacgaacgcgtcgtcaccaggcaatgcagccgcgggcccgtatcgtcctgatgaccagcggcctcgcgctcggctgcttccacaccgacaatgtcgggatccaacacgtagctctgcgagttggtgtcgtagcggtgtagcaccaacgtgttggggtccagacgctcccacgcgccctcgtgcgggtcaaaacgctccgttaaacagagccagtcatactgctgctgcagaatacgccgctcgcgctcgcgtcgctcatcgggcaacgcggcgtcttcgttgaagagaatgtcccgcttgtggtctacggcacgctcgtggtggtgcgggcacaggtgacggtgttccatacgcgtctgacgttgacgctcgcgctcaaaacgccggtgtcgaaagaccattttcagcaaccccatgcggaaaaactccgtgatggtgttggcaacgcgccgcacatagtggttggggtcgtccatctggatggcgtacacggcaccgaaccagtccagcagtaccagcacttcggccacaaaactgcgtcccggtcgcggacgtcccgtcacgcctagcacataccacggcgtggccagattagcacggacagcccaccaccaacgacggctctccacctcggtgagcgcacaaaagggccaaatgcggtgtaactgctgtaccgttttcatcaaccgcataatcaccgtaccgtaacccggtgtatgcaactttacgtcgcaacccaggattcgttcggccgtggcgtacgagccctcgggcgtggtgtcattgagaaacaaaacatgcatggtacgcgcgcccttaggatatcgtcgcggaacgggtaccgtcattctccgcagagtggtgtgaatcacgtcgcgatacgcaatctccgaacgtgacacaccgtaacgtgccagttcgtccaggttgtgcgataccaacaccatgtacttttcacgagtgtcgtaggcgtagacgcgagaaaagcgacccataaaaaccacgtacggagtagccaccatgccatcatggtgatcgcgacgtggctcgggcaacaaaataacagcgtatcccaacggcgtcaacggctcgcggcaacagatgagctttgacgccgcctgtttggcggcggtaatgatcccgtcctccgtacgtaacatcacatgccagcccttggggggacccaaggacagacagcgtccctcgttacgatgaacgtaacgcgtgatttccattggctccaggcaaaagaacagttccttaaaatcccgcaacacttgtcggtataacgccatgggatcctcggccgccacaggcagcgcggggagctccggcggcataactgcagcgccgtcagggccagaacccgcagccggatccatcattgagcgacactctcagccggacaaccggcgtcactgacagaagccgagccaaatacagagaaagcaacgctacaccgtcaccccgctcccaagcgccgcggaaagtgctccgatttttcaccgtcgttcgcgacgttgatttgcctcggtctgagaaccgacctagcgttcggaccggtgcgcagaaacagccggcggtccgagccactgagcggttcacagccccggccgccgatagttaccggagagacgttcgagctgcaggtacatcagcgcttcccgcttcgccaccccgcgcccgccccagtttatactctccgacgccccgtccaacgcgcctgtggagggccaatcggaccgcgggagctctccaagtggatgacaggcacagccgagtgcccgaccgtgaagagccctcatccacctgaacagaccgctaaccgaaggaccccgagtcgcgtccgtcggtcccgacgtccgtcgccatctggctccctgctgttggctacctctcggatttcaaaaaagagcacgtgccgatgacggtgcacaggaaagagccaaagtgtcacggcgtctttttttatttgtattcctttcctgttttgtactcgtaaactgttgatgttgtttttacatccaaaagggcaagtaagaaacaggatgaggcatggtaggtttgggcgtggggcggccctccagcacggcggcccgggccgcccggcgggtgagcacccggcgttgcgccgtgtctatcttgtgtttcttctgtgtctttttcctatcttgttccgcgacggcctctttcatcacgttcagcatgcgttcctcgacgccctccagggatcctggggaggagggagtcctagtgaggcttccaatgttgttttgtggattttcggtttccttttcttggtcgtcatcgtcggacgtgtcgtcttcctcttgatcctcttcttcgtccgagtagtagacgcatagtccttggttcatcaggctgggattcatcaggttctgacggggaatccgctgttgtagacgtttaaccgcccgttccaggcgagagctcatgccgcaccagacgctgtaacgccgcacgggcccgtagcgggctgtttgttcgcgtacatgatcgttgagctcttgccaatattgtttggcacactccagatcggaggtttgtggatagtcgggtcggatccgcggatcccaactgacatcggcggtgccggagacttcgtccagactgttacgcatagagcaccagtcgggtcggacgataaacctgtccttgcggattaaccatttataacgtagttcgtgatggcgtgtagaggcccgtacacgctccacggtcccaaagcggtcccagaagggaaagttttcgtgggggcagcgacccggcacttccagacgttcggcgtcgtccacggcgtagtgaaaacgccggccggcctggtaaattttgagcagacccactgttaacaacatatccacgctgtcagccaaccgccagatctcgcggcgagacacgtcaaaatagaaaaattcgcaggctcggtcgaccaggatcacgaaatcggcgtgaaagacgccggagggtagcgattcgcccaccacacccattatcatggtttcacagcataagcggtccacaaagaacttcaacaggtcgttgaattgctccgtctccatacagatgaagggccagacgcctttgaggttctcggcctggccgcagagcagtagcggacgtgtcatctcgcccggagtgcgcagaggcacgcattcgccgcgataacgacaggtcacacgctgtagttcgctgatgctgttgtcgtgcaggcgaaggtcgcagataatatgatccggttgcgtggttagcagcggcgtgcgcatttgctcgccgtagatggcctcgcagtgcaacagcccgtgtcgcgcaaaatcgtccaaactgtgcgccaggtagtaaagcaccccgcgatcgcggtctagacaccacacggtttcgtaacgtcctaacaggagcaccagacgggcctggctaggtggctcaatttcctctacatacacgaaaaagtcgtcatcgtccgagtcctcgtcctcagaagaggaccgcggcccgtgtactctgggcaacacggtggtagagaactgcaggacgcccagagactcgagcgattcttcgcagcagatgagctgaccccagggcgtttcgggcccgtcggtgacagccgcgctgccaaagatgtcctcaaactctacaaaatctagacgccatccgggtggcgctgaaatgggaaggctaatgttcatatcagcatagctacgaactaagtggcggatgtcctgccgcaagtcttggcagagaatgagctttcgtaaacccttgagggtcctccgaacaacggccccagacgcgtagcgataggactggcgcatggtgccgcggcgtggagcggcacttggcagcctattttatggagtttcttcagtgacgtggcttgttcacgtcgttcgtgggctgcggttggcagctccggtctgtaaaccacccgaaaagactgacatcgacgtcaaagactcacgtaatttggaacatgtgcgaccgcaaagtgcgtcagaatagcacgtggctttaggacataaaaagtaccgtgaggtctagacgtgggttttgtgattgacacttacaccaggtaagccaagggacggtgaaactgtatgtgaggaatctgggtgcttagacgactaacgtgtaatgctttttacaggactgttcgacaggtgatagtacctgtaaggtgatgaccacctctacaaataatcaaaccttaacgcaggtgagcaacatgacaaaccacaccttaaacagcaccgaaatttatcagttgttcgagtacactcggttgggggtatggttgatgtgcatcgtgggcacgtttctgaacgtgctggtgattaccaccatcctgtactaccgtcgtaagaaaaaatctccgagcgatacctacatctgcaacctggctgtagccgatctgttgattgtcgtcggcctgccgttttttctagaatatgccaagcatcaccctaaactcagccgagaggtggtttgttcgggactcaacgcttgtttctacatctgtctttttgccggcgtttgttttctcatcaacctgtcgatggatcgctactgcgtcatcgtttggggtgtagaattgaaccgcgtgcgaaataacaagcgggccacctgttgggtggtgattttttggatactagccgtgctcatggggatgccacattacctgatgtacagccataccaacaacgagtgtgttggtgaattcgctaacgagacgtcgggttggttccccgtgtttttgaacaccaaagttaacatttgcggctacctggcgcccatcgcgctgatggcgtacacgtacaaccgtatggtgcggtttatcattaactacgttggtaaatggcacatgcagacgctccacgttcttttggttgtggttgtgtcttttgccagcttttggtttcctttcaacctggcgctatttttagaatccatccgtcttctggcgggagtgtacaatgacacacttcaaaacgttattatcttctgtctatacgtcggtcagtttttggcctacgttcgcgcttgtctgaatcctgggatctacatcctagtaggcactcaaatgaggaaggacatgtggacaaccctaagggtattcgcctgttgctgcgtgaagcaggagataccttaccaggacattgatattgagctacaaaaggacatacaaagaagggccaaacacaccaaacgtacccattatgacagaaaaaatgcacctatggagtccggggaggaggaatttctgttgtaattcgatcctctctcacgcgtccgccgcacatctatttttgctaattgcacgtttcttcgtggtcacgtcggctcgaagaggttggtgtgaaaacgtcatctcgccgacgtggtgaaccgctcatatagaccaaaccggacgctgcctcagtctctcggtgcgtggaccagacggcgtccatgcaccgagggcagaactggtgctatcatgacaccgacgacgacgaccgcggaactcacgacggagtttgactacgatgaagacgcgactccttgtgttttcaccgacgtgcttaatcagtcaaagccagttacgttgtttctgtacggcgttgtctttctcttcggttccatcggcaacttcttggtgatcttcaccatcacctggcgacgtcggattcaatgctccggcgatgtttactttatcaacctcgcggccgccgatttgcttttcgtttgtacactacctctgtggatgcaatacctcctagatcacaactccctagccagcgtgccgtgtacgttactcactgcctgtttctacgtggctatgtttgccagtttgtgttttatcacggagattgcactcgatcgctactacgctattgtttacatgagatatcggcctgtaaaacaggcctgccttttcagtattttttggtggatctttgccgtgatcatcgccattccacactttatggtggtgaccaaaaaagacaatcaatgtatgaccgactacgactacttagaggtcagttacccgatcatcctcaacgtagaactcatgcttggtgctttcgtgatcccgctcagtgttatcagctactgctactaccgcatttccagaatcgttgcggtgtctcagtcgcgccacaaaggtcgcattgtacgggtacttatagcggtcgtgcttgtctttatcatcttttggctgccgtaccacctaacgctgtttgtggacacgttaaaactcctcaaatggatctccagcagctgcgagttcgaaagatcgctcaaacgtgcgctcatcttgaccgagtcgctcgccttttgtcactgttgtctcaatccgctgctgtacgtcttcgtgggcaccaagtttcggcaagaactacactgtctgctggccgagtttcgccagcgactcttttcccgcgatgtatcctggtaccacagcatgagcttttcgcgtcggagctcgccgagtcgaagagagacatcttccgacacgctgtccgacgaggtgtgtcgcgtctcacaaattataccgtaatataacttcgtatagcatacattatacgaagttatctcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgaggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgccacaacccgggatccaccggtcgccaccatgggcggtaggcgtgtacggtgggaggtctatataagcagagctctctggctaactagagaacccactgcttactggcttatcgaaattaatacgactcactatagggagacccaagcttctaggaagatcggggccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatgtaagtatcaaggttacaagacaggtttaaggagaccaatagaaactgggcttgtcgagacagagaagactcttgcgtttctgataggcacctattggtcttactgacatccactttgcctttctctccacagatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaaagcggccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggcagataacttcgtatagcatacattatacgaagttattaaaaaagcgctacctcggccttttcatacaaaccccgtgtccgcccctcttttccccgtgcccgatatacacgatattaaacccacgaccatttccgtgcgattagcgaaccggaaaagtttatggggaaaaagacgtaggaaaggatcatgtagaaaaacatgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcagcacctctttccattcttccgtgtcccctgccacctcagtggaccgtcaatttcgacggaccacgtacgaccgttgggacggtcgacgttggctgcgcacccgctacgggaacgccagcgcctgcgtgacgggcacccaatggagcaccaacttttttttctctcagtgtgagcactaccctagtttcgtgaaactcaacggggtgcagcgctggacacctgttcggagacctatgggcgaggttgcctactacgggggttgttgtatggtgggcgggggtaatcgtgcgtacgtgatactcgtgagcggttacgggaccgccagctacggcaacgctttacgcgtgaattttgggcgcggcaactgcacggcgccgaaacgcacctaccctcggcgcttggaactgcacgatggccgcacagaccctagccgttgcgatccctaccaagtgtatttctacggtctgcagtgtcctgagcaactggttatcaccgcccacggcggcgtgggtatgcgccgctgtcctaccggctctcgtcccaccccgtcccggccccaccggcatgacttggagaacgagctacatggtctgtgtgtggatcttctggtgtgcgtccttttattagctctgctgctgttggagctcgttcccatggaagccgtgcgtcacccgctgcttttctggcgacgcgtggcgttatcgccgtccacttccaaggtggatcgcgccgtcaagctgtgtcttcggcgcatgtttggtctgccgccgccaccgtcagtcgcaccacctggggaaaagaaggagctaccggctcaggcggccttgtcgccgccactgaccacctggtcactaccgccgtttccgtccacgcggatacctgacagtccgccgccaccgtaccagcttcgtcacgccacgtcactagtgacggtacccacgctgctgttatatacgtcatccgacatcggtgacacagcttcagaaacaacgtgtgtggcgcacgctacttatggggaacccccggagcccgctcgatcgacggctacggttcaggaatgtaccgttcttaccgccccgaattgcggcatcgtcaacaacgacggcgcggtctctgaaggccaagaccatggagatgcggttcaccatagcctggatgtggtttcccagtgtgctgctgatactggggttgttgacacctccgagtaacgggtgcaccgtcgatgttggacgaaacgtatccattggagaacagtgccgccttcgaaacggtgcgacgttctccaagggagacatcgaaggtaacttcagtgggcccgtcgtcgtggagttggactacgaagatatcgatattactggcgaacggcagcgacttcggttccatctcagcggactcgggtgtcctacaaaggaaaatataagaaaagacaatgaaagcgacgtcaacggtggaattcgctgggctctatatatacaaaccggcgacgccaagtacggtattcgtaaccagcatttgagtatacggttaatgtatcctggggaaaaaaatacacaacagctgttggattctgatttcagttgcgaacgtcaccggagaccgtccacgccgttgggaaagaacgccgaagtgcctcccgcgacccgcacgtcttctacatacagcgtcctcagcgcttttgtagtgtggatcggatccggcctcaatatcatctggtggaccggcatcgtgcttctggcggtggacgctctcggacttggcgagcgttggctgaggttagcactgtctcaccgggacaaacatcacgcatcgcgaaccgcggcgctccagtgtcaacgcgacatgttacttcggcaacgtcgacgggctcggcggctgcacgccgtttctgaaggcaaactgcaggaagagaagaaacgacagtctgctctggtctggaacgttgaggcgcgaccctttccgtccacacatcagctgattgtgctgccccctcctgtagcgtcagctcctcctgcggttccctcgcagccccccgagtattcgtctgtgtttccgcctgtataaaaataaagagacgggaggctgatcgcggccttcagcgtctcatttgtctttactctcgagtgcggtcggtgtctcgtcggtgagacgaggccgccgcccgacaagttcgatctcatgtcgctcttggagcgcgaagagagttggcgtcgcgtagtcgactactcgcacaacctgtggtgtacgtgcggtaactggcagagccacgttgagattcaggacgaggagccaaactgcgagcagccggagcccgcacactggctggaatacgtggcggtccagtggcaggcccgggttcgcgattctcacgatcgctggtgtctctgcaacgcctggcgtgatcacgccttgcgcggccgttggggtacggcgtattcctcgggttcctcggcctcttcctccggtttcgtcgcggagagcaagttcacctggtggaaacgactgcgccacagtacccggcgctggttgtttcgccgccggcgagctcgatacactccgtctaactgtggggaaagtagcactagcagcggccagagtagcggtgacgagagtaactgcagtctacgcacccacggcgtgtacacacggggtgaacaacactaatcgataagtcgcgtgtaggcgactggctacatcaaccggatatctgcggggatttaaaaagacgacccgttgtcatccggcttagaccaaaccgtccttttatcatcttccgtcgccatggctatgtacacatccgaatccgaacgcgactggcgtcgtgtaatccacgactcgcacggcctgtggtgcgactgcggcgactggcgagagcacctctattgtgtgtacgacagccattttcagcgacgacccacgacccgagccgaacggagggccgccaattggcggcgacagatgcggcggttacaccgtttgtggtgtttttgtcaggactggaagtgtcacgcgttatacgccgagtgggacggcaaagaatccgacgacgagtcgtcggcgtcttcctcgggcgaagcgccagagcaacaggtccccgcttggaagaccgtgcgggccttctcgcgggcctaccaccaccgcattaaccggggtctgcggggcacgcccccaccgcgcaacttgccgggatacgagcacgcctccgagggctggcggttttgcagtcgacgggaacggcgagaggacgatcttcgcacgcgggctgagccggaccgcgtggtgttccagttagggggagtaccccctcgccgtcaccgggaaacttacgtgtaagaacacggcgtgacaataaacaacatagcgtaaatccccgtgtgatgtgtgtgattgacgttcgggaaacatgtccccatcatcagcgtcacaattgacgtgggttggtcactgacgtgcaggatgttacgcgagtcagagaatcgcataagaacggagtggtgagcgggttcccacaggagtctctggcgcaaaagcaccatgagcctcaggttccccgagagggtgggttacgagaaactgggataccgcccgcatgccaaacgcgtgcgggtgcatgactcgttgggattgacgcggtttatcatgaggcaactcatgatgtacccgctggtgttgccgttcacttttccgttttacgtgccgcggtcctagcacgtcagtggtgacgctgataattgcaacatggcccatgacgaacccgcttgggacgaacgtcaataccacgtcaaaccaccgtgacttggctgaacgttgaaacataaagccaaagcgccgtcggcacttggcttcagagcagcgcctcggggcgatgcgacggcgatgaacttagagcaactcatcaacgtccttggtctgctcgtctggattgccgctcgtgctgtcagccgcgttggtccgcatggctccggactcgtttatcgtgagcttcatgatttctacgggtatctgcagctggaccttctgggaccagtggtggcggggaatcgctcagtccggacctggagagagcaggcggaccgagccagagggaccttcgcttggcgttcaggccttaatactagccgcatcttacctgtcggcagcatgtatcggggctccgacgccttacccgccggcctgtatcgtcccgaagaagaggtgttcctcctcttgaatcgctgccatgggccactgtcaacgccgaaaaatgcttgtctggctgaggttggtgtcgctaatgccacttttttgtctcgcttcaatgtcggtgattttcacggagcgtcatgggaaaacggtaccgctcccgatggagagcccggggtatgctgaaattcctcttaaaatttcgtaaacgacgtcgtccagtcgttgtgccgcgattcgtacggttcatcgtctacgtcgttttgttcaccgtcgctgtgcaacgcgtgaaacaagagcgtgatgcgcaccttcggcggtatgaagaacgattacggaaaaaccgcgcacggcgtcggcagtcttttccgtgacttggggcgatgggtccgagctgcggtatgggtcacggcggcgtgtgttttattgacgaagatgccgatgtgtgactaaaaacgtcccagccccagagcgatatgtttcaataaaaaaaatatgtagtatcatattatgcgtgtcctggtttttcatttttggatgtatgtatcgcataaagggtggcgaggtgtgaggatgaaacatatgcagatacgcagtgttgttatccgaacgaaacccgtgtaatgcgtacaacggtacttcagtatgaaagtcccgtgtgtggggggggggggcaaatagttgcgtttgccgttgggcgtacgctacgtttgtatttctggctataatatgtgcggtcatgtgtcgatgttcctattgggaagggtgtgactgtagggtgtataaagtacggtgggacgcagagggacattgatagaaacaggttgagcgctgtacgagtttcacacgctgaatcggcgccaagaataaaacagtggttattcgtaaaagtatgggggggggggggatgttgtcgacggttgttagatgcattgcgtatctgtattagtagttttgcaagccgtggtgcgtgttattgtgacgtagcaattatcgtattgtgcatgtgtcgttcatcacagagtttagtatactaatatgaagcgtcgcgagtattaaagcaattggtgtctctgtgctagtctaacaacacctgtgtaatgcgtacaacgagaaaaaagacgcgaaagcaacgtgtatgggggggggggggaataatattgctaatcatgcgtcttgcagtacagatagccgctgtatcttacgcgtattgtcgcaacagttccacatcggtgtaattggatgtctggtacttatcactggcgtcgttataacattgtaaaacaagttttcgaaacataacgacagctgcaaaagaaaaccagtttattgagcattgtaatggtagtgtgtggctatattagaaaacgtgacgcgtcgcatgtcgcggcacaatctggcagcggggtcggggtagggtacggtgggaggcatgtacacagatggaacaaaagcagaagtaacgtgagaaggagcatacagtccagtatccagcggttcctgagtagcaccacccatcaactgaatgccctcatgagtaaaagtctgcgggcgacagcccttggggaccgttggcatgggacgatcaatctccaaaccacagcgtaacaccgttttcttccaacgtcgttgatagacgtcgtttttacggttactcccaagaacccagaaagtctcgtccaagtcgtaccaggaatcttctccggggagacgcgacggtttccaatcctcgtcgtctcgtctcaaagcacgtcccaaactggcttgaggagtcaacggtggttctgtgggtcgggtgtagcgcgagtgttttcccttcatgagcgattcatcctccttgcctttaggctttttggtctttttgtgtatcatctggccgccggcctccataaccaccgtggccaagtccagtcccagagcttgagcgtcggcgcggcgtcgggcgtcttgcaggtagtcttccacatttgcacagatggccgggtgtttggtggctagggtgaggacctcagcctcgccgcgacccggacgtagcaaaaaagccaactgcccgtgcggctcgcgcgcccacagcgcggcgcgcgggtgcaggtgcagcgcgtcccagcgcggccgctcccactgctcgcggtccagctcgggcagcagccgccgcgcggcctcggcggcgggcgccgactcgcgtcccagcgccagcgcgcccagcacgcccgcgcgcagaaagtgcgacagctccgccgccagcgggtacacgtgcccgtccagcgggcagtacccgaacacggcgcccagctcgtccagcagcaccaccagcatggcgcgcggcacggtccccgacgccgccggacccgccatcgctgtcgaacccaccatcaccgtcggcgccgctgctgctgccgcgtccgccccgaccaccgcgtgcgcgtccgcgttaggcacgcaaatcgcgcccccgccggcggcgccgtacggctgcggaggcacaggtacagcggaccccacggctcccgctatcgcgcacggcgcgtccccgccggcggcctccgtctccgtgccgctcgccgctggcggcgacgtcgtccccgccgccgtccccgtcgccggccccgccgcgcagcccagccaccgcgcgggcagcaccgcgcccagcgccagccagccgcagcacagacgctggttcaggtgccgacgcacggccgtcagcagcgacgcggggtgcggcgccgacgcgaacggctcgtactgcgccagctcctgccacgcgcccagcagtaccatcggctgcagtcgcctgcccggcgtctgcagcgccaccgtcgtgccggcccaccgccggcgcagctcccgtccgagcgccgtcgcctcctcggcgcgcagcaacgtctgtcggagcgccggctgaggcagcagcgtcgcgcgcggggtgcccacgcccagccggttgcagcggtacagccgcaccacctcgcccgcgccgtgccgaaaccactcgtccgcgtcgcgcgccgctaggatcagcgtgttgtttgccaggtcgtacacgaacacgcggaacccggcgcccagcgccaggtacagtccgtcctgcgcgcacagaccctcgggatggccggccttgtcgcccaacgtcgggtcggctgcggggtccacctcgtgcaccacggtagccaccagtacgatccacgcgtcccgcggcgacagttgacgcaggtccgtcgcgcccacgccgttcatctggctgcgcggcgtcacccgcgcgtagaatccgtacggccgtccgagcggcagcagcgtgcccgcgtcgcgctgcgaccacttgcgcatggcgcggcccgtgctgttggccaaaaacgccgcgcgccacacggcgcccatggcctggtattccagctccgtcagcgcctggcgctccaccggaatctgagacagcaacaggcgctccgggccgtgccaaaagttgctattgttgccgctactcggaggggcgcccggcggcccgcggggttctacccggtggacgccgtggcccggcgtcgtcgtagccgcagcactcgcaccagtgcccgctgtggacggcgctcccatcggcacacaagaagaaggaggagaggaaccaacccccgaaggccctccggccccgcggccgcgaccaaggggcggggggcgcggcgacatgccgttgcgctgggccatgggcgccggacacctccgacgtccactatataggaagcaaacccgcgtcagcgagcacgcggtttagacacgcggacgccttcgtcgcccgtgtgccgcgggcgacacgcagctggcttttataggcagcgacgtgcacggcgcgtgctggcgccgccttggcgccacgcagtctggaaggccgtggactgggaaaggcagctacccgaaggaagacgcgcggcaggcgcgtaccactggagcgcacagccgcctcccgggcgcgcacccatctaggtggacgcccgacatccattccgggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgaggg

SEQ ID NO: 29 - protein, 107 aa, Artificial Sequence
  FKBP
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 30 - protein, 18 aa, Artificial Sequence
  FKBP (F15S) fragment
GVQVEISPGDGRTSPKRG

SEQ ID NO: 31 - protein, 107 aa, Artificial Sequence
  FKBP (E31G, F36V, R71G, K105E)
GVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 32 - protein, 490 aa, cytomegalovirus
ESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 33 - protein, 486 aa, cytomegalovirus
SPVYVNLLGSVGLLAFWYFSYRWIQRKRLEDPLPPWLRKKKACALTRRSRHRLRRQHGVIDGENSETERSVDLVAALLAEAGEESVTEDTEREDTEEEREDEEEENEARTPEVNPIDAEGLSGLAREACEALKKALRRHRFLWQRRQRARMLQHNGPQQSHHAAVFCRVHGLRGFQVSVWLLLTLLWSTGHGVSVRCTYHGTDVNRTSNTTSMNCHLNCTRNHTQIYNGPCLGTEARLPLNVTFNQSRRKWHSVMLKFGFQYHLEGWFPLRVLNESREINVTEVHGEVACFRNDTNVTVGQLTLNFTGHSYVLRAIAHTSPFESYVRWEETNVTDNATSSENTTTVMSTLTKYAESDYIFLQDMCPRFLKRTVKLTRNKTKHNVTVTGNNMTTLPVWTPECKGWTYWTTLSVMWRNRRSALLRAKSRALGHWALLSICTVAAGSIALLSLFCILLIGLRRDLLEDFRYICRDEGSSSTKNDVHRIV

SEQ ID NO: 34 - protein, 433 aa, cytomegalovirus
MDRKTRLSEPPTLALRLKPYKTAIQQLRSVIRALKENTTVTFLPTPSLILQTVRSHCVSKITFNSSCLYITDKSFQPKTINNSTPLLGNFMYLTSSKDLTKFYVQDISDLSAKISMCAPDFNMEFSSACVHGQDIVRESENSAVHVDLDFGVVADLLKWIGPHTRVKRNVKKAPCPTGTVQILVHAGPPAIKFILTNGSELEFTANNRVSFHGVKNMRINVQLKNFYQTLLNCAVTKLPCTLRIVTEHDTLLYVASRNGLFAVENFLTEEPFQRGDPFDKNYVGNSGKSRGGGGGGGSLSSLANAGGLHDDGPGLDNDLMNEPMGLGGLGGGGGGGGKKHDRGGGGGSGTRKMSSGGGGGDHDHGLSSKEKYEQHKITSYLTSKGGSGGGGGGGGGGLDRNSGNYFNDAKEESDSEDSVTFEFVPNTKKQKCG

SEQ ID NO: 35 - protein, 156 aa, cytomegalovirus
SWAKQRVPFLDDDDGEEENDVQDDVDSPVPTRPLVIDEDAEPAAGTSGGLEGGGGDDEDGEDGHALPDLDDDLLLQFEPMLPRVYDLLLPSLDARLNFVNAGQKYAAFLKYVHGDCATCSHGEILREKTQLLTAIVSKLMDINGILEGKDESAPGK

SEQ ID NO: 36 - protein, 667 aa, cytomegalovirus
NPSTHVSSNGPTTPPHGPHTTFLPPTSPAPSTSSVAAATLCSPQRQAVSRYSGWSTEYTQWHSDLTTELLWHAHPRQVPMDEALAAAAAASYQVNPQHPANRYRHYEFQTLSLGTSEVDELLNCCAEETTCGGTQSTVLTNATNTTSCGGAVAGSSNVGPAGASAACDLDAELAGLETSAADFEQLRRLCAPLAIDTRCNLCAIISICLKQDCDQSWLLEYSLLCFKCSYAPRAALSTLIIMSEFTHLLQQHFSDLRIDDLFRHHVLTVFDFHLHFFINRCFEKQVGDAVDNENVTLNHLAVVRAMVMGEDTVPYNKPRRHPQQKQKNNPYHVEVPQELIDNFLEHSSPSRDRFVQLLFYMWAGTGVMSTTPLTELTHTKFARLDALSTASEREDARMMIEEEEDEEGGEKGGDDPGRHNGGGTSGGFSESTLKKNVGPIYLCPVPAFFTKNQTSTVCLLCELMACSYYDNVVLRELYRRVVSYCQNNVKMVDRIQLVLADLLRECTSPLGAAHEDVARCGLEAPTSPGGDSDYHGLSGVDGALARPDPVFCHVLRQAGVTGIYKHFFCDPQCAGNIRVTNEAVLFGRLHPHHVQEVKLAICHDNYYISRLPRRVWLCITLFKAFQITKRTYKGKVHLADFMRDFTQLLESCDIKLVDPTYVIDKYV

SEQ ID NO: 37 - protein, 376 aa, cytomegalovirus
MSSVSGVRTPRERRSALRSLLRKRRQRELASKVASTVNGATSANNHGEPPSPADARPRLTLHDLHDIFREHPELELKYLNMMKMAITGKESICLPFNFHSHRQHTCLDISPYGNEQVSRIACTSCEDNRILPTASDAMVAFINQTSNIMKNRNFYYGFCKSSELLKLSTNQPPIFQIYYLLHAANHDIVPFMHAEDGRLHMHVIFENPDVHIPCDCITQMLTAAREDYSVTLNIVRDHVVISVLCHAVSASSVKIDVTILQRKIDEMDIPNDVSESFERYKELIQELCQSSGNNLYEEATSSYAIRSPLTASPLHVVSTNGCGPSSSSQSTPPHLHPPSQATQPHHYSHHQSQSQQHHHRPQSPPPPLFLNSIRAP

SEQ ID NO: 38 - protein, 849 aa, cytomegalovirus
EMNLLQKLCVVCSKCNEYAMELECLKYCDPNVLLAESTPFKRNAAAIVYLYRKIYPEVVAQNRTQSSLLTLYLEMLLKALHEDTALLDRALMAYSRQPDRAAFYRTVLRLDRCDRHHTVELQFTDNVRFSVSLATLNDIERFLCKMNYVYGILAPEAGLEVCAQLLELLRRLCGISPVARQEVYVEGTTCAQCYEELTIIPNQGRSLNKRLQGLLCNHIAVHRPSSQSDVNIQTVEQDLLDLTTRIPHLAGVLSALKSLFSSSSAYHSYIQEAEEALREYNLFTDIPERIYSLSDFTYWSRTSEVIVKRVGITIQQLNVYHQLCRALMNGISRHLYGEDVEDIFVLGEKALDGEERMFVGSVFAAPNRIIDLITSLSIQAFEDNPVFNKLHESNEMYTKIKHILEEIRRPLPDGTGGDGPEGEAIHLRGREAMSGTGTTLMTASNSSNSSTHSQRNNGGGGRARGGGKKVVGGGVNGQDGDGSENGLRVRNCDEHEALDLVDARSRIHNVTREVNVRKRAYLQKVSEVGYGKVIRCIKTQERLTSKLIDVNLVGPLCLDFISKLMNGFLYRSQYHQDQDVVDVGDQFTYDEHLYVVNNLIHKSLPVESLPLLGQQIYELCNGPLFTHCTDRYPLSHNVDMAYACDNAGVLPHVKDDLVKCAEGTVYPSEWMVVKYMGFFNFSDCQDLNVLQKEMWMHVRELVLSVALYNETFGKQLSIACLRDELHPDRDVILTYNKEWPLLLRHEGSLYKSKDLYLLLYRHLSRPDESGDVPTAPVAKPSTLTAAAAVSGVFREPDRPWLPSPYPSSSTAGVSRRVRATRKRPRRASSLLDLARDEHGIQDLVPGSLR

SEQ ID NO: 39 - protein, 642 aa, cytomegalovirus
MSLLHTFWRLPVAVFFEPHEENVLRCPERVLRRLLEDAAVTMRGGGWREDVLMDRVRKRYLRQELRDLGHRVQTYCEDLEGRVSEAEALLNQQCELDEGPSPRTLLQPPCRPRSSSPGTGVAGASAVPHGLYSRHDAITGPAAAPSDVVAPSDAVAASAAAGASSTWLAQCAERPLPGNVPSYFGITQNDPFIRFHTDFRGEVVNTMFENASTWTFSFGIWYYRLKRGLYTQPRWKRVYHLAQMDNFSISQELLLGVVNALENVTVYPTYDCVLSDLEAAACLLAAYGHALWEGRDPPDSVATVLGELPQLLPRLADDVSREIAAWEGPVAAGNNYYAYRDSPDLRYYMPLSGGRHYHPGTFDRHVLVRLFHKRGVIQHLPGYGTITEELVQERLSGQVRDDVLSLWSRRLLVGKLGRDVPVFVHEQQYLRSGLTCLAGLLLLWKVTNADSVFAPRTGKFTLADLLGSDAVAGGGLPGGRAGGEEEGYGGRHGRVRNFEFLVRYYIGPWYARDPAVTLSQLFPGLALLAVTESVRSGWDPSRREDSAGGGDGGGAVLMQLSKSNPVADYMFAQSSKQYGDLRRLEVHDALLFHYEHGLGRLLSVTLPRHRVSTLGSSLFNVNDIYELLYFLVLGFLPSVAVL

SEQ ID NO: 40 - protein, 294 aa, cytomegalovirus
MARDEENPAVPRVRTGKFSFTCANHLILQISEKMSRGQPLSSLRLEELKIVRLICVLLFHRGLETLLLRETMNNLGVSDHAVLSRKTPQPYWPHLYRELRQAFPGLDFEAAVFDETRAARLSQRLCHPRLSGGLLTRFVQRHTGLPVVFPEDLARNGNILFSLGTLYGHRLFRLAAFFTRHWGAEAYEPLIRIICQKMWYFYLIGTGKMRITPDAFEIQRSRHETGIFTFIMEDYRTFAGTLSRHPHRPHPQQQQHHHPGPPHPPLSHPASSCLSPEAVLAARALHMPTLANDV

SEQ ID NO: 41 - protein, 586 aa, cytomegalovirus
MPRVDPNLRNRARRPRARRGGGGGVGSNSSRHSGKCRRQRRALSAPPLTFLATTTTTTMMGVASTDDDSLLLKTPDELDKYSGSPQTILTLTDKHDIRQPRVHRGTYHLIQLHLDLRPEELRDPFQILLSTPLQLGEANDESQTAPATLQEEETAASHEPEKKKEKQEKKEEDEDDRNDDRERGILCVVSNEDSDVRPAFSLFPARPGCHILRSVIDQQLTRMAIVRLSLNLFALRIITPLLKRLPLRRKAAHHTALHDCLALHLPELTFEPTLDINNVTENAASVADTAESTDADLTPTLTVRVRHALCWHRVEGGISGPRGLTSRISARLSETTAKTLGPSVFGRLELDPNESPPDLTLSSLTLYQDGILRFNVTCDRTEAPADPVAFRLRLRRETVRRPFFSDAPLPYFVPPRSGAADEGLEVRVPYELTLKNSHTLRIYRRFYGPYLGVFVPHNRQGLKMPVTVWLPRSWLELTVLVSDENGATFPRDALLGRLYFISSKHTLNRGCLSAMTHQVKSTLHSRSTSHSPSQQQLSVLGASIALEDLLPMRLASPETEPQDCKLTENTTEKTSPVTLAMVCGDL

SEQ ID NO: 42 - protein, 940 aa, cytomegalovirus
AGAAPRRLGCDALIVVGGSAMPRRVLHVPVHVRACNLTQELSTGEDARFCRPRPVNVERVRAVFAALYRACPIHVRTEPERVKLVLGRLLLGPVAVPCFCDGEVEGHGEHLVPTTQFCRGPLLYVHRRCCCGSVTAGRALSYHVLENHVATHVLRGLLSLTEWNRELPSLFCDCPGGGGASGTEERYAMACLPRDLSLHLDDYPYLMVEIGRVLSVSEVDDYVTAVSGYLGEAAAPRIQVHYKLLFGLNVRPQAPCALDATRDFFLLELQKLWLGVEYHHEVTSEFFGRVLAQLHRDRARVMMALRLPEQTVCHLSTFVLSRFKRQVLYFKLQVSYGKCRTGHADRSGGGGNGGNQGHHNLLCYRRLSVTFADTDTVWRNLFYVYYELARDLGSHGTEDRPVSRGYGVSCASRTSRLSPSESTVVSANGHALSSTALPTTSAGHKLSLPRDPAADRVRRYVCIISRLMYARYGERWRKHCQRRSETGEEEEEETLESGETDATPPFDFTGQQLRRAYQEHRRRKHLAVQRYAPCRRKLIGGMEFAEVTGVSLDRIAVNAFNTNRVINMKAALSSIAASGLGVRAPRLPKNMTHSFVMYKHTFKEPACTVSTFVSNDAVYINSLNVNIRGSYPEFLYSLGVYRLHVNIDHFFLPAVVCNSNSSLDVHGLEDQAVIRSERSKVYWTTNFPCMISHTNNVNVGWFKAATAIVPRVSGADLEAILLKELSCIKNMRDVCIDYGLHRVFTQLELRNSYQIPFLAKQLVLFLRACLLKLHGREKRLQLDRLVFEAAQRGLFDYSKNLTAHTKIKHTCALIGSRLANNVPKILARNKKVKLDHLGRNANVLTVCRHVEAHKIPRTRLKVLVEVLGALQSISGTPHTREVIHQTLFRLCSAAAATSGLCSSPPPLCVSSSSSVPSVPTSVSVDGSSEPTSPRARFASR

SEQ ID NO: 43 - protein, 956 aa, cytomegalovirus
MSMTASSSTPRPTPKYDDALILNLSSAAKIERIVDKVKSLSRERFAPEDFSFQWFRSISRVERTTDNNPSAATTAAATTTVHSSASSSAAAAASSEAGGTRVPCVDRWPFFPFRALLVTGTAGAGKTSSIQVLAANLDCVITGTTVIAAQNLSAILNRTRSAQVKTIYRVFGFVSKHVPLADSAVSHETLERYRVCEPHEETTIQRLQINDLLAYWPVIADIVDKCLNMWERKAASASAAAAAAACEDLSELCESNIIVIDECGLMLRYMLQVVVFFYYFYNALGDTRLYRERRVPCIICVGSPTQTEALESRYDHYTQNKSVRKGVDVLSALIQNEVLINYCDIADNWVMFIHNKRCTDLDFGDLLKYMEFGIPLKEEHVAYVDRFVRPPSSIRNPSYAAEMTRLFLSHVEVQAYFKRLHEQIRLSERHRLFDLPVYCVVNNRAYQELCELADPLGDSPQPVELWFRQNLARIINYSQFVDHNLSSEITKEALRPAADVVATNNSSVQAHGGGGSVIGSTGGNDETAFFQDDDTTTAPDSRETLLTLRITYIKGSSVGVNSKVRACVIGYQGTVERFVDILQKDTFIERTPCEQAAYAYSLVSGLLFSAMYYFYVSPYTTEEMLRELARVELPDVSSLCAAAAATAAAPAWSGGENPINNHVDADSSQGGQSVPVSQRMEHGQEETHDIPCLSNHHDDSDAITDAELMDHTSLYADPFFLKYVKPPSLALLSFEETVHMYTTFRDIFLKRYQLMQRLTGGRFATLPLVTYNRRNVVFKANCQISSQTGSFVGMLSHVSPAQTYTLEGYTSDNVLSLPSDRHRIHPEVVQRGLSRLVLRDALGFLFVLDVNVSRFVESAQGKSLHVCTTVDYGLTSRTAMTIAKSQGLSLEKVAVDFGDHPKNLKMSHIYVAMSRVTDPEHLMMNVNPLRLPYEKNTAITPYICRALKDKRTTLIF

SEQ ID NO: 44 - protein, 107 aa, Artificial Sequence
  FKBP (F36V, K105E)
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLELE

(19) United States
(12) Patent Application Publication
(10) Pub. No.: US 2015/0307850 A1
(43) Pub. Date: Oct. 29, 2015

(54) CONDITIONAL REPLICATING VIRAL VECTORS

(71) Applicants: Tong-ming FU, West Point, PA (US); Dai WANG, West Point, PA (US); Danilo CASIMIRO, West Point, PA (US); Daniel FREED, West Point, PA (US); Fengsheng LI, West Point, PA (US); MERCK SHARP & DOHME CORP., Rahway, NJ (US)

(72) Inventors: Tong-Ming Fu, Ambler, PA (US); Dai Wang, Blue Bell, PA (US); Danilo Casimiro, Harleysville, PA (US); Daniel C. Freed, Limerick, PA (US); Fengsheng Li, Robbinsville, NJ (US)

(73) Assignee: Merck Sharp & Dohme Corp., Rahway, NJ (US)

(21) Appl. No.: 14/649,669

(22) PCT Filed: Dec. 4, 2013
(86) PCT No.: PCT/US13/73017

§ 371 Date: Jun. 4, 2015

Related U.S. Application Data
(60) Provisional application No. 61/733,179, filed on Dec. 4, 2012.

(51) Int. Cl. C12N 7/00; A61K 39/245; C07K 14/005

(52) U.S. Cl. CPC C12N 7/00; C07K 14/005; A61K 39/245; C12N 2710/16121; C12N 2710/16134; C12N 2710/16162; C12N 2710/16152; A61K 2039/5256

(57) ABSTRACT

The present invention relates to a cytomegalovirus (CMV) which has been recombinantly altered to express a heterologous polypeptide and to allow for external control of viral replication. The heterologous polypeptide may be a polypeptide of interest such as an antigen, antibody or immune modulator. The CMV vectors of the invention are replication defective, or chemically controllable replication capable, or replication competent. The present invention also relates to uses of the CMV vectors such as inducing an immune response to an antigen or expressing an antibody or immune modulator in vivo. Compositions comprising the CMV expressing the heterologous polypeptide are also encompassed by the present invention.

### FIELD OF INVENTION

The present invention relates to a cytomegalovirus (CMV) which has been recombinantly altered to express a heterologous polypeptide and to allow for external control of viral replication. The heterologous polypeptide may be a polypeptide of interest such as an antigen, antibody or immune modulator. The present invention also relates to uses of the CMV vectors such as inducing an immune response to an antigen or expressing an antibody or immune modulator in vivo.

### CROSS-REFERENCE TO RELATED APPLICATIONS

This application claims the benefit of U.S. Provisional Application No. 61/733,179, filed Dec. 4, 2012, the contents of which are hereby incorporated by reference in their entirety.

### REFERENCE TO SEQUENCE LISTING SUBMITTED ELECTRONICALLY

The sequence listing of the present application is submitted electronically via EFS-Web as an ASCII formatted sequence listing with a file name “23373-WO-PCT-SEQLIST-22NOV2013.TXT”, creation date of Nov. 22, 2013, and a size of 471 KB. This sequence listing submitted EFS-Web is part of the specification and is herein incorporated by reference in its entirety.

### BACKGROUND OF THE INVENTION

Recombinant viral vectors have been researched as gene delivery vehicles either as vaccines for presentation of complex antigens or for transient expression of recombinant proteins with biological functions. Adenovirus vector and adenovirus-associated virus (AAV) are two widely used viral vectors for the purposes, but both have limitations for gene transfer applications. For example, transfection efficiency and expression are difficult to control in adenovirus and AAV vectors. Also, immune responses to the viral vectors make repeated use of these systems impossible. Finally, the size of these viruses makes the inclusion of the genetic sequence of a large heterologous protein technically challenging.

### SUMMARY OF THE INVENTION

The present invention relates to a cytomegalovirus (CMV) that comprises a nucleotide sequence encoding a heterologous polypeptide (CMVhet) and the use of the CMVhet as a vector to express the heterologous sequence. In some embodiments of the invention the CMVhet is a human CMV that is able to replicate in vivo or in vitro. In alternative embodiments, the CMVhet is replication defective (rdCMVhet).

In specific embodiments of the present invention, the CMVhet is a conditional replication defective CMV that expresses a heterologous polypeptide (rdCMVhet) and use of the rdCMVhet, such as in compositions and methods of treating and/or decreasing the likelihood of a pathology (including, but not limited to, pathogen infection, cancer and genetic disorders) in a patient. The rdCMVhet described herein is replication defective due to comprising a nucleic acid encoding one or more fusion proteins that comprise an essential protein fused to a destabilizing protein. In the absence of a stabilizing agent, the fusion protein is degraded. Thus, the rdCMVhet can be grown in tissue culture under conditions that allow for replication (i.e., in the presence of the stabilizing agent) but replication can be reduced, and preferably prevented, when administered to a patient (in the absence of the stabilizing agent). When replication does not occur in vivo, the rdCMVhet retains the ability to infect host cells leading to expression of the heterologous polypeptide in vivo.

The invention provides a conditional replication defective CMV comprising a nucleotide sequence encoding a heterologous protein. The rdCMV comprises a nucleic acid encoding one or more fusion proteins that comprise an essential protein fused to a destabilizing protein. The nucleic acids encoding the wild type essential protein are no longer present in the rdCMV and thus the fusion protein is required for viral replication. In preferred embodiments, the essential proteins are selected from the group consisting of UL51, UL52 and UL87 and the destabilizing protein is FKBP or a derivative thereof.

The invention also relates to a composition comprising an isolated rdCMVhet and a pharmaceutically acceptable carrier (“rdCMVhet composition”). The composition can further comprise an adjuvant including, but no limited to ISCOMATRIX® adjuvant (CSL, Ltd. Parkville, Australia) and/or an aluminum salt adjuvant, which in some embodiments is an aluminum phosphate adjuvant.

Another aspect of the present invention is a method of inducing an immune response to a heterologous polypeptide in a patient, said method comprising administering a rdCMVhet composition to the patient, wherein the rdCMVhet comprises a nucleotide sequence that encodes the polypeptide. Patients can be treated prophylactically or therapeutically using the methods of administration of the rdCMVhet of the present invention. Prophylactic treatment provides sufficient protective immunity to reduce the likelihood or severity of an infection to the pathogen that expresses the heterologous polypeptide, or reduces the severity, duration, onset or likelihood of the clinical manifestations thereof. Therapeutic treatment can be performed to reduce the duration/severity of a current infection, or the clinical symptoms thereof.

Another aspect of the present invention is a method for generating passive immunity against an antigen in a patient by administering a rdCMVhet composition to the patient, wherein the rdCMVhet comprises a nucleotide sequence that encodes an antigen binding protein (e.g. an antibody fragment) or an antibody that binds to the antigen. The heterologous polypeptide can be an antibody or portion thereof that can neutralize the antigen, which can be either a pathogen or a toxin produced by a pathogen. Said method can provide protection (i.e. induce a protective immune response) from infectious challenge in animal models. Patients can be treated prophylactically or therapeutically by administration of the rdCMVhet of the present invention, which elicits expression of the antibody, thereby providing protective immunity against the antigen to reduce the likelihood or severity of an infection or the clinical manifestations thereof or to reduce the length/severity of a current infection or the clinical manifestations thereof.

Another aspect of the present invention is the use of a rdCMVhet composition in a method of gene therapy. In said method, the rdCMVhet comprises a nucleotide sequence that encodes a heterologous polypeptide or portion thereof that can benefit a patient that is suffering a pathology associated with the lack of expression of the wild-type polypeptide or lack of a required level of expression of the polypeptide, or wherein the patient can otherwise benefit from expression of the polypeptide in vivo. In a specific embodiment, the heterologous polypeptide is a polypeptide that is absent, decreased, mutated and/or not functioning properly in the patient. In another specific embodiment, the heterologous polypeptide is a polypeptide that aids in the treatment of a pathology but does not contribute to the underlying pathology. Patients can be treated prophylactically or therapeutically by administration of the rdCMVhet of the present invention to cause expression of the heterologous polypeptide.

The invention also relates to a method of making the rdCMVhet of the invention comprising propagating the rdCMVhet on epithelial cells, such as ARPE-19 cells (ATCC Accession No. CRL-2302) or fibroblast cells, such as MRC-5 cells (ATCC Accession No. CCL-171), in the presence of Shield-1. In some embodiments, the rdCMVhet is propagated on epithelial cells or fibroblast cells on microcarriers or other high density cell culture systems.

As used throughout the specification and in the appended claims, the singular forms “a,” “an,” and “the” include the plural reference unless the context clearly dictates otherwise.

As used throughout the specification and appended claims, the following definitions and abbreviations apply:

The term “CMV” refers to human cytomegalovirus.

The term “conditional replication defective virus” refers to virus particles that can replicate in a certain environments but not others, e.g., is missing an essential component needed for efficient viral replication which, when restored, can replicate in a host that a wild type virus can replicate. In preferred embodiments, a virus is made a conditional replication defective virus by destabilization of one or more proteins essential for viral replication. The nucleic acids encoding the wild type, non-destabilized essential proteins are no longer present in the conditional replication defective virus. Under conditions where the one or more essential proteins are destabilized, viral replication is decreased by preferably greater than 50%, 75%, 80%, 85%, 90%, 95%, 99%, or 100% as compared to a virus with no destabilized essential proteins. However, under conditions that stabilize the destabilized essential proteins, viral replication can occur at preferably at least 75%, 80%, 90%, 95%, 99% or 100% of the amount of replication of a CMV that does not contain a destabilized essential protein. In more preferred embodiments, one or more essential proteins are destabilized by fusion with a destabilizing protein such as FKBP or a derivative thereof. Such fusion proteins can be stabilized by the presence of a stabilizing agent such as Shield-1.

The term “rdCMV” refers to a conditional replication defective cytomegalovirus. International Patent Application Publication No. WO 2013/036465 discloses conditional replication defective CMV (rdCMV) and the use of rdCMV to decrease the likelihood of an infection by CMV or pathology associated with such an infection in a patient.

The terms “fused” or “fusion protein” refer to two polypeptides arranged in-frame as part of the same contiguous sequence of amino acids. Fusion can be direct such there are no additional amino acid residues between the polypeptides or indirect such that there is a linker to improve performance or add functionality. In some embodiments, the linker can comprise amino acids for convenience of genetic manipulation. In preferred embodiments, the fusion is direct.

The term “essential protein” or “essential CMV protein” refers to a viral protein that is needed for viral replication in vivo and in tissue culture. Examples of essential proteins in CMV include, but are not limited to, IE1/2, UL37x1, UL44, UL51, UL52, UL53, UL56, UL77, UL79, UL84, UL87 and UL105.

The term “non-essential protein” refers to a viral protein that is not needed for viral replication in vivo and in tissue culture. A virus with a non-essential gene deleted or inactivated can replicate at a level substantially similar to the level at which the virus containing a functional non-essential gene replicates.

In embodiments of the invention, the replication-defective CMV vectors comprise a nucleic acid comprising a sequence of nucleotides that encodes a fusion protein, wherein the fusion protein comprises an essential CMV protein fused to a destabilizing protein. In general, these fusion proteins are referred to as “ddCMV fusion proteins.” The specific fusion proteins to be used in the rdCMVhet of the invention are referred to herein by the designation “dd” followed by the name of the gene encoding the essential protein, e.g., ddUL51, and ddUL52, which refer to destabilizing proteins fused to UL51 and UL52, respectively. A conditional replication defective CMV comprising both a nucleic acid encoding a fusion protein as described herein and one or more nucleic acids that encode one or more heterologous polypeptides is referred to herein as a “rdCMVhet”, which may be further referred to by the designation rdCMVhet, followed by the name of the fusion protein it comprises, e.g. “rdCMVhet-ddUL51” refers to a replication defective CMV that comprises (1) one or more nucleic acids encoding one or more heterologous proteins, and (2) a nucleic acid encoding a fusion protein comprising a destabilization protein fused to UL51.

The term “destabilized essential protein” refers to an essential protein that is expressed and performs its function in viral replication and is degraded in the absence of a stabilizing agent. In preferred embodiments, the essential protein is fused to a destabilizing protein such as FKBP or a derivative thereof. Under normal growth conditions (i.e., without a stabilizing agent present) the fusion protein is expressed but degraded by host cell machinery. The degradation does not allow the essential protein to function in viral replication thus the essential protein is functionally knocked out. Under conditions where a stabilizing agent such as Shield-1, is present the fusion protein is stabilized and can perform its function at a level that can sustain viral replication that is preferably at least 75%, 80%, 90%, 95%, 99% or 100% of the amount of replication of a CMV that does not contain a destabilized essential protein.

The term “FKBP” refers to a destabilizing domain (“dd”) protein otherwise known as FK506-binding protein (see U.S. Pat. No. 8,173,792).

“Effective amount” means sufficient CMVhet is introduced to a patient to produce a desired effect such as inducing an immune response in the patient against the protein encoded by the heterologous DNA or preventing or reducing the likelihood of infection with a pathogen associated with the protein encoded by the heterologous DNA. One skilled in the art recognizes that this level may vary.

“An immunologically effective amount” refers to the amount of an immunogen that can induce an immune response against the heterologous polypeptide when administered to a patient that can protect the patient from infection by the pathogen that expresses the heterologous polypeptide (including primary, recurrent and/or super-infections) and/or ameliorate at least one pathology associated with infection and/or reduce the severity/length of infection in the patient. The amount should be sufficient to significantly reduce the likelihood or severity of an infection. Animal models known in the art can be used to assess the protective effect of administration of immunogen. For example, immune sera or immune T cells from individuals administered the immunogen can be assayed for neutralizing capacity by antibodies or cytotoxic T cells or cytokine producing capacity by immune T cells. The assays commonly used for such evaluations include but not limited to viral neutralization assay, anti-viral antigen ELISA, interferon-gamma cytokine ELISA, interferon-gamma (IFN-γ) ELISPOT, intracellular multi-cytokine staining (ICS), and ⁵¹Chromium release cytotoxicity assay Animal challenge models can also be used to determine an immunologically effective amount of immunogen.

“Induce an immune response” refers to the ability of a conditional replication defective CMV expressing a heterologous polypeptide to produce an immune response in a patient, preferably a mammal, more preferably a human, to which it is administered, wherein the response includes, but is not limited to, the production of elements (such as antibodies) which specifically bind, and preferably neutralize, a pathogen and/or cause T cell activation. A “protective immune response” is an immune response that reduces the likelihood that a patient will contract an infection (including primary, recurrent and/or super-infection) and/or ameliorates at least one pathology associated with infection and/or reduces the severity/length of infection.

In preferred embodiments, the immune response induced by a replication defective virus as compared to its live virus counterpart is the same or substantially similar in degree and/or breadth. In other preferred embodiments, the morphology of a replication defective virus by electron microscopy analysis is indistinguishable or substantially similar to its live virus counterpart.

“Passive immunity” refers to the transfer of active humoral immunity in the form of antibodies. Passive immunity provides immediate protective effect to the patient from the pathogen recognized by the administered antibodies and/or ameliorates at least one pathology associated with pathogen infection. However, the patient does not develop an immunological memory to the pathogen and therefore must continue to receive the administered antibodies for protection from the pathogen to persist. In preferred embodiments, monoclonal antibodies, more preferably human or humanized antibodies, are administered to a patient to confer passive immunity.

“Gene therapy” refers to the expression of a polypeptide of interest or portion thereof in a patient that is suffering a pathology and can benefit from expression of the polypeptide. The polypeptide of interest can be one that is absent, decreased, mutated and/or not functioning properly in the patient and thus causing or contributing to the pathology. Alternatively, the polypeptide of interest can be one that aids in the treatment of a pathology but does not contribute to the underlying pathology. One or more nucleic acids encoding the polypeptide of interest are introduced into the patient by a delivery vehicle (such as rdCMVhet).

A “patient” refers to a mammal capable of suffering a disorder that can be prevented and or treated by the heterologous polypeptide within the rdCMVhet of the invention. Patients can benefit from an immunological response to a pathology (e.g., infection with a virus, bacteria or parasite or cancer), to the modulation of the immune response, to the restoration of an impaired biological function (e.g., gene therapy).

The terms “pentameric gH complex” or “gH complex” refer to a complex of five viral proteins on the surface of the CMV virion. The complex is made up of proteins encoded by UL128, UL130, and UL131 assembled onto a gH/gL scaffold (Wang and Shenk, 2005 Proc Natl Acad Sci USA 102:1815; Ryckman et al., 2008 J. Virol. 82:60).

### BRIEF DESCRIPTION OF THE DRAWINGS

FIGS. 1A-1B shows a schematic diagram of the construction of a strain of CMV with restored expression of the pentameric gH complex. (A) Strategy for generation of self-excisable Bacterial Artificial Chromosome (BAC) to manipulate AD169 viral genome. (B) Repair of the frame shift mutation in UL131 to restore its expression. (C) Replacement of GFP with a cre recombinase gene to create a self excisable CMV BAC.

FIG. 2 shows the Shield 1 concentration dependent progeny virus production of pentameric gH complex expressing CMV with various FKBP fusion proteins. FKBP fusion proteins used in this study comprise an essential protein (IE1/2, UL51, UL52, UL79, UL84, UL87, IE1/2-UL51) fused to an FKBP derivative (ddIE1/2, ddUL51, ddUL52, ddUL79, ddUL84, ddUL87, and ddIE1/2-UL51). ARPE-19 cells were infected with the various rdCMV viruses comprising the noted fusion proteins, at a multiplicity of 0.01 PFU/cell for 1 hour, washed twice with fresh medium, and incubated in the growth medium containing 0, 0.05, 0.1 0.5 or 2 μM of Shield-1. Seven days post infection, the cell free virus was collected, and virus titers were determined by TCID50 assay on ARPE-19 cells in the presence of 2 μM of Shield 1.

FIGS. 3A-3D show the growth kinetics of rdCMV in ARPE-19 cells. Cells were infected with viruses containing (A) IE1/2, (B) UL51, (C) IE1/2-UL51 fusion proteins or the (D) parental beMAD virus at multiplicity of 0.01 PFU/cell. After one hour, the cells were washed twice with fresh medium, and incubated in the absence (open circle) or presence (closed circle) of 2 μM of Shield-1. Cell-free virus was collected at the indicated time points after infection, and infectious virus was quantified by TCID50 assay on ARPE-19 cells in the medium containing 2 μM of Shield-1.

FIGS. 4A-4E show growth kinetics of the ddIE1/2-UL51 rdCMV in different cell types. (A) MRC-5 (B) HUVEC (C) AoSMC (D) SKMC (E) CCF-STTG1 cells were infected with the rdCMV virus and incubated for one hour. The cells were washed twice with fresh medium, and then incubated in the absence (open circle) or presence (closed circle) of 2 μM of Shield-1. Cell-free virus was collected at the indicated time points after infection, and infectious virus was quantified by TCID50 assay on ARPE-19 cells in the medium containing 2 μM of Shield-1.

FIGS. 5A-5C provides an immunogenicity analysis of the ddIE1/2-UL51 rdCMV in mice, rabbits and rhesus macaques. (A) Mice were immunized at weeks 0 and ˜4 with beMAD (open circles) or the IE1/2-UL51 rdCMV (closed circles). (B) Rabbits were immunized at weeks 0, 3 and 8 with 10 μg beMAD or the indicated rdCMV. (C) Rhesus macaques were immunized at weeks 0 and 8 with 100 μg beMAD or the IE1/2-UL51rdCMV. In each case, serum samples were collected and analyzed by CMV micro-neutralization assay on ARPE-19 cells. Lines indicate the geometric mean titers of the neutralization (NT50) in each group.

FIG. 6 shows longitudinal neutralizing titers in rhesus macaques vaccinated with the double fusion virus ddIE1/2-UL51. Groups of rhesus monkeys (n=5) were vaccinated with the indicated vaccine dose or formulations at week 0, 8, and 24 (shown as black triangles), while one group received gb/mf59 (30 mg/dose) at week 0, 4 and 24. The immune sera were collected at indicated time points and evaluated in a viral neutralization assay. The GMT of NT50 titers is plotted longitudinally with the standard error for the group. AAHS: amorphous aluminum hydroxylphosphate sulfate; IMX: ISCOMATRIX®; HNS: base buffer.

FIGS. 7A-7D show IFN-γ ELISPOT in rhesus macaques with the double fusion virus ddIE1/2-UL51 vaccination with either a 100 μg (A) or 10 μg (B-D) per dose. Either no adjuvant (A-B), AAHS (C) or ISCOMATRIX® (D) were used. PBMC were stimulated with peptide pools representing HCMV antigens. Gray bars representing GMT for each antigen of the group (n=5). Responder rate for each antigen is shown at the top of each antigen within the panels.

FIGS. 8A-8B show vaccination of the double fusion virus ddIE1/2-UL51 is able to induce T-cell responses of both CD8+(A) and CD4+(B) phenotypes in rhesus macaques. PBMC were collected from monkeys given either a 100 μg or 10 μg dose of vaccine with ISCOMATRIX® as adjuvant. PBMCs were stimulated with peptide pools representing HCMV antigens, followed by staining for IFN-γ and CD4+/CD8+ surface T-cell markers. The data are presented as number of CD4+/CD8+ positive, IFN-γ positive cells per million PBMC. The lines represent the geometric means (GMT) of the group receiving the same vaccine (n=5). The numbers at the bottom of the graphs represent the GMT of both vaccinated groups (n=10). CMV: purified virus; SEB: mitogen used as positive control agent; IMX: ISCOMATRIX® (CSL Ltd., Parkville, Australia).

FIG. 9 shows Merck aluminum phosphate adjuvant (MAPA) can enhance neutralizing antibody titers in monkeys. Rhesus monkeys were immunized with a 30 μg dose of the double fusion virus vaccine formulated in HNS (base buffer), AAHS or MAPA at week 0 and 8. The serum samples were collected at week 12 and evaluated for neutralizing titers. The lines represent geometric means for the group.

FIG. 10 shows a comparison of the proteomic composition of fibroblast-tropic AD169 virus and epithelial-tropic vaccine virus. The BAC-derived epithelial tropic AD169 (beMAD), also used as an experimental vaccine, was constructed by repairing the mutation in the UL131 ORF, existing in the parental AD169 virus. The protein compositions of AD169 and vaccine virus (beMAD) were determined by semi-quantitative, label-free shotgun proteomics. Tryptic digests of each strain were analyzed in triplicate by nano LC-MS/MS. The analysis identified 50 viral proteins at a false discovery rate of less than 0.5%. Label-free quantification was performed based on the peak height of identified MS signals and fold change values were calculated to differentiate ad169 and the vaccine. An analysis of variance (ANOVA) was performed to identify statistically significant changes (p-value <0.01). Fold changes were considered to be significant if they were higher than 2-fold and the p-value of the ANOVA analysis was below 0.01. Fold changes shown by striped bars are not significant, whereas fold changes shown by hatched bars are significant. Proteins that were exclusively identified in either one of the samples were artificially set to a fold change of +/−25 for visualization. Members of the pentameric gH complex are indicated with an asterisk. Guiding dashed lines indicate 2-fold change limits.

FIG. 11 shows the luciferase activity in cells infected with CMV-gLuc virus, as described in Example 7. ARPE-19 cells were infected with CMV-gLuc virus at a MOI of 0.01 and the supernatant was sampled as indicated. The samples were stored at −70° C. until assayed. The luciferase activity of the samples was determined using a Pierce gaussia luciferase glow kit.

FIG. 12 shows the luciferase activity in rabbits inoculated with CMV-gLuc virus, as described in Example 7. Two New Zealand white rabbits were inoculated with ˜100 μg of CMV-gLuc virus by intramuscular injection. Plasma samples were collected as indicated and stored at −70° C. until assayed. The luciferase activity of the plasma samples was determined using a Pierce gaussia luciferase glow kit.

FIG. 13 shows the detection of HIV-1 GAG (p24) in viral cultures of CMV-GAG211 (panel A) and CMV-GAG602 (panel B). ARPE-19 cells infected with CMV-GAG vectors at MOI of 0.01. The virus comprising the GAG211 construct was cultured with or without Shld-1 (2 μM) as indicated. Samples were collected as indicated and stored at −70° C. until assayed. Relative GAG signal was determined with culture samples diluted at 1:1000, using a quantitative ELISA kit (PerkinElmer HIV-1 p24 ELISA). Estimated peak p24 concentrations were ˜30 ng/mL for GAG211 and ˜1 ng/mL for GAG602. Shown in panel C are ARPE-19 cells infected with CMV-GAG vectors at MOI of 0.01. The cells were fixed and permealized at 72 hours post infection and stained with a polyclone rabbit IgG to HIV-1 p55 GAG.

FIG. 14A-14B shows that vaccination with a CMV vector expressing HIV-1 GAG elicits T-cell responses in mice. (A). Female BALB/c mice were immunized with 10, 3 or 1 μg/dose CMV-GAG211 vaccine i.m. at week 0 and 3. The control group received 10 μg/dose ddUL51 virus. At week 7, spleen cells pooled from three mice of each group and of three naïve mice were tested in IFN-γ ELISPOT assay. (B) Female C57BL/6×BALB/c F1 mice were immunized with 3 μg/dose CMV-GAG211 vaccine or 10 μg/dose ddUL51 virus at week 0 and 3. At week 4, spleen cells pooled from three mice of each group and of three naïve mice were tested in IFN-γ ELISPOT assay. The antigens used were medium (striped bars), GAG pool (hatched bars; a pool of 15-mer peptides overlapping by 11 amino acids for the entire ORF of HIV-1 gag), or CD8 peptide (open bars; a peptide of a known CD8 T-cell epitope by H-2K^{d}, Meta et al., J. Immunol. 1998 161:2985), or a peptide pool representing CMV pp65 antigen (striped bars).

FIG. 15 shows Western blot analysis of HIV GAG expression in cells infected with CMV vectors. ARPE-19 cells were infected with respected CMV-gag expressing viruses were cultured until cytopathic effects (CPE) shown in ˜90% of cells. Samples of cell lysate and the supernatant were harvested, reduced and denatured at 95° C. before analyzed on SDS PAGE. The gel was transferred to nitrocellulose membrane and blotted with a mouse monoclonal antibody to HIV-gag (Abeam AB9071). A recombinant HIV gag p55 from E. coli was included as positive control (r-gag protein p55) and ddUL51 virus (labeled as u151dd) as negative control.

### DETAILED DESCRIPTION OF THE INVENTION

The present invention relates to a human cytomegalovirus (CMV) that comprises a nucleotide sequence encoding a heterologous polypeptide (CMVhet) and the use of the CMVhet as a vector to express the heterologous sequence. The CMV vectors of the invention are replication defective, or chemically controllable replication capable, or replication competent. Thus, in some embodiments of the invention, the CMVhet is a human CMV that is able to replicate in vivo or in vitro. In alternative embodiments, the CMVhet is a replication defective CMV (rdCMVhet).

In some embodiments of the invention, the CMVhet is a conditional replication defective CMV expressing a heterologous polypeptide (rdCMVhet) and the use of rdCMVhet in compositions and methods such as methods of treating and/or decreasing the likelihood of a pathology in a patient, wherein the pathology (1) is associated with the lack of normal levels of the polypeptide in the patient, the presence of a mutated form of the polypeptide in the patient, or the lack of a properly functioning/expressing polypeptide in the patient; (2) is caused by or otherwise associated with expression of the polypeptide in the patient, wherein the method comprises inducing an immune response against the polypeptide in the patient; (3) is caused by an antigen to which the polypeptide binds, wherein the method comprises inducing passive immunity against the antigen; or (4) would otherwise be beneficially affected by the provision of the polypeptide to the patient. In particular embodiments of the invention, the rdCMVhet is lacking in at least one functional essential protein and instead comprises a nucleotide sequence encoding one or more fusion proteins, wherein the one or more fusion proteins comprise the essential protein fused to a destabilizing protein. The rdCMVhet of the invention further comprises a nucleotide sequence that encodes one or more heterologous polypeptides. In the absence of a stabilizing agent, the fusion protein is degraded by host cell machinery. In the presence of a stabilizing agent, the fusion protein is stabilized and not degraded.

Suitable heterologous polypeptides for use in the present invention are useful in the prevention and/or treatment of a disorder to which an immunologic response is desired. In some embodiments, the heterologous polypeptide is an antigen useful in eliciting an immunological response (e.g., eliciting antibodies, CD4⁺ T cells and/or CD 8⁺ T cells) for preventing (e.g., decreasing the likelihood) and/or treating a pathogen infection (e.g. viral, bacterial or parasitic infection). In other embodiments, the heterologous polypeptide is a monoclonal antibody or portion thereof useful in inducing passive immunity to an antigen. In other embodiments, the heterologous polypeptide is a polypeptide that can benefit a patient that is suffering a pathology (e.g., an immune modulator, a polypeptide that is deficient in a genetic condition, or a tumor-associated antigen).

Embodiments of the invention include the recombinant CMVhet or compositions thereof, described herein, or a vaccine comprising or consisting of said CMVhet or compositions (i) for use in, (ii) for use as a medicament for, or (iii) for use in the preparation of a medicament for: (a) therapy (e.g., of the human body); (b) medicine; (c) inhibition of a pathology (e.g., caused by a pathogen, cancer or genetic disorder); (d) treatment or prophylaxis of a pathology (e.g., caused by a pathogen, cancer or genetic disorder) or, (e) treatment, prophylaxis of, or delay in the onset or progression of pathology associated with a pathogen, cancer or genetic condition. In these uses, the recombinant CMVhet, compositions thereof, and/or vaccines comprising or consisting of said CMVhet or compositions can optionally be employed in combination with one or more anti-pathogen agents (e.g., anti-viral compounds, anti-viral immunoglobulins or antibiotics; combination vaccines, described infra).

### Replication Defective CMV

One aspect of the invention is a conditional replication defective CMV, or “rdCMVhet,” which comprises:

(a) a nucleic acid encoding a fusion protein, wherein the fusion protein comprises an essential CMV protein fused to a destabilizing protein; and

(b) a nucleic acid encoding a heterologous polypeptide. Said rdCMVhet is useful as a vector to express the heterologous gene and allow production of the heterologous polypeptide.

In some embodiments of this aspect of the invention, the essential protein is selected from the group consisting of UL51, UL52 and UL87.

a. CMV Strains

CMV, also known as human herpesvirus 5 (HHV-5), is a herpes virus classified as a member of the beta subfamily of herpesviridae. According to the Centers for Disease Control and Prevention, CMV infection is found fairly ubiquitously in the human population, with an estimated 40-80% of the United States adult population having been infected. The virus establishes life-long persistent infection in its host, but its infection is generally asymptomatic. CMV is a large DNA virus with genome size of ˜240 kb, and its genome sequence and structural and functional gene maps are known (Chee et al., 1990, Curr. Top. Microbiol. Immunol. 154:125-69; Yu et al., 2003, Proc. Natl. Acad. Sci. USA 100:12396-12401; Dunn et al., 2003, Proc. Natl. Acad. Sci. USA 100:14223-14228). The virus can be genetically manipulated in E. coli with bacterial artificial chromosome technology (Borst et al., 2003, Hum Gene Ther 14:959-70). Interestingly, although natural CMV infection can elicit robust humoral and cellular immunity to CMV in humans, seropositive subjects have been shown to be susceptible to viral challenge (Plotkin et al., 1989, J Infect Dis 159:860-5). In addition, CMV encodes many immune evasion and modulation proteins important for establishing persistent infection in vivo (Mocarski et al., Cytomegaloviruses. In: Knipes D M, Howley P M, editors. Fields Virology: Lippincott Williams & Wilkins, 2007: 2701-72).

Although clinical CMV isolates replicate in a variety of cell types, laboratory strains AD169 (Elek & Stern, Lancet 1:1 (1974)) and Towne (Plotkin et al., Infect. Immun. 12:521 (1975)) replicate almost exclusively in fibroblasts (Hahn et al., J. Virol. 78:10023 (2004)). The restriction in tropism, which results from serial passages and eventual adaptation of the virus in fibroblasts, is stipulated as a marker of attenuation (Gerna et al., J. Gen. Virol. 86:275 (2005); Gerna et al, J. Gen Virol. 83:1993 (2002); Gerna et al, J. Gen Virol. 84:1431 (2003); Dargan et al, J. Gen Virol. 91:1535 (2010)). Mutations causing the loss of epithelial cell, endothelial cell, leukocyte, and dendritic cell tropism in human CMV laboratory strains have been mapped to three open reading frames (ORFs): UL128, UL130, and UL131 (Hahn et al., J. Virol. 78:10023 (2004); Wang and Shenk, J. Virol. 79:10330 (2005); Wang and Shenk, Proc Natl Acad Sci USA 102:18153 (2005)). Biochemical and reconstitution studies show that UL128, UL130 and UL131 assemble onto a gH/gL scaffold to form a pentameric gH complex (Wang and Shenk, Proc Natl Acad Sci USA 102:1815 (2005); Ryckman et al, J. Virol. 82:60 (2008)). Restoration of this complex in virions restores the viral epithelial tropism in the laboratory strains (Wang and Shenk, J. Virol. 79:10330 (2005)).

In some embodiments, the recombinant viruses of the present invention can express the five viral proteins that make up the pentameric gH complex and assemble the pentameric gH complex on the viral envelope. The sequences of the complex proteins from CMV strain AD169 are provided at GenBank Accession Nos. NP_{—}783797.1 (UL128), NP_{—}040067 (UL130), CAA35294.1 (UL131), NP_{—}040009 (gH, also known as UL75) and NP_{—}783793 (gL, also known as UL115). In other embodiments, the recombinant virus to be used in the method of the invention does not display a pentameric gH complex on its virion. Some attenuated CMV strains have one or more mutations in UL128, UL130 and/or UL131 such that one or more of the proteins are not expressed and therefore the gH complex is not formed. In such cases, the mutations can be repaired (using methods such as those in Wang and Shenk, 2005 J. Virol. 79:10330) if the expression of the gH complex is desired in the rdCMV of the invention. In one embodiment, the attenuated CMV is AD169 that has restored gH complex expression due to a repair of a mutation in the UL131 gene (e.g. beMAD169, described in Example 1). Additionally, attenuated CMV strains that express the pentameric gH complex can be made replication defective according to the methods of the invention. In another embodiment, the attenuated CMV is an AD169 that does not express the pentameric gH complex, e.g. MAD169, as described in Example 1. In such embodiments, the rdCMV is propagated on non-epithelial cell types including, but not limited to, fibroblasts such as MRC-5 cells.

In the present invention, a conditionally replication defective virus is a mutant in which one or more essential viral proteins have been replaced by a destabilized counterpart of the essential proteins. The destabilized counterpart is encoded by a nucleic acid that encodes a fusion protein between the essential protein and a destabilizing protein. The destabilized essential protein can only efficiently function to support viral replication when a stabilizing agent is present. In preferred embodiments, methods described in U.S. Pat. No. 8,173,792 are used to confer a conditionally replication defective phenotype to a pentameric gH complex expressing CMV. Briefly, one or more proteins essential for CMV replication are fused to a destabilizing protein, a FKBP or FKBP derivative. The nucleic acids encoding the wild type essential protein are no longer present in the rdCMV. In the presence of an exogenously added, cell permeable small-molecule stabilizing agent, Shield-1 (Shld-1), the fusion protein is stabilized and the essential protein can function to support viral replication. Replication of the rdCMV in the presence of the stabilizing agent is preferably at least 75%, 80%, 90%, 95%, 99% or 100% of the amount of replication of a CMV that does not contain a destabilizing fusion protein (e.g, the parental attenuated CMV used to construct the rdCMV). In the absence of Shield-1, the destabilizing protein of the fusion protein directs the fusion protein to be substantially degraded by host cell machinery. With no or minimal amounts of essential protein present, the CMV cannot replicate at an amount to produce or maintain a CMV infection in a patient. Replication of the rdCMV in the absence of the stabilizing agent does not take place or is not efficient (e.g., reduced by preferably greater than 50%, 75%, 90%. 95%, or 99%) as compared to a CMV that does not contain a destabilizing fusion protein (e.g, the parental attenuated CMV used to construct the rdCMV).

b. ddCMV Fusion Proteins

Suitable fusion proteins to be included in the rdCMVhet of the present invention retain sufficient essential protein activity to facilitate viral replication in a host cell in the presence of a stabilizing agent. In the absence of the stabilizing agent, replication of the CMV is decreased (preferably greater than 50%, 75%, 80%, 85%, 90%, 95%, or 99% reduction) or prevented, relative to a CMV that does not contain the fusion protein and instead comprises a wild-type gene encoding the essential protein.

The ddCMV fusion proteins to be included in the rdCMVhet of the invention comprise an essential protein or derivative thereof fused to a destabilizing protein. The essential proteins targeted for destabilization by fusion with the destabilization protein, e.g., FKBP or a derivative thereof: 1) are essential for viral replication; 2) can accommodate the fusion of the destabilizing protein without substantially disrupting function of the essential protein; and 3) can accommodate the insertion of a nucleic acid encoding the destabilization protein or derivative thereof at the 5′ or 3′ end of the viral ORF encoding the essential protein without substantially disrupting the ORFs of other surrounding viral genes. Table 1 shows CMV genes that meet the aforementioned criteria and provides exemplary sequences of these essential proteins fused to a destabilization protein, in this case, an FKBP derivative.

The viral proteins essential for replication that are targeted for destabilization can encode structural or non-structural proteins. In some embodiments, the essential protein genes for use in the ddCMV fusion proteins encode non-structural proteins and are thus not packaged into the rdCMV virions. In other embodiments, the essential protein genes for use in the ddCMV fusion proteins encode structural proteins. In still other embodiments where more than one essential protein is targeted for destruction, the essential protein genes for use in the fusion proteins encode both non-structural proteins and structural proteins.

TABLE 1
Viral genes selected for construction of FKBP fusion

| | | | Fusion | Sequence of Fusion |
|---|---|---|---|---|
| | | Kinetic | of | Protein (odd no's) & |
| Viral Gene | Function* | Phase | FKBP | DNA (even no's) |
| IE1/2 | viral tran- | Immediate | N-term | SEQ ID NOS: 1-2 |
| (UL123/122) | scriptional | early | | |
| | modulators | | | |
| UL37x1 | Viral gene | Immediate | N-term | SEQ ID NOS: 3-4 |
| | regulations | early | | |
| UL44 | DNA | Early | C-term | SEQ ID NOS: 5-6 |
| | replication | | | |
| UL51 | DNA | Late | N-term | SEQ ID NOS: 7-8 |
| | packaging | | | |
| UL52 | DNA | Late | N-term | SEQ ID NOS: 9-10 |
| | packaging | | | |
| | and cleavage | | | |
| UL53 | Capsid egress; | Early | C-term | SEQ ID NOS: 11-12 |
| | nuclear egress | | | |
| UL56 | DNA | Late | N-term | SEQ ID NOS: 13-14 |
| | packaging | | | |
| | and cleavage | | | |
| UL77 | DNA | Early | C-term | SEQ ID NOS: 15-16 |
| | packaging | | | |
| UL79 | Unknown | Early | N-term | SEQ ID NOS: 17-18 |
| UL84 | DNA | Early | C-term | SEQ ID NOS: 19-20 |
| | replication | | | |
| UL87 | Unknown | Late | N-term | SEQ ID NOS: 21-22 |
| UL105 | DNA | Early | C-term | SEQ ID NOS: 23-24 |
| | replication | | | |

*according to Mocarski, Shenk and Pass, Cytomegalovirus, in Field Virology, 2701-2772, Editor: Knipes and Howley, 2007

In some embodiments, the one or more viral proteins essential for viral replication targeted for destabilization are selected from the group consisting of IE1/2, UL51, UL52, UL84, UL79, UL87, UL37x 1, UL77, UL53, UL44, UL56, UL105 or derivatives thereof. In other embodiments, the essential protein comprises a sequence of amino acids as set forth in SEQ ID NO: 32 (IE1/2), SEQ ID NO: 33 (UL37x1), SEQ ID NO: 34 (UL44), SEQ ID NO: 35 (UL51), SEQ ID NO: 36 (UL52), SEQ ID NO: 37 (UL53), SEQ ID NO: 38 (UL56), SEQ ID NO: 39 (UL77), SEQ ID NO: 40 (UL79), SEQ ID NO: 41 (UL84), SEQ ID NO: 42 (UL87), or SEQ ID NO: 43 (UL105). In a specific embodiment, the one or more viral proteins essential for viral replication targeted for destabilization are selected from the group consisting of SEQ ID NO:35 (UL51), SEQ ID NO:36 (UL52), SEQ ID NO:41 (UL84), SEQ ID NO:40 (UL79), and SEQ ID NO:42 (UL87). In a more specific embodiment, the one or more viral proteins essential for viral replication targeted for destabilization are selected from the group consisting of SEQ ID NO:35 (UL51), SEQ ID NO:36 (UL52), and SEQ ID NO:42 (UL87).

Essential protein derivatives contain one or more amino acid substitutions, additions and/or deletions relative to the wild type essential protein yet can still provide the activity of the essential protein at least well enough to support viral replication in the presence of Shield-1. Examples of methods to measure virus activity are provided in the Examples, infra. Methods known in the art can be used to determine the degree of difference between the CMV essential protein of interest and a derivative. In one embodiment, sequence identity is used to determine relatedness. Derivatives of the invention will be preferably at least 85% identical, at least 90% identical, at least 95% identical, at least 97% identical, at least 99% identical to the reference sequence. The percent identity is defined as the number of identical residues divided by the total number of residues and multiplied by 100. If sequences in the alignment are of different lengths (due to gaps or extensions), the length of the longest sequence will be used in the calculation, representing the value for total length.

Thus, the invention provides a rdCMVhet comprising a nucleic acid encoding a fusion protein, wherein the fusion protein comprises an essential CMV protein fused to a destabilizing protein, wherein the essential protein is selected from the group consisting of: IE1/2, UL51, UL52, UL84, UL79, UL87, UL37x 1, UL77, UL53, UL44, UL56, UL105 or derivatives thereof and wherein the destabilizing protein is FKBP, as set forth in SEQ ID NO:29, or an FKBP derivative comprising one or more substitutions relative to SEQ ID NO:29, wherein the substitutions are selected from the group consisting of: F15S, V24A, H25R, E31G, F36V, E60G, M66T, R71G, D100G, D100N, E102G, K105I, K105E, and L106P.

In some embodiments of the invention, the essential protein portion of the ddCMV fusion protein may be a few residues shorter or longer than a full-length essential protein reference sequence, based on the cloning strategy used to fuse the nucleic acid encoding the essential protein to the nucleic acid encoding the destabilizing protein. In such cases, the sequence will be shorter or longer than a reference sequence at the end that is fused to the destabilizing protein. In some embodiments, the essential CMV protein portion of the ddCMV fusion protein is 5, 4, 3, 2, or 1 amino acids shorter than the essential protein reference sequence. Similarly, the destabilizing protein portion of the ddCMV fusion protein may also be 1, 2, 3, 4, or 5 amino acid residues shorter or longer than a reference sequence, especially at the end that is fused to the essential CMV protein.

More than one essential protein can be destabilized by fusion to the destabilization protein, e.g., FKBP or derivative thereof. In some embodiments, the essential proteins function at different stages of CMV replication and/or infection (including but not limited to, immediate early, early or late stages). In other embodiments, the essential proteins function at the same stage of CMV replication and/or infection. In preferred embodiments, the combination of viral proteins essential for viral replication targeted for destabilization are selected from the late stages of viral kinetics. In specific preferred embodiments, the combination of viral proteins essential for viral replication targeted for destabilization are selected from the group consisting of UL51/UL52, UL51/UL87, UL52/UL87.

In one embodiment, at least UL51 is targeted for destabilization in the rdCMV. In another embodiment, a fusion protein comprising UL51 fused to an FKBP derivative (ddUL51) is SEQ ID NO:7. An exemplary nucleic acid encoding the ddUL51 set forth in SEQ ID NO:7 comprises a sequence of nucleotides as set forth in SEQ ID NO:8. An exemplary genome of a rdCMV with a destabilized UL51 is shown in SEQ ID NO:28.

ddCMV fusion proteins should not be based on non-essential CMV proteins. Examples of non-essential proteins in CMV are disclosed herein and further provided in Yu et al., 2003, Proc. Natl. Acad. Sci. USA 100:12396-12401):

An example of a destabilizing protein and stabilizing agent is described in U.S. Pat. No. 8,173,792, which discloses compositions, systems and methods for modulating the stability of proteins using a small-molecule. Briefly, a protein is fused to a stability-affecting protein, FKBP or a derivative thereof. An exogenously added, cell permeable small-molecule, Shield-1 (Shld-1), interacts with the FKBP or derivative thereof and stabilizes the fusion protein. In the absence of Shield-1, the FKBP or derivative thereof directs the fusion protein to be degraded by host cell machinery.

In embodiments of the present invention, the destabilization protein of the ddCMV fusion protein is FKBP or an FKBP derivative. In exemplary embodiments of this embodiment of the invention, the rdCMV comprises a sequence of nucleotides that encodes a ddCMV fusion protein, such as the exemplary ddCMV nucleotide sequence that encode ddIE1/2 (SEQ ID NO:2), ddUL37x1 (SEQ ID NO:4), ddUL44 (SEQ ID NO:6), ddUL51 (SEQ ID NO:8), ddUL52 (SEQ ID NO:10), ddUL53 (SEQ ID NO:12), ddUL56 (SEQ ID NO:14), ddUL77 (SEQ ID NO:16), ddUL79 (SEQ ID NO:18), ddUL84 (SEQ ID NO:20), ddUL87 (SEQ ID NO:22), and ddUL105 (SEQ ID NO:24), which comprise an FKBP derivative fused to an essential CMV protein. In further embodiments, the rdCMVhet comprises a nucleotide sequence that encodes a ddCMV fusion protein having a sequence of amino acids as set forth in: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, or SEQ ID NO:23.

In still further embodiments, the rdCMV comprises a nucleotide sequence encoding a protein that is at least 90%, at least 95%, at least 96%, at least 97%, at least 98% or at least 99% identical to a ddCMV fusion reference protein having a sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, or SEQ ID NO:23. In still further embodiments, the ddCMV fusion protein has 1, 2, 3, 4, 5, 6, 7, 8, 9, 10, 11, 12, 13, 14, 15, 20, or 25 amino acid differences relative to a ddCMV fusion reference protein having a sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, and SEQ ID NO:23.

In alternative embodiments, the destabilizing protein is based on the ligand binding domain of the estrogen receptor that can be regulated by one of two synthetic ligands, CMP8 or 4-hydroxytamoxifen (see, e.g., Miyazaki et al., 2012, J. Am. Chem. Soc. 134:3942-3945). In other embodiments, the destabilizing protein is based on E. coli dehydrofolate reductase (ecDHFR) that can be regulated by a chemical ligand Guard-1 (Iwamoto et al., 2010, Chem & Biol 17:981-8).

More than one destabilization protein, e.g. FKBP or derivative thereof, can be fused to the essential protein. In embodiments where there is more than one FKBP or derivative thereof fused to the essential protein, each of the individual FKBP or derivatives thereof can be the same or different. In another embodiment, there is one FKBP or derivative thereof fused to the essential protein.

Using recombinant DNA methods well known in the art, the nucleic acid encoding an essential protein for CMV replication and/or establishment/maintenance of CMV infection is attached to a nucleic acid that encodes the destabilization protein, e.g., FKBP or a derivative thereof. The destabilization protein or derivative thereof can be fused to the essential protein either directly or indirectly. In an embodiment, the destabilization protein or derivative thereof is fused in-frame to the essential protein directly. In alternative embodiments, the essential protein is fused indirectly, i.e. through a linker, to the destabilization protein. The destabilization protein or derivative thereof can be fused to the essential protein either at either the N- or C-terminus of the essential protein. In different embodiments, the destabilization protein is fused to the N-terminus of the essential protein.

An exemplary FKBP reference sequence, useful in generating FKBP fusion proteins, is provided below:

(SEQ ID NO: 29)
GVQVETISPGDGRTFPKRGQTCVVHYTGMLEDGKKFDSSRDR
NKPFKFMLGKQEVIRGWEEGVAQMSVGQRAKLTISPDYAYGA
TGHPGIIPPHATLVFDVELLKLE

An exemplary nucleotide sequence encoding the FKBP reference sequence is provided by SEQ ID NO:25. Fusion proteins comprising FKBP or an FKBP derivative are degraded by host cell machinery. As used herein, the term “FKBP derivative” refers to a FKBP protein or portion thereof that has been altered by one or more amino acid substitutions, deletions and/or additions, relative to the FKBP reference sequence provided by SEQ ID NO:29. In preferred embodiments of the invention, the FKBP derivative is at least 90% identical to the FKBP reference sequence set forth in SEQ ID NO:29. In addition to structural similarity defined by 90% or greater identity, preferred FKBP derivatives of the invention retain substantially all of the destabilizing properties of FKBP when fused to a heterologous protein, e.g., an essential CMV protein, and also retain substantially all of the ability of FKBP to be stabilized by Shield-1.

In one embodiment, an FKBP derivative is a polypeptide that has an amino acid sequence which differs from the reference sequence provided by SEQ ID NO:29 by one or more amino acid substitutions. In some embodiments, the FKBP protein derivative has 1, 2, 3, 4, 5, 6, 7, 8, 9, or 10 amino acid substitutions relative to the reference sequence. Amino acid substitutions may be “conservative” (i.e. the amino is replaced with a different amino acid from the same class of amino acids (non-polar, polar/neutral, acidic and basic), an amino acid with broadly similar properties, or with similar structure (aliphatic, hydroxyl or sulfur-containing, cyclic, aromatic, basic, and acidic) or “non-conservative” (i.e. the amino acid is replaced with an amino acid of a different type). Broadly speaking, fewer non-conservative substitutions will be possible without altering the biological activity of the polypeptide.

In embodiments of the invention, the FKBP derivative has one or more amino acid substitutions at amino acid position(s) selected from the group consisting of: 15, 24, 25, 31, 36, 60, 66, 71, 100, 102, 105, and 106, with amino acid position #1 corresponding to the first G (glycine) in SEQ ID NO:29. As defined herein, numbering of FKBP derivative amino acid positions are relative to the amino acid position in SEQ ID NO:29—numbering is maintained relative to SEQ ID NO:29 even if there is a gap in sequence in the derivative. For example, an FKBP derivative beginning with GVQVE-ISPGDGRTSPKRG (SEQ ID NO:30) as the first 20 amino acid has an F15S substitution, despite this F15S position (underlined) being the 14^{th} amino acid in the derivative sequence, because the numbering of the reference sequence is maintained after the gap.

In particular embodiments of the invention, the FKBP derivative has one or more amino acid substitutions selected from the group consisting of: F15S, V24A, H25R, E31G, F36V, E60G, M66T, R71G, D100G, D100N, E102G, K105I, K105E, and L106P. In some embodiments, the FKBP is the polypeptide sequence encoded by SEQ ID NO:25. In a another embodiment, the FKBP is an FKBP derivative which is encoded by SEQ ID NO:27. In some embodiments, the FKBP derivative comprises the substitutions F36V and/or L106P, as shown in SEQ ID NO:26, which has both the F36V and L106P substitutions. In alternative embodiments, the FKBP derivative has only one of the F36V and L106P substitutions. In further embodiments, the FKBP derivative has the amino acid substitutions F36V and K105E, for example, the FKBP derivative set forth in SEQ ID NO:44. In still further embodiments, the FKBP derivative has the amino acid substitutions E31G, F36V, R71G, K105E, such as an FKBP derivative having an amino acid sequence set forth in SEQ ID NO:31.

In some embodiments, the nucleic acid that encodes the FKBP or FKBP derivative contains at least some codons that are not commonly used in humans for endogenous FKBP. Including codons that are less commonly used to encode proteins in humans decreases the likelihood that the FKBP or FKBP derivative of the fusion protein will rearrange or recombine with its counterpart in the human genome. An exemplary nucleotide sequence is provided by SEQ ID NO:27, which (1) encodes an FKBP derivative having the substitutions F36V and L106P relative to the FKBP reference sequence provided by SEQ ID NO:29 (i.e. encodes a protein with an amino acid sequence as set forth in SEQ ID NO:26); and (2) was modified to contain codons that are not commonly used in humans to reduce the risk of recombination.

In the presence of Shield-1, ddCMV fusion proteins comprising FKBP or an FKBP derivative is stabilized. However, in the absence of Shield-1, the FKBP or derivative thereof directs the fusion protein to be degraded by host cell machinery. As used herein, the terms “Shield-1” or “Shld1” refer to a synthetic small molecule that binds to wild-type FKBP and derivatives thereof and acts as a stabilizing agent.

Shield-1 has the following structure:

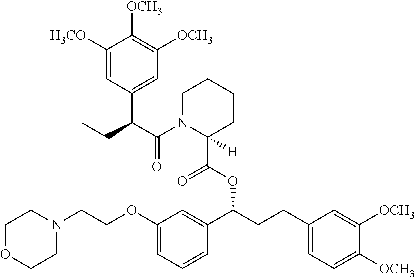 [SMILES: CC[C@H](C(=O)N1CCCC[C@H]1C(=O)O[C@H](CCc1ccc(OC)c(OC)c1)c1cccc(OCCN2CCOCC2)c1)c1cc(OC)c(OC)c(OC)c1]

Binding is about 1,000-fold tighter to the F36V derivative compared to wild-type FKBP (Clackson et al., 1998, Proc. Natl. Acad. Sci. USA 95:10437-42). Shield-1 can be synthesized (essentially as described in Holt et al., 1993, J. Am. Chem. Soc. 115:9925-38 and Yang et al., 2000, J. Med. Chem. 43:1135-42 and Grimley et al., 2008, Bioorganic & Medicinal Chemistry Letters 18:759) or is commercially available from Cheminpharma LLC (Farmington, Conn.) or Clontech Laboratories, INC. (Mountain View, Calif.). Salts of Shield-1 can also be used in the methods of the invention.
c. Heterologous Polypeptides

The rdCMV can be made to express one or more heterologous polypeptides using recombinant DNA technology known in the art (“rdCMVhet”).

Nucleic acids that encode the heterologous polypeptide(s) can be cloned into gene expression cassettes that contain a promoter and polyadenylation sequences. Typically a gene expression cassette includes: (a) nucleic acid encoding a heterologous protein or antigen of interest; (b) a promoter operatively linked to the nucleic acid encoding the protein; and (c) a transcription termination (polyadenylation) signal. The expression cassette can optionally have an enhancer element. If a complete cassette is to be inserted in the CMV genome, the promoter should not be of any one derived from human CMV, mainly due to concern of homologous recombination with the native promoter sequence. The promoters to be used in a cassette could be of cellular origin or other viral promoters.

In some embodiments of the invention, the heterologous protein is fused to at least one dominant CMV antigen, which is known for potent T-cell responses. Thus, the nucleic acid encoding the heterologous protein is fused in-frame to a nucleic acid encoding a dominant CMV antigen, either directly or indirectly. In such embodiments, the promoter is advantageously a native CMV promoter. Said fusion with dominant CMV antigens confer prominent immune responses to the heterologous polypeptide and thus are particularly useful in application wherein the heterologous polypeptide is an immunogen or antigen against which an immune response in desired.

In embodiments of the invention wherein the heterologous sequence is fused to a dominant CMV antigen, a nucleotide sequence encoding any CMV polypeptide that is associated with potent CD4+ or CD8+ T cell responses may be fused to the heterologous sequence, depending on the particular type of immune response desired. For example, if a CD4+ response is desired, the heterologous sequence may be fused to a nucleotide sequence encoding a dominant CMV antigen selected from: UL55 (gB), UL83 (pp65), UL86, UL99 (pp28), UL122 (IE2), UL36, UL48, UL32, and UL113. In alternative embodiments, if a CD8+ response against the heterologous sequence is desired, the heterologous sequence may be fused to a nucleotide sequence encoding a dominant CMV antigen selected from: UL48, UL83, UL123, UL122, US32, UL28, US29, US3, UL32, UL55, UL94, and UL69. Additional dominant CMV antigens that may be fused to the heterologous sequence of interest are disclosed in Sylwester et al. J. Experimental Medicine 202(5): 673-685 (2005).

Decisions must also be made regarding the site within the backbone where the heterologous sequence (i.e. “transgene”) will be introduced and the orientation of the transgene. More specifically, the transgene can be inserted parallel or anti-parallel. In addition, appropriate transcriptional regulatory elements that are capable of directing expression of the transgene in the host cells that the vector is being prepared for use as a vaccine carrier in need to be identified and operatively linked to the transgene. “Operatively linked” sequences include both expression control sequences that are contiguous with the nucleic acid sequences that they regulate and regulatory sequences that act in trans, or at a distance to control the regulated nucleic acid sequence.

Regulatory sequences include: appropriate expression control sequences, such as transcription initiation, termination, enhancer and promoter sequences; efficient RNA processing signals, such as splicing and polyadenylation signals; sequences that enhance translation efficiency (e.g., Kozak consensus sequences); sequences that enhance protein stability, and optionally sequences that promote protein secretion. Selection of these and other common vector elements are conventional and many suitable sequences are well known to those of skill in the art (see, e.g., Sambrook et al, and references cited therein at, for example, pages 3.18-3.26 and 16.17-16.27 and Ausubel et al., Current Protocols in Molecular Biology, John Wiley & Sons, New York, 1989).

CMV infects various cells in vivo, including monocytes, macrophages, dendritic cells, neutrophils, endothelial cells, epithelial cells, fibroblasts, neurons, smooth muscle cells, hepatocytes, and stromal cells (Plachter et al. 1996, Adv. Virus Res. 46:195). The promoter used in the expression cassette should be able to support expression of a nucleic acid operably linked to it in at least one of the above-identified cell types. Preferably, the promoter used in the expression cassette to express the heterologous antigen is active in more than one of the above-identified cell types. Examples of promoters used in the methods of the invention include, but are not limited to, CMV (including HCMV, MCMV, RHCMV, and GPCMV) major IE promoters, SV40 promoter, HSV-1 ICPO or TK promoter, actin promoter, EF1-α promoter, Ubc promoter and PGK promoter. In preferred embodiments, HCMV IE promoter is used in the expression cassette.

In some embodiments of the invention, the nucleic acid encoding the heterologous polypeptide is operably linked to a native CMV viral promoter.

Polyadenylation sequences for use in the methods of the invention include, but are not limited to, bovine growth hormone (BGH) polyadenylation sequences and SV40 polyadenylation sequences.

In embodiments where one or more enhancers are included in the expression cassette, the enhancer(s) are compatible with the promoter used in the expression cassette.

The rdCMVhet can evaluated in vitro for the levels of heterologous polypeptide expression using techniques known in the art (including, but not limited to, enzyme-linked immune assay, immunocytochemistry, immunoblots, FACS, etc.). For example, rdCMVhet can be used to infect epithelial or fibroblast cells in the presence of Shield-1. Levels of heterologous polypeptide expressed can be assayed in the cells either after continued Shield-1 administration or in cultures that have had Shield-1 administration discontinued in order to examine whether the transgene expression is dependent on Shield-1.

In embodiments of the invention, the heterologous polypeptide is an immunogen (antigenic molecule) delivered by the rdCMVhet of the invention, which comprises a polypeptide, protein, or enzyme product that is encoded by a transgene (i.e. heterologous nucleotide sequence) in combination with a nucleotide sequence which provides the necessary regulatory sequences to direct transcription and/or translation of the encoded product in a host cell. The composition of the transgene depends upon the intended use of the vector. For example, if the immunogenic composition is being designed to elicit an antibody response or a cell-mediated immune response in a mammalian host which is specific for an infectious agent, then it is appropriate to utilize a nucleic acid sequence encoding at least one immunogenic product that is predicted to confer pathogen-specific immunity to the recipient. Alternatively, if the composition is being prepared for use as a cancer vaccine, a suitable transgene may comprise an immunogenic portion of a self-antigen, such as a tumor associated antigen (TAA), which has been selected with the goal of eliciting a protective immune response of sufficient potency to both break host tolerance to a particular TAA and to elicit a long-lived (e.g., memory) response that will be sufficient to prevent the initiation of cancer or to prevent tumor progression. Accordingly, suitable immunogenic gene products may be obtained from a wide variety of pathogenic agents (such as, but not limited to viruses, parasites, bacteria and fungi) that infect mammalian hosts, or from a cancer or tumor cell.

In an embodiment of the invention, the heterologous polypeptide expressed by the rdCMVhet is a polypeptide that is useful in the treatment and/or prevention of a pathology. In one embodiment, the pathology is one that can be prevented and/or treated by an immunologic response. In specific embodiments, the heterologous polypeptide is an antigen useful in eliciting an immunological response (e.g., eliciting antibodies, CD4⁺ T cells and/or CD 8⁺ T cells) for preventing and/or treating a disorder (i.e. viral, bacterial or parasitic infection). In other specific embodiments, the heterologous polypeptide is an antibody or portion thereof useful in passive immunity. In another embodiment, the heterologous polypeptide is a polypeptide useful in gene therapy.

In some embodiments, the heterologous polypeptide is an antigen useful in eliciting an immunological response (i.e., eliciting antibodies, CD4⁺ T cells and/or CD8⁺ T cells) for preventing and/or treating a disorder (i.e. viral, bacterial or parasitic infection). In other embodiments, the heterologous polypeptide is an antibody or portion thereof useful in generating passive immunity.

In one embodiment, the rdCMVhet and methods of the invention can be used to prevent and/or treat HIV infection. Antigens that can be used in the methods of the invention to provide an immunologic response in a patient include, but are not limited to, Gag, Nef, Env, Pol, Tat, Rev, Vif, Vpr, gp120 (especially the CD4 binding site, V2V3 loops and MPER region), gp160, gp140 and gp143 (Betts et al., 2002, DNA Cell Biol. 21:665-70).

In another embodiment, the rdCMVhet and methods of the invention can be used to prevent and/or treat malaria. Malaria is a mosquito-borne infectious disease caused by infection by the protist Plasmodium. Antigens that can be used in the methods of the invention to provide an immunologic response in a patient include, but are not limited to, Circumsporozoite protein (CS), SSP2, Liver stage antigen 1 (LSA-1), Liver stage antigen 3 (LSA-3), Exported protein 1 (Exp1), Sporozoite threonine and asparagine rich protein (STARP), Pf5/6, Merozoite surface protein 1 (MSP-1), Merozoite surface protein 2 (MSP-2), Merozoite surface protein 3 (MSP-3), Apical membrane antigen 1 (AMA 1), EBA175, SERA 5, Glutamine rich protein (GLURP), Ring-infected erythrocyte surface antigen (RESA), Pfs25 and Pvs25 (Schwartz et al., 2012, Malaria J. 11:11).

In another embodiment, the rdCMVhet and methods of the invention can be used to prevent and/or treat tuberculosis (TB). TB is a common, and in many cases lethal, infectious disease caused by various strains of mycobacteria, usually Mycobacterium tuberculosis. Antigens that can be used in the methods of the invention to provide an immunologic response in a patient include, but are not limited to, one or more surface antigens of baciller Calmette Guérin (BCG) vaccine strain (attenuated live bovine tuberculosis bacillus Mycobacterium bovis), Ag85A, Ag85B, Mtb32, Mtb39, ESAT-6, TB10.4, Heparin binding hemagglutinin antigen (HBHA), CPF-10, Rv1196, Rv0125, Rv3407 and Rv2660 (Hoft, 2008, Lancet 372:164-75; Kaufmann, 2011, Lancet 11:633-40; Ottenhoff and Kaufmann, 2012, PLoS Pathogens 8:1-12; Anderson and Doherty, 2005, Nature 3:656-662).

In another embodiment, the rdCMVhet and methods of the invention can be used to prevent and/or treat HBV infection. Antigens that can be used in the methods of the invention to provide an immunologic response in a patient include, but are not limited to, surface proteins (including HBsAg, MHBsAg and LHBsAg), core protein (HBcAg), X protein and viral polymerase.

In another embodiment, the rdCMVhet and methods of the invention can be used in cancer immunotherapy. In such embodiments, the invention contemplates an immunogenic composition (e.g., a cancer vaccine) which can be used to induce an immune response against tumor antigens. A suitable composition would contain a rdCMVhet comprising a nucleic acid sequence encoding a tumor antigen and a pharmaceutically acceptable carrier. In a particular embodiment, the coding sequence element of the cassette may encode a single immunogen, such as an immunogenic peptide sequence derived from a self-antigen, such as a tumor-associated antigen. In some embodiments, the nucleic acid sequence encoding the immunogen (i.e., the transgene) may be codon optimized for expression in a particular mammalian species. In other embodiments, the coding sequence may encode more than one immunogen, such as one or more codon optimized tumor antigens. For example, a cancer vaccine utilizing the disclosed CMV vectors may encode a combination of self-antigens such as: PD-1 HER2/neu, CEA, Hepcam, PSA, PSMA, Telomerase, gp100, Melan-A/MART-1, Muc-1, NY-ESO-1, Survivin, Stromelysin 3, Tyrosinase, MAGE3, CML68, CML66, OY-TES-1, SSX-2, SART-1, SART-2, SART-3, NY-CO-58, NY-BR-62, hKLP2, VEGF.

In a specific embodiment, cervical cancer is a disorder prevented or treated using the methods of the invention. Human papillomavirus (HPV) antigens are used in such embodiments. Antigens that can be used in the methods of the invention to provide an immunologic response in a patient include, but are not limited to, E5, E6, E7, L1 and L2. In preferred embodiments, HPV antigens are derived from any HPV strain that is associated with a pathological condition, e.g. cervical cancer, or precancerous dysplastic lesions including cervical adenocarcinoma in situ, cervical intraepithelial neoplasia (CIN) grades 1, 2, and 3. In preferred embodiments, the HPV antigen is derived from an HPV selected from the group consisting of: HPV16, HPV18, HPV26, HPV31, HPV33, HPV35, HPV39, HPV45, HPV51, HPV52, HPV53, HPV55, HPV56, HPV58, HPV59, HPV66, HPV68, HPV73, and HPV82; if more preferred embodiments, the HPV is selected from HPV16, HPV18, HPV31, HPV33, HPV52, and HPV58.

In another specific embodiment, EBV associated cancer is a disorder prevented or treated using the methods of the invention. EBV-associated cancers include, but are not limited to, AIDS-related lymphomas, Burkitt's lymphoma, primary central nervous system lymphoma, polymorphic B-cell lymphoma, Hodgkin's lymphoma, diffuse large B-cell lymphoma, post-transplantation lymphoma, T-/NK-cell lymphoma, nasopharyngeal carcinomas and gastric carcinoma. Antigens that can be used in the methods of the invention to provide an immunologic response in a patient include, but are not limited to, EBV lytic cycle antigens (including BZLF1, BRLF1, BNLF2, VCA, BCRF1, gp350, gp110), gp42, BMRF-2, MVA-EL, gp220, LMP1 and LMP2.

In another embodiment, the rdCMVhet and methods of the invention can be used in methods of generating passive immunity to an antigen. The heterologous polypeptide expressed by the rdCMVhet is an antigen binding protein or antibody chain useful in the prevention and/or treatment of a disorder. In one embodiment, the disorder is one in which there is no effective vaccine but monoclonal antibodies have been identified that can neutralize either the pathogen or toxins produced by pathogens. For example, the antigen binding proteins expressed neutralizes HIV-1 such as 2F5 and 4E10 (Guenapa and Wyatt, PloS pathogen 2012 8(7): e1002806) and D5 (International Publication No. WO2005/118887), neutralizes influenza (Lingwood et al, Nature, 2012, 489:566-570), neutralizes anthrax toxin (Maynard et al, Nat Biotech, 2002, 20, 597-601, or neutralizes botulinum toxin (Nowakowski et al, Proc. Natl. Acad. Sci. USA 2002 99(17): 11346-11350). In another embodiment, the disorder is one which can benefit from modulation of the immune system. For example, the antibody or portion thereof expressed blocks PD-1 (Kline and Gajewski, 2010, Curr Opin Investig Drugs 11:1354-9; Topalian et al., 2012, New Engl. J. Med. 366:2443-54; Brahmer et al., 2012, New Engl. J. Med. 366:2455-65), blocks CTLA-4 (Agarwala et al., 2010, J. Immunother. 33:557-69), agonizes CD28 (International Publication WO 2010/007276; U.S. Pat. No. 8,168,759) or agonizes CD40 (US Patent Publication 2012/0251494; U.S. Pat. No. 7,927,596; U.S. Pat. No. 7,563,443).

In another embodiment, the rdCMVhet and methods of the invention can be used in gene therapy. In said embodiments, the heterologous polypeptide expressed by the rdCMVhet is a polypeptide or portion thereof that is absent, decreased, mutated and/or not functioning properly in a patient thereby causing a pathology/disorder in the patient. Administration of the rdCMVhet at least partially restores expression/function of the polypeptide that is absent, decreased, mutated and/or not functioning properly in the patient. In more specific embodiments, the disorder is cystic fibrosis with CFTR or a portion thereof as the heterologous polypeptide (Oakland et al., 2012, Mol. Ther. 20:1108-15; Griesenbach and Alton, 2012, Curr Pharm Des 18:642-62; Hull, 2012, JR Soc Med 105 (Suppl. 2):S2-8); hemophilia with factor VIII and/or factor IX or a portion thereof as the heterologous polypeptide (High, 2012, Blood 120:4482-7; Franchini and Mannucci, 2012, Orphanet J Rare Dis 7:24; Scott and Lozier, 2012, Br. J. Haematol 156:295-302); muscular dystrophy with dystrophin or a portion thereof as the heterologous polypeptide (Van Deutekom and van Ommen, 2003, Nature 4:774); type I diabetes with insulin or a portion thereof as the heterologous polypeptide (Sanlioglu et al., 2012, Expert Rev Mol Med 14:e18; Tuduri et al., 2012, J Diabetes 4:319-31); Leber's Congenital Amaurosis with RPE65 or a portion thereof as the heterologous polypeptide (Bainbridge et al., 2008, New Engl J Med 358:2231-9); beta-thalassemia with β-globin or a portion thereof as the heterologous polypeptide (May et al., 2002, Blood, 99:1902-8; Rund and Rachmilewitz, 2005, New Engl J Med 353:1135-46; Sadelain, 2006, Curr Opin Hematol 13:142-8); sickle cell anemia with hemoglobin or a portion thereof as the heterologous polypeptide (Sadelain, 2006, Curr Opin Hematol 13:142-8).

In another specific embodiment, the heterologous polypeptide expressed by the rdCMVhet is a polypeptide that can benefit a patient suffering from a pathology, e.g., by modulating the immune system. For example, cytokines or chemokines can be expressed including, but not limited to, IL-2, IL-7, IL-12, IL-15, IL-21 and IL-23.

d. Insertion of Nucleic Acids Encoding Heterologous Polypeptides

Gene cassettes comprising the nucleic acids encoding the heterologous polypeptides are inserted into the rdCMV genome in regions encoding non-essential genes. The cassettes can be inserted in the ORF of a non-essential gene, replace the ORF of a non-essential gene or be inserted between two ORFs encoding non-essential genes.

Non-essential genes in CMV are (according to Yu et al., 2003, Proc. Natl. Acad. Sci. USA 100:12396-12401):

US1
UL2
UL3
UL4
UL5
UL6
UL7
UL8
UL9
UL10
UL11
UL13
UL14
UL15
UL16
UL17
UL18
UL19
UL20
UL23
UL24
UL25
UL27
UL31
UL33
UL35
UL36
UL37.3
UL40
UL41
UL42
UL43
UL45
UL65
UL78
UL83
UL88
UL111a
UL116
UL118
UL119
UL120
UL121
UL124
UL128
UL130
UL132
UL146
UL147
US1
US2
US3
US5
US6
US7
US8
US9
US10
US11
US12
US13
US14
US15
US16
US17
US18
US19
US20
US21
US22
US24
US25
US27
US28
US29
US30
US31
US32
US33
US34
RL1
RL2
RL4
RL6
RL9
RL10
RL11
RL12
RL13

In some embodiments of the invention, the expression cassette is inserted between ORFs encoding non-essential genes. In a specific embodiment, the expression cassette is inserted in the UL1-UL20 region of the CMV genome or the US1-US10 region of the CMV genome.

In embodiments of the invention, the heterologous sequence is designed to replace native CMV sequence at a region of the CMV genome that is associated with producing high transcript levels. For example, the heterologous sequence/gene insert can be inserted into the UL21.5 open reading frame or the 5 kb transcript region (Chambers et al. J. Virol. 73(7): 5757-5766 (1999); Gatherer et al. Proc. Natl. Acad. Sci. USA 108(49): 19755-19760 (2011)).

The size of the genome of the rdCMVhet should not be altered by more than 5%-10% as compared to the size of the rdCMV genome. More preferably, the size of the genome of the rdCMVhet should not be altered by more than 2%-3% as compared to the size of the rdCMV genome. In embodiments using large or multiple expression cassettes inserted into the rdCMV, non-essential genes or regions containing non-essential genes should be deleted from the genome to allow for the overall size of the genome to be maintained within +/−5%-10%, or more preferably +/−2%-3%, as compared to the size of the rdCMV genome.

### Evaluation of Viral Replication

One skilled in the art can use viral replication assays to confirm the activity of a particular essential protein fused to FKBP or derivative thereof. Because gene expression/encoded product function should not be substantially affected by the attachment of the FKBP or derivative thereof to the essential protein in the presence of Shield-1, the rdCMVhet should replicate at a rate that is comparable to the parental CMV and/or rdCMV in the presence of Shield-1 (preferably at least 75%, 80%, 90%, 95%, 99% or 100% of the parental virus levels). Replication of the rdCMVhet is substantially altered from the parental CMV in the absence of Shield-1 (reduced by preferably greater than 50%, 75%, 90%. 95%, 99% or 100% as compared to a CMV that does not contain a destabilizing fusion protein).

In different embodiments, the rdCMVhet in the presence of at least 2 μM Shield-1 replicates preferably at least 90%, more preferably at least 95%, most preferably at least 99%, of the amount that a non-rdCMV replicates.

In one embodiment, a composition comprising the rdCMVhet of the invention has a viral titer of at least 10⁵ pfu/ml, more preferably at least 10⁷ pfu/ml, in the presence of at least 2 μM Shield-1.

Conversely, rdCMVhet should not replicate substantially in the absence of Shield-1. The quality of a replication defective mechanism is judged by how stringent the control is under the conditions not permissive for viral replication, i.e., the infectious titers of progeny virions under these conditions. The rdCMVhet of the present invention cannot replicate substantially (either in cell culture or within a patient) without Shield-1 present. Its replication in ARPE-19 cells and other types of human primary cells is conditional, and a molar concentration of Shield-1 greater than 0.1 μM, preferable at least 2 μM, in the culture medium is required to sustain viral replication.

In one embodiment, a composition comprising the rdCMVhet of the invention has a viral titer of less than 2 pfu/ml, more preferably less than 1 pfu/ml, in the absence of Shield-1.

Methods to assess CMV replication can be used to assess rdCMV replication either in the absence or presence of Shield-1. However, in preferred embodiments, a 50% Tissue Culture Infective Dose (TCID50) assay is used.

In another embodiment, rdCMVhet titers are determined by a 50% Tissue Culture Infective Dose (TCID50) assay. Briefly, this dilution assay quantifies the amount of virus required to kill 50% of infected hosts. Host cells (e.g., ARPE-19 cells) are plated and serial dilutions of the virus are added. After incubation, the percentage of cell death (i.e. infected cells) is observed and recorded for each virus dilution. Results are used to mathematically calculate the TCID50.

In another embodiment, the rdCMVhet titers are determined using a plaque assay. Viral plaque assays determine the number of plaque forming units (pfu) in a virus sample. Briefly, a confluent monolayer of host cells (e.g., ARPE-19 cells) is infected with the rdCMV at varying dilutions and covered with a semi-solid medium, such as agar or carboxymethyl cellulose, to prevent the virus infection from spreading indiscriminately. A viral plaque is formed when a virus infects a cell within the fixed cell monolayer. The virus infected cell will lyse and spread the infection to adjacent cells where the infection-to-lysis cycle is repeated. The infected cell area will create a plaque (an area of infection surrounded by uninfected cells) which can be seen visually or with an optical microscope. Plaques are counted and the results, in combination with the dilution factor used to prepare the plate, are used to calculate the number of plaque forming units per sample unit volume (pfu/mL). The pfu/mL result represents the number of infective particles within the sample and is based on the assumption that each plaque formed is representative of one infective virus particle.

In another embodiment, a hu-SCID mouse model is used to evaluate the ability of an rdCMV to replicate in vivo. Briefly, pieces of human fetal tissues (such as thymus and liver) are surgically implanted in kidney capsules of SCID mice. The rdCMV is inoculated 2-3 months later when the human tissues are vascularized. Viral titers are assessed 3-4 weeks after inoculation in plaque assays. The animal experiments can be performed in the absence or presence of Shield-1 by supplementing Shield-1 through daily intraperitoneal injections.

### Evaluation of Immune Response

Administration of rdCMVhet of the invention to a patient can be used to elicit an immune response to the heterologous polypeptide, preferably a protective immune response, that can treat and/or decrease the likelihood of an infection by the pathogen associated with the heterologous polypeptide (i.e., virus, bacteria, parasite) or pathology associated with such an infection in a patient or can induce an immune response against a self-antigen associated with a cancer (i.e. tumor-associated antigen).

The immune response elicited by the rdCMVhet can be assessed using methods known in the art.

Animal models known in the art can be used to assess the protective effect of administration of the rdCMVhet. In one embodiment, immune sera from individuals administered the rdCMVhet can be assayed for neutralizing capacity, including but not limited to, blockage of pathogen attachment or entry to a host cell. In other embodiments, T cells from individuals administered the rdCMVhet can be assayed for cytokine producing capacity including, but not limited to, interferon gamma, in the presence of an antigen of interest. Animal challenge models can also be used to determine an immunologically effective amount of immunogen.

Neutralization refers to pathogen specific antibodies capable of interrupting pathogen entry and/or replication in cultures. The common assay for measuring neutralizing activities for viruses is viral plaque reduction assay. Neutralizing activity for pathogens that do not enter cells can be assays by reduction in pathogen replication rates. NT50 titers are defined as reciprocal serum dilutions to block 50% of input pathogen in pathogen neutralization assays. NT50 titers are obtained from nonlinear logistic four-parameter curve fitting.

### Passive Immunity

Despite years of research and numerous technological and immunological advances, no effective vaccines are currently available for many important infectious diseases. However, for many diseases, monoclonal antibodies have been identified that can neutralize either the pathogen or toxins produced by pathogens and can provide protection from infectious challenge in animal models. Methods of the invention comprise the use of an rdCMVhet expressing one or more antigen binding protein to provide protective immunity against antigens such as pathogens or tumor-associated antigens for defined intervals, e.g. a portion of an antibody comprising a single chain of an antibody, a heavy chain variable region, a light chain variable region, an antigen binding region of an antibody. An antibody may also provide protective immunity by manipulating signaling pathways, agonizing or antagonizing signals, blocking ligand/receptor interactions, or stimulating the immune system through adjuvant-like properties when used in conjunction with vaccines.

The advantage of this system is that immunization would take a much shorter time as the expression of the heterologous polypeptide from the rdCMVhet would produce antibody in less than a week. Expression of the antibody could be increased by allowing the rdCMVhet to be replicated in vivo by administration of Shield-1 to the patient after administration of the rdCMVhet. A dimer version of Shld-1 has been used in a clinical study and shown well tolerated (Di Stasi et al., 2011, New Engl J Med 2011 365:1673-83). rdCMVhet replication would stop as soon as the patient is no longer administered Shield-1.

As used herein, the term “antigen binding protein” includes the portion of an antibody that binds to a target region or binding region of an antigen, single-chain Fvs (scFv) (including bi-specific scFvs), single chain antibodies, Fab fragments, F(ab′) fragments, disulfide-linked Fvs (sdFv), and epitope-binding fragments of any of the above, so long as they exhibit the desired biological activity. Antigen binding proteins can contain an antibody variable region providing for specific binding to an epitope. In preferred embodiments, antibodies of the invention used to provide passive immunity in a patient are humanized or human antigen binding proteins. The antibody variable region can be present in, for example, a complete antibody, an antibody fragment, and a recombinant derivative of an antibody or antibody fragment.

Different classes of antibodies have different structures. Different antibody regions can be illustrated by reference to IgG. An IgG molecule contains four amino acid chains: two longer length heavy chains and two shorter light chains. The heavy and light chains each contain a constant region and a variable region. Within the variable regions are three hypervariable regions responsible for antigen specificity. (See, for example, Breitling et al., Recombinant Antibodies, John Wiley & Sons, Inc. and Spektrum Akademischer Verlag, 1999; and Lewin, Genes IV, Oxford University Press and Cell Press, 1990.)

The hypervariable regions (also referred to as complementarity determining regions) are interposed between more conserved flanking regions (also referred to as framework regions) Amino acids associated with framework regions and complementarity determining regions (CDRs) can be numbered and aligned as described by Kabat et al., Sequences of Proteins of Immunological Interest, U.S. Department of Health and Human Services, 1991.

The two heavy chain carboxyl regions are constant regions joined by disulfide binding to produce an Fc region. The Fc region is important for providing effector functions. (Presta, Advanced Drug Delivery Reviews 58:640-656, 2006.) Each of the two heavy chains making up the Fc region extend into different Fab regions through a hinge region.

In higher vertebrates there are two classes of light chains and five classes of heavy chains. The light chains are either κ or λ. The heavy chains define the antibody class and are either α, δ, ε, γ, or μ. For example, IgG has a γ heavy chain. Subclasses also exist for different types of heavy chains such as human γ₁, γ₂, γ₃, and γ₄. Heavy chains impart a distinctive conformation to hinge and tail regions. (Lewin, Genes IV, Oxford University Press and Cell Press, 1990.)

Antibody fragments containing an antibody variable region include Fv, Fab and Fab₂ regions. Each Fab region contains a light chain made up of a variable region and a constant region, as well as a heavy chain region containing a variable region and a constant region. A light chain is joined to a heavy chain by disulfide bonding through constant regions. The light and heavy chain variable regions of a Fab region provide for an Fv region that participates in antigen binding.

The antibody variable region can be present in a recombinant derivative. Examples of recombinant derivatives include single-chain antibodies, diabody, triabody, tetrabody, and miniantibody. (Kipriyanov et al, Molecular Biotechnology 26:39-60, 2004.)

The antigen binding protein can contain one or more variable regions recognizing the same or different epitopes. (Kipriyanov et al., Molecular Biotechnology 26:39-60, 2004.)

“Humanized” forms of non-human (e.g., murine) antigen binding proteins are immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antigen binding proteins or antibodies are human immunoglobulins or human antibody fragments or chains (recipient antibody) in which residues from a complementarity-determining region (CDR) of the recipient are replaced by residues from a CDR of a non-human species (donor antibody) such as mouse, rat or rabbit having the desired specificity, affinity, and capacity. Thus, a humanized antibody may be a recombinant protein in which the CDRs from an antibody from one species; e.g., a rodent antibody, is transferred from the heavy and light variable chains of the rodent antibody into human heavy and light variable domains. The constant domains of the antibody molecule are derived from those of a human antibody. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin (see, e.g., Yamashita et al., 2007, Cytotech. 55:55; Kipriyanov and Le Gall, 2004, Mol. Biotechnol. 26:39 and Gonzales et al., 2005, Tumour Biol. 26:31).

Completely human antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893 and WO 98/16654, each of which is incorporated herein by reference in its entirety. Human antibodies can also be produced using transgenic mice which are incapable of expressing functional endogenous immunoglobulins, but which can express human immunoglobulin genes, see, e.g., PCT publications WO 98/24893; European Patent No. 0 598 877; U.S. Pat. Nos. 5,916,771; and 5,939,598, which are incorporated by reference herein in their entireties.

The CMVhet or rdCMVhet of the invention may be used to produce an antibody by inclusion of a one or more nucleotide sequences encoding one or more portions or chains of an antibody. Monoclonal antibodies can be produced, for example, from a recombinant cell containing one or more rdCMVhet comprising one or more recombinant genes encoding the antibody. The antibody may be encoded by more than one recombinant gene where, for example, one gene encodes the heavy chain and one gene encodes the light chain.

Another aspect of the present invention describes a CMVhet or rdCMVhet comprising a nucleic acid comprising one or more recombinant genes encoding either, or both of, an antigen binding protein Vₕ region or V₁ region, wherein the antigen binding protein binds to the target region of an antigen of interest. Multiple recombinant genes are useful, for example, where one gene encodes an antibody heavy chain or fragment thereof containing the Vₕ region and another gene encodes an antibody light chain or fragment thereof containing the V₁ region. Said multiple recombinant genes can be incorporated into a single CMVhet or rdCMVhet, for example, by using a promoter operatively linked to each nucleotide sequence encoding an antigen binding protein, or can be incorporated into more than one CMVhet or rdCMVhet.

In some embodiments, Fc engineered variants antibodies of the invention are also encompassed by the present invention. Such variants include antibodies or antigen binding proteins thereof which have been engineered so as to introduce mutations or substitutions in the Fc region of the antibody molecule so as to improve or modulate the effector functions of the underlying antibody molecule relative to the unmodified antibody. In general, improved effector functions refer to such activities as CDC, ADCC and antibody half life (see, e.g., U.S. Pat. Nos. 7,371,826; 7,217,797; 7,083,784; 7,317,091; and 5,624,821, each of which is incorporated herein in its entirety).

There are five classes of immunoglobulins: IgA, IgD, IgE, IgG, and IgM. The IgG and IgA classes are further divided into subclasses on the basis of relatively minor differences in the constant heavy region sequence and function, e.g., humans express the following subclasses: IgG1, IgG2, IgG3, IgG4, IgA1, and IgA2. In some embodiments, the antibodies of the invention are IgG1.

In some embodiments of the invention, the rdCMVhet comprises a nucleotide sequences that encodes an antigen binding protein that blocks the function of PD-1. Programmed Cell Death 1 (PD-I) is a 50-55 kDa type I transmembrane receptor originally identified by subtractive hybridization of a mouse T cell line undergoing apoptosis (Ishida et al., 1992, Embo J. 11:3887-95). A member of the CD28 gene family, PD-1 is expressed on activated T, B, and myeloid lineage cells (Greenwald et al., 2005, Annu. Rev. Immunol. 23:515-48; Sharpe et al., 2007, Nat. Immunol. 8:239-45). Two ligands for PD-1 have been identified, PD ligand 1 (PD-L1) and ligand 2 (PD-L2). Both belong to the B7 superfamily (Greenwald et al., 2005, supra). PD-L1 is expressed on many cell types, including T, B, endothelial and epithelial cells. In contrast, PD-L2 is narrowly expressed on professional antigen presenting cells, such as dendritic cells and macrophages.

PD-1 negatively modulates T cell activation, and this inhibitory function is linked to an immunoreceptor tyrosine-based inhibitory motif (ITIM) of its cytoplasmic domain (Greenwald et al., supra; Parry et al., 2005, Mol. Cell Biol 25:9543-53). Disruption of this inhibitory function of PD-I can lead to autoimmunity. For example, PD-1 knockout in C57BL/6 mice leads to a lupus-like syndrome, whereas in BALB/c mice it leads to development of dilated cardiomyopathy (Nishimura et al., 1999, Immunity 11:141-51; Okazaki et al., 2003, Nat. Med. 9:1477-83). In humans, a single nucleotide polymorphism in PD-1 gene locus is associated with higher incidences of systemic lupus erythematosus, type 1 diabetes, rheumatoid arthritis, and progression of multiple sclerosis. The reverse scenario can also be deleterious. Sustained negative signals by PD-1 have been implicated in T cell dysfunctions in many pathologic situations, such as tumor immune evasion and chronic viral infections.

Host anti-tumor immunity is mainly affected by tumor-infiltrating lymphocytes (TILs) (Galon et al., 2006, Science 313:1960-4). Multiple lines of evidence have indicated that TILs are subject to PD-I inhibitory regulation. First, PD-L1 expression is confirmed in many human and mouse tumor lines and the expression can be further upregulated by IFN-γ in vitro (Dong et al., 2002, Nat. Med. 8:793-800). Second, expression of PD-L1 by tumor cells has been directly associated with their resistance to lysis by anti-rumor T cells in vitro (Dong et al., supra; Blank et al., 2004, Cancer Res. 64:1 140-5). Third, PD-I knockout mice are resistant to tumor challenge (Iwai et al., 2005, Int. Immunol. 17:133-44) and T cells from PD-1 knockout mice are highly effective in tumor rejection when adoptively transferred to tumor-bearing mice (Blank et al., supra). Fourth, blocking PD-I inhibitory signals by a monoclonal antibody can potentiate host anti-tumor immunity in mice (Iwai et al., supra; Hirano et al., 2005, Cancer Res. 65:1089-96). Fifth, high degrees of PD-L1 expression in tumors (detected by immunohistochemical staining) are associated with poor prognosis for many human cancer types (Hamanishi et al., 2007, Proc. Natl. Acad Sci USA 104:3360-5).

Thus, one aspect of the invention is an rdCMVhet that comprises a nucleotide sequence that encodes an antigen binding protein that blocks PD-1 signaling, e.g, by binding PD-1, or a PD-1 ligand. In some embodiments of the invention, the rdCMVhet encodes an antigen binding protein that blocks binding of the PD-L1 ligand to the PD-1 receptor. Exemplary nucleotide sequences encoding antigen binding proteins that are useful in such embodiments are disclosed in WO 2009/114335.

### Manufacture of Replication Defective CMV

The present invention encompasses methods of making the rdCMVhet. In some embodiments of the invention, the rdCMVhet are propagated in the presence of a stabilizing agent such as Shield-1 on epithelial cells, preferably human epithelial cells, and more preferably human retinal pigmented epithelial cells or fibroblasts, more preferable human fibroblasts. In specific embodiments, the human retinal pigmented epithelial cells are ARPE-19 cells deposited with the American Type Culture Collection (ATCC) as Accession No. CRL-2302. In other specific embodiments, the human fibroblasts are MRC-5 cells deposited with the ATCC as Accession No. CCL-171.

In some embodiments, Shield-1 is present at a concentration of at least 0.5 μM in the tissue culture media. In preferred embodiments, Shield-1 is present at a concentration of at least 2.0 μM in the tissue culture media.

In some embodiments, the cells used to propagate the rdCMVhet are grown on microcarriers. A microcarrier is a support matrix allowing for the growth of adherent cells in spinner flasks or bioreactors (such as rotating wall microgravity bioreactors and fluidized bed bioreactors). Microcarriers are typically 125-250 μM spheres with a density that allows them to be maintained in suspension with gentle stirring. Microcarriers can be made from a number of different materials including, but not limited to, DEAE-dextran, glass, polystyrene plastic, acrylamide, and collagen. The microcarriers can have different surface chemistries including, but not limited to, extracellular matrix proteins, recombinant proteins, peptides and charged molecules. Other high density cell culture systems, such as Corning HyperFlask® and HyperStack® systems can also be used.

The cell-free tissue culture media can be collected and rdCMVhet can be purified from it. CMV viral particles are about 200 nm in diameter and can be separated from other proteins present in the harvested media using techniques known in the art including, but not limited to ultracentrifugation through a density gradient or a 20% Sorbitol cushion. The protein mass of the vaccines can be determined by Bradford assay.

Shield-1 can be used to control replication of the rdCMVhet in conjunction with FKBP. After the desired amount of viral propagation in tissue culture cells is completed, the ability to replicate is no longer desirable. Shield-1 is withdrawn from the rdCMVhet to make the virus replication deficient (e.g., in order to be administered to a patient). In one embodiment, the rdCMVhet is purified from Shield-1 by washing one or more times. In another embodiment, the rdCMVhet is purified from Shield-1 through ultracentrifugation. In another embodiment, the rdCMVhet is purified from Shield-1 through diafiltrations. Diafiltrations is commonly used to purify viral particles. In one embodiment, filters are used with pore size of approximately 750 kilodalton, which would only allow Shield-1 to pass through the pores.

After purification of rdCMVhet from Shield-1, there may a small amount be of residual Shield-1 remaining in the rdCMVhet composition. In one embodiment, the level of Shld-1 in the rdCMVhet composition after purification is at least 100-fold below the level needed to sustain replication in tissue culture. In another embodiment, the level of Shield-1 in the rdCMVhet composition after purification is 0.1 μM or less. In another embodiment, the level of Shield-1 in the rdCMVhet composition after purification is undetectable.

Determination of Shield-1 levels in a composition can be detected using a LC/MS (liquid chromatography-mass spectroscopy) or HPLC/MS (high performance liquid chromatography-mass spectroscopy) assays. These techniques combine the physical separation capabilities of LC or HPLC with the mass analysis capabilities of and can detect chemicals of interest in complex mixtures.

### Pharmaceutical Compositions

A further feature of the invention is the use of a CMVhet described herein in a composition, preferably an immunogenic composition or vaccine, for treating patients with an infection and/or reducing the likelihood of an infection. Suitably, the composition comprises a pharmaceutically acceptable carrier. Thus, the invention provides a composition comprising an effective amount of a CMVhet of the invention and a pharmaceutically acceptable carrier.

A “pharmaceutically-acceptable carrier” is a substance that facilitates administration of the composition, including, but not limited to: a liquid filler, diluent or encapsulating substance that may be safely used in systemic administration. Depending upon the particular route of administration, a variety of pharmaceutically acceptable carriers, well known in the art may be used. These carriers may be selected from a group including sugars, starches, cellulose and its derivatives, malt, gelatin, talc, calcium sulfate, vegetable oils, synthetic oils, polyols, alginic acid, phosphate buffered solutions including phosphate buffered saline, emulsifiers, isotonic saline, and pyrogen-free water. In particular, pharmaceutically acceptable carriers may contain different components such as a buffer, sterile water for injection, normal saline or phosphate-buffered saline, sucrose, histidine, salts and polysorbate. Terms such as “physiologically acceptable”, “diluent” or “excipient” can be used interchangeably.

Procedures for vaccine formulations are disclosed, for example, in New Generation Vaccines (1997, Levine et al., Marcel Dekker, Inc. New York, Basel, Hong Kong), which is incorporated herein by reference.

Compositions of the invention may include additional components such as one or more adjuvants, as described, infra, and/or one or more additional active ingredients.

### Adjuvants

Adjuvants are substances that can assist an immunogen in producing an immune response. In addition to increasing the immune response against the antigen of interest, some adjuvants may be used to decrease the amount of antigen necessary to provoke the desired immune response or decrease the number of injections needed in a clinical regimen to induce a durable immune response and provide protection from disease. Adjuvants can function by different mechanisms such as one or more of the following: increasing the antigen biologic or immunologic half-life; improving antigen delivery to antigen-presenting cells; improving antigen processing and presentation by antigen-presenting cells; achieving dose-sparing, and, inducing production of immunomodulatory cytokines (Vogel, 2000, Clin Infect Dis 30:S266).

Accordingly, the invention includes compositions that comprise a CMVhet, such as a human CMVhet or an rdCMVhet, and an adjuvant. A variety of types of adjuvants can be employed to assist in the production of an immune response. In some embodiments of the invention, the particular adjuvant includes an aluminum salt such as aluminum hydroxide; aluminum phosphate, aluminum hydroxyphosphate, amorphous aluminum hydroxyphosphate sulfate adjuvant (AAHS). Aluminum-based compounds were determined to possess adjuvant activity over 60 years ago (for review, see Lindblad, E. B. Immunol. and Cell Biol. 82: 497-505 (2004); Baylor et al. Vaccine 20: S18-S23 (2002)). Aluminum adjuvants are generally regarded as safe when used at appropriate dosages. Many have been approved for administration into humans by regulatory agencies worldwide. In some embodiments of the invention, the aluminum adjuvant is AAHS. In alternative embodiments, the aluminum adjuvant is an aluminum phosphate adjuvant, such as Merck Aluminum Phosphate Adjuvant (MAPA). In other embodiments, the adjuvant is aluminum hydroxide.

One of skill in the art will be able to determine an optimal dosage of aluminum adjuvant that is both safe and effective at increasing the immune response to the targeted dengue viruses. For a discussion of the safety profile of aluminum, as well as amounts of aluminum included in FDA-licensed vaccines, see Baylor et al., Vaccine 20: S18-S23 (2002). Generally, an effective and safe dose of aluminum adjuvant varies from 50 μg to 1.25 mg elemental aluminum per dose (100 μg/mL to 2.5 mg/mL concentration).

Other adjuvants that may be used in conjunction with the CMVhet compositions of the invention, include, but are not limited to, adjuvants containing CpG oligonucleotides, or other molecules acting on toll-like receptors such as TLR 4 and TLR9 (for reviews, see, Daubenberger, C. A., Curr. Opin. Mol. Ther. 9(1):45-52 (2007); Duthie et al., Immunological Reviews 239(1): 178-196 (2011); Hedayat et al., Medicinal Research Reviews 32(2): 294-325 (2012)), including lipopolysaccharide, monophosphoryl lipid A, and aminoalkyl glucosaminide 4-phosphates. Additional adjuvants useful in the compositions of the invention include immunostimulatory oligonucleotides (IMO's; see, e.g. U.S. Pat. No. 7,713,535 and U.S. Pat. No. 7,470,674); T-helper epitopes, lipid-A and derivatives or variants thereof, liposomes, calcium phosphate, cytokines, (e.g. granulocyte macrophage-colony stimulating factor (GM-CSF) IL-2, IFN-α, Flt-3L), CD40, CD28, CD70, IL-12, heat-shock protein (HSP) 90, CD134 (OX40), CD137, nonionic block copolymers, incomplete Freund's adjuvant, chemokines, cholera toxin; E. coli heat-labile enterotoxin; pertussis toxin; muramyl dipeptide, muramyl peptide analogues, Freund's incomplete adjuvant; MF59, SAF, immunostimulatory complexes, biodegradable microspheres, saponins; nonionic block copolymers; muramyl peptide analogues; polyphosphazene; synthetic polynucleotides; IFN-γ; IL-2; IL-12; and ISCOMS. (Vogel, 2000, Clin Infect Dis 30:S266; Klein et al., 2000, J Pharm Sci 89:311; Rimmelzwaan et al., 2001, Vaccine 19:1180; Kersten, 2003, Vaccine 21:915; O'Hagen, 2001, Curr. Drug Target Infect. Disord. 1:273.)

In some embodiments of the invention, the compositions include a CMVhet and an adjuvant, wherein the adjuvant is not an oil-based adjuvant including, but not limited to, incomplete Freund's adjuvant and MF59.

In other embodiment of the invention, the composisitons include a saponin-based adjuvant such as ISCOMATRIX® adjuvant (CSL Ltd., Parkville, Australia) or other saponin-based adjuvant that comprises a saponin, either alone, or together with cholesterol and a phospholipid. In further embodiments, the compositions include a saponin-based adjuvant and an aluminum salt adjuvant such as aluminum phosphate, aluminum hydroxide, or AAHSA.

### Formulations

In some embodiments, the rdCMVhet of the invention is administered to a patient to elicit an immune response. It is desirable to minimize or avoid the loss of the rdCMVhet composition potency during storage of the immunogenic composition. The conditions to support such an aim include but not limited to (1) sustained stability in storage, (2) resistant to stressed freezing-thawing cycles, (3) stable at ambient temperatures for up to a week, (4) maintenance of immunogenicity, (5) compatible with adjuvanting strategy. Conditions that affect rdCMVhet stability include, but are not limited to, buffer pH, buffer ionic strength, presence/absence of particular excipients and temperature. The compositions comprise buffers to increase the stability of purified rdCMVhet viral particles suitable as vaccine composition.

The preservation of the integrity of viral particles can be assessed by immunogenicity assays in mice and/or viral entry assays. Viral entry events dependent on the integrity and functions of viral glycoproteins, including the pentameric gH complex.

In some embodiments, the rdCMVhet is stored in buffer comprising 15-35 mM Histidine and 100-200 mM NaCl at a pH of between 5 and 7. In a more specific embodiment, the buffer comprises 25 mM Histidine and 150 mM NaCl at pH6.

In other embodiments, sugars can be added to provide further stability, such as polyols (including, but not limited to, mannitol and sorbitol); monosaccharides (including, but not limited to, glucose, mannose, galactose and fructose); disaccharides (including, but not limited to, lactose, maltose, maltose, sucrose, lactulose and trehalose) and trisaccharides (including, but not limited to, raffinose and melezitose). In a more specific embodiment, the sugar is sucrose. In an even more specific embodiment, the sucrose is between 5-15%.

In preferred embodiments, the rdCMV is stored in buffer comprising 25 mM Histidine, 150 mM NaCl, 9% Sucrose at pH 6.

### Administration

A rdCMVhet described herein can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Vaccines can be administered by different routes such as subcutaneous, intramuscular, intravenous, mucosal, parenteral, transdermal or intradermal. Subcutaneous and intramuscular administration can be performed using, for example, needles or jet-injectors. In an embodiment, the vaccine of the invention is administered intramuscularly. Transdermal or intradermal delivery can be accomplished through intradermal syringe needle injection, or enabling devices such as micron-needles or micron array patches.

The compositions described herein may be administered in a manner compatible with the dosage formulation, and in such amount as is immunogenically-effective to treat and/or reduce the likelihood of an infection (including primary, recurrent and/or super). The dose administered to a patient, in the context of the present invention, should be sufficient to effect a beneficial response in a patient over time such as a reduction in the level of infection, ameliorating the symptoms of disease associated with an infection and/or shortening the length and/or severity of an infection, or to reduce the likelihood of infection (including primary, recurrent and/or super).

Suitable dosing regimens may be readily determined by those of skill in the art and are preferably determined taking into account factors well known in the art including age, weight, sex and medical condition of the patient; the route of administration; the desired effect; and the particular composition employed. In determining the effective amount of the rdCMVhet to be administered in the treatment or prophylaxis against a pathogen, the physician may evaluate circulating plasma levels of virus, progression of disease, and/or the production of anti-pathogen antibodies. The dose for a vaccine composition consists of the range of 10³ to 10¹² plaque forming units (pfu). In different embodiments, the dosage range is from 10⁴ to 10¹⁰ pfu, 10⁵ to 10⁹ pfu, 10⁶ to 10⁸ pfu, or any dose within these stated ranges. When more than one vaccine is to be administered (i.e., in combination vaccines), the amount of each vaccine agent is within their described ranges.

The vaccine composition can be administered in a single dose or a multi-dose format. Vaccines can be prepared with adjuvant hours or days prior to administrations, subject to identification of stabilizing buffer(s) and suitable adjuvant composition. Vaccines can be administrated in volumes commonly practiced, ranging from 0.1 mL to 0.5 mL.

The timing of doses depends upon factors well known in the art. After the initial administration one or more additional doses may be administered to maintain and/or boost antibody titers and T cell immunity. Additional boosts may be required to sustain the protective levels of immune responses, reflected in antibody titers and T cell immunity such as ELISPOT. The levels of such immune responses are subject of clinical investigations.

For combination vaccinations, each of the immunogens can be administered together in one composition or separately in different compositions. A rdCMVhet described herein is administered concurrently with one or more desired immunogens. The term “concurrently” is not limited to the administration of the therapeutic agents at exactly the same time, but rather it is meant that the rdCMVhet described herein and the other desired immunogen(s) are administered to a subject in a sequence and within a time interval such that the they can act together to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route.

### Patient Population

The invention herein includes methods for the treatment of a patient by administering a rdCMVhet of the invention, wherein the patient would benefit from the protein encoded by the heterologous nucleotide sequence within the rdCMVhet. The methods include generation of an immune response, generation of a protective immune response, vaccining against a pathogen, gene therapy, and generation of passive immunity. In some embodiments, the patient is a human. In additional embodiments, the patient is a non-human mammal, such as a companion animal (e.g. dog or cat) or livestock (e.g. horses, cattle, poultry). A patient can be treated prophylactically or therapeutically. Prophylactic treatment provides sufficient response to reduce the likelihood or severity of pathology (e.g., infection including primary infections, recurrent and super-infections). Therapeutic treatment can be performed to reduce the severity of a pathology (e.g., reduce or lesson the clinical symptoms associated with the pathology), or to delay the onset or progression of the pathology, or to lessen, ameliorate or eliminate an infection.

Treatment can be performed using a pharmaceutical composition comprising a rdCMVhet expressing a heterologous polypeptide as described herein. Pharmaceutical compositions can be administered to the general population, especially to those persons at an increased risk of infection (either primary, recurrent or super) or to specific patient populations in which an infection would be particularly problematic (such as immunocompromised individuals, transplant patients or pregnant women).

Those in need of treatment include those already with a pathology, as well as those prone to have a pathology or in which a reduction in the likelihood of pathology is desired. Treatment can ameliorate the symptoms of pathology and/or shorten the length and/or severity of the pathology.

Persons with an increased risk of a pathology include patients with a weakened immunity or patients facing therapy leading to a weakened immunity (e.g., undergoing chemotherapy or radiation therapy for cancer or taking immunosuppressive drugs). As used herein, “weakened immunity” refers to an immune system that is less capable of battling infections because of an immune response that is not properly functioning or is not functioning at the level of a normal healthy adult. Examples of patients with weakened immunity are patients that are infants, young children, elderly, pregnant or a patient with a disease that affects the function of the immune system including patients suffering from hematologic malignancy, patients undergoing immunosuppressive therapies, patients who have received a hematopoietic stem cell transplant or solid organ transplant, HIV-infected patients, and patients with autoimmune diseases.

For rdCMVhet expressing a monoclonal antibody or portion thereof, the patient population is those individuals where expression of the monoclonal antibody is useful for a limited time immunization in unique situations. In some embodiments, the patient population includes travelers who require short-term immunizations for trips or active-duty service members who are being mobilized in theater. In other embodiments, the patient population is broader, such as during pandemics of infectious agents where rapid immunization of large numbers of individuals is required.

### EXAMPLES

Examples are provided below to further illustrate different features of the present invention. The examples also illustrate useful methodology for practicing the invention. These examples do not limit the claimed invention.

### Example 1

### Restoration of the Pentameric gH Complex

An infectious CMV bacterial artificial chromosome clone was constructed so that the encoded virion that expressed the pentameric gH complex consisting of UL128, UL130 and UL131 assembled onto a gH/gL scaffold.

CMV strain AD169 strain was originally isolated from the adenoids of a 7-year-old girl (Elek and Stern, 1974, Lancet, 1:1). The virus was passed 58 times in several types of human fibroblasts to attenuate the virus (Neff et al, 1979, Proc Soc Exp Biol Med, 160:32, with the last 5 passages in WI-38 human fibroblasts. This passaged variant of AD169 virus, referred in this study as Merck AD169 (MAD169), was used as the parental virus to construct the infectious BAC clone. Neither the parental virus AD169 nor the passaged variant virus MAD169 expressed UL131 or the pentameric gH complex.

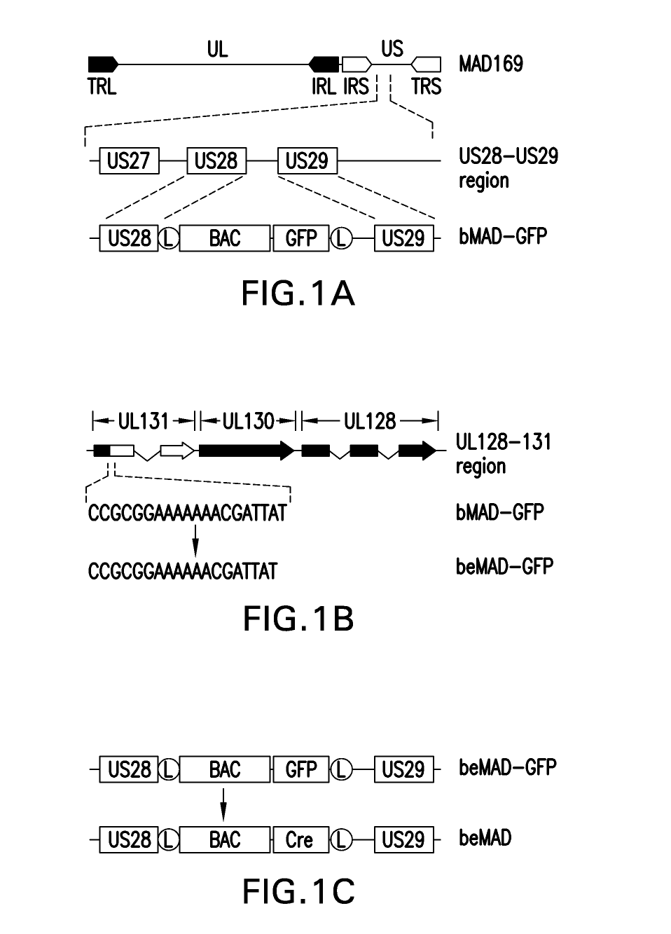

The MAD169 was used as the parental virus to construct an infectious bacterial artificial chromosome (BAC) clone. A BAC vector is a molecular tool that allows the genetic manipulation of a large size DNA fragment, such as the CMV genome (˜230 Kb), in E. coli. A BAC element along with a GFP marker gene was inserted immediately after the stop codon of US28 open reading frame (between US28 and US29 ORFs in the viral genome) with a LoxP site created at the both ends of the fragment (FIG. 1A). Briefly, a DNA fragment containing a GFP expression cassette flanked by two loxP sites and CMV US28-US29 sequences were synthesized and cloned into pBeloBAC11 vector. The BAC vector was linearized with restriction enzyme PmeI, and cotransfected into MRC-5 cells with MAD169 DNA extracted from purified virions. The recombinant variants, identified by green fluorescence expression, were plaque purified. After one round of amplification, the circular form of viral genome was extracted from the infected cells, and electroporated into E. coli DH10 cells. The bacterial colonies were screened by PCR for the presence of US28 and US29 regions. Candidate clones were further examined by EcoRI, EcoRV, HindIII, SpeI and BamHI restriction analyses. After screening, one clone, bMAD-GFP, showed identical restriction pattern with the parental MAD169 virus.

The frame-shift mutation in the first exon of UL131 underlying the epithelial tropism deficiency in MAD169 was repaired genetically in E. coli (FIG. 1B). Specifically, one adenine nucleotide (nt) from the 7 nt A-stretch in the UL131 gene was deleted (FIG. 1B). Deletion of the 1-nt frame-shift mutation was sufficient to rescue the epithelial and endothelial cell tropism, and expression of the pentameric gH complex as a result. Expression was confirmed by ELISA and Western blot (data not shown). This clone was further modified by removing the BAC segment by LoxP/Cre recombination. The BAC DNA was transfected in ARPE-19 cells, human retinal pigmented epithelial cells (ATCC Accession No. CRL-2302), to recover the infectious virus (FIG. 1C). The resultant infectious virus, termed BAC-derived epithelial-tropic MAD169 virus (beMAD), differs from MAD169 only in two loci, (1) UL131 ORF where a single adenine nucleotide was deleted and (2) a 34 bp LoxP site inserted between US28 and US29 ORFs (see Table 2).

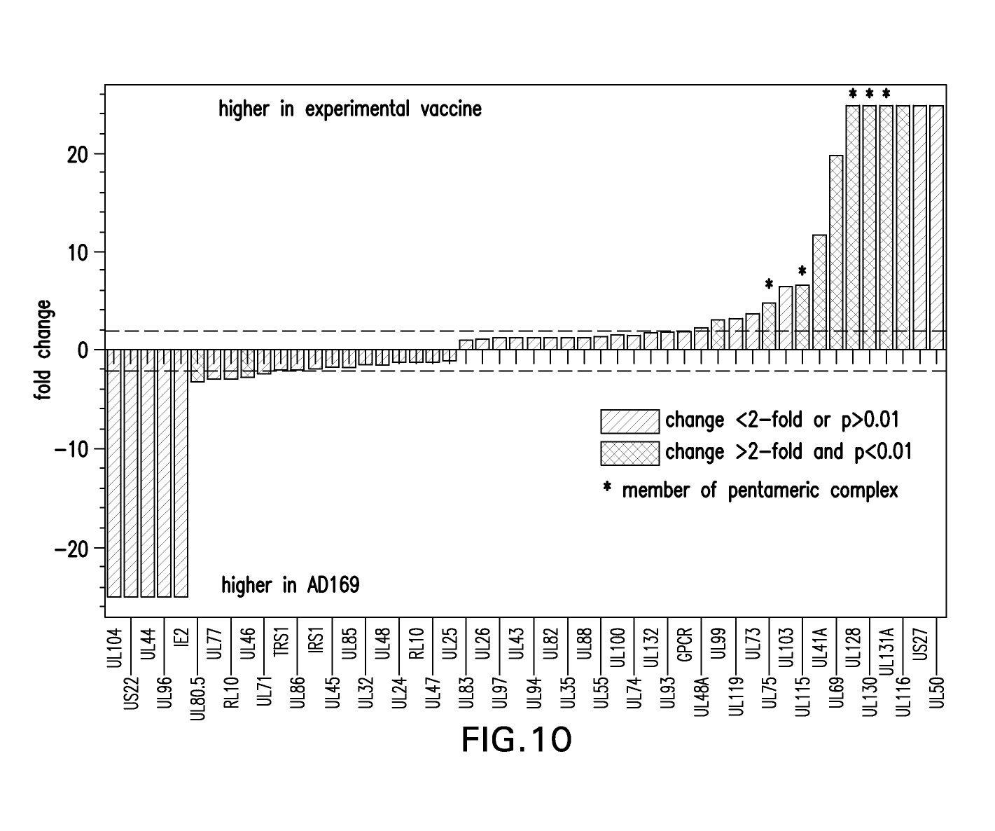

The genomic and proteomic compositions of the MAD169 and beMAD were comparable. To determine the proteomic composition, MAD169 and beMAD virions were purified by two rounds of ultracentrifugation through a Sorbital cushion. The protein content was determined by semi-quantitative, label-free shotgun proteomics, following tryptic digestion (FIG. 10). Each strain sample was analyzed in triplicate by nano LC-MS/MS. The analysis identified 50 viral proteins. Label-free quantification was performed based on the peak height of identified MS signals and fold change values were calculated to differentiate MAD169 and beMAD. An analysis of variance (ANOVA) was performed to identify statistically significant changes (p-value <0.01). The results confirmed that the pentameric gH complex [gH (UL75), gL (UL115), UL128, UL130, UL131] were present in the beMAD virions. Three other viral proteins of significant increase in beMAD were UL41A, UL69 and UL116. The significance of these proteins was not known.

TABLE 2
Molecular difference and tropism of CMV viruses

| Virus ID | Genetic composition | Proteins in virions |
|---|---|---|
| ATCC | ATCC laboratory strain | |
| AD169 | containing frame-shift mutation | |
| | in UL131 causing deficiency | |
| | in epithelial tropism | |
| MAD169 | Contains frame-shift mutation | Projected to be identical |
| | in UL131 identical to ATCC | to ATCC AD169. |
| | AD169 | |
| beMAD | Repaired frame-shift mutation | Mostly identical to |
| | in UL131; LoxP sequence (34 | MAD169, with addition of |
| | bp) between US28 and US29 | the pentameric gH complex |
| | ORFs | |

The pentameric gH containing CMV virus could be desirable for improved antigen processing and presentation, scine the pentameric gH complex is required for infection of immature Dendritic cells (DCs) (Gema et al., 2005, J. Gen. Virol. 86:275-84). However, a recent study showed the broad T-cell responses can be elicited with a fibroblast-cultured, replication competent rhesus CMV vector in rhesus macaques. The results further suggested that the breadth of such MHC class II-restricted CD8 T-cell responses was associated with missing the pentameric gH complex in the studied rhesus CMV vector (Hansen et al., 2013, Science, 340:940). Thus, an ideal vaccine vector based on human CMV virus could be experimentally selected pertinent to these parameters relevant to viral epithelial tropisim, determined by the expression of the pentameric gH complex. The vectors could be rationally designed and rescued in either ARPE-19 cells or MRC-5 cells based on the desired phenotype of the viral tropisim.

### Example 2

### Construction and Screening of FKBP-Essential Protein Fusions

A conditionally replicative defective CMV was constructed using the attenuated AD169 strain as backbone (MAD169).

Viral proteins to be fused to the FKBP derivative were selected based on two criteria. First, the proteins of interest were not detected in CMV virions by proteomics analysis (Varnum et al., 2004, J. Virol. 78:10960), thus decreasing the likelihood that the FKBP fusion protein will be incorporated into virus. Second, the proteins of interest are essential for viral replication in tissue culture.

Examples are provided using beMAD as the parental virus. The FKBP derivative (SEQ ID NO:26) was fused to 12 essential viral proteins individually, yielding the fusion proteins FKBP-IE1/2 (SEQ ID NO:1), FKBP-UL37x1 (SEQ ID NO:3), FKBP-UL44 (SEQ ID NO:5), FKBP UL51 (SEQ ID NO:7), FKBP-UL52 (SEQ ID NO:9), FKBP-UL53 (SEQ ID NO:11), FKBP-UL56 (SEQ ID NO:13), FKBP-UL77 (SEQ ID NO:15), FKBP-UL79 (SEQ ID NO:17), FKBP-UL84 (SEQ ID NO:19), FKBP L87 (SEQ ID NO:21) and FKBP-UL105 (SEQ ID NO:23). A virus with two different essential proteins fused to FKBP was also constructed that fused each of IE1/2 and UL51 the FKBP derivative (the genome of the rdCMV with the destabilized IE1/2 and UL51 is shown as SEQ ID NO:14 in International Application No. PCT/US12/053599 filed Sep. 4, 2012, published as WO 2013/036465). After construction, all recombinant BAC DNAs were transfected into ARPE-19 cells, and cultured in the medium containing Shld-1.

The dependence of viral growth on Shld-1 was examined. The IE1/2, UL51, UL52, UL84, UL79 and UL87 fusion viruses were readily rescued in 2 μM Shld-1 in plaque assays (data not shown). The UL37x 1, UL77 and UL53 fusion viruses also produced plaques, but the plaques were small, and they grew significantly slower, comparing to the parental beMAD. Increasing the Shld-1 concentration to 10 μM did not significantly expedite the viral growth (data not shown). The UL56 and UL105 fusions were not recovered, suggesting that tagging of these proteins disrupts the function of these proteins, or expression of neighboring genes.

Varying concentrations of Shld-1 were used in additional experiments to further assess viral replication in the presence or absence of Shld-1. ARPE-19 cells were infected by the gH expressing CMV that also contained a FKBP derivative fused to an essential protein at MOI of 0.01 pfu/ml. After infection for 1 hour, the cells were washed twice with fresh medium to remove Shld-1. The infected ARPE-19 cells were then cultured in medium containing 0.05, 0.1, 0.5 or 2 μM of Shield-1. Seven days post infection, the cell-free progeny virus was collected and titrated on ARPE-19 cells supplemented with 2 mM of Shield-1. Virus titers were determined by a 50% Tissue Culture Infective Dose (TCID50) assay. Briefly, this dilution assay quantifies the amount of virus required to kill 50% of infected hosts. ARPE-19 cells were plated and serial dilutions of the virus were added. After incubation, the percentage of cell death (i.e. infected cells) was manually observed and recorded for each virus dilution. Results were used to mathematically calculate the TCID50.

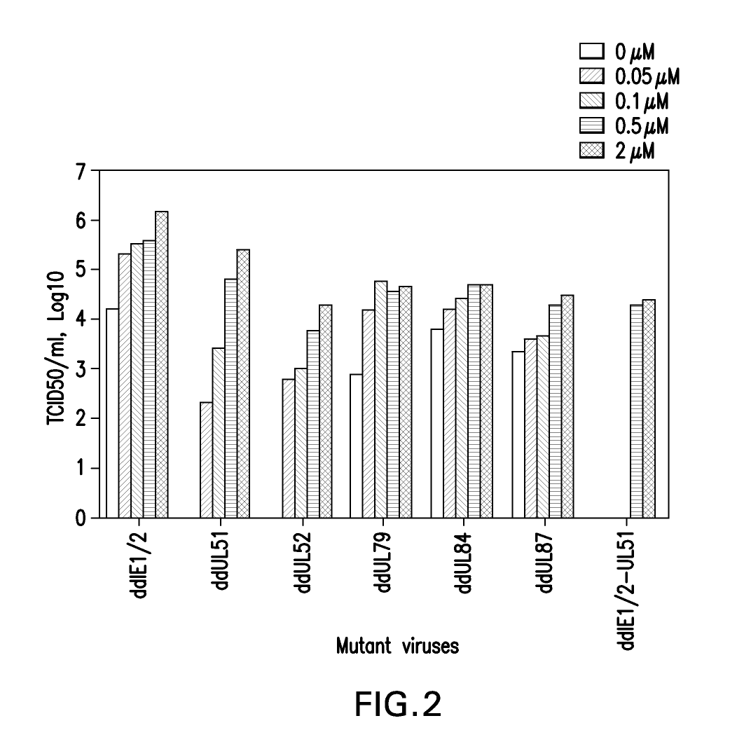

As shown in FIG. 2, efficient replication of all FKBP fusion containing CMV depended on Shield-1 concentration, albeit to varying degrees. Lower concentration of Shield-1 in general reduced the titer of progeny virus production. Among the viruses, only ddUL51 and ddUL52 absolutely required Shield-1 for replication. Other viruses with a single fusion, ddIE1/2, ddUL84, ddUL79, and ddUL87, could produce detectable progeny virus in the absence of Shield-1. The regulation was tightest when the FKBP derivative was fused to UL51 or UL52. Thus, results show that low concentration of Shield-1, as low as 50 nM, can effectively turn on or off viral replication when the FKBP derivative was fused to UL51 or UL52. This suggested a CMV vector, with the FKBP derivative fused to either UL51 or UL52, can be potentially regulated with Shld-1 in vivo.

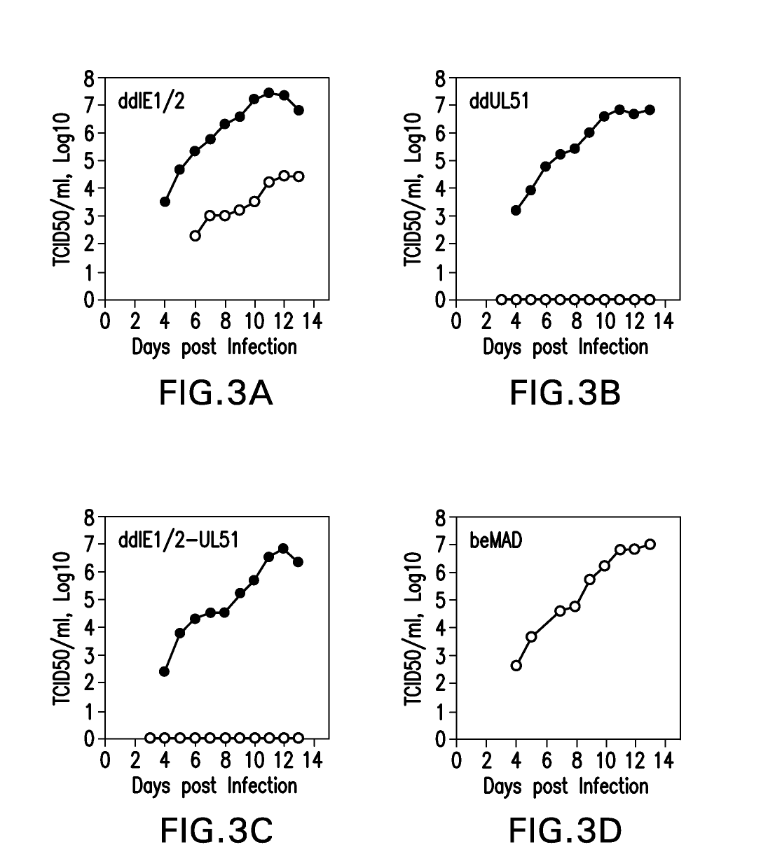

The growth kinetics of viruses with IE1/2, UL51, IE1/2-UL51 fusions were compared to the parental beMAD virus in the presence or absence of 2 μM of Shld-1. As shown in FIG. 3, in the presence of Shld-1, the single or double fusions had growth kinetics comparable to the parental beMAD. However, in the absence of Shld-1, only the ddIE1/2 virus could replicate, albeit at a lower and slower rate than the parental beMAD.

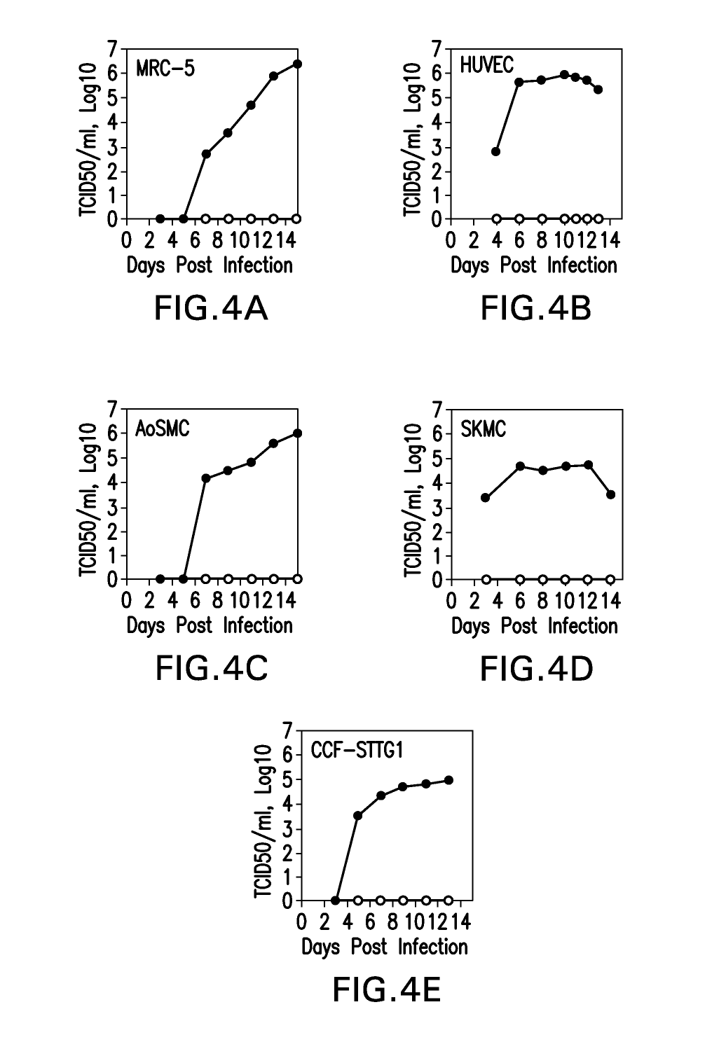

The tightness of the control of virus replication in the double fusion virus was also tested in different cell types (FIG. 4). These cells included human umbilical vein cells (HUVECs), MRC-5 fibroblasts, aortic smooth muscle cells (AoMCs), skeletal muscle cells (SKMCs) and CCF-STTG1 astrocytoma cells. The cells were infected by the IE1/2-UL51 fusion virus at MOI of 0.01 pfu/cell (except for CCF-STTG1 which was infected with a MOI of 5 pfu/cell), and then incubated in the medium in the presence or absence of Shield-1. All cell types were able to support lytic viral replication in the presence of Shield-1. No virus production was detected in the absence of Shield-1.

### Example 3

### Immunogenicity of the IE1/2-UL51 Double Fusion Virus in Animals

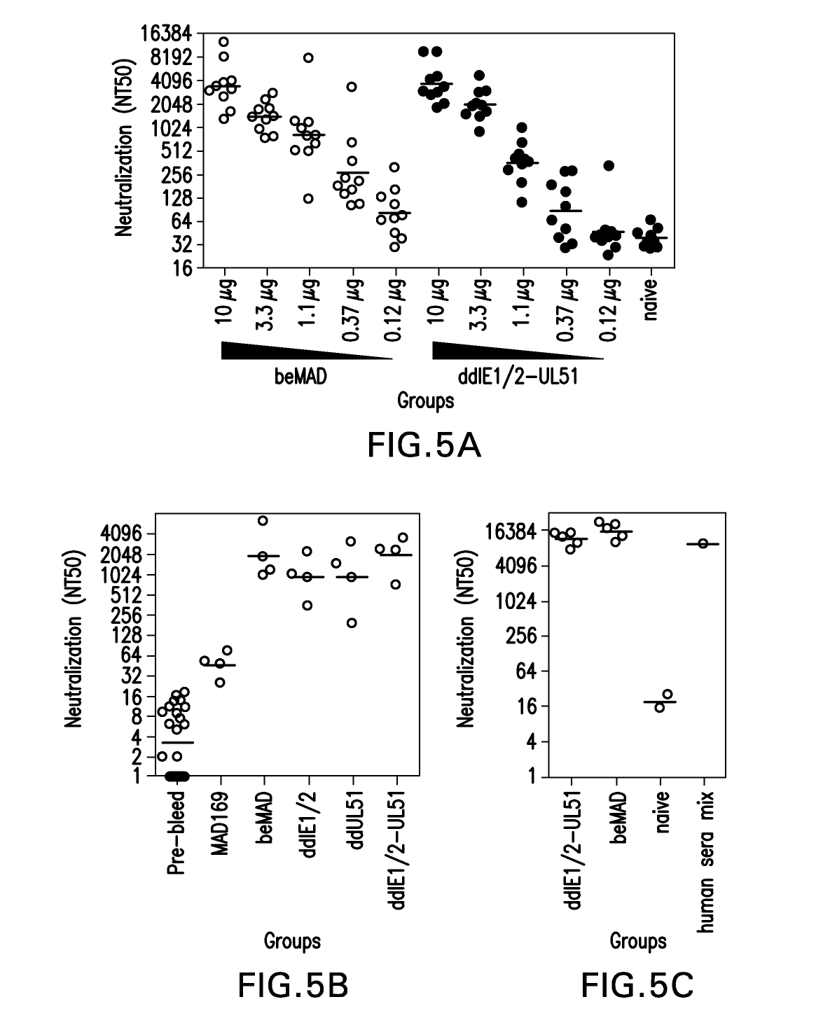

The immunogenicity of the IE1/2-UL51 double fusion virus was evaluated in mice, rabbits and rhesus monkeys. Dose dependent neutralizing response against the IE1/2-UL51 double fusion virus or the parental beMAD virus in mice was first compared (FIG. 5A). Six-week-old female BALB/c mice were immunized at weeks 0 and 4 with beMAD or the IE1/2-UL51 double fusion virus at doses ranging from 0.12 μg to 10 μg. Serum samples from week 6 were collected and analyzed by CMV micro-neutralization assay on ARPE-19 cells as described previously (Tang et al, Vaccine, “A novel throughput neutralization assay for supporting clinical evaluations of human cytomegalovirus vaccines” e-published Aug. 30, 2011 at doi:10.1016/j.vaccine.2011.08.086). The responses were compared at doses of 0.12, 0.37, 1.1, 3.3 and 10 μg. At the low dose range (0.12 to 1.1 μg), the beMAD was slightly more immunogenic with neutralizing antibodies consistently detected when dosage levels were above 0.37 μg. At the high dose range (3.3 and 10 μg), the neutralizing antibody titers induced by the two viruses were comparable.

Next, the immunogenicity of different viruses in rabbits at dose of 10 μg was compared. Female NZW rabbits were immunized at weeks 0, 3 and 8 with 10 μg of beMAD or the indicated fusion viruses. Week 10 sera were collected and analyzed by CMV micro-neutralization assay on ARPE-19 cells (FIG. 4B). The beMAD, single fusion viruses IE1/2 or UL51 and the double fusion virus IE1/2-UL51 could induce significantly higher titers of neutralizing antibodies than MAD169, a virus similar to AD169 and lacking the pentameric gH complex. This confirmed that expression of the gH complex by the virus significantly increased the immunogenicity of recombinant CMV.

Next, the immunogenicity of 100 μg of the double fusion IE1/2-UL51 virus or the parental beMAD virus was tested in rhesus macaques. Week 12 sera was collected and analyzed by CMV micro-neutralization assay on ARPE-19 cells. The GMT NT50 titers at week 12 (post dose 3) were 11500 or 15600, respectively. These titers were comparable to the NT50 titers seen in naturally infected individuals (FIG. 4C).

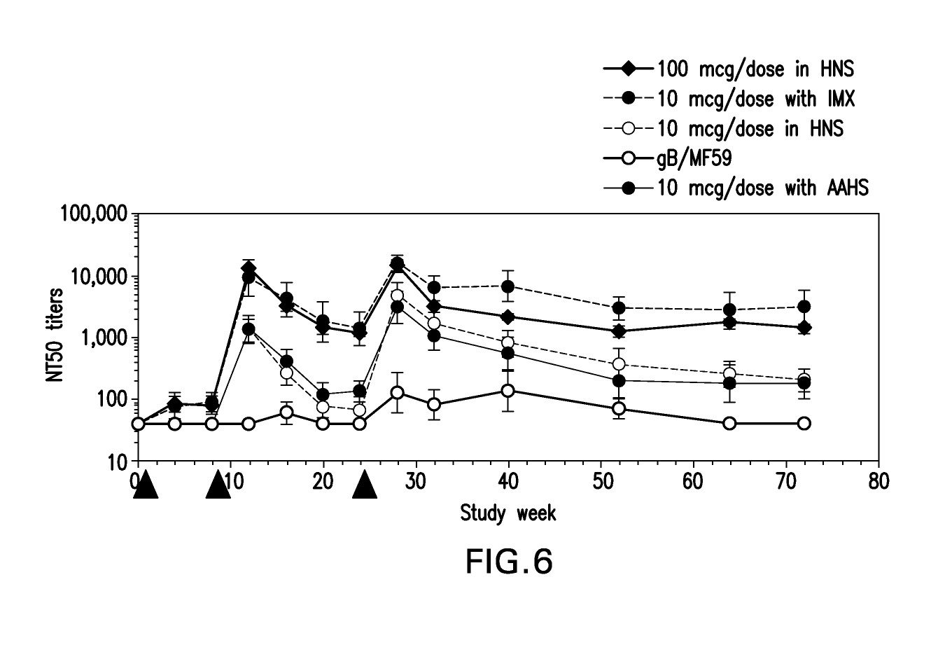

The longevity of the double fusion virus IE1/2-UL51 CMV vaccine-induced immune response was demonstrated in rhesus macaques. Animals were vaccinated with either 10 μg/dose or 100 μg/dose double fusion virus IE1/2-UL51 (based on total protein mass). Formulations of 10 μg/dose vaccine with amorphous aluminum hydroxylphosphate sulfate (AAHS) or ISCOMATRIX® adjuvant were also included. Vaccines were administered at weeks 0, 8, and 24 in rhesus macaques (n=5). For comparison, a control group received recombinant gB at 30 μg/dose formulated with MF59 adjuvant at weeks 0, 4 and 24. Geometric means for reciprocal NT50 titers (GMT) for all groups are presented longitudinally (FIG. 6). Prior to vaccination, there was no detectable neutralizing antibody titer >40 for any of the monkeys. Minimal neutralizing activity was detected after the first dose at week 4 for all groups with the neutralizing antibody titers peaking around week 12 and week 28 (four weeks after the second and the third vaccination, respectively). The peak GMT at week 28 for the 100 μg/dose group was 14,500 (about 3-fold higher than the titer of 4,660 for the 10 μg/dose group). ISCOMATRIX® adjuvant, but not AAHS, provided adjuvanting benefit when compared with the 10 μg/dose group. The GMT at week 28 for the ISCOMATRIX® group measured 15,800 whereas the AAHS group was 3,000 and the 10 μg/dose group was 4,660. Minimal neutralizing activity was detected for the control (gB/MF59) group, with the peak GMT never exceeding 200. At study week 72, close to 1 year after completion of the vaccination regimen at weeks 0, 8 and 24, the GMT for the 100 μg/dose group and the ISCOMATRIX® formulation group were maintained at 1400 and 3000, respectively. At this time, the GMT for the 10 μg/dose group and the AAHS group was around 200.

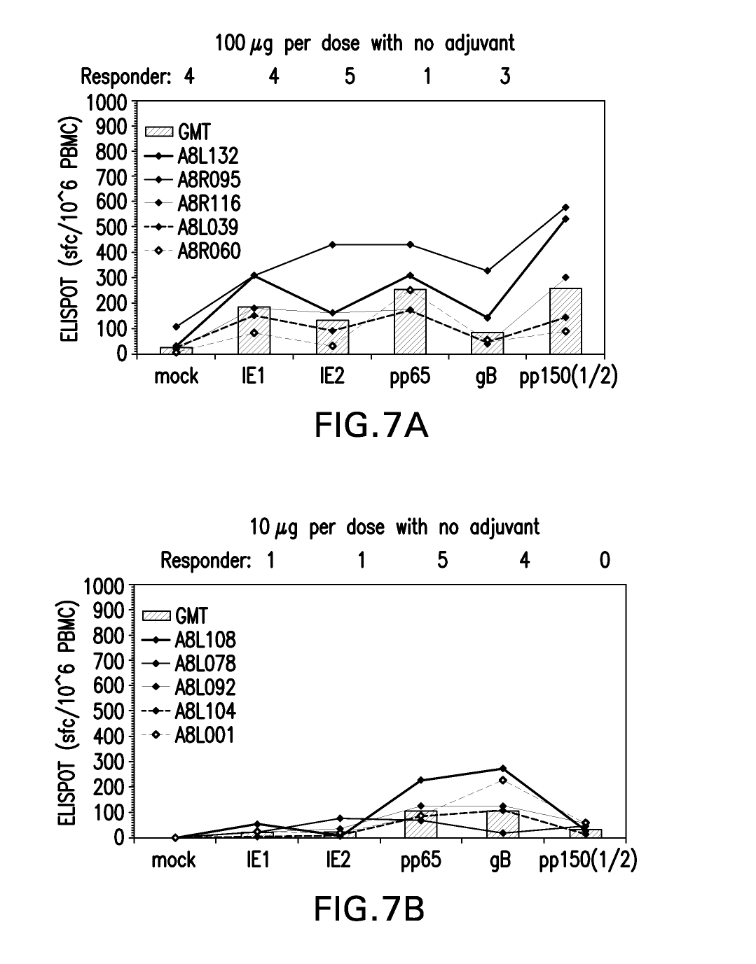

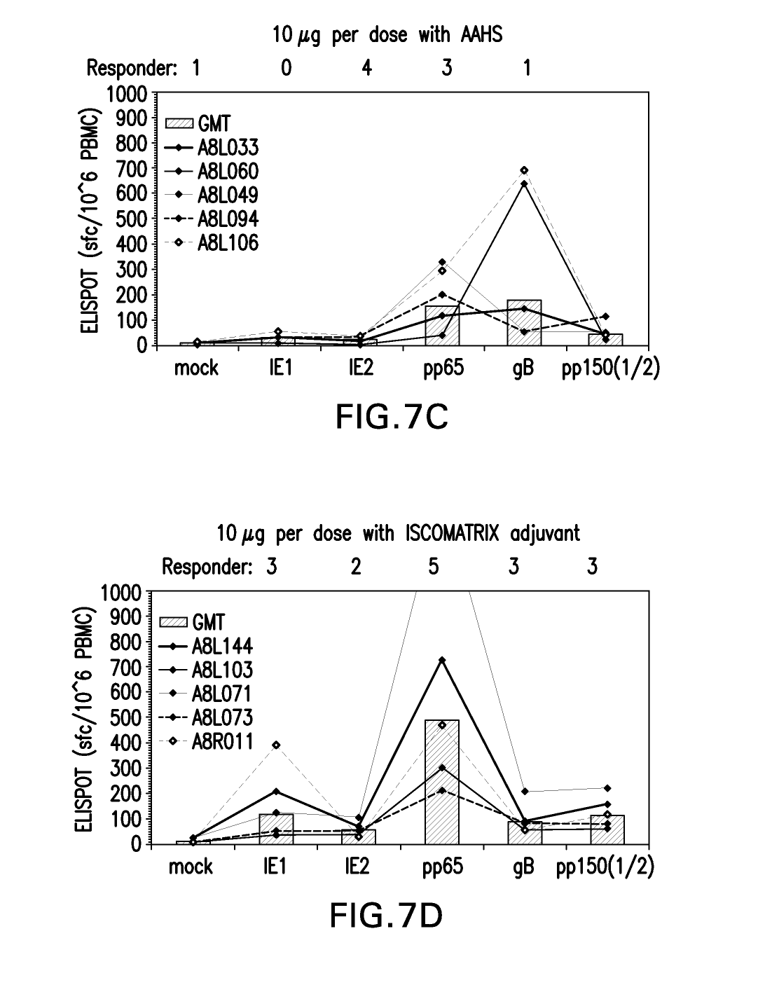

Peripheral blood mononuclear cells (PBMC) from rhesus macaques were collected at week 28 (4 weeks postdose 3) of the vaccination regimen and were evaluated in the IFN-γ ELISPOT assay. Monkeys were vaccinated with either 100 μg/dose (FIG. 7A) or 10 μg/dose (FIGS. 7B-7D) of the double fusion virus IE1/2-UL51. Additionally, the 10 μg/dose was formulated either with no adjuvant (FIG. 7B) or with AAHS (FIG. 7C) or ISCOMATRIX® (FIG. 7D) adjuvant. The antigens of pooled overlapping peptides representing five HCMV antigens were used to stimulate IFN-γ production ex-vivo. The HCMV antigens used were IE1 and IE2 (both viral regulatory proteins) and pp65, gB and pp150 (predominant viral structural antigens). Quality of the T-cell responses was assessed by the magnitude (geometric means) of ELISPOT responses as well as the responder rate to viral antigens. Prior to vaccination, there was no antigen-specific ELISPOT titer in any monkey (data not shown).

At week 28, the geometric means for ELISPOT responses to the five HCMV antigens (i.e., IE1, IE2, pp65, gB and pp150) were 186, 132, 253, 87, 257 spot-forming cells (SFC)/10⁶ PBMC for the 100 μg/dose group versus 21, 24, 107, 111, 33 SFC/10⁶ PBMC for the 10 μg/dose group, respectively (FIGS. 7A and 7B). A responder in each group (n=5) was scored based on cutoff criteria of more than 55 SFC/10⁶ PBMC and more than 3-fold rise in antigen-specific response over dimethyl sulfoxide (DMSO) response. The number of responders to the five HCMV antigens (i.e., IE1, IE2, pp65, gB and pp150) were 4, 4, 5, 1, 3 for the 100 μg/dose group versus 1, 1, 5, 4, 0 for the 10 μg/dose group.

The effect of ISCOMATRIX® adjuvant on T-cell responses to a 10 μg/dose of the double fusion virus IE1/2-UL51 is shown in FIG. 7D. Geometric means of ELISPOT responses to the five HCMV antigens (i.e., IE1, IE2, pp65, gB and pp150) were 114, 53, 491, 85, 113 SFC/10⁶ PBMC, respectively, and the number of responders in the group (n=5) are 3, 2, 5, 3, 3, respectively. The magnitude and breadth of the T-cells responses in the group with ISCOMATRIX® adjuvant were similar to those in the 100 μg/dose group.

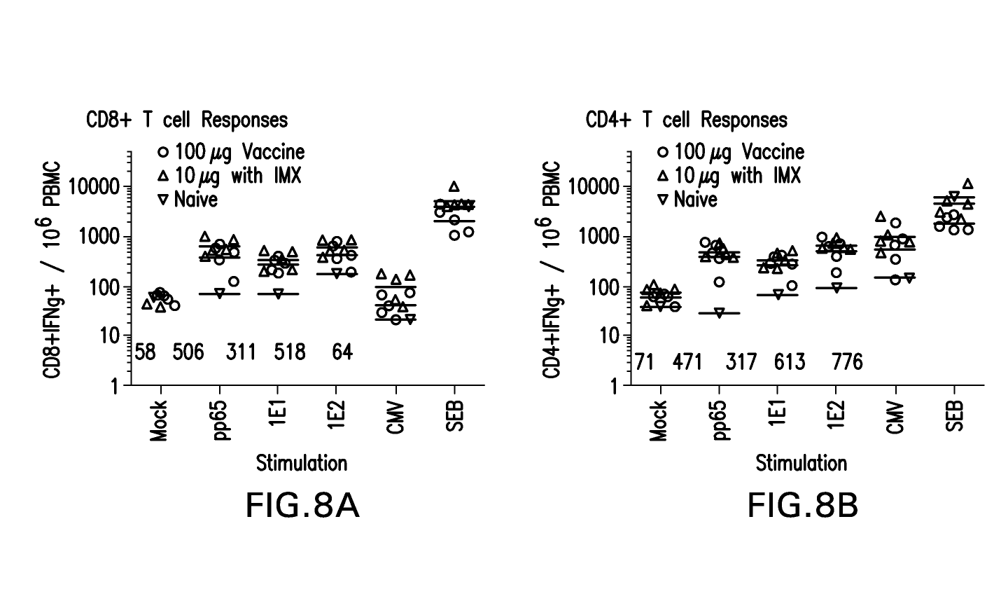

The PBMC from animals vaccinated with either a 10 μg/dose or 100 μg/dose double fusion virus IE1/2-UL51 (based on total protein mass) with ISCOMATRIX® were further analyzed in intracellular cytokine staining after being stimulated with HCMV antigens (pp65, IE1, IE2 or whole HCMV virion). The negative control was one naïve monkey not vaccinated with double fusion virus IE1/2-UL51 while the positive control was staphalococcus enterotoxin B (SEB). FIG. 8 shows that the negative control showed minimal responses to all antigen stimulations but responded to the positive control agent staphalococcus enterotoxin B (SEB) as expected. All ten vaccinated monkeys from both groups responded to HCMV-specific antigens with similar magnitude and patterns. The geometric mean values to each antigen were computed for all ten monkeys. All monkeys showed comparable CD8+(FIG. 8A) and CD4+(FIG. 8B) T-cell responses when their PBMCs were stimulated with CMV antigen peptide pools (i.e., pp65, IE1 and IE2) but preferentially showed CD4+ T-cell responses when stimulated with whole HCMV virions. This was not unexpected since whole virions are protein antigens and are likely processed as exogenous antigens and presented by MHC class II molecules to CD4+ T-cells. The double fusion virus IE1/2-UL51 can elicit T-cell responses of both CD4+ and CD8+ phenotypes, similar to those commonly seen in healthy subjects with HCMV infection.

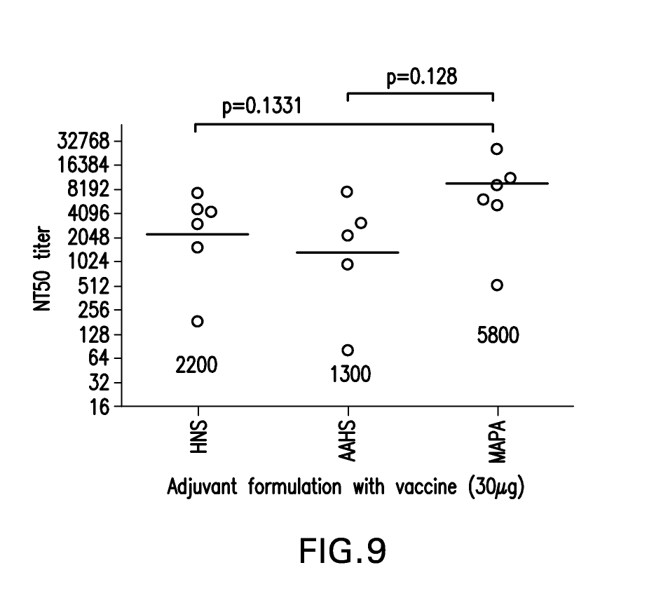

Different formulations of the double fusion virus IE1/2-UL51 with aluminum salts were compared for their ability to generate neutralizing antibodies in rhesus macaques (FIG. 9). 30 μg/dose double fusion virus IE1/2-UL51 was formulated with either HNS (base buffer), amorphous aluminum hydroxylphosphate sulfate (AAHS) or Merck Aluminum Phosphate Adjuvant (MAPA) and administered at weeks 0 and 8. Serum samples collected at week 12 showed that although MAPA enhanced the neutralizing antibody induction, the enhancement was not statistically significant (two-tailed unpaired t-test).

The results of T cell responses to IE1, IE2 as well as CD8⁺ T cell response to viral proteins are a strong indication that there were de novo viral antigen expression expressions in rhesus macaques. Thus, it suggests that replication of the CMV vector is not required for efficient expression of viral antigens.

### Example 4

### Identification of Buffers for Storage

The CMV virus in HBSS (Hank's Balanced Salt Solution) and stored at −70° C. until used was diluted ˜10× with appropriate buffer. The residual components of the HBSS buffer in each sample included potassium chloride 0.533 mM, potassium phosphate monobasic 0.044 mM, sodium phosphate dibasic 0.034 mM, sodium chloride 13.79 mM, sodium bicarbonate 0.417 mM and glucose 0.1% w/v. The samples were then stored at room temperature or between 2° C.-8° C. temperatures for 4 days or freeze thawed. For freezing-thawing, the sample was stored at −70° C. for at least 1 hour and thawed at RT for 30 minutes for either one or three cycles. The stability of the samples was tested on day 4 using a viral entry assay. Briefly, the assay was performed using several different sample dilutions to obtain a response curve and EC50 (μg/mL) values were obtained from the viral entry assay results by non-linear curve fitting. Lower EC50 values represent better stability. EC50 values of the stability samples were compared against −70° C. frozen control sample.

Viral entry assay measures the ability of CMV to infect ARPE-19 cells and express IE1 (immediate early protein 1). The assay is performed in transparent 96-well plates. The IE1 specific primary antibodies and biotinylated secondary antibodies are used to detect target proteins in fixed cells and fluorescent signal from each well is quantified using an IR Dye 800CW Streptavidin together with Sapphire 700/DRAQ5 (for cell input normalization). The results were plotted as 800/700 Integrated Intensity Ratio (Integ. Ratio) vs. CMV concentration (total protein, μg/mL). EC50 values were also obtained from the infectivity assay results using non-linear curve fitting. Since viral infection of ARPE-19 cells relies on integrity of viral glycoprotein antigens, in particular the pentameric gH complex, the EC50 values reflect how well the viral particles are preserved under these conditions.

The CMV loses infectivity when stored for four days in HBSS at RT (data not shown). Moreover, 3 cycles of freezing-thawing in HBSS lead to complete loss of infectivity when assessed by viral entry assay. Thus, HBSS was not an optimal buffer for CMV storage.

The effect of pH on CMV stability at room temperature was examined using the pH range of 3 to 8. The following buffers were utilized: Citrate buffer (25 mM), pH 3.0; Acetate buffer (25 mM), pH 4; Acetate buffer (25 mM), pH 5; Histidine buffer (25 mM), pH 6; HEPES buffer (25 mM), pH 7; Hanks' Balanced Salt Solution (HBSS), pH 7.5 and Tris buffer (25 mM), pH 8.

The samples were prepared by dilution of the viral bulk 10 times with the appropriate buffer. The samples were stored at RT (25° C.) for 4 days. On day 4, the stability of the samples was measured by utilizing the viral entry assay. The CMV in HBSS stored frozen at −70° C. was treated as a control. The UV-Vis spectra for each of the samples were obtained at time 0 and on day 4 to examine the structural changes and aggregation that occurred during storage.

25 mM Histidine buffer at pH 6 provided better stability for CMV by retaining higher infectivity at RT compared to other pH tested (data not shown). The second derivative of the UV-spectra indicated similar structural profile of the virus at all pHs (data not shown). No significant aggregation was observed at any of the pH tested as measured by optical density at 350 nm (data not shown).

The effect of urea alone or in combination with sodium chloride on CMV virus stability was tested in 25 mM Histidine buffer, pH 6. Addition of 2% urea alone did not have an effect on CMV stability. However, 2% urea in combination with 150 mM NaCl improved the stability of CMV at RT (data not shown).

The effect of ionic strength on CMV stability was examined at pH 6. Increasing concentrations of NaCl (0 mM, 75 mM, 150 mM and 320 mM NaCl) were added to 25 mM Histidine buffer at pH 6. The CMV stability was dependent on ionic strength where higher ionic strength led to better stability (data not shown). Presence of urea had no or minimal effect on CMV stability (data not shown).

Additionally, several other excipients (sucrose, sorbitol, glycerol, and proline) were screened for their effect on gH expressing CMV stability at room temperature. Exipients to be tested were added to CMV in 25 mm Histidine buffer, pH 6 at room temperature for 4 days before CMV virus stability was measured using a viral entry assay. EC₅₀ values were calculated for the samples. Among all the excipients tested, 150 mM NaCl alone or in combination with 9% w/v sucrose provided better stability at pH 6 (data not shown). Therefore, the recommended buffer for CMV storage at RT is 25 mM Histidine (pH 6) with 150 mM NaCl with or without 9% w/v sucrose.

The effect of cryoprotectants on CMV stability during freezing-thawing was investigated. As indicated previously, CMV in HBSS completely lost its infectivity when subjected to three freezing-thawing cycles. Several cryoprotectants (including sucrose, sorbitol, glycerol) were screened for the ability to diminish the freeze-thaw stress on CMV. For each freeze-thawing cycle, the samples were frozen at −70° C. for at least 1 hour and thawed at RT for 30 minutes. The addition of cryoprotectants led to increased stability of the virus. Moreover, 9% w/v sucrose in combination with 150 mM sodium chloride led to significantly enhanced stability of the virus when compared to other cryoprotectants tested (data not shown). Therefore, the recommended buffer composition for CMV storage at −70° C. or up to 3 freezing-thawing cycles is 25 mM Histidine, 150 mM NaCl and 9% sucrose (HNS buffer).

HNS buffer was compared with HBSS buffer for protection of CMV stability during three freeze-thaw cycles, refrigeration (2-8° C.) and RT (25° C.). The HNS buffer provided better stability for CMV live virus at all the storage conditions tested (data not shown).

### Example 5

### CMV Stability in HNS Buffer

The double fusion IE1/2-UL51 CMV virus stock was supplied in HNS buffer and stored at −70° C. until used. The stability study was performed at a concentration of 100 μg/mL (based on total protein content measured by Bradford assay). The bulk virus was diluted with HNS buffer to obtain the final virus concentration. The samples were then stored at appropriate temperatures and tested as described for up to 3 months. For freezing-thawing, the samples were frozen at −70° C. for at least 1 hour and thawed at room temperature for 30 minutes. The samples were pulled at different time points and kept stored frozen at −70° C. until analyzed.

Total protein content of the samples was measured using a Bradford assay. The total protein content of the samples did not change over the 3 month period (data not shown).

Particle size of the CMV in the samples over time was monitored by measuring the hydrodynamic diameter of the sample using DLS method. This method monitored any aggregation or disruption of the virus particles over time and at different storage temperatures. No real trending was observed with sporadic changes in the particle size of certain samples (data not shown). The results indicated that the virus particles were intact and not aggregated at elevated temperatures.

### Example 6

### Effect of Storage Conditions on Viral Entry and Immunogenicity

Significant changes in viral entry titers (EC50 values) were observed by subjecting the CMV samples to different storage temperatures (data not shown). Storage at −20° C. resulted in lower viral entry titers compared to 2-8° C. and 25° C. The titers of 2-8° C. samples were found to be lower viral entry titers compared to 25° C. storage. Based on the EC50 values the storage temperatures were ranked in the following order (from most stable to least stable): 25° C.>2-8° C.>−20° C. up to a 1 month time point. The viral entry titers were not detectable at the 3 month time point for the samples stored at −20° C., 2-8° C. and 25° C.

A mouse immunogenicity study was initiated at the end of the stability study to determine the effect of storage temperature on the ability of CMV to induce CMV neutralizing antibodies. The mice were immunized with 2.5 μg per dose vaccine i.m. on day 0 and boosted on day 21 followed by bleeding on day 28. The mouse serum was tested for neutralizing antibodies against a gH expressing CMV using ARPE 19 cells and NT50 titers were obtained by non-linear curve fitting.

The effect of storage at different temperatures for 3 months on the IE1/2-UL51 double fusion CMV immunogenicity was evaluated. The NT50 titers were dependent on the storage temperature, with higher temperatures resulting in decreased titers compared to −70° C. frozen control although not significantly (p=0.2584, one way ANOVA) (data not shown). The NT50 titer for formulations stored at −20° C. was lower by less than 2-fold, but the viral entry assay titers for these samples were significantly affected compared to −70° C. frozen control. The trending of NT50 titers for −20° C., 2-8° C. and 25° C. stability samples follows the CMV mass ELISA titers obtained for these samples.

The effect of storage at different temperatures for 8 hours after thawing on the IE1/2-UL51 double fusion CMV immunogenicity was evaluated. The NT50 titers of the formulations were compared to a −70° C. frozen control. The NT50 titers were not affected (p=0.5865, one way ANOVA) by storing the samples for 8 hours at any of the temperatures tested (data not shown).

The effect of the double fusion IE1/2-UL51 CMV storage at 25° C. for different time points after thawing the samples was evaluated in a mouse immunogenicity study. The NT50 titers of these formulations were compared to a −70° C. frozen control. The NT50 titers were not affected (p=0.1848, unpaired two-tailed t-test) by storing the samples at 25° C. for up to a week. At 3 months, the NT 50 titers dropped by a little over 2-fold indicating possible stability issues of the formulation at 25° C. for longer time (data not shown).

The effect of 3 cycles of freeze-thaw on the double fusion IE1/2-UL51 CMV formulated in HNS buffer was evaluated by mouse immunogenicity. Three cycles of freeze-thaw (F/T) of the double fusion CMV formulation did not affect the immunogenicity (p=0.2103, unpaired two tailed t-test) compared to a −70° C. frozen control (data not shown).

Other embodiments are within the following claims. While several embodiments have been shown and described, various modifications may be made without departing from the spirit and scope of the present invention.

### Example 7

### A Recombinant CMV Containing an Expression Cassette Comprising the Luciferase Gene

To demonstrate feasibility of a CMV vector based on MAD169, a cassette comprising the HSV TK gene promoter/enhancer driving a Gaussian luciferase (gLuc) gene was inserted in the CMV UL21.5 locus by replacing the entire UL21.5 open reading frame with the TK promoter-gLuc DNA fragment.

CMV-luciferase virus (CMV-gLuc) was cultured in ARPE-19 cells and the cell free virus was collected when full CPE was observed. Following one-round of purification by ultracentrifugation through 20% Sorbital cushion, the virus was resuspended in HNS buffer. Total viral protein mass and viral infectivity titers were measured as previously described.

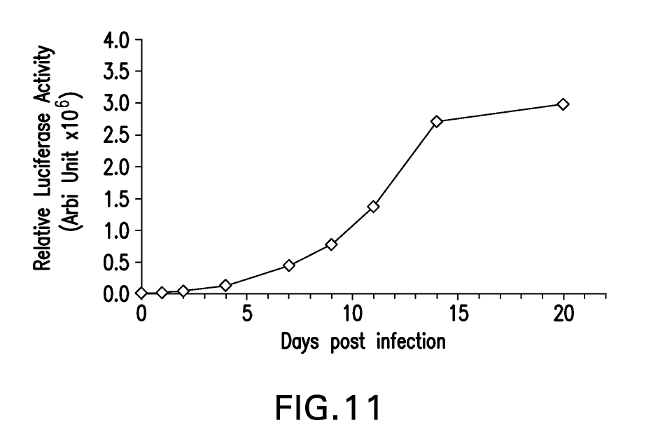

The expression of the luciferase transgene by the CMV vector was examined for kinetics of luciferase activity in cultured ARPE-19 cells infected with CMV-gLuc. Cells were infected with virus at ˜MOI of 0.01, and supernatants collected at indicated time points. The activity was measured using Pierce Gaussia Luciferase Glow Kit (Fisher Scientific). As shown in FIG. 11, the luciferase activity was detected around day 4 post infection and reached steady state around day 14 when the CPE was prominent.

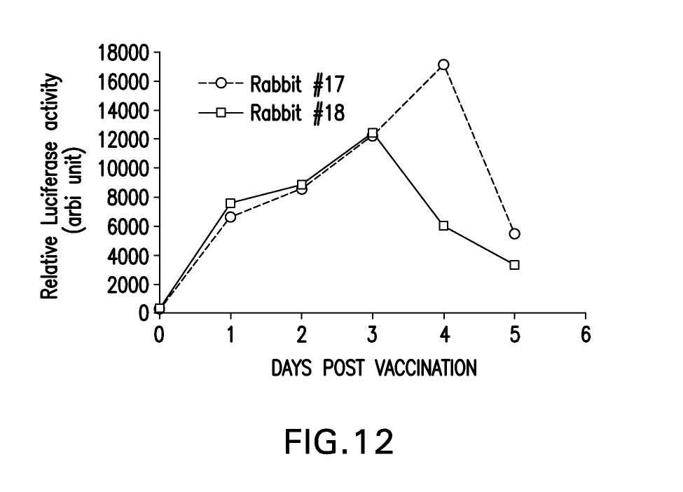

To evaluate the transgene activity by the CMV vector in vivo, two NZW rabbits were inoculated with CMV-gLuc at 100 μg, approximately 6.3E+07 pfu, i.m. The plasma samples were collected daily for 5 days, and the samples were measured for luciferase activity with the Pierce Gaussia Luciferase Glow Kit (Fisher Scientific). The luciferase activity was detected at day 1 post inoculation and peaked around day 3-4 (FIG. 12). At day 5, the luciferase activity in both rabbits declined to 2000-4000 units.

### Example 8

### Recombinant CMV Vectors for HIV-1 Gag Antigen

With HIV-1 gag as a model antigen, four vectors were designed as described in Table 3. The base vector for each of the constructs described below was the beMAD CMV strain comprising ddUL51, in which ddFKBP was fused to UL51. Thus the vectors were designed to be replication defective, and their replication is dependent on Shield-1. The HIV-1 gag sequence was codon-optimized for mammalian expression and disclosed previously (See WO98/034640; WO2002/022080).

TABLE 3
Design and growth characteristics of CMV vectors for HIV-1 GAG

| | | | Gene | | | Growth | Virus |
|---|---|---|---|---|---|---|---|
| Clone # | Virus name | Promoter | Insert | Poly A | Insertion site | phenotype | ID |
| 1 | EF1α- | EF1α | HIV GAG | BGH | UL21.5 ORF | Did not | N/A |
| | GAGsubUL21.5 | | | | substitution | grow | |
| 2 | GAGsubUL21.5 | native | HIV GAG | native | UL21.5 ORF | Wild type | GAG211 |
| | | UL21.5 | | UL21.5 | substitution | like | |
| 3 | EF1α-GAG- | EF1α | HIV GAG | BGH | 5 kb substitution | Not viable | N/A |
| | BGHsub5kb | | | | | | |
| 4 | EF1α- | EF1α | GAG-2A- | BGH | 5 kb substitution | Not viable | N/A |
| | GAG2AgLuc- | | gLuc | | | | |
| | BGHsub5kb | | | | | | |
| 5 | 2A- | native MIE | 2A-GAG | native | UL122 C termal in | Growth | N/A |
| | GAGinUL122C | | | UL122/123 | frame insertion | defect | |
| 6 | GAG- | native UL83 | GAG-2A | native | UL83 N terminal in | Wild type | GAG602 |
| | 2AinUL83N | | | UL83 | frame insertion | like | |
| 7 | 2A- | native UL83 | 2A-GAG | native | UL83 C terminal in | Wild type | GAG702 |
| | GAGinUL83C | | | UL83 | frame insertion | like | |
| 8 | GAG- | native 5 kb | GAG | BGH | 5 kb substitution | Not viable | N/A |
| | BGHsub5kb | | | | | | |

Abbrievation:
EF1α, elongation factor 1α;
BGH, bovine growth hormone;
gLuc, Gaussia luciferase;
MIE, major immediate early.

There are several design features for the constructs shown in Table 3. First, clones 1, 3 and 4 were designed with a cellular promoter, EF1α, and bovine growth hormone (BGH) poly-adenylation signal; whereas clones 2, 5, 6, 7 and 8 were designed with the transgene placed downstream of native CMV promoters, and in combination with either the native polyadenylation signals or BGH (clone #8). Second, clones 1, 2, 3 and 8 were designed with GAG to be expressed, while in other clones (#4, 5, 6, and 7), GAG was expressed as a fusion polypeptide linked to other proteins via a 2A peptide (Fang et al., 2005, Nat Biotechnol. 23:584-590). In clone #4, GAG was expressed with Gaussia luciferase, while in clones 5, 6 and 7, GAG was linked to dominant CMV antigens. Third, in clones 1, 2, 3, 4 and 8, the gene inserts were designed to replace either the UL21.5 ORF or the 5 kb transcript regions, while in clones 5, 6, and 7 the gene inserts were fused in-frame to CMV antigens known for potent T-cell responses, i.e., pp65 (UL83) and IE1/2 (UL123/UL122), to allow expression of fusion polypeptides.

The viral constructs were recovered as described in EXAMPLE 2 and the viable viruses were identified as GAG211, GAG602 or GAG702 (Table 3). Recombinant virus was cultured in ARPE-19 cells and the viral culture supernatant was collected when full CPE was observed. The virus was purified by one-round ultracentrifugation through 20% Sorbital cushion. The virus was resuspended in HNS buffer and total viral protein mass and viral infectivity titers were measured as previously described.

### Example 9

### Foreign Gene Expression by CMV Vector In Vitro

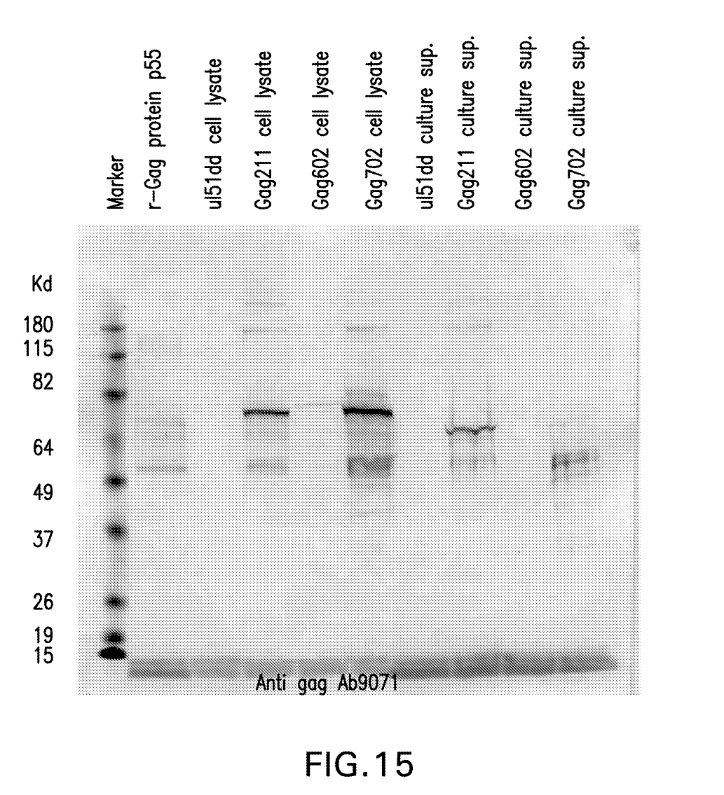

For CMV-GAG211 and CMV-GAG702 viruses (see Table 3, clones 2 and 6), the expression of HIV-1 gag was first confirmed by Western blot analysis, using a monoclonal antibody specific for HIV gag p55. As shown in FIG. 15, bands corresponding to molecular weight of ˜55 kDa, were detected in cell lysates and supernatant of ARPE-19 cells infected with CMV-GAG211 and GAG702 vectors. There was a rather faint band in cell lysate from CMV-GAG602 vector around 55 kDa, but was not definitive for GAG expression.

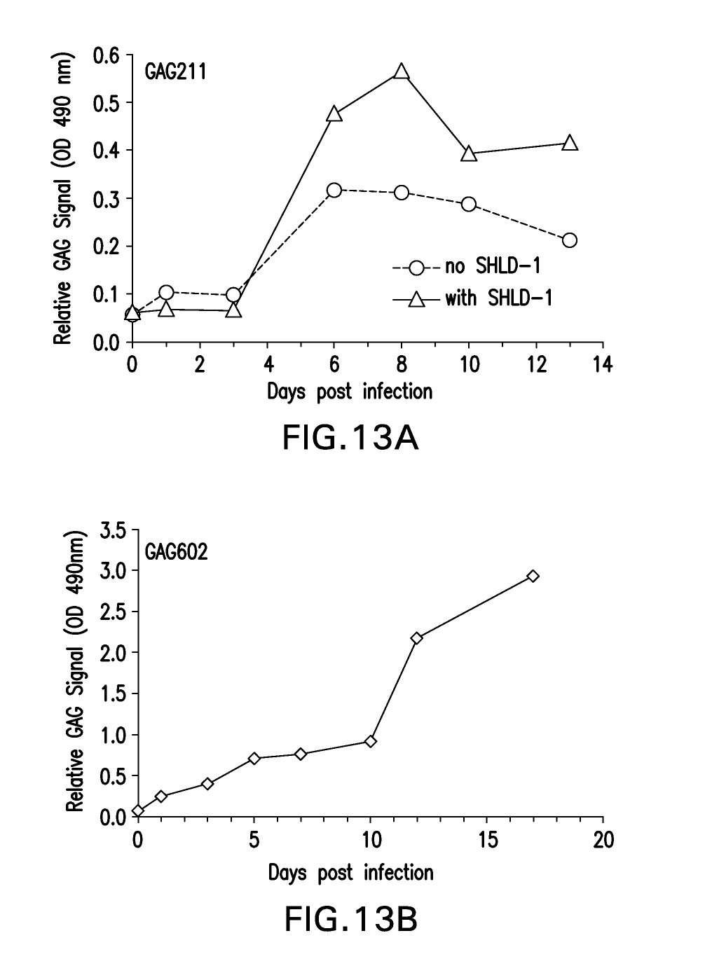

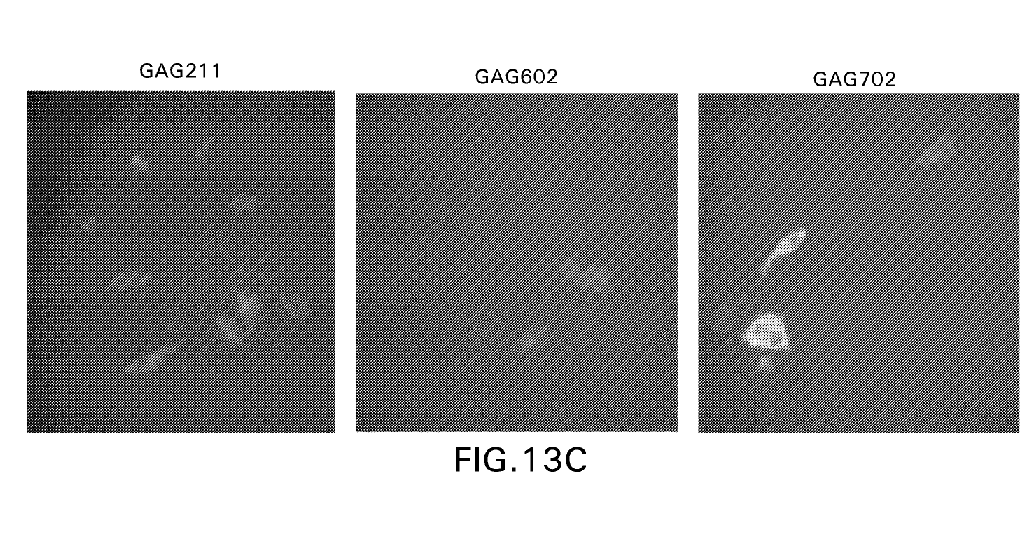

The kinetic expression of HIV-1 gag was further examined by p24 quantitative ELISA (PerkinElmer) (FIG. 13). Results indicate that HIV-1 gag signals were peaked around days 6-8 post infection and the peak was higher when GAG211 was cultured with Shield-1. For GAG602, the expression kinetics were delayed comparing to GAG211.

### Example 10

### GAG-Specific T-Cell Responses Induced by CMV-GAG Vector in Mice

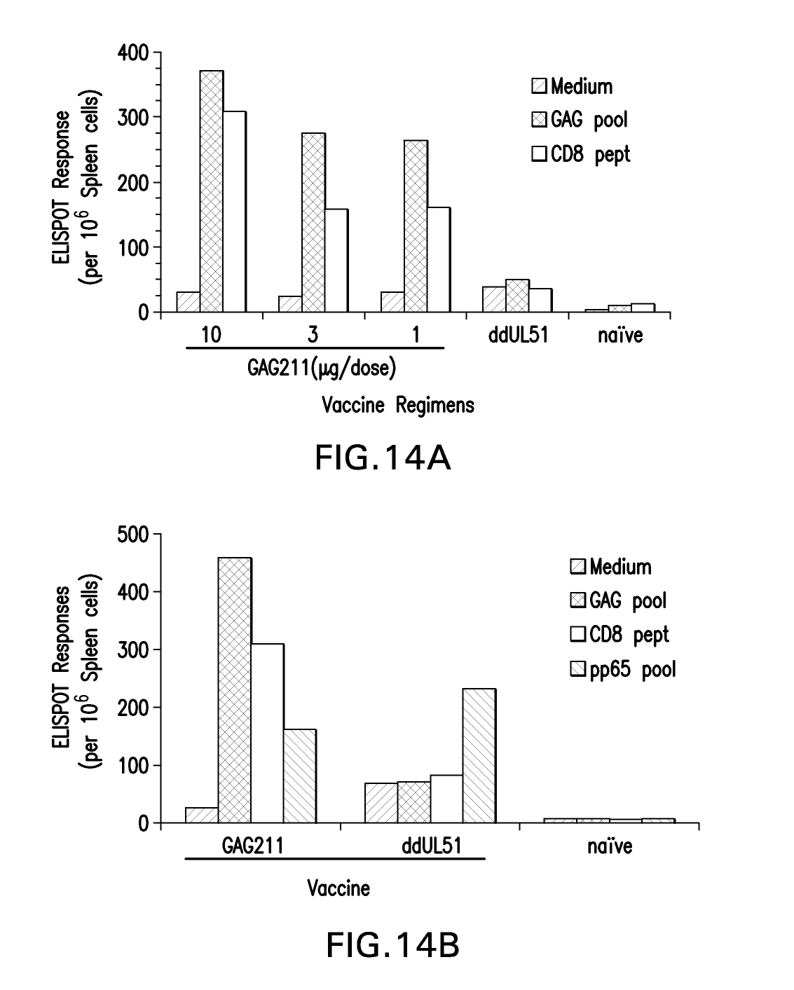

Two mouse experiments were conducted. In the first experiment, female BALB/c mice were immunized with 10, 3 or 1 μg/dose CMV-GAG211 vaccine intramuscularly at weeks 0 and 3. The spleen cells from three mice from each group were pooled four weeks post the second injection and tested an in IFN-γ ELISPOT assay. There is only one CD8 T-cell epitope in HIV-1 GAG in BALB/c background (Meta et al, 1998, J. Immunol 161:2985). A CD8 peptide, restricted by H-2K^{d}, and a “GAG pool”, which is a pool of 15-mer peptides overlapping by 11-amino acids for the entire length of GAG ORF, were used for ex vivo T-cell stimulation for IFN-γ production (FIG. 14A). As expected, there were no GAG-specific T-cell responses detected in ddUL51 or naive groups. For the three vaccine groups, GAG-specific IFN-γ ELISPOT responses corresponding to titrated GAG211 dose were observed. Thus, GAG expression of a CMV vector correlated with its ability to induce T-cell responses in mice. The finding was duplicated in female C57BL/6× Balb/c F1 mice (FIG. 14B).

1. A conditional replication defective cytomegalovirus (rdCMVhet) comprising:
(a) a nucleic acid encoding a fusion protein, wherein the fusion protein comprises an essential CMV protein, or a derivative thereof, fused to a destabilizing protein, wherein the essential protein is selected from the group consisting of IE1/2, UL37x1, UL44, UL51, UL52, UL53, UL56, UL77, UL79, UL84, UL87, and UL105; and
(b) a nucleic acid encoding a heterologous polypeptide, and
(c) a promoter operably linked to the nucleic acid encoding the heterologous polypeptide.

2. The rdCMVhet of claim 1 further comprising a pentameric gH complex comprising UL128, UL130, UL131, gH and gL.

3. The rdCMVhet of claim 1, wherein
the essential protein is selected from the group consisting of: UL51, UL52, and UL87, or a derivative thereof.

4. The rdCMVhet of claim 1, wherein the essential protein:
(a) comprises a sequence of amino acids as set forth in SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41. SEQ ID NO:42, or SEQ ID NO:43, or
(b) is an essential protein derivative that is at least 95% identical to an amino acid sequence set forth in (a).

5. The rdCMVhet of claim 3, wherein the essential protein comprises a sequence of amino acids as set forth in SEQ ID NO:35, SEQ ID NO:36 or SEQ ID NO:42, or is a protein derivative that is at least 95% identical to a protein having an amino acid sequence set forth in SEQ ID NO:35, SEQ ID NO:36 or SEQ ID NO:42.

6. The rdCMVhet of claim 1, wherein the destabilizing protein is FKBP or an FKBP derivative, wherein the FKBP protein comprises a sequence of amino acids as set forth in SEQ ID NO:29 and wherein the FKBP derivative is an FKBP protein which has one or more amino acid substitutions relative to SEQ ID NO:29, wherein the substitutions are selected from the group consisting of: F15S, V24A, H25R, E31G, F36V, E60G, M66T, R71G, D100G, D100N, E102G, K105I, K105E and L106P.

7. The rdCMVhet of claim 6, wherein the destabilizing protein is an FKBP derivative which has the amino acid substitutions F36V and L106P or the amino acid substitutions F36V and K105E.

8. The rdCMVhet of claim 7, wherein the FKBP derivative comprises a sequence of amino acids as set forth in SEQ ID NO:26, SEQ ID NO:44, or SEQ ID NO:31.

9. The rdCMVhet of claim 6, wherein the essential CMV protein is UL51.

10. The rdCMVhet of claim 1, further comprising a nucleic acid encoding a second fusion protein, wherein the second fusion protein comprises an essential CMV protein, or a derivative thereof, fused to a destabilizing protein, wherein the essential protein is selected from the group consisting of IE1/2, UL37x1, UL44, UL51, UL52, UL53, UL56, UL77, UL79, UL84, UL87, and UL105, wherein the essential proteins in each of the fusion proteins are different.

11. The rdCMVhet of claim 1, wherein the fusion protein comprises a sequence of amino acids as set forth in SEQ ID NO:7 or an amino acid sequence that is at least 95% identical to SEQ ID NO:7.

12. (canceled)

13. The rdCMVhet of claim 6, wherein the nucleic acid encoding the heterologous polypeptide is fused in-frame to a dominant CMV antigen known for potent T-cell responses and the promoter is a native CMV promoter.

14. The rdCMVhet of claim 13, wherein the dominant CMV antigen is selected from the group consisting of: UL83, IE1/2, UL55, UL86, UL99, UL122, UL36, UL48, UL32, UL113, UL123, US32, UL28, US29, US3, UL94, and UL69.

15-19. (canceled)

20. A composition comprising the rdCMVhet of claim 1 and a pharmaceutically acceptable carrier.

21. The composition of claim 20, further comprising an adjuvant.

22. (canceled)

23. A method of inducing a prophylactic or therapeutic immune response against an antigen in a patient comprising administering to the patient an immunologically effective amount of the composition of claim 20.

24-26. (canceled)

27. A method of making the rdCMVhet of claim 6 comprising propagating the recombinant rdCMVhet in epithelial cells or fibroblast cells in the presence of Shield-1.

28. The method of claim 27, wherein the epithelial cells are human pigmented retinal epithelial cells.

29. The method of claim 27, wherein the cells are ARPE-19 cells or MRC-5 cells.

30. The method of claim 27 wherein the Shield-1 is present at a concentration of at least 0.5 μM.

31-36. (canceled)
